(19) United States
(12) Patent Application Publication
(10) Pub. No.: US 2026/0053104 A1
(43) Pub. Date: Feb. 26, 2026

(54) LOW GLUCOSINOLATE PENNYCRESS MEAL AND METHODS OF MAKING

(71) Applicants: COVERCRESS INC., St. Louis, MO (US); BOARD OF TRUSTEES OF ILLINOIS STATE UNIVERSITY, Normal, IL (US); REGENTS OF THE UNIVERSITY OF MINNESOTA, Minneapolis, MN (US)

(72) Inventors: Tim Ulmasov, Chesterfield, MO (US); John C. Sedbrook, Bloomington, IL (US); Michael David Marks, Roseville, MN (US); Ratan Chopra, St. Paul, MN (US)

(21) Appl. No.: 19/380,621

(22) Filed: Nov. 5, 2025

Related U.S. Application Data
(63) Continuation of application No. 18/185,179, filed on Mar. 16, 2023, now Pat. No. 12,484,502.
Application No. 18/185,179 is a division of application No. 17/249,431, filed on Mar. 2, 2021, now Pat. No. 11,685,927.

(51) Int. Cl. A01H 6/20; A01H 1/02; A01H 5/10

(52) U.S. Cl. CPC A01H 6/20; A01H 1/02; A01H 5/10

(57) ABSTRACT

Pennycress (Thlaspi arvense) seed, seed lots, seed meal, and compositions with reduced glucosinolate content as well as plants that yield such seed, seed lots, seed meal, and compositions are provided. Methods of making and using the pennycress plants and/or seed that provide such seed, seed lots, seed meal, and compositions are also provided.

### CROSS REFERENCE TO RELATED APPLICATIONS

This is a continuation patent application which claims priority to U.S. Ser. No. 18/185,179, filed Mar. 16, 2023, which is a divisional patent application which claims priority under 35 U.S.C. § 120 to U.S. Ser. No. 17/249,431, filed Mar. 2, 2021, now U.S. Pat. No. 11,685,927, issued Jun. 27, 2023, which is a divisional patent application of U.S. Ser. No. 16/251,247, filed Jan. 18, 2019, now U.S. Pat. No. 10,988,772, issued Apr. 27, 2021, which claims the benefit of U.S. Provisional Patent Application No. 62/619,360, filed Jan. 19, 2018, all of which are incorporated herein by reference in their entireties.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Mar. 13, 2023, is named “P13413US04_SequenceListing.xml” and is 406,727 bytes in size.

### STATEMENT REGARDING FEDERAL FUNDING

This invention was made with government support under Grant Number 2014-67009-22305 awarded by the National Institute of Food and Agriculture, USDA. The government has certain rights in the invention.

### BACKGROUND

Different plants have seed contents that make them desirable for feed compositions. Examples are soybean, canola, rapeseed and sunflower. After crushing the seeds and recovering the oil, the resulting meal has a protein content making the meal useful as a feed ingredient for ruminants and other animals. Nevertheless, there remains a desire for improved plant seeds that can provide additional sources of nutrition to animals.

Field Pennycress Thlaspi arvense L. (common names: fanweed, stinkweed, field pennycress), hereafter referred to as Pennycress or pennycress, is a winter cover crop that helps to protect soil from erosion, prevent the loss of farm-field nitrogen into water systems, and retain nutrients and residues to improve soil productivity. While it is well established that cover crops provide agronomic and ecological benefits to agriculture and environment, only 5% of U.S. farmers today are using them. One reason is economics—it requires on average ˜$30-50/acre to grow a cover crop on the land that is otherwise idle between two seasons of cash crops such as corn and soy. In the last 5 years, it has been recognized that pennycress could be used as a novel cover crop, because in addition to providing cover crop benefits, it produces harvestable seeds rich in oil and protein having value for feed, food, fuel, and industrial applications. Extensive testing indicates that pennycress can be interseeded over standing corn in early fall and harvested in spring prior to soybean planting (in appropriate climates). As such, its growth and development require minimal incremental inputs (e.g., no/minimum tillage, no/low nitrogen, insecticides or herbicides). Pennycress also does not directly compete with existing crops when intercropped e.g., for energy production, and the recovered oil and meal can provide an additional source of income for farmers.

Pennycress is a winter annual belonging to the Brassicaceae (mustard) family. It's related to cultivated crops, rapeseed and canola, which are also members of the Brassicaceae family. Pennycress seeds are smaller than those of canola, but they are also high in oil and protein content. They typically contain 36% oil, which is roughly twice the level found in soybean, and the oil has a very low saturated fat content (˜4%). Pennycress represents a clear opportunity for sustainable optimization of agricultural systems. For example, in the U.S. Midwest, ˜35M acres that remain idle could be planted with pennycress near the time of corn crop harvest, with pennycress seeds and/or plants harvested before the next soybean crop is planted. Pennycress can serve as an important winter cover crop working within the no/low-till corn and soybean rotation to guard against soil erosion and improve overall field soil nitrogen and pest management.

Pennycress seeds contain oil that is highly desirable as a feedstock for biofuels and/or chemicals and potentially as a food oil. Once the oil is obtained from pennycress seeds, either from mechanical expeller pressing or hexane extraction, the resulting meal has a high protein level with a favorable amino acid profile that could provide nutritional benefits to animals. However, studies of pennycress processing have consistently demonstrated that the meal produced has a high level of the anti-nutrient compound sinigrin (allyl-glucosinolate or 2-propenyl-glucosinolate), and as a result, without additional treatments, may not be competitive with high-value products like soybean and canola meals, ingredients commonly used in animal feed. Glucosinolates (GSLs) are secondary plant metabolites that are found in all Brassica plants such as rapeseed, canola, camelina, carinata and pennycress. Content and composition of GSLs vary due to plant species, agronomic practices and environmental conditions (Tripathi and Mishra, 2007). Glucosinolates and their breakdown products that are a result of hydrolysis during the processing of the seeds into animal feed can result in negative effects on animal nutrition. The toxicity of glucosinolates for animals has been primarily associated with the metabolites thiocyanates, oxazolidinethiones and nitriles. These compounds interfere with iodine uptake (thiocyanates) and the synthesis of the thyroid hormones T3 and T4 (oxazolidinethiones), leading eventually to hypothyroidism and enlargement of the thyroid gland (EFSA, 2008). The major clinical signs of toxicity described in farm animals include growth retardation, reduction in performance (milk and egg production), impaired reproductive activity, and impairment of liver and kidney functions (EFSA, 2008). A comprehensive review of the effects of glucosinolates in animal nutrition has been published by Tripathi and Mishra (2007) and EFSA (2008).

### SUMMARY

Compositions comprising non-defatted pennycress seed meal that comprises less than 30, 28, 25, 16, or 15 micromoles sinigrin per gram by dry weight or about 1, 2.5, 5, or 10 to about 15, 16, 18, 20, 25, 28, or 30 micromoles sinigrin per gram by dry weight are provided herein.

Non-defatted pennycress seed meal that comprise less than 30, 28, 25, 16, or 15 micromoles sinigrin per gram by dry weight are provided herein.

Defatted pennycress seed meal comprising less than 30, 28, 25, 16, or 15 micromoles sinigrin per gram by dry weight or about 1, 2.5, 5, or 10 to about 15, 16, 18, 20, 25, 28, or 30 micromoles sinigrin per gram by dry weight are provided herein.

Compositions comprising defatted pennycress seed meal that comprise less than 30, 28, 25, 16, or 15 micromoles sinigrin per gram by dry weight or about 1, 2.5, 5, or 10 to about 15, 16, 18, 20, 25, 28, or 30 micromoles sinigrin per gram by dry weight are provided herein.

Pennycress seed comprising less than 30, 28, 25, 16, or 15 micromoles sinigrin per gram by dry weight or about 1, 2.5, 5, or 10 to about 15, 16, 18, 20, 25, 28, or 30 micromoles sinigrin per gram by dry weight are provided herein.

Pennycress seed lots comprising pennycress seed with less than 30, 28, 25, 16, or 15 micromoles sinigrin per gram by dry weight or about 1, 2.5, 5, or 10 to about 15, 16, 18, 20, 25, 28, or 30 micromoles sinigrin per gram by dry weight are provided herein.

In one embodiment, this disclosure provides methods for producing low glucosinolate pennycress seeds and meal. In certain embodiments, the methods comprise genetically modifying pennycress seed (e.g., using gene editing, mutagenesis, or a transgenic approach) to suppress expression of one or more genes involved in sinigrin biosynthesis, transport, and/or hydrolysis. Genetically altered seed lots with lower sinigrin content in comparison to control seed lots that lack the genetic alteration can be obtained by these methods.

### BRIEF DESCRIPTION OF THE DRAWINGS

The accompanying drawings, which are incorporated in and form a part of the specification, illustrate the embodiments of the present disclosure and together with the description, serve to explain the principles of the disclosure. In the drawings:

FIG. 1A, B illustrate glucosinolate (GSL) biosynthetic pathways for many Brassica plants. Panel A: A schematic diagram of aliphatic GSL pathway which begins with amino acid methionine as the precursor and is relevant for GSL modification in pennycress seed. Panel B: Various GSL forms found in Brassica are shown.

FIG. 2A, B, C, illustrates pARV1 (SS32_GTR1), Agrobacterium CRISPR-Cas9 vector and its gene editing sgRNA cassette, for targeting pennycress homolog of Glucosinolate transporter 1 (GTR1 or Glut1) gene. Panel A: Plasmid map of pARV1 (SS32_GTR1). Panel B: sgRNA cluster in pARV1, targeting nucleotides 2503-2522 and 2538-2557 of SEQ ID NO: 14. Panel C: Sequence example of one of gRNA cassettes targeting pennycress homolog of Glucosinolate transporter 1 (GTR1 or Glut1) gene.

FIG. 3A, B illustrates pDe-SpCas9 and pDe-SaCas9, Agrobacterium CRISPR-Cas9 base vectors for editing plant genome. gRNA cassette stuffers are inserted at the multiple cloning site between the Cas9 and HygR cassettes, replacing a small fragment of the vector with synthetic gRNA cassette.

FIG. 4A, B, C, illustrates pARV145, Agrobacterium CRISPR-Cas9 vector and its gene editing sgRNA cassettes, for targeting pennycress homolog of MYB28 (HAG1) gene. Panel A: Plasmid map of pARV145. Panel B: sgRNA cluster in pARV145, targeting nucleotides 719-738 and 793-812 of SEQ ID NO: 20. Panel C: Sequence examples of gRNA cassettes targeting pennycress homolog of MYB28 (HAG1) gene.

FIG. 5 illustrates pARV187, Agrobacterium CRISPR-FnCpf1 base vector for editing plant genome. gRNA cassette stuffers are inserted at the dual AarI site, replacing a small fragment of the vector with synthetic gRNA cassette.

FIG. 6 illustrates pARV190, Agrobacterium CRISPR-SmCms1 base vector for editing plant genome. gRNA cassette stuffers are inserted at the dual AarI site, replacing a small fragment of the vector with synthetic gRNA cassette.

FIG. 7A, B, C, D, E, F, G, H, I, J, K, gRNA cassettes targeting pennycress homologs of multiple genes in glucosinolate biosynthetic/metabolic pathway. FIG. 5A illustrates a gRNA cassette stuffer, designed for insertion into the AarI-digested plant genome editing vector (such as pARV187 or pARV190) for targeting pennycress AOP2 gene, nucleotides 2367-2389 and 2419-2441 of SEQ ID NO: 2; FIG. 5B: gRNA cassette stuffer for targeting pennycress BCAT4 gene, nucleotides 2984-3006 and 3048-3070 of SEQ ID NO: 5; FIG. 5C: gRNA cassette stuffer for targeting pennycress BCAT6 gene, nucleotides 1932-1954 and 2387-2409 of SEQ ID NO: 8; FIG. 5D: gRNA cassette stuffer for targeting pennycress CYP79 gene, nucleotides 2914-2936 and 2968-2990 of SEQ ID NO: 11; FIG. 5E: gRNA cassette stuffer for targeting pennycress GTR1 gene, nucleotides 2483-2505 and 2541-2563 of SEQ ID NO: 14; FIG. 5F: gRNA cassette stuffer for targeting pennycress GTR2 gene, nucleotides 2317-2339 and 2404-2426 of SEQ ID NO: 18; FIGS. 5G and 5H: gRNA cassette stuffers for targeting pennycress MYB28 gene, nucleotides 948-970, 1001-1023, 1045-1067 and 1315-1337 of SEQ ID NO: 20; FIG. 5I: gRNA cassette stuffer for targeting pennycress MYB29 gene, nucleotides 2573-2595 and 2625-2647 of SEQ ID NO: 23; FIG. 5J: gRNA cassette stuffer for targeting pennycress MYB76 gene, nucleotides 1539-1561 and 1570-1592 of SEQ ID NO: 26; FIG. 5K: gRNA cassette stuffer for targeting pennycress TFP gene, nucleotides 2170-2192 and 2559-2581 of SEQ ID NO: 29.

### DETAILED DESCRIPTION

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “include,” “includes,” and “including” are to be construed as at least having the features to which they refer while not excluding any additional unspecified features.

Where a term is provided in the singular, other embodiments described by the plural of that term are also provided.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Reductions in sinigrin content of various pennycress plants, seeds, seed lots, seed meals, and compositions obtained therefrom as well as associated methods of obtaining and using such plants, seeds, seed lots, seed meals, and compositions is provided herein by suppression of certain endogenous pennycress genes. The endogenous pennycress genes that can be suppressed to provide such reductions in sinigrin content include, but are not limited to, endogenous pennycress genes set forth in the following Table 1 and allelic variants of those genes.

Suppression of certain endogenous pennycress gene expression to provide for reductions in sinigrin content can be affected by a variety of techniques including, but not limited to, loss-of-function (LOF) mutations in endogenous genes, with transgenes, or by using gene-editing- or mutagenesis-mediated genome rearrangements. In certain embodiments, the pennycress plants, seeds, seed lots, seed meals (which can be defatted or non-defatted), and related compositions can comprise one or more LOF mutations that suppress or otherwise alter expression and/or function of one or more genes, coding sequences, and/or proteins, thus resulting in reduced sinigrin content in comparison to control or wild-type pennycress seed, seed lots, and plant lots. Such LOF mutations include, but are not limited to, INDELS (insertions, deletions, and/or substitutions or any combination thereof), translocations, inversions, duplications, or any combination thereof in a promoter, and/or other regulatory elements including enhancers, a 5′ untranslated region, coding region, an intron of a gene, and/or a 3′ UTR of a gene. Such INDELS can introduce one or more mutations including, but not limited to, frameshift mutations, missense mutations, pre-mature translation termination codons, splice donor and/or acceptor mutations, regulatory mutations, and the like that result in a LOF mutation. In certain embodiments, the LOF mutation will result in: (a) a reduction in the enzymatic, transport, or other biochemical activity associated with the encoded polypeptide in the plant comprising the LOF mutation in comparison to a wild-type control plant; or (b) both a reduction in the enzymatic, transport or other biochemical activity (e.g., transcription factor) and a reduction in the amount of a transcript (e.g., mRNA) or polypeptide in the plant comprising the LOF mutation in comparison to a wild-type control plant. Such reductions in activity or activity and transcript levels can, in certain embodiments, comprise a reduction of at least 20%, 30%, 40%, 50%, 60%, 70%, 75%, 80%, 85%, 90%, 95%, 98%, 99%, or 100% of activity or activity and transcript levels in the LOF mutant in comparison to the activity or transcript levels in a wild-type control plant. In certain embodiments, the pennycress plants, seeds, seed lots, seed meals (which can be defatted or non-defatted), and related compositions will comprise one or more transgenes or genetic modifications that suppress or otherwise alter expression of one or more genes, coding sequences, and/or proteins, thus resulting in reduced sinigrin content in comparison to control or wild-type pennycress seed lots. Transgenes or genetic modifications that can provide for such suppression or alteration include, but are not limited to, transgenes or genome rearrangements introduced via gene editing or other mutagenesis techniques that produce small interfering RNAs (siRNAs), miRNA, or artificial miRNAs targeting a given gene or gene transcript for suppression. Such genome rearrangements include, but are not limited to, deletions, duplications, insertions, inversions, translocations, and combinations thereof. Useful genome rearrangements include, but are not limited to, rearrangements that place an endogenous promoter and/or transcriptional enhancer in proximity to 3′ end of a target gene or coding sequence (e.g., a gene or coding sequence of Table 1) or within the target gene or coding sequence such that the endogenous promoter and/or enhancer drive expression of an siRNA or miRNA that suppresses or otherwise alters expression of the target gene. In certain embodiments, the transgenes or genetic modifications that suppress expression will result in: (a) a reduction in the enzymatic, transport, or other biochemical activity associated with the encoded polypeptide in the plant comprising the transgene or genome rearrangement in comparison to a wild-type control plant; or (b) both a reduction in the enzymatic or other biochemical activity and a reduction in the amount of a transcript (e.g., mRNA) or polypeptide in the plant comprising the transgene or genome rearrangement in comparison to a wild-type control plant. Such reductions in activity and transcript levels can in certain embodiments comprise a reduction of at least 20%, 30%, 40%, 50%, 60%, 70%, 75%, 80%, 85%, 90%, 95%, 98%, 99%, or 100% of activity and/or transcript levels in the transgenic plant in comparison to the activity or transcript levels in a wild-type control plant. In certain embodiments, certain genes, coding sequences, and/or proteins that can be targeted for introduction of LOF mutations or that are targeted for transgene- or genome rearrangement-mediated suppression are provided in the following Table 1 and accompanying Sequence Listing. In certain embodiments, allelic variants of the wild-type genes, coding sequences, and/or proteins provided in Table 1 and the sequence listing are targeted for introduction of LOF mutations or are targeted for transgene- or genome rearrangement-mediated suppression. Allelic variants found in distinct pennycress isolates or varieties that exhibit wild-type seed sinigrin content can be targeted for introduction of LOF mutations or are targeted for transgene- or genome rearrangement-mediated suppression to obtain seed lots having reduced sinigrin content in comparison to sinigrin content of the control seed lots of wild-type pennycress. Such allelic variants can comprise polynucleotide sequences that have at least 80%, 85%, 90%, 95%, 96%, 97%, 98%, or 99% sequence identity across the entire length of the polynucleotide sequences of the wild-type coding regions or wild-type genes of Table 1 and the sequence listing. Such allelic variants can comprise polypeptide sequences that have at least 80%, 85%, 90%, 95%, 96%, 97%, 98%, or 99% sequence identity across the entire length of the polypeptide sequences of the wild-type proteins of Table 1 and the sequence listing. Pennycress seed lots having reduced sinigrin content as described herein can comprise one or more LOF mutations in one or more genes that encode polypeptides involved in GSL biosynthesis, in GSL transport, in GSL hydrolysis, regulating expression of genes encoding GSL biosynthetic and/or transport genes (e.g., transcription factors) or can comprise transgenes or genome rearrangements that suppress expression of those biosynthetic, transporter, hydrolysis, or expression regulator (e.g., transcription factor) encoding genes. Polypeptides affecting these traits include, without limitation, AOP2, BCAT4, BCAT6, CYP79F1, CYP83A1, GTR1, GTR2, MYB28 (HAG1), MYB29, MYB76, TFP, BHLH05, IMD1, CYP79B3, MAM1, FMO-GS-Ox1, and UGT74B-1 polypeptides disclosed in Table 1 and allelic variants thereof. In certain embodiments, pennycress seed lots, plants, seeds, as well as the seed meals and compositions obtained therefrom, all having reduced sinigrin content, can comprise one or more LOF mutations found in the pennycress mutant E3 196, E5 444P1, E5 356P5, I87113, E5 543, or I87207 germplasm. Compositions comprising defatted or non-defatted seed meal obtained from any of the aforementioned seed lots, and seed cakes obtained from any of the aforementioned seed lots are also provided herein. Methods of making any of the aforementioned seed lots, compositions, seed meals, or seed cakes are also provided herein. As used herein, the phrase “seed cake” refers to the material obtained after the seeds are crushed, ground, heated, and expeller pressed or extruded prior to solvent extraction.

In certain embodiments, reductions in sinigrin content of seed lots, seed meal compositions, seed meal, or seed cake are in comparison to sinigrin content of control or wild-type seed lots, seed meal compositions, seed meal, or seed cake. Such controls include, but are not limited to, seed lots, seed meal compositions, seed meal, or seed cake obtained from control plants that lack the LOF mutations or transgene- or genome rearrangement-mediated gene suppression. In certain embodiments, control plants that lack the LOF mutations or transgene or genome rearrangement mediated gene suppression will be otherwise isogenic to the plants that contain the LOF mutations or transgene- or genome rearrangement-mediated gene suppression. In certain embodiments, the controls will comprise seed lots, seed meal compositions, seed meal, or seed cake obtained from plants that lack the LOF mutations or transgene or genome rearrangement mediated gene suppression and that were grown in parallel with the plants having the LOF mutations or transgene or genome rearrangement-mediated gene suppression. In certain embodiments, the pennycress seed lots, plants, seeds, as well as the defatted or non-defatted seed meals and compositions obtained therefrom, can comprise a less than 30, 28, 25, 16, or 15 micromoles sinigrin per gram by dry weight or about 1, 2.5, 5, or 10 to about 15, 16, 18, 20, 25, 28, or 30 micromoles sinigrin per gram by dry weight.

In certain embodiments, pennycress seed lots, plants, seeds, as well as the seed meals and compositions obtained therefrom, all having reduced sinigrin content, can include at least one loss-of-function mutation in a GSL biosynthetic coding sequence or gene (e.g., SEQ ID NO: 1, 2, 4, 5, 7, 8, 10, 11, 92, 93, 162, 163, 165, 166, 168, 169, 171, 172, 174, 175, or allelic variant thereof) and/or at least one loss-of-function mutation in a GSL transport (SEQ ID NO: 13, 14, 16, 18, or allelic variant thereof), in a GSL hydrolysis (SEQ ID NO: 28, 29, or allelic variant thereof), and/or in an expression regulator (e.g., transcription factor; SEQ ID NO: 19, 20, 22, 23, 25, 26, 159, 160, allelic variant thereof) coding sequence or gene. In certain embodiments, pennycress seed lots, plants, seeds, as well as the seed meals and compositions obtained therefrom, all having reduced sinigrin content, can include at least one loss-of-function mutation in a GSL transport coding sequence or gene (e.g., SEQ ID NO: 13, 14, 16, 18, or allelic variant thereof) and at least one loss-of-function mutation in a GSL hydrolysis (SEQ ID NO: 28, 29, or allelic variant thereof), and/or in a expression regulator (e.g., transcription factor; SEQ ID NO: 19, 20, 22, 23, 25, 26, allelic variant thereof) coding sequence or gene. In certain embodiments, pennycress seed lots, plants, seeds, as well as the seed meals and compositions obtained therefrom, all having reduced sinigrin content, can include at least one loss-of-function mutation in a GSL hydrolysis (SEQ ID NO: 28, 29, or allelic variant thereof) coding sequence or gene and/or at least one loss-of-function mutation in an expression regulator (e.g., transcription factor; SEQ ID NO: 19, 20, 22, 23, 25, 26, allelic variant thereof) coding sequence or gene. In certain embodiments, pennycress seed lots, plants, seeds, as well as the seed meals and compositions obtained therefrom, all having reduced sinigrin content, can be obtained from pennycress plants comprising the mutant E3 196, E5 444P1, E5 356P5, I87113, E5 543, or I87207 germplasm.

TABLE 1
Wild-type (WT) coding regions, encoded proteins, and genes that
can be targeted for introduction of LOF mutations or transgene-
or genome rearrangement-mediated suppression, their mutant
variants and representative genetic elements for achieving
suppression of gene expression.

| | | | | Names Used and/or |
|---|---|---|---|---|
| | | | | Representative |
| SEQ | | | | Pennycress LOF |
| ID | Sequence | | Function/Nature of the | Mutants Disclosed |
| NO: | Name | Type | mutation | Herein |
| 1 | AOP2-CDS | WT Coding | Plays a role in the | ALKENYL |
| | | region | secondary modification of | HYDROXALKYL |
| 2 | AOP2- | WT Gene | aliphatic (methionine- | PRODUCING 2 - CAPE |
| | Genomic | | derived) GSLs, namely | VERDE ISLANDS, |
| | locus | | the conversion of | AOP2-CVI, GSL ALK |
| 3 | AOP2-PRT | WT Protein | methylsulfinylalkyl GSLs | enzyme, AOP (2- |
| | | | to form alkenyl GSLs, and | oxoglutarate- dependent |
| | | | also influences aliphatic | dioxygenase) |
| | | | GSL accumulation | |
| 4 | BCAT4-CDS | WT Coding | Involved in the | BCAT4 (BRANCHED-CHAIN |
| | | region | methionine chain | AMINOTRANSFERASE 4) |
| 5 | BCAT4- | WT Gene | elongation pathway that | |
| | Genomic | | leads to the ultimate | |
| | locus | | biosynthesis of | |
| 6 | BCAT4-PRT | WT Protein | methionine-derived | |
| | | | glucosinolates | |
| 7 | BCAT6-CDS | WT Coding | Encodes a cytosolic | BCAT6 (BRANCHED-CHAIN |
| | | region | branched-chain | AMINOTRANSFERASE 6) |
| 8 | BCAT6- | WT Gene | aminotransferase that acts | |
| | Genomic | | on Leu, Ile, Val and also | |
| | locus | | on Met. Together with | |
| 9 | BCAT6-PRT | WT Protein | BCAT4 and BCAT3, it is | |
| | | | involved in methionine | |
| | | | salvage and glucosinolate | |
| | | | biosynthesis | |
| 10 | CYP79F1- | WT Coding | Catalyzes the first | CYP79F1 |
| | CDS | region | committed step in | (CYTOCHROME P45079F1), |
| 11 | CYP79F1- | WT Gene | biosynthesis of the core | BUS1, BUSHY 1, |
| | Genomic | | structure of GSLs, | SPS1, SUPERSHOOT 1 |
| | locus | | Modulates the level of | |
| 12 | CYP79F1- | WT Protein | short chain methionine | |
| | PRT | | derived aliphatic GSLs | |
| 13 | GTR1-CDS | WT Coding | GTR1 encodes high- | GTR1 (Glucosinolate |
| | | region | affinity, proton-dependent | Transporter 1), NPF2.10, |
| 14 | GTR1- | WT Gene | GSL-specific transporter | NRT1/PTR FAMILY |
| | Genomic | | essential for the | 2.10 |
| | locus | | accumulation of GSLs in | |
| 15 | GTR1-PRT | WT Protein | seeds | |
| 16 | GTR2-CDS | WT Coding | GTR2 encodes high- | GTR2 (Glucosinolate |
| | | region | affinity, proton-dependent | Transporter 2), NPF2.11, |
| 17 | GTR2-PRT | WT Protein | GSL-specific transporter | NRT1/PTR FAMILY |
| 18 | GTR2- | WT Gene | essential for the | 2.11 |
| | Genomic | | accumulation of GSLs in | |
| | locus | | seeds | |
| 19 | MYB28-CDS | WT Coding | Principal regulator of | HAG1 (High Aliphatic |
| | | region | aliphatic glucosinolate | Glucosinolate 1), MYB |
| 20 | MYB28- | WT Gene | biosynthesis and affects | DOMAIN PROTEIN 28, |
| | Genomic | | the production of both | PMG1 (Production of |
| | locus | | short- and long-chain | Methionine-derived |
| 21 | MYB28-PRT | WT Protein | aliphatic glucosinolates | Glucosinolate 1) |
| 22 | MYB29-CDS | WT Coding | MYB DOMAIN | HAG3 (High Aliphatic |
| | | region | PROTEIN 29, a Myb | Glucosinolate 3), PMG2 |
| 23 | MYB29- | WT Gene | transcription factor affects | (Production of |
| | Genomic | | biosynthesis of short- | Methionine-derived |
| | locus | | chain aliphatic | Glucosinolate 2), RAO7, |
| 24 | MYB29-PRT | WT Protein | glucosinolates | (Regulator of Alternative |
| | | | | Oxidase 1A 7) |
| 25 | MYB76-CDS | WT Coding | MYB DOMAIN | HAG2 (High Aliphatic |
| | | region | PROTEIN 76, a Myb | Glucosinolate 2), PMG2, |
| 26 | MYB76- | WT Gene | transcription factor affects | (Production of |
| | Genomic | | biosynthesis of short- | Methionine-derived |
| | locus | | chain aliphatic | Glucosinolate 2), RAO7 |
| 27 | MYB76-PRT | WT Protein | glucosinolates. | (Regulator of Alternative |
| | | | | Oxidase 1A 7) |
| 28 | TFP-CDS | WT Coding | Promotes the formation of | Thiocyanate-forming |
| | | region | allylthiocyanate as well as | protein (TFP) |
| 29 | TFP- | WT Gene | the epithionitrile upon | |
| | Genomic | | myrosinase-catalyzed | |
| | locus | | hydrolysis of | |
| 30 | TFP-PRT | WT Protein | allylglucosinolate, the | |
| | | | major glucosinolate | |
| 31 | AOP2_scaffold_ | Oligonucleotide | AOP2 CDS targeted for | |
| | 16_117170_ | | cleavage by Cpf1 enzyme; | |
| | 125481_−_ | | part of gRNA cassette | |
| | CRISPR_64 | | | |
| 32 | AOP2_scaffold_ | Oligonucleotide | AOP2 CDS targeted for | |
| | 16_117170_ | | cleavage by Cpf1 enzyme; | |
| | 125481_−_ | | part of gRNA cassette | |
| | Cfp1_226 | | | |
| 33 | AOP2_scaffold_ | Oligonucleotide | AOP2 CDS targeted for | |
| | 16_117170_ | | cleavage by Cpf1 enzyme; | |
| | 125481_−_ | | part of gRNA cassette | |
| | Cfp1_627 | | | |
| 34 | AOP2_scaffold_ | Oligonucleotide | AOP2 CDS targeted for | |
| | 16_117170_ | | cleavage by Cpf1 enzyme; | |
| | 125481_−_ | | part of gRNA cassette | |
| | CRISPR_66 | | | |
| 35 | BCAT4_scaffold_ | Oligonucleotide | BCAT4 CDS targeted for | |
| | 7_1484532_ | | cleavage by Cpf1 enzyme; | |
| | 1492822_+_ | | part of gRNA cassette | |
| | Cfp1_566 | | | |
| 36 | BCAT4_scaffold_ | Oligonucleotide | BCAT4 CDS targeted for | |
| | 7_1484532_ | | cleavage by Cpf1 enzyme; | |
| | 1492822_+_ | | part of gRNA cassette | |
| | CRISPR_184 | | | |
| 37 | BCAT4_scaffold_ | Oligonucleotide | BCAT4 CDS targeted for | |
| | 7_1484532_ | | cleavage by Cpf1 enzyme; | |
| | 1492822_+_ | | part of gRNA cassette | |
| | CRISPR_185 | | | |
| 38 | BCAT4_scaffold_ | Oligonucleotide | BCAT4 CDS targeted for | |
| | 7_1484532_ | | cleavage by Cpf1 enzyme; | |
| | 1492822_+_ | | part of gRNA cassette | |
| | Cfp1_172 | | | |
| 39 | BCAT6_scaffold_ | Oligonucleotide | BCAT6 CDS targeted for | |
| | 18_731862_ | | cleavage by Cpf1 enzyme; | |
| | 739845_+_ | | part of gRNA cassette | |
| | CRISPR_63 | | | |
| 40 | BCAT6_scaffold_ | Oligonucleotide | BCAT6 CDS targeted for | |
| | 18_731862_ | | cleavage by Cpf1 enzyme; | |
| | 739845_+_ | | part of gRNA cassette | |
| | CRISPR_64 | | | |
| 41 | BCAT6_scaffold_ | Oligonucleotide | BCAT6 CDS targeted for | |
| | 18_731862_ | | cleavage by Cpf1 enzyme; | |
| | 739845_+_ | | part of gRNA cassette | |
| | Cfp1_558 | | | |
| 42 | BCAT6_scaffold_ | Oligonucleotide | BCAT6 CDS targeted for | |
| | 18_731862_ | | cleavage by Cpf1 enzyme; | |
| | 739845_+_ | | part of gRNA cassette | |
| | Cfp1_577 | | | |
| 43 | CYP79F1_ | Oligonucleotide | CYP79F1 CDS targeted | |
| | scaffold_1_ | | for cleavage by Cpf1 | |
| | 3018995_ | | enzyme; part of gRNA | |
| | 3027106_+_ | | cassette | |
| | CRISPR_86 | | | |
| 44 | CYP79F1_ | Oligonucleotide | CYP79F1 CDS targeted | |
| | scaffold_1_ | | for cleavage by Cpf1 | |
| | 3018995_ | | enzyme; part of gRNA | |
| | 3027106_+_ | | cassette | |
| | Cfp1_493 | | | |
| 45 | CYP79F1_ | Oligonucleotide | CYP79F1 CDS targeted | |
| | scaffold_1_ | | for cleavage by Cpf1 | |
| | 3018995_ | | enzyme; part of gRNA | |
| | 3027106_+_ | | cassette | |
| | CRISPR_87 | | | |
| 46 | CYP79F1_ | Oligonucleotide | CYP79F1 CDS targeted | |
| | scaffold_1_ | | for cleavage by Cpf1 | |
| | 3018995_ | | enzyme; part of gRNA | |
| | 3027106_+_ | | cassette | |
| | Cfp1_495 | | | |
| 47 | GTR1_scaffold_ | Oligonucleotide | GTR1 CDS targeted for | |
| | 63_146888_ | | cleavage by Cpf1 enzyme; | |
| | 155577_−_ | | part of gRNA cassette | |
| | Cfp1_88 | | | |
| 48 | GTR1_scaffold_ | Oligonucleotide | GTR1 CDS targeted for | |
| | 63_146888_ | | cleavage by Cpf1 enzyme; | |
| | 155577_−_ | | part of gRNA cassette | |
| | Cfp1_92 | | | |
| 49 | GTR1_scaffold_ | Oligonucleotide | GTR1 CDS targeted for | |
| | 63_146888_ | | cleavage by Cpf1 enzyme; | |
| | 155577_−_ | | part of gRNA cassette | |
| | Cfp1_506 | | | |
| 50 | GTR1_scaffold_ | Oligonucleotide | GTR1 CDS targeted for | |
| | 63_146888_ | | cleavage by Cpf1 enzyme; | |
| | 155577_−_ | | part of gRNA cassette | |
| | Cfp1_525 | | | |
| 51 | GTR1_scaffold_ | Oligonucleotide | GTR1 CDS targeted for | |
| | 63_146888_ | | cleavage by Cpf1 enzyme; | |
| | 155577_−_ | | part of gRNA cassette | |
| | Cfp1_574 | | | |
| 52 | GTR1_scaffold_ | Oligonucleotide | GTR1 CDS targeted for | |
| | 63_146888_ | | cleavage by Cpf1 enzyme; | |
| | 155577_−_ | | part of gRNA cassette | |
| | Cfp1_214 | | | |
| 53 | GTR2_scaffold_ | Oligonucleotide | GTR2 CDS targeted for | |
| | 0_1964427_ | | cleavage by Cpf1 enzyme; | |
| | 1972843_+_ | | part of gRNA cassette | |
| | Cfp1_513 | | | |
| 54 | GTR2_scaffold_ | Oligonucleotide | GTR2 CDS targeted for | |
| | 0_1964427_ | | cleavage by Cpf1 enzyme; | |
| | 1972843_+_ | | part of gRNA cassette | |
| | Cfp1_537 | | | |
| 55 | GTR2_scaffold_ | Oligonucleotide | GTR2 CDS targeted for | |
| | 0_1964427_ | | cleavage by Cpf1 enzyme; | |
| | 1972843_+_ | | part of gRNA cassette | |
| | Cfp1_174 | | | |
| 56 | GTR2_scaffold_ | Oligonucleotide | GTR2 CDS targeted for | |
| | 0_1964427_ | | cleavage by Cpf1 enzyme; | |
| | 1972843_+_ | | part of gRNA cassette | |
| | Cfp1_265 | | | |
| 57 | GTR2_scaffold_ | Oligonucleotide | GTR2 CDS targeted for | |
| | 0_1964427_ | | cleavage by Cpf1 enzyme; | |
| | 1972843_+_ | | part of gRNA cassette | |
| | Cfp1_267 | | | |
| 58 | MYB28_scaffold_ | Oligonucleotide | MYB28 CDS targeted for | |
| | 0_2473389_ | | cleavage by Cpf1 enzyme; | |
| | 2480758_+_ | | part of gRNA cassette | |
| | CRISPR_170 | | | |
| 59 | MYB28_scaffold_ | Oligonucleotide | MYB28 CDS targeted for | |
| | 0_2473389_ | | cleavage by Cpf1 enzyme; | |
| | 2480758_+_ | | part of gRNA cassette | |
| | CRISPR_172 | | | |
| 60 | MYB28_scaffold_ | Oligonucleotide | MYB28 CDS targeted for | |
| | 0_2473389_ | | cleavage by Cpf1 enzyme; | |
| | 2480758_+_ | | part of gRNA cassette | |
| | Cfp1_569 | | | |
| 61 | MYB28_scaffold_ | Oligonucleotide | MYB28 CDS targeted for | |
| | 0_2473389_ | | cleavage by Cpf1 enzyme; | |
| | 2480758_+_ | | part of gRNA cassette | |
| | Cfp1_147 | | | |
| 62 | MYB28_scaffold_ | Oligonucleotide | MYB28 CDS targeted for | |
| | 0_2473389_ | | cleavage by Cpf1 enzyme; | |
| | 2480758_+_ | | part of gRNA cassette | |
| | Cfp1_573 | | | |
| 63 | MYB28_scaffold_ | Oligonucleotide | MYB28 CDS targeted for | |
| | 0_2473389_ | | cleavage by Cpf1 enzyme; | |
| | 2480758_+_ | | part of gRNA cassette | |
| | Cfp1_157 | | | |
| 64 | MYB29_scaffold_ | Oligonucleotide | MYB29 CDS targeted for | |
| | 3_2545596_ | | cleavage by Cpf1 enzyme; | |
| | 2553101_−_ | | part of gRNA cassette | |
| | CRISPR_156 | | | |
| 65 | MYB29_scaffold_ | Oligonucleotide | MYB29 CDS targeted for | |
| | 3_2545596_ | | cleavage by Cpf1 enzyme; | |
| | 2553101_−_ | | part of gRNA cassette | |
| | Cfp1_606 | | | |
| 66 | MYB29_scaffold_ | Oligonucleotide | MYB29 CDS targeted for | |
| | 3_2545596_ | | cleavage by Cpf1 enzyme; | |
| | 2553101_−_ | | part of gRNA cassette | |
| | CRISPR_161 | | | |
| 67 | MYB29_scaffold_ | Oligonucleotide | MYB29 CDS targeted for | |
| | 3_2545596_ | | cleavage by Cpf1 enzyme; | |
| | 2553101_−_ | | part of gRNA cassette | |
| | Cfp1_247 | | | |
| 68 | MYB76_scaffold_ | Oligonucleotide | MYB76 CDS targeted for | |
| | 3_2536681_ | | cleavage by Cpf1 enzyme; | |
| | 2543895_−_ | | part of gRNA cassette | |
| | CRISPR_55 | | | |
| 69 | MYB76_scaffold_ | Oligonucleotide | MYB76 CDS targeted for | |
| | 3_2536681_ | | cleavage by Cpf1 enzyme; | |
| | 2543895_−_ | | part of gRNA cassette | |
| | Cfp1_493 | | | |
| 70 | MYB76_scaffold_ | Oligonucleotide | MYB76 CDS targeted for | |
| | 3_2536681_ | | cleavage by Cpf1 enzyme; | |
| | 2543895_−_ | | part of gRNA cassette | |
| | CRISPR_56 | | | |
| 71 | MYB76_scaffold_ | Oligonucleotide | MYB76 CDS targeted for | |
| | 3_2536681_ | | cleavage by Cpf1 enzyme; | |
| | 2543895_−_ | | part of gRNA cassette | |
| | Cfp1_495 | | | |
| 72 | Ta_TFP_ | Oligonucleotide | TFP CDS targeted for | |
| | scaffold_1_ | | cleavage by Cpf1 enzyme; | |
| | 4920343_ | | part of gRNA cassette | |
| | 4927356_+_ | | | |
| | CRISPR_164 | | | |
| 73 | Ta_TFP_ | Oligonucleotide | TFP CDS targeted for | |
| | scaffold_1_ | | cleavage by Cpf1 enzyme; | |
| | 4920343_ | | part of gRNA cassette | |
| | 4927356_+_ | | | |
| | Cfp1_482 | | | |
| 74 | Ta_TFP_ | Oligonucleotide | TFP CDS targeted for | |
| | scaffold_1_ | | cleavage by Cpf1 enzyme; | |
| | 4920343_ | | part of gRNA cassette | |
| | 4927356_+_ | | | |
| | CRISPR_167 | | | |
| 75 | Ta_TFP_ | Oligonucleotide | TFP CDS targeted for | |
| | scaffold_1_ | | cleavage by Cpf1 enzyme; | |
| | 4920343_ | | part of gRNA cassette | |
| | 4927356_+_ | | | |
| | Cfp1_198 | | | |
| 76 | GTR1_115 | Oligonucleotide | GTR1 CDS targeted for | |
| | | | cleavage by SpCAS9 | |
| | | | enzyme; part of gRNA | |
| | | | cassette | |
| 77 | GTR1_116 | Oligonucleotide | GTR1 CDS targeted for | |
| | | | cleavage by SpCAS9 | |
| | | | enzyme; part of gRNA | |
| | | | cassette | |
| 78 | GTR2_scaffold_ | Oligonucleotide | GTR2 CDS targeted for | |
| | 0_1966458_ | | cleavage by SpCAS9 | |
| | 1970958_+_ | | enzyme; part of gRNA | |
| | CRISPR_43 | | cassette | |
| 79 | GTR2_scaffold_ | Oligonucleotide | GTR2 CDS targeted for | |
| | 0_1966458_ | | cleavage by SpCAS9 | |
| | 1970958_+_ | | enzyme; part of gRNA | |
| | CRISPR_46 | | cassette | |
| 80 | MYB28-m1-CDS | Mutant Coding | Mutant hag1-1 allele (−G | hag1-1 |
| | | region | deletion) | |
| 81 | MYB28-m1-PRT | Mutant Protein | Mutant hag1-1 protein (−G | |
| | | | deletion) | |
| 82 | MYB28-m2-CDS | Mutant Coding | Mutant hag1-2 allele (+A | hag1-2, 2172A |
| | | region | insertion) | |
| 83 | MYB28-m2-PRT | Mutant Protein | Mutant hag1-2 protein | |
| | | | (+A insertion) | |
| 84 | MYB28-m3-CDS | Mutant Coding | Mutant hag1 allele (+G | |
| | | region | insertion) | |
| 85 | MYB28-m3-PRT | Mutant Protein | Mutant hag1 protein (+G | |
| | | | insertion) | |
| 86 | MYB28-m4-CDS | Mutant Coding | Mutant hag1 allele (A -> | |
| | | region | G mutation) | |
| 87 | MYB28-m4-PRT | Mutant Protein | Mutant hag1 allele (A -> | |
| | | | G mutation) | |
| 88 | MYB28-m5-CDS | Mutant Coding | Mutant hag1 allele (+A | |
| | | region | insertion) | |
| 89 | MYB28-m5-PRT | Mutant Protein | Mutant hag1 protein (+A | |
| | | | insertion) | |
| 90 | MYB28-m6-CDS | Mutant Coding | Mutant hag1 allele (−AG | |
| | | region | deletion) | |
| 91 | MYB28-m6-PRT | Mutant Protein | Mutant hag 1 protein (−AG | |
| | | | deletion) | |
| 92 | CYP83A1-CDS | WT Coding | Biosynthetic enzyme and | REF2 |
| | | region | a member of cytochrome | |
| 93 | CYP83A1- | WT Gene | P450 family. Catalyzes | |
| | Genomic | | conversion of aliphatic | |
| | locus | | aldoximes to nitrile oxides | |
| 94 | CYP83A1-PRT | WT Protein | or aci-nitro compounds | |
| 95 | CYP83A1-m1- | Mutant Coding | Mutant cyp83a1 allele (G | |
| | CDS | region | insertion) | |
| 96 | CYP83A1-m1- | Mutant Protein | Mutant cyp83a1 allele (G | |
| | PRT | | insertion) | |
| 97 | CYP83A1-m2- | Mutant Coding | Mutant cyp83a1 allele | |
| | CDS | region | (T -> G mutation) | |
| 98 | CYP83A1-m2- | Mutant Protein | Mutant cyp83a1 allele | |
| | PRT | | (T -> G mutation) | |
| 99 | AOP2_sp_PS3 | Oligonucleotide | AOP2 CDS targeted for | |
| | | | cleavage by SpCas9 | |
| | | | enzyme; part of gRNA | |
| | | | cassette | |
| 100 | AOP2_sp_PS1 | Oligonucleotide | AOP2 CDS targeted for | |
| | | | cleavage by SpCas9 | |
| | | | enzyme; part of gRNA | |
| | | | cassette | |
| 101 | AOP2_sp_PS4 | Oligonucleotide | AOP2 CDS targeted for | |
| | | | cleavage by SpCas9 | |
| | | | enzyme; part of gRNA | |
| | | | cassette | |
| 102 | AOP2_sp_PS2 | Oligonucleotide | AOP2 CDS targeted for | |
| | | | cleavage by SpCas9 | |
| | | | enzyme; part of gRNA | |
| | | | cassette | |
| 103 | AOP2_sp_PS6 | Oligonucleotide | AOP2 CDS targeted for | |
| | | | cleavage by SpCas9 | |
| | | | enzyme; part of gRNA | |
| | | | cassette | |
| 104 | AOP2_sa_PS1 | Oligonucleotide | AOP2 CDS targeted for | |
| | | | cleavage by SaCas9 | |
| | | | enzyme; part of gRNA | |
| | | | cassette | |
| 105 | AOP2_sa_PS2 | Oligonucleotide | AOP2 CDS targeted for | |
| | | | cleavage by SaCas9 | |
| | | | enzyme; part of gRNA | |
| | | | cassette | |
| 106 | HAG1_513 | Oligonucleotide | HAG1 CDS targeted for | |
| | | | cleavage by SpCas9 | |
| | | | enzyme; part of gRNA | |
| | | | cassette | |
| 107 | HAG1_sp_PS1_F | Oligonucleotide | HAG1 CDS targeted for | |
| | | | cleavage by SpCas9 | |
| | | | enzyme; part of gRNA | |
| | | | cassette | |
| 108 | HAG1/3_sp_R | Oligonucleotide | HAG1/HAG3 CDS | |
| | | | targeted for cleavage by | |
| | | | SpCas9 enzyme; part of | |
| | | | gRNA cassette | |
| 109 | HAG1/ | Oligonucleotide | HAG1/HAG2 CDS | |
| | 2_sa_PS1_F | | targeted for cleavage by | |
| | | | SaCas9 enzyme; part of | |
| | | | gRNA cassette | |
| 110 | HAG2_sp_PS1_F | Oligonucleotide | HAG2 CDS targeted for | |
| | | | cleavage by SpCas9 | |
| | | | enzyme; part of gRNA | |
| | | | cassette | |
| 111 | HAG2_sp_PS2_F | Oligonucleotide | HAG2 CDS targeted for | |
| | | | cleavage by SpCas9 | |
| | | | enzyme; part of gRNA | |
| | | | cassette | |
| 112 | HAG2_sp_PS3_F | Oligonucleotide | HAG2 CDS targeted for | |
| | | | cleavage by SpCas9 | |
| | | | enzyme; part of gRNA | |
| | | | cassette | |
| 113 | HAG3_sp_PS2_F | Oligonucleotide | HAG3 CDS targeted for | |
| | | | cleavage by SpCas9 | |
| | | | enzyme; part of gRNA | |
| | | | cassette | |
| 114 | HAG3_431 | Oligonucleotide | HAG3 CDS targeted for | |
| | | | cleavage by SaCas9 | |
| | | | enzyme; part of gRNA | |
| | | | cassette | |
| 115 | HAG3_sp_ | Oligonucleotide | HAG3 CDS targeted for | |
| | knockout_1 | | cleavage by SpCas9 | |
| | | | enzyme; part of gRNA | |
| | | | cassette | |
| 116 | HAG3_sp_ | Oligonucleotide | HAG3 CDS targeted for | |
| | knockout_2 | | cleavage by SpCas9 | |
| | | | enzyme; part of gRNA | |
| | | | cassette | |
| 117 | CYP83A1_ | Oligonucleotide | CYP83A1 CDS targeted | |
| | sp_PS3_F | | for cleavage by SpCas9 | |
| | | | enzyme; part of gRNA | |
| | | | cassette | |
| 118 | GTR1_sp_PS1 | Oligonucleotide | GTR1 CDS targeted for | |
| | | | cleavage by SpCas9 | |
| | | | enzyme; part of gRNA | |
| | | | cassette | |
| 119 | GTR1/2_sp_PS1 | Oligonucleotide | GTR1/GTR2 CDS targeted | |
| | | | for cleavage by SpCas9 | |
| | | | enzyme; part of gRNA | |
| | | | cassette | |
| 120 | GTR1/2_sp_PS2 | Oligonucleotide | GTR1/GTR2 CDS targeted | |
| | | | for cleavage by SpCas9 | |
| | | | enzyme; part of gRNA | |
| | | | cassette | |
| 121 | GTR1/2_sa_PS1 | Oligonucleotide | GTR1/GTR2 CDS targeted | |
| | | | for cleavage by SaCas9 | |
| | | | enzyme; part of gRNA | |
| | | | cassette | |
| 122 | GTR1/2_sa_PS2 | Oligonucleotide | GTR1/GTR2 CDS targeted | |
| | | | for cleavage by SaCas9 | |
| | | | enzyme; part of gRNA | |
| | | | cassette | |
| 123 | GTR1_sp_PS2_F | Oligonucleotide | GTR1 CDS targeted for | |
| | | | cleavage by SpCas9 | |
| | | | enzyme; part of gRNA | |
| | | | cassette | |
| 124 | GTR1_sp_PS3_F | Oligonucleotide | GTR1 CDS targeted for | |
| | | | cleavage by SpCas9 | |
| | | | enzyme; part of gRNA | |
| | | | cassette | |
| 125 | GTR1_sp_ | Oligonucleotide | GTR1 CDS targeted for | |
| | knockout_1 | | cleavage by SpCas9 | |
| | | | enzyme; part of gRNA | |
| | | | cassette | |
| 126 | GTR1_sp_ | Oligonucleotide | GTR1 CDS targeted for | |
| | knockout_2 | | cleavage by SpCas9 | |
| | | | enzyme; part of gRNA | |
| | | | cassette | |
| 127 | MYB76-m1ARV- | Mutant Coding | TAAAGAAAGGAGCAT | A427A |
| | CDS | Region | GGACGT (nt 35-55 of | |
| | | | SEQ ID NO: 25) → | |
| | | | TAAAGAAAGG- | |
| | | | GCATGGACGT (nt 35-54 | |
| | | | of SEQ ID NO: 127) | |
| 128 | MYB76-m1ARV- | Mutant Protein | Frameshift caused by 1 bp | |
| | PRT | | deletion | |
| 129 | MYB76-m2ARV- | Mutant Coding | CTGTATCGGAGAAGG | A430B |
| | CDS | Region | GTTAAAGAAAGGAGC | |
| | | | AT (nt 18-50 of SEQ ID | |
| | | | NO: 25) -> CT-------------- | |
| | | | -------------AT (nt 18-21 | |
| | | | of SEQ ID NO: 129) | |
| 130 | MYB76-m2ARV- | Mutant Protein | Presumed loss of function | |
| | PRT | | caused by 27 bp deletion | |
| 131 | MYB29-m1ARV- | Mutant Coding | (nt 86-690 of SEQ ID | A264A, A296A, A316B, |
| | CDS | Region | NO: 22) TCCATGAA--- | A329B |
| | | | 598 bp deletion--- | |
| | | | AAGGAACC (nt 72-82 of | |
| | | | SEQ ID NO: 131) | |
| 132 | MYB29-m1ARV- | Mutant Protein | Truncated protein caused | |
| | PRT | | by large deletion | |
| 133 | MYB29-m2ARV- | Mutant Coding | (nt 72-709 of SEQ ID | A361B |
| | CDS | Region | NO: 22) ACTCATCT--- | |
| | | | 603 bp deletion--- | |
| | | | ACCGCACTG (nt 72-87 | |
| | | | of SEQ ID NO: 133) | |
| 134 | MYB29-m2ARV- | Mutant Protein | Truncated protein caused | |
| | PRT | | by large deletion | |
| 135 | MYB29-m3ARV- | Mutant Coding | ATCCATGAACATGGC | A262A, A275A |
| | CDS | Region | GAAG (nt 85-103 of SEQ | |
| | | | ID NO: 22) → | |
| | | | ATCCATGAA(A)CATG | |
| | | | GCGAAG (nt 85-104 of | |
| | | | SEQ ID NO: 135), and | |
| | | | TCAGCGTCCATGGAA | |
| | | | GGAACCTT (nt 670-692 | |
| | | | of SEQ ID NO: 22) → | |
| | | | TCAGCGTCCATGGAA | |
| | | | (A)GGAACCTT (nt 670-693 | |
| | | | of SEQ ID NO: 135) | |
| 136 | MYB29-m3ARV- | Mutant Protein | Frameshift caused by 1 bp | |
| | PRT | | insertion (second edit also | |
| | | | 1 bp insertion) | |
| 137 | MYB29-m4ARV- | Mutant Coding | TCAGCGTCCATGGAA | A261C |
| | CDS | Region | GGAACCTT (nt 670-692 | |
| | | | of SEQ ID NO: 22) → | |
| | | | TCAGCGTCCA--- | |
| | | | AAGGAACCTT (nt 670-689 | |
| | | | of SEQ ID NO: 137) | |
| 138 | MYB29-m4ARV- | Mutant Protein | Missing M227, E228->K | |
| | PRT | | | |
| 139 | MYB29-m5ARV- | Mutant Coding | TCAGCGTCCATGGAA | A268A |
| | CDS | Region | GGAACCTT (nt 670-692 | |
| | | | of SEQ ID NO: 22) → | |
| | | | TCAGCGTCC---- | |
| | | | AAGGAACCTT (nt 670-689 | |
| | | | of SEQ ID NO: 139) | |
| 140 | MYB29-m5ARV- | Mutant Protein | Frameshift caused by 4 bp | |
| | PRT | | deletion | |
| 141 | MYB29-m6ARV- | Mutant Coding | TCAGCGTCCATGGAA | A263A, A347D |
| | CDS | Region | GGAACCTT (nt 670-692 | |
| | | | of SEQ ID NO: 22) → | |
| | | | TCAGCGTCCATGGA- | |
| | | | GGAACCTT (nt 670-691 | |
| | | | of SEQ ID NO: 141) | |
| 142 | MYB29-m6ARV- | Mutant Protein | Frameshift caused by 1 bp | |
| | PRT | | deletion | |
| 143 | GTR1-m1ARV- | Mutant Coding | CCTCTGCGACACTTAC | A382A |
| | CDS | Region | TTTG (nt 321-340 of SEQ | |
| | | | ID NO: 13) → CCT-------- | |
| | | | -----TTTG (nt 321-327 of | |
| | | | SEQ ID NO: 143) | |
| 144 | GTR1-m1ARV- | Mutant Protein | Frameshift caused by | |
| | PRT | | 13 bp deletion | |
| 145 | GTR2-m1ARV- | Mutant Coding | AGTGCATTGTGAGAG | A412A |
| | CDS | Region | TGCT (nt 1037-1055 of | |
| | | | SEQ ID NO: 16) → | |
| | | | AGTGCATT(T)GTGAG | |
| | | | AGTGCT (nt 1037-1056 | |
| | | | of SEQ ID NO: 145) | |
| 146 | GTR2-m1ARV- | Mutant Protein | Frameshift caused by 1 bp | |
| | PRT | | insertion | |
| 147 | AOP2-m1ARV- | Mutant Coding | TTTCCGAGAGTATGG | A368A |
| | CDS | Region | GGATC (nt 275-294 of | |
| | | | SEQ ID NO: 1) → | |
| | | | TTTCCGAGAG(A)TAT | |
| | | | GGGGATC (nt 275-295 | |
| | | | of SEQ ID NO: 147) | |
| 148 | AOP2-m1ARV- | Mutant Protein | Frameshift caused by 1 bp | |
| | PRT | | insertion | |
| 149 | AOP2-m2ARV- | Mutant Coding | TTTCCGAGAGTATGG | A377A |
| | CDS | Region | GGATC (nt 275-294 of | |
| | | | SEQ ID NO: 1) → | |
| | | | TTTCCGAGA-- | |
| | | | ATGGGGATC (nt 275-292 | |
| | | | of SEQ ID NO: 149) | |
| 150 | AOP2-m2ARV- | Mutant Protein | Frameshift caused by 2 bp | |
| | PRT | | deletion | |
| 151 | AOP2-m3ARV- | Mutant Coding | TTTCCGAGAGTATGG | A390A |
| | CDS | Region | GGATC (nt 275-294 of | |
| | | | SEQ ID NO: 1) → | |
| | | | TTTCCGAGAGT-- | |
| | | | GGGGATC (nt 275-292 | |
| | | | of SEQ ID NO: 151) | |
| 152 | AOP2-m3ARV- | Mutant Protein | Frameshift caused by 2 bp | |
| | PRT | | deletion | |
| 153 | AOP2-m4ARV- | Mutant Coding | TTTCCGAGAGTATGG | A402A |
| | CDS | Region | GGATC (nt 275-294 of | |
| | | | SEQ ID NO: 1) → | |
| | | | TTTCCGAGAGT---- | |
| | | | GGATC (nt 275-290 of | |
| | | | SEQ ID NO: 153) | |
| 154 | AOP2-m4ARV- | Mutant Protein | Frameshift caused by 4 bp | |
| | PRT | | deletion | |
| 155 | AOP2-m5ARV- | Mutant Coding | TTTCCGAGAGTATGG | A378A, A379A, A385A, |
| | CDS | Region | GGATC (nt 275-294 of | A394A, A403B |
| | | | SEQ ID NO: 1) → | |
| | | | TTTCCGAGAGT(T)ATG | |
| | | | GGGATC (nt 275-295 of | |
| | | | SEQ ID NO: 155) | |
| 156 | AOP2-m5ARV- | Mutant Protein | Frameshift caused by 1 bp | |
| | PRT | | insertion | |
| 157 | AOP2-m6ARV- | Mutant Coding | TTTCCGAGAGTATGG | A375A |
| | CDS | Region | GGATC (nt 275-294 of | |
| | | | SEQ ID NO: 1) → TTTC-- | |
| | | | ------TGGGGATC (nt | |
| | | | 275-286 of SEQ ID NO: 157) | |
| 158 | AOP2-m6ARV- | Mutant Protein | Frameshift caused by 8 bp | |
| | PRT | | deletion | |
| 159 | BHLH05- | WT Coding | basic helix-loop-helix | BHLH05, MYC3, |
| | WT-CDS | region | transcription factor05 | bHLH05 |
| 160 | BHLH05-WT- | WT Gene | (bHLH05) transcription | |
| | Genomic | | factor affects the | |
| | Locus | | biosynthesis of | |
| 161 | BHLH05-WT- | WT Protein | glucosinolates | |
| | PRT | | | |
| 162 | IMD1-WT- | WT Coding | ISOPROPYLMALATE | IMD1, |
| | CDS | region | DEHYDROGENASE 1 | ISOPROPYLMALATE |
| 163 | IMD1-WT- | WT Gene | (IMD1) is involved in | DEHYDROGENASE 1 |
| | Genomic | | leucine biosynthesis and | |
| | Locus | | methionine chain | |
| 164 | IMD1-WT- | WT Protein | elongation required for | |
| | PRT | | glucosinolate biosynthesis | |
| 165 | CYP79B3-WT- | WT Coding | Encodes cytochrome P450 | CYP79B3, CYTOCHROME |
| | CDS | region | family 79 and is involved | P450, FAMILY 79, |
| 166 | CYP79B3-WT- | WT Gene | in biosynthesis of | SUBFAMILY B, |
| | Genomic | | glucosinolates | POLYPEPTIDE 3 |
| | Locus | | | |
| 167 | CYP79B3-WT- | WT Protein | | |
| | PRT | | | |
| 168 | MAM1-WT- | WT Coding | Encodes | MAM1, |
| | CDS | region | METHYLTHIOALKYL | METHYLTHIOALKYL |
| 169 | MAM1-WT- | WT Gene | MALATE SYNTHASE 1 | MALATE SYNTHASE 1, |
| | Genomic | | is involved in biosynthesis | gsm1 |
| | Locus | | of glucosinolates | |
| 170 | MAM1-WT- | WT Protein | | |
| | PRT | | | |
| 171 | Ta_FMO-GS- | WT Coding | FLAVIN- | FMO GS-Ox1, FLAVIN- |
| | Ox1-WT-CDS | region | MONOOXYGENASE | MONOOXYGENASE |
| 172 | Ta_FMO-GS- | WT Gene | GLUCOSINOLATE S- | GLUCOSINOLATE S- |
| | Ox1-WT- | | OXYGENASE 1 | OXYGENASE 1, |
| | Gemonic | | catalyzes the conversion | |
| | Locus | | of methylthioalkyl | |
| 173 | Ta_FMO-GS- | WT Protein | glucosinolates to | |
| | Ox1-WT-PRT | | methylsulfinylalkyl | |
| | | | glucosinolates | |
| 174 | Ta_UGT74B1- | WT Coding | UDP- | UGT74B1, UDP- |
| | WT-CDS | region | glucose:thiohydroximate | GLUCOSYL |
| 175 | Ta_UGT74B1- | WT Gene | S-glucosyltransferase | TRANSFERASE 74B1 |
| | WT- | | involved in glucosinolate | |
| | Gemonic | | biosynthesis | |
| | Locus | | | |
| 176 | Ta_UGT74B1- | WT Protein | | |
| | WT-PRT | | | |
| 177 | Ta_FMO-GS- | Mutant Coding | TTGAGCCTCGTCTAGC | |
| | Ox1-1-CDS | Region | TGAA (nt 653-672 of | |
| | | | SEQ ID NO: 171)→ | |
| | | | TTGAGCCTC<A>TC | |
| | | | TAGCTGAA (nt 653-672 | |
| | | | of SEQ ID NO: 177) | |
| 178 | Ta_FMO-GS- | Mutant Protein | Amino acid change | |
| | Ox1-1-PRT | | | |
| 179 | Ta_MAM1-1- | Mutant Coding | GCAAACATAGAGACA | E5 543 |
| | CDS | Region | TTGAG (nt 464-483 of | |
| | | | SEQ ID NO: 168)→ | |
| | | | GCAAACATA<A>AG | |
| | | | ACATTGAG (nt 464-483 | |
| | | | of SEQ ID NO: 179) | |
| 180 | Ta_MAM1-1- | Mutant Protein | Amino acid change | |
| | PRT | | | |
| 181 | Ta_MAM1-2- | Mutant Coding | TGTGTGTGCTGGAGC | D0956 |
| | CDS | Region | AAGAC (nt 891-910 of | |
| | | | SEQ ID NO: 168)→ | |
| | | | TGTGTGTGCTGGA<A> | |
| | | | CAAGAC (nt 891-910 | |
| | | | of SEQ ID NO: 181) | |
| 182 | Ta_MAM1-2- | Mutant Protein | Amino acid change | |
| | PRT | | | |
| 183 | Ta_AOP2- | Mutant Coding | CCGAGAGTATGGGGA | E3196, Nutty, aop2-1 |
| | like-1MAR- | Region | TCCAG (nt 278-297 of | |
| | CDS | | SEQ ID NO: 1)→ | |
| | | | CCGAGAGTATG<A> | |
| | | | GGATCCAG (nt 278-297 | |
| | | | of SEQ ID NO: 183) | |
| 184 | Ta_AOP2- | Mutant Protein | Amino acid change | |
| | like_-_1MAR_- | | | |
| | PRT | | | |
| 185 | Ta_bhlh05-1- | Mutant Coding | AGAAGGCTGGACCTA | D3 N13P3 |
| | CDS | Region | CGCGA (nt 189-208 of | |
| | | | SEQ ID NO: 159) → | |
| | | | AGAAGGCTG<A>CCT | |
| | | | ACGCGA (nt 189-208 of | |
| | | | SEQ ID NO: 185) | |
| 186 | Ta_bhlh05-1- | Mutant Protein | Truncated protein caused | |
| | PRT | | by premature stop codon | |
| 187 | Ta_bhlh05-2- | Mutant Coding | CGGAGACAACACAGT | E5 202P2 |
| | CDS | Region | GATTCT (nt 246-266 of | |
| | | | SEQ ID NO: 159) → | |
| | | | CGGAGACAAC- | |
| | | | CAGTGATTCT (nt 246-265 | |
| | | | of SEQ ID NO: 187) | |
| 188 | Ta_bhlh05-2- | Mutant Protein | Frameshift caused by 1 bp | |
| | PRT | | deletion | |
| 189 | Ta_bhlh05-3- | Mutant Coding | GGCGGAACCGGAGTT | E5 133P2, fad2-2 |
| | CDS | Region | TCCGA (nt 394-413 of | |
| | | | SEQ ID NO: 159) → | |
| | | | GGCGGAACCG<A>AG | |
| | | | TTTCCGA (nt 394-413 of | |
| | | | SEQ ID NO: 189) | |
| 190 | Ta_bhlh05-3- | Mutant Protein | Amino acid change | |
| | PRT | | | |
| 191 | Ta_myb28-5SED- | Mutant Coding | CATCCACGAGCACGG | |
| | CDS | Region | TGAA (nt 84-103 of | |
| | | | SEQ ID NO: 22) → | |
| | | | CATCCACG- | |
| | | | GCACGGTGAA (nt 84-102 | |
| | | | of SEQ ID NO: 191) | |
| 192 | Ta_myb28- | Mutant Protein | Frameshift due to 1 bp | |
| | 5SED-PRT | | deletion | |
| 193 | myb76-1SED- | Mutant Coding | TAAAACGGTGTGGAA | |
| | CDS | Region | AGAG (nt 137-157 of | |
| | | | SEQ ID NO: 25) → | |
| | | | TAAAACGGT(T)GTGG | |
| | | | AAAGAG (nt 137-156 of | |
| | | | SEQ ID NO: 193) | |
| 194 | myb76-1SED- | Mutant Protein | Frameshift due to 1 bp | |
| | PRT | | insertion | |
| 195 | myb29-1SED- | Mutant Coding | GCCACTTGCCCCTAG | 2172A |
| | CDS | Region | CCCTAGTCCGGCCAC | |
| | | | GCTA (nt 381-413 of | |
| | | | SEQ ID NO: 22) → | |
| | | | GCCACTTG------------- | |
| | | | TCCGGCCACGCT (nt | |
| | | | 381-400 of SEQ ID | |
| | | | NO: 195) | |
| 196 | myb29-1SED- | Mutant Protein | Frameshift due to 13 bp | |
| | PRT | | deletion | |
| 197 | myb29-2SED- | Mutant Coding | TAGCCCTAGTCCGGC | 2180A |
| | CDS | Region | CACGCTC (nt 393-414 | |
| | | | of SEQ ID NO: 22) → | |
| | | | TAGCCCTA------ | |
| | | | CCACGCTC (nt 393-408 | |
| | | | of SEQ ID NO: 197) | |
| 198 | myb29-2SED- | Mutant Protein | Presumed loss of function | |
| | PRT | | due to 6 bp deletion | |
| 199 | Ta_imd1-1- | Mutant Coding | AGAGCCCAGAGGCAT | A795, tt4-1 |
| | CDS | Region | TAAGA (nt 663-682 of | |
| | | | SEQ ID NO: 162) → | |
| | | | AGAGCCCA<A>AGGC | |
| | | | ATTAAGA (nt 663-682 | |
| | | | of SEQ ID NO: 199) | |
| 200 | Ta_imd1-1- | Mutant Protein | Amino acid change | |
| | PRT | | | |
| 201 | Ta_imd1-2- | Mutant Coding | TCGGTGTATCGGGAC | D322 |
| | CDS | Region | CTGGA (nt 1040-1059 of | |
| | | | SEQ ID NO: 162) → | |
| | | | TCGGTGTAT<T>GGGA | |
| | | | CCTGGA (nt 1040-1059 | |
| | | | of SEQ ID NO: 201) | |
| 202 | Ta_imd1-2- | Mutant Protein | Amino acid change | |
| | PRT | | | |
| 203 | Ta_cyp79b3- | Mutant Coding | CTTTCCAACGGCTAC | I87207 |
| | 1-CDS | Region | AAAAC (nt 412-431 of | |
| | | | SEQ ID NO: 165) → | |
| | | | CTTTCCAAC<A>GCTA | |
| | | | CAAAAC (nt 412-431 of | |
| | | | SEQ ID NO: 203) | |
| 204 | Ta_cyp79b3- | Mutant Protein | Amino acid change | |
| | 1-PRT | | | |
| 205 | Ta_cyp79b3- | Mutant Coding | GGTCTGATCCACTTA | E5519 |
| | 2-CDS | Region | GCTTT (nt 1328-1347 of | |
| | | | SEQ ID NO: 165) → | |
| | | | GGTCTGAT<T>CACTT | |
| | | | AGCTTT (nt 1328-1347 | |
| | | | of SEQ ID NO: 205) | |
| 206 | Ta_cyp79b3- | Mutant Protein | Amino acid change | |
| | 2-PRT | | | |
| 207 | Ta_cyp83a1- | Mutant Coding | TTCAGGCCCGAGAGG | A766 |
| | 1-CDS | Region | TTTC (nt 1240-1258 of | |
| | | | SEQ ID NO: 97) → | |
| | | | TTCAGGCCC<A>AGA | |
| | | | GGTTTC (nt 1240-1258 | |
| | | | of SEQ ID NO: 207) | |
| 208 | Ta_cyp83a1- | Mutant Protein | Amino acid change | |
| | 1-PRT | | | |
| 209 | Ta_cyp83a1- | Mutant Coding | TTATCATACAAGATA | |
| | 2-CDS | Region | GGAAA (nt 196-215 of | |
| | | | SEQ ID NO: 97) → | |
| | | | TTATCATACAA(A)G | |
| | | | ATAGGAAA (nt 196-216 | |
| | | | of SEQ ID NO: 209) | |
| 210 | Ta_cyp83a1- | Mutant Protein | Frameshift caused by 1 bp | |
| | 2-PRT | | insertion | |
| 211 | Ta cyp83a1- | Mutant Coding | TTATCATACAAGATA | |
| | 3-CDS | Region | GGAAA (nt 196-215 of | |
| | | | SEQ ID NO: 97) → | |
| | | | TTATCATACAA(T)G | |
| | | | ATAGGAAA (nt 196-216 | |
| | | | of SEQ ID NO: 211) | |
| 212 | Ta_cyp83a1- | Mutant Protein | Frameshift caused by 1 bp | |
| | 3-PRT | | insertion | |
| 213 | Ta_AOP2- | Mutant Coding | (nt 270-318 of SEQ ID | |
| | like aop2- | Region | NO: 1) → CGGTCTTT-- | |
| | 2SED-CDS | | 35 bp deletion -- | |
| | | | TGGACAAA (nt 270-285 | |
| | | | of SEQ ID NO: 213) | |
| 214 | Ta_AOP2- | Mutant Protein | Presumed loss of function | |
| | like aop2- | | due to 33 bp deletion | |
| | 2SED-PRT | | | |
| 215 | Ta_AOP2- | Mutant Coding | TCCTCATGTTTTGGAC | |
| | like aop2- | Region | AAAGTTTA (nt 300-323 | |
| | 3SED-CDS | | of SEQ ID NO: 1) → | |
| | | | TCCTCAT-- | |
| | | | TTTGGACAAAGTTA | |
| | | | (nt 300-319 of SEQ ID | |
| | | | NO: 215) | |
| 216 | Ta_AOP2- | Mutant Protein | Frameshift caused by 2 bp | |
| | like aop2- | | deletion | |
| | 3SED-PRT | | | |
| 217 | Ta_AOP2- | Mutant Coding | TCCTCATGTTTTGGAC | |
| | like aop2- | Region | AAAGTTTA (nt 300-323 | |
| | 4SED-CDS | | of SEQ ID NO: 1) → | |
| | | | TCCTCATGTTT- | |
| | | | GACAAAGTTTA (nt | |
| | | | 300-322 of SEQ ID | |
| | | | NO: 217) | |
| 218 | Ta_AOP2- | Mutant Protein | Frameshift caused by 1 bp | |
| | like aop2- | | deletion | |
| | 4SED-PRT | | | |
| 219 | Ta_AOP2- | Mutant Coding | TCCTCATGTTTTGGAC | |
| | like aop2- | Region | AAAGTTTA (nt 300-323 | |
| | 5SED-CDS | | of SEQ ID NO: 1) → | |
| | | | TCCTCATGTTTT(T)G | |
| | | | GACAAAGTTTA (nt | |
| | | | 300-324 of SEQ ID | |
| | | | NO: 219) | |
| 220 | Ta_AOP2- | Mutant Protein | Frameshift caused by 1 bp | |
| | like aop2- | | insertion | |
| | 5SED-PRT | | | |
| 221 | Ta_gtr1-1- | Mutant Coding | CCGCAGCTCTTGCTTG | I87113, gtr1-1 |
| | CDS | Region | CAGG (nt 1561-1580 of | |
| | | | SEQ ID NO: 13) → | |
| | | | CCGCAGCTCTTGTT<T> | |
| | | | GCAGG (nt 1561-1580 | |
| | | | of SEQ ID NO: 221) | |
| 222 | Ta_gtr1-1- | Mutant Protein | Amino acid change | |
| | PRT | | | |
| 223 | Ta_gtr1-2- | Mutant Coding | TGAAATGCATTGTGA | 3A5K, gtr1-2 |
| | CDS | Region | GAGT (nt 1145-1163 of | |
| | | | SEQ ID NO: 13) → | |
| | | | TGAAATGCATGTGTG | |
| | | | AGAGT (nt 1145-1164 of | |
| | | | SEQ ID NO: 223) | |
| 224 | Ta_gtr1-2- | Mutant Protein | Frameshift caused by 1 bp | |
| | PRT | | insertion | |
| 225 | Ta_gtr2-1- | Mutant Coding | AAAGAAAGTGATGAT | AX17D |
| | CDS | Region | GATCA (nt 1762-1781 of | |
| | | | SEQ ID NO: 16) → | |
| | | | AAAGAAAGT<A>ATG | |
| | | | ATGATCA (nt 1762-1781 | |
| | | | of SEQ ID NO: 225) | |
| 226 | Ta_gtr2-1- | Mutant Protein | Amino acid change | |
| | PRT | | | |
| 227 | Ta_gtr2-2- | Mutant Coding | AGTGCATTGTGAGAG | 3A5C, 3A5K, gtr2-2, |
| | CDS | Region | TGCT (nt 1037-1055 of | A427A |
| | | | SEQ ID NO: 16) → | |
| | | | AGTGCAT(A)TGTGAG | |
| | | | AGTGCT (nt 1037-1056 | |
| | | | of SEQ ID NO: 227) | |
| 228 | Ta_gtr2-2- | Mutant Protein | Frameshift caused by 1 bp | A427A |
| | PRT | | insertion | |
| 229 | Ta_gtr2-3- | Mutant Coding | AGTGCATTGTGAGAG | 3A5K, gtr2-3 |
| | CDS | Region | TGCT (nt 1037-1055 of | |
| | | | SEQ ID NO: 16) → | |
| | | | AGTGCAT(G)TGTGAG | |
| | | | AGTGCT (nt 1037-1056 | |
| | | | of SEQ ID NO: 229) | |
| 230 | Ta_gtr2-3- | Mutant Protein | Frameshift caused by 1 bp | 3A5K |
| | PRT | | insertion | |
| 231 | MYB28- | Mutant Coding | Mutant hag1 allele (−GT | 2180A |
| | 2180A-CDS | region | deletion) | |
| 232 | MYB28- | Mutant Protein | Mutant hag1 protein (−GT | 2180A |
| | 2180A-PRT | | deletion) | |
| 233 | MYB28- | Mutant Coding | Mutant hag1 allele (−TG | 2172A |
| | 2172A-CDS | region | deletion) | |
| 234 | MYB28- | Mutant Protein | Mutant hag1 protein (−TG | 2172A |
| | 2172A-PRT | | deletion) | |
| 235 | Ta_gtr1-3- | Mutant Coding | TGAAATGCATTGTGA | 3A5C, gtr1-3 |
| | CDS | Region | GAGT (nt 1145-1163 of | |
| | | | SEQ ID NO: 13) → | |
| | | | TGAAATGCAT- | |
| | | | GTGAGAGT (nt 1145-1164 | |
| | | | of SEQ ID NO: 235) | |
| 236 | Ta_gtr1-3- | Mutant Protein | Frameshift caused by 1 bp | 3A5C |
| | PRT | | deletion | |

In certain embodiments, pennycress plant seeds, seed lots, seed meal, and compositions having reduced sinigrin content as described herein can be obtained from the E3 196, E5 444P1, E5 356P5, I87113, E5 543, or I87207 pennycress mutant lines provided herein, from progeny derived from those mutant lines, from hybrids derived from those mutant lines, or from germplasm from the mutants that provide seed or seed meal comprising less than 30, 28, 25, 16, or 15 micromoles sinigrin per gram by dry weight. In certain embodiments, germplasm from the mutants that provides seed or seed meal comprising less than 30, 28, 25, 16, or 15 micromoles sinigrin per gram by dry weight can be obtained by outcrossing the E3 196, E5 444P1, E5 356P5, I87113, E5 543, or I87207 pennycress mutant lines to other pennycress lines with wild-type sinigrin levels, selfing progeny of the cross, and selecting for progeny of the self that provide seed or seed meal having less than 30, 28, 25, 16, or 15 micromoles sinigrin per gram by dry weight. In certain embodiments, germplasm from the mutants that provides seed meal comprising less than 30, 28, 25, 16, or 15 micromoles sinigrin per gram can be introgressed into the genetic background of a second pennycress line with wild-type sinigrin levels by using the second pennycress line as a recurrent parent in a series of backcrosses followed by selfs, where progeny of the selfs that seed or seed meal comprising less than 30, 28, 25, 16, or 15 micromoles sinigrin per gram by dry weight are selected and carried forward into additional crosses to the recurrent parent. In certain embodiments, the pennycress mutant E3 196, E5 444P1, E5 356P5, I87113, E5 543, and/or I87207 germplasm that provides seed meal comprising less than 30, 28, 25, 16, or 15 micromoles sinigrin per gram by dry weight can be combined in a pennycress plant to provide pennycress plant seeds, seed lots, seed meal, and compositions having reduced sinigrin content as described herein. In certain embodiments, the pennycress mutant E3 196, E5 444P1, E5 356P5, I87113, E5 543, and/or I87207 germplasm can provide pennycress plant seeds, seed lots, seed meal, and compositions comprising 1, 2.5, 5, or 10 to about 15, 16, 18, 20, 25, 28, or 30 μmol sinigrin/gm dw (gram dry weight). Germplasm combinations comprising any of: (i) E3 196 and E5 444P1 germplasm; (ii) E3 196 and I87113 or E5 444P1 germplasm; (iii) E3 196 and I87207 or E5 444P1 germplasm; (iv) I87113 and I87207 or E5 444P germplasm; (iv) E3 196, I87113, E5 444P1, and I87207 germplasm; (v) E5 356P5 and Æ5 543 germplasm; or (vi) any combination of E3 196, E5 444P1, E5 356P5, I87113, E5 543, and/or I87207 germplasm that provide seed or seed meal comprising less than 30, 28, 25, 16, or 15 micromoles sinigrin per gram by dry weight are provided herein. Also provided herein is the combination of any of the germplasms of the F3 196, E5 444P1, E5 356P5, I87113, E5 543, and/or I87207 pennycress mutant lines that provides for reduced sinigrin content or any of the aforementioned germplasm combinations of (i), (ii), (iii), (iv), or (v) with germplasm comprising loss-of function mutations in a GSL biosynthetic coding sequence or gene (e.g., SEQ ID NO: 1, 2, 4, 5, 7, 8, 10, 11, 92, 93, 162, 163, 165, 166, 168, 169, 171, 172, 174, 175, or allelic variant thereof), at least one loss-of-function mutation in a GSL transport coding sequence or gene (SEQ ID NO: 13, 14, 16, 18, or allelic variant thereof), in a GSL hydrolysis coding sequence or gene (SEQ ID NO: 28, 29, or allelic variant thereof), and/or in an expression regulator (e.g., transcription factor; SEQ ID NO: 19, 20, 22, 23, 25, 26, 159, 160, allelic variant thereof) coding sequence or gene.

A representative wild-type (WT) pennycress MYB28 (HAG1) coding sequence is as shown in sequence listing (SEQ ID NO: 19). The terms “MYB28” and “HAG1” are used interchangeably herein. In certain embodiments, a WT pennycress MYB28 coding sequence can have a sequence that deviates from the coding sequence set forth above (e.g., SEQ ID NO: 19), and is referred to as an allelic variant sequence. In certain embodiments, a MYB28 coding sequence allelic variant can have at least 80, at least 85, at least 90, at least 95, at least 98, or at least 99 percent sequence identity to SEQ ID NO: 19. A representative wild-type pennycress MYB28 polypeptide is shown in sequence listing (SEQ ID NO: 21). In certain embodiments, a WT pennycress MYB28 polypeptide can have a sequence that deviates from the polypeptide sequence set forth above (SEQ ID NO: 21), and is referred to as an allelic variant sequence. In certain embodiments, a WT pennycress MYB28 polypeptide can have a sequence that deviates from the polypeptide sequence set forth above (SEQ ID NO: 21), referred to herein as an allelic variant sequence, provided the polypeptide maintains its wild-type function. For example, a MYB28 polypeptide can have at least 80, at least 85, at least 90, at least 95, at least 98, or at least 99) percent sequence identity to SEQ ID NO: 21. A MYB28 polypeptide of an allelic variant can have one or more (e.g., 2, 3, 4, 5, 6, 7, 8, 9, or 10) amino acid modifications (e.g., substitutions) relative to SEQ ID NO: 21.

In certain embodiments, pennycress seed lots, plants, seeds, as well as the seed meals and compositions obtained therefrom, all having reduced sinigrin content, can include at least one loss-of-function modification in a MYB28 gene (e.g., in a MYB28 coding sequence, in a MYB28 regulatory sequence including the promoter, 5′ UTR, intron, 3′ UTR, or in any combination thereof) or a transgene or genome rearrangement that suppresses expression of the MYB28 gene. As used herein, a loss-of-function mutation in a MYB28 gene can be any modification that is effective to suppress MYB28 polypeptide expression or MYB28 polypeptide function. In certain embodiments, suppressed MYB28 polypeptide expression and/or MYB28 polypeptide function can comprise elimination or a reduction in such expression or function in comparison to a wild-type plant (i.e., can be complete or partial). Examples of genetic modifications that can provide for a loss-of-function mutation include, without limitation, deletions, insertions, substitutions, translocations, inversions, duplications, or any combination thereof. In certain embodiments, any of the aforementioned loss-of-function (LOF) modifications in the MYB28 gene can be combined with a loss-of-function modification in a MYB29 gene or allelic variant thereof, and/or a loss-of-function modification in a MYB76 gene or allelic variant thereof to obtain pennycress plant seeds, seed lots, seed meal, and compositions having reduced sinigrin content described herein. Plants, germplasm, seed, seed lots, seed meal, and compositions comprising: (i) MYB28 and MYB29 LOF modifications; (ii) MYB28 and MYB76 LOF modifications; (iii) MYB29 and MYB76 LOF modifications; and (iii) MYB28, MYB29 and MYB76 LOF modifications are also provided herein.

In certain embodiments, pennycress seed lots, plants, seeds, as well as the seed meals and compositions obtained therefrom, all having reduced sinigrin content, can include a deletion (e.g., a single base-pair deletion) relative to the WT pennycress MYB28 coding sequence. In certain embodiments, a modified MYB28 coding sequence can include a single base-pair deletion of the guanine (G) at nucleotide residue 20 in a WT pennycress MYB28 coding sequence (e.g., SEQ ID NO: 19 or an allelic variant thereof). For example, a single base-pair deletion of the guanine (G) at nucleotide residue at nucleotide residue 20 in a WT pennycress MYB28 coding sequence thereby producing a premature stop codon. A representative modified pennycress MYB28 coding sequence having a loss-of-function single base pair deletion is presented in SEQ ID NO: 80.

A modified or mutated pennycress MYB28 coding sequence having a loss-of-function single base pair deletion mutation (e.g., SEQ ID NO: 80) can encode a modified MYB28 polypeptide (e.g., a modified MYB28 polypeptide having suppressed MYB28 polypeptide expression and/or reduced MYB28 polypeptide function). For example, a modified pennycress MYB28 coding sequence having a single base-pair deletion (e.g., SEQ ID NO:80) can encode a modified MYB28 polypeptide. In certain embodiments, a modified MYB28 polypeptide can include a truncation resulting from the introduction of a stop codon at codon position 20 within the MYB28 open reading frame (e.g., SEQ ID NO: 19). A representative truncated pennycress MYB28 polypeptide is presented in SEQ ID NO:81. The aforementioned loss-of-function modifications in a MYB28 encoding gene or a transgene or genome rearrangement that suppresses expression of the MYB28 gene thus include loss-of-function modifications in a gene encoding an MYB28 allelic variant gene, or a transgene or genome rearrangement that suppresses expression of a MYB28 allelic variant gene.

A representative WT pennycress CYP83A1 coding region is presented in SEQ ID NO: 92. Two protospacer locations and adjacent protospacer-adjacent motif (PAM) sites that can be targeted by, for example, CRISPR-SpCAS9, correspond to nucleotides 190-209 (protospacer) and 210-212 (PAM site).

In certain embodiments, a WT pennycress CYP83A1 coding sequence can have a sequence that deviates from the coding sequence set forth above (e.g., SEQ ID NO:92), and is referred to as an allelic variant sequence. In certain embodiments, a CYP83A1 coding sequence allelic variant can have at least 80, at least 85, at least 90, at least 95, at least 98, or at least 99 percent sequence identity to SEQ ID NO:92. A representative WT pennycress CYP83A1 polypeptide is presented in SEQ ID NO:94.

In certain embodiments, a WT pennycress CYP83A1 polypeptide can have a sequence that deviates from the polypeptide sequence set forth above (SEQ ID NO:94), and is referred to as an allelic variant sequence, provided the polypeptide maintains its wild-type function. For example, a CYP83A1 polypeptide can have at least 80, at least 85, at least 90, at least 95, at least 98, or at least 99 percent sequence identity to SEQ ID NO:94. A CYP83A1 polypeptide can have one or more (e.g., 2, 3, 4, 5, 6, 7, 8, 9, or 10) amino acid modifications (e.g., substitutions) relative to SEQ ID NO:94.

In certain embodiments, pennycress seed lots, plants, seeds, as well as the seed meals and compositions obtained therefrom, all having reduced sinigrin content, can include a loss-of-function modification in a CYP83A1 gene (e.g., in a CYP83A1 coding sequence) or a transgene or genome rearrangement that suppresses expression of the CYP83A1 gene. As used herein, a loss-of-function mutation in a CYP83A1 gene can be any modification that is effective to suppress CYP83A1 polypeptide expression or CYP83A1 polypeptide function. In certain embodiments, suppressed CYP83A1 polypeptide expression and/or CYP83A1 polypeptide function can comprise elimination or a reduction in such expression (i.e., can be complete or partial). Examples of genetic modifications include, without limitation, deletions, insertions, substitutions, translocations, inversions, duplications, and any combination thereof. The aforementioned loss-of-function modifications in a CYP83A1 encoding gene or a transgene or genome rearrangement that suppresses expression of the CYP83A1 gene thus include loss-of-function modifications in a gene encoding an CYP83A1 allelic variant gene, or a transgene or genome rearrangement that suppresses expression of an CYP83A1 allelic variant gene.

In certain embodiments, a WT pennycress AOP2 coding sequence can have a sequence that deviates from the coding sequence set forth above (e.g., SEQ ID NO: 1 or 2), and is referred to as an allelic variant sequence. In certain embodiments, a AOP2 coding sequence allelic variant can have at least 80, at least 85, at least 90, at least 95, at least 98, or at least 99 percent sequence identity to SEQ ID NO: 1 or 2. In certain embodiments, a WT pennycress AOP2 polypeptide can have a sequence that deviates from the polypeptide sequence set forth above (SEQ ID NO:3), and is referred to as an allelic variant sequence provided the polypeptide maintains its wild-type function. For example, a AOP2 polypeptide allelic variant can have at least 80, at least 85, at least 90, at least 95, at least 98, or at least 99 percent sequence identity to SEQ ID NO:3. An AOP2 polypeptide allelic variant can have one or more (e.g., 2, 3, 4, 5, 6, 7, 8, 9, or 10) amino acid modifications (e.g., substitutions) relative to SEQ ID NO:3.

In certain embodiments, pennycress seed lots, plants, seeds, as well as the seed meals and compositions obtained therefrom, all having reduced sinigrin content, can include a loss-of-function modification in a AOP2 encoding gene or a transgene or genome rearrangement that suppresses expression of the AOP2 gene. As used herein, a loss-of-function mutation in a AOP2 gene can be any modification that is effective to reduce AOP2 polypeptide expression or AOP2 polypeptide function. In certain embodiments, suppressed AOP2 polypeptide expression and/or AOP2 polypeptide function can comprise elimination or a reduction in such expression or function (i.e., can be complete or partial). Examples of genetic modifications include, without limitation, deletions, insertions, substitutions, translocations, inversions, duplications, and any combination thereof. The aforementioned loss-of-function modifications in an AOP2 encoding gene or a transgene or genome rearrangement that suppresses expression of the AOP2 gene thus include loss-of-function modifications in a gene encoding an AOP2 allelic variant gene, or a transgene or genome rearrangement that suppresses expression of an AOP2 allelic variant gene.

In certain embodiments, a WT pennycress BCAT4 coding sequence can have a sequence that deviates from the coding sequence set forth above (e.g., SEQ ID NO:4), and is referred to as an allelic variant sequence. In certain embodiments, a BCAT4 coding sequence allelic variant can have at least 80, at least 85, at least 90, at least 95, at least 98, or at least 99 percent sequence identity to SEQ ID NO:4. In certain embodiments, a WT pennycress BCAT4 polypeptide can have a sequence that deviates from the polypeptide sequence set forth above (SEQ ID NO:6), and is referred to as an allelic variant sequence provided the polypeptide maintains its wild-type function. For example, a BCAT4 polypeptide allelic variant can have at least 80, at least 85, at least 90, at least 95, at least 98, or at least 99 percent sequence identity to SEQ ID NO:6. A BCAT4 polypeptide allelic variant can have one or more (e.g., 2, 3, 4, 5, 6, 7, 8, 9, or 10) amino acid modifications (e.g., substitutions) relative to SEQ ID NO:76.

In certain embodiments, pennycress seed lots, plants, seeds, as well as the seed meals and compositions obtained therefrom, all having reduced sinigrin content, can include a loss-of-function modification in a BCAT4 encoding gene or a transgene or genome rearrangement that suppresses expression of the BCAT4 gene. As used herein, a loss-of-function mutation in a BCAT4 gene can be any modification that is effective to reduce BCAT4 polypeptide expression or BCAT4 polypeptide function. In certain embodiments, suppressed BCAT4 polypeptide expression and/or BCAT4 polypeptide function can comprise elimination or a reduction in such expression or function (i.e., can be complete or partial). Examples of genetic modifications include, without limitation, deletions, insertions, substitutions, translocations, inversions, duplications, and any combination thereof. The aforementioned loss-of-function modifications in a BCAT4 encoding gene or a transgene or genome rearrangement that suppresses expression of the BCAT4 gene thus include loss-of-function modifications in a gene encoding a BCAT4 allelic variant gene, or a transgene or genome rearrangement that suppresses expression of a BCAT4 allelic variant gene.

In certain embodiments, a WT pennycress BCAT6 coding sequence can have a sequence that deviates from the coding sequence set forth above (e.g., SEQ ID NO:7), and is referred to as an allelic variant sequence. In certain embodiments, a BCAT6 coding sequence allelic variant can have at least 80, at least 85, at least 90, at least 95, at least 98, or at least 99 percent sequence identity to SEQ ID NO:7. In certain embodiments, a WT pennycress BCAT6 polypeptide can have a sequence that deviates from the polypeptide sequence set forth above (SEQ ID NO:9), and is referred to as an allelic variant sequence provided the polypeptide maintains its wild-type function. For example, a BCAT6 polypeptide allelic variant can have at least 80, at least 85, at least 90, at least 95, at least 98, or at least 99 percent sequence identity to SEQ ID NO:9. A BCAT6 polypeptide allelic variant can have one or more (e.g., 2, 3, 4, 5, 6, 7, 8, 9, or 10) amino acid modifications (e.g., substitutions) relative to SEQ ID NO:9.

In certain embodiments, pennycress seed lots, plants, seeds, as well as the seed meals and compositions obtained therefrom, all having reduced sinigrin content, can include a loss-of-function modification in a BCAT6 encoding gene or a transgene or genome rearrangement that suppresses expression of the BCAT6 gene. As used herein, a loss-of-function mutation in a BCAT6 gene can be any modification that is effective to reduce BCAT6 polypeptide expression or BCAT6 polypeptide function. In certain embodiments, suppressed BCAT6 polypeptide expression and/or BCAT6 polypeptide function can comprise elimination or a reduction in such expression or function (i.e., can be complete or partial). Examples of genetic modifications include, without limitation, deletions, insertions, substitutions, translocations, inversions, duplications, and any combination thereof. The aforementioned loss-of-function modifications in a BCAT6 encoding gene or a transgene or genome rearrangement that suppresses expression of the BCAT6 gene thus include loss-of-function modifications in a gene encoding a BCAT6 allelic variant gene, or a transgene or genome rearrangement that suppresses expression of a BCAT6 allelic variant gene.

In certain embodiments, pennycress seed lots, plants, seeds, as well as the seed meals and compositions obtained therefrom, all having reduced sinigrin content, can include a loss-of-function modification in a CYP79F1 encoding gene or a transgene or genome rearrangement that suppresses expression of the CYP79F1 gene. As used herein, a loss-of-function mutation in a CYP79F1 gene can be any modification that is effective to reduce CYP79F1 polypeptide expression or CYP79F1 polypeptide function. In certain embodiments, suppressed CYP79F1 polypeptide expression and/or CYP79F1 polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress CYP79F1 coding sequence can have a sequence that deviates from the coding sequence set forth above (e.g., SEQ ID NO:10), and is referred to as an allelic variant sequence. In certain embodiments, a CYP79F1 coding sequence allelic variant can have at least 80, at least 85, at least 90, at least 95, at least 98, or at least 99 percent sequence identity to SEQ ID NO: 10. In certain embodiments, a WT pennycress CYP79F1 polypeptide can have a sequence that deviates from the polypeptide sequence set forth above (SEQ ID NO:46), and is referred to as an allelic variant sequence provided the polypeptide maintains its wild-type function. In certain embodiments, a CYP79F1 polypeptide allelic variant can have at least 80, at least 85, at least 90, at least 95, at least 98, or at least 99 percent sequence identity to SEQ ID NO: 12. A CYP79F1 polypeptide allelic variant can have one or more (e.g., 2, 3, 4, 5, 6, 7, 8, 9, or 10) amino acid modifications (e.g., substitutions) relative to SEQ ID NO: 12. Loss-of-function modifications in a CYP79F1 encoding gene or a transgene or genome rearrangement that suppresses expression of the CYP79F1 gene thus include loss-of-function modifications in a gene encoding a CYP7911 allelic variant gene, or a transgene or genome rearrangement that suppresses expression of a CYP79F1 allelic variant gene.

In certain embodiments, pennycress seed lots, plants, seeds, as well as the seed meals and compositions obtained therefrom, all having reduced sinigrin content, can include a loss-of-function modification in a GTR1 encoding gene or a transgene or genome rearrangement that suppresses expression of the GTR1 gene. As used herein, a loss-of-function mutation in a GTR1 gene can be any modification that is effective to reduce GTR1 polypeptide expression or GTR1 polypeptide function. In certain embodiments, suppressed GTR1 polypeptide expression and/or GTR1 polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress GTR1 coding sequence can have a sequence that deviates from the coding sequence set forth above (e.g., SEQ ID NO:13), and is referred to as an allelic variant sequence. In certain embodiments, a GTR1 coding sequence allelic variant can have at least 80, at least 85, at least 90, at least 95, at least 98, or at least 99 percent sequence identity to SEQ ID NO: 13. In certain embodiments, a WT pennycress GTR1 polypeptide can have a sequence that deviates from the polypeptide sequence set forth above (SEQ ID NO:15), and is referred to as an allelic variant sequence provided the polypeptide maintains its wild-type function. In certain embodiments, a GTR1 polypeptide allelic variant can have at least 80, at least 85, at least 90, at least 95, at least 98, or at least 99 percent sequence identity to SEQ ID NO: 15. A GTR1 polypeptide allelic variant can have one or more (e.g., 2, 3, 4, 5, 6, 7, 8, 9, or 10) amino acid modifications (e.g., substitutions) relative to SEQ ID NO: 15. The aforementioned loss-of-function modifications in a GTR1 encoding gene or a transgene or genome rearrangement that suppresses expression of the GTR1 gene thus include loss-of-function modifications in a gene encoding a GTR1 allelic variant gene, or a transgene or genome rearrangement that suppresses expression of a GTR1 allelic variant gene.

In certain embodiments, pennycress seed lots, pennycress seed lots, plants, seeds, as well as the seed meals and compositions obtained therefrom, all having reduced sinigrin content can include a complete or partial loss-of-function modification in a GTR2 encoding gene or a transgene or genome rearrangement that suppresses expression of the GTR2 gene. As used herein, a loss-of-function mutation in a GTR2 gene can be any modification that is effective to reduce GTR2 polypeptide expression or GTR2 polypeptide function. In certain embodiments, suppressed GTR2 polypeptide expression and/or GTR2 polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress GTR2 coding sequence can have a sequence that deviates from the coding sequence set forth above (e.g., SEQ ID NO: 16), and is referred to as an allelic variant sequence. In certain embodiments, a GTR2 coding sequence allelic variant can have at least 80, at least 85, at least 90, at least 95, at least 98, or at least 99 percent sequence identity to SEQ ID NO:16. In certain embodiments, a WT pennycress GTR2 polypeptide can have a sequence that deviates from the polypeptide sequence set forth above (SEQ ID NO:17), and is referred to as an allelic variant sequence provided the polypeptide maintains its wild-type function. In certain embodiments, a GTR2 polypeptide allelic variant can have at least 80, at least 85, at least 90, at least 95, at least 98, or at least 99 percent sequence identity to SEQ ID NO: 17. A GTR2 polypeptide allelic variant can have one or more (e.g., 2, 3, 4, 5, 6, 7, 8, 9, or 10) amino acid modifications (e.g., substitutions) relative to SEQ ID NO:17. The aforementioned loss-of-function modifications in a GTR2 encoding gene or a transgene or genome rearrangement that suppresses expression of the GTR2 gene thus include loss-of-function modifications in a gene encoding a GTR2 allelic variant gene, or a transgene or genome rearrangement that suppresses expression of a GTR2 allelic variant gene.

In certain embodiments, pennycress seed lots, plants, seeds, as well as the seed meals and compositions obtained therefrom, all having reduced sinigrin content can include a complete or partial loss-of-function modification in a TFP encoding gene or a transgene or genome rearrangement that suppresses expression of the TIP gene. As used herein, a loss-of-function mutation in a TFP gene can be any modification that is effective to reduce TFP polypeptide expression or TFP polypeptide function. In certain embodiments, suppressed TFP polypeptide expression and/or TFP polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress TFP coding sequence can have a sequence that deviates from the coding sequence set forth above (e.g., SEQ ID NO:28), and is referred to as an allelic variant sequence. In certain embodiments, a TFP coding sequence allelic variant can have at least 80, at least 85, at least 90, at least 95, at least 98, or at least 99 percent sequence identity to SEQ ID NO:28. In certain embodiments, a WT pennycress TFP polypeptide can have a sequence that deviates from the polypeptide sequence set forth above (SEQ ID NO:30), and is referred to as an allelic variant sequence provided the polypeptide maintains its wild-type function. In certain embodiments, a TFP polypeptide allelic variant can have at least 80, at least 85, at least 90, at least 95, at least 98, or at least 99 percent sequence identity to SEQ ID NO:30. A TFP polypeptide allelic variant can have one or more (e.g., 2, 3, 4, 5, 6, 7, 8, 9, or 10) amino acid modifications (e.g., substitutions) relative to SEQ ID NO:30. The aforementioned loss-of-function modifications in a TFP encoding gene or a transgene or genome rearrangement that suppresses expression of the TFP gene thus include loss-of-function modifications in a gene encoding a TFP allelic variant gene, or a transgene or genome rearrangement that suppresses expression of a TFP allelic variant gene.

In certain embodiments, pennycress seed lots, plants, seeds, as well as the seed meals and compositions obtained therefrom, all having reduced sinigrin content, can include a loss-of-function modification in a BHLH05 encoding gene or a transgene or genome rearrangement that suppresses expression of the BHLH05 gene. As used herein, a loss-of-function mutation in a BHLH05 gene can be any modification that is effective to reduce BHLH05 polypeptide expression or BHLH05 polypeptide function. In certain embodiments, suppressed BHLH05 polypeptide expression and/or BHLH05 polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress BHLH05 coding sequence can have a sequence that deviates from the coding sequence set forth above (e.g., SEQ ID NO:159 or 160), and is referred to as an allelic variant sequence. In certain embodiments, a BHLH05 coding sequence allelic variant can have at least 80, at least 85, at least 90, at least 95, at least 98, or at least 99 percent sequence identity to SEQ ID NO: 159 or 160. In certain embodiments, a WT pennycress BHLH05 polypeptide can have a sequence that deviates from the polypeptide sequence set forth above (SEQ ID NO: 161), and is referred to as an allelic variant sequence provided the polypeptide maintains its wild-type function. For example, a BHLH05 polypeptide allelic variant can have at least 80, at least 85, at least 90, at least 95, at least 98, or at least 99 percent sequence identity to SEQ ID NO: 161. An BHLH05 polypeptide allelic variant can have one or more (e.g., 2, 3, 4, 5, 6, 7, 8, 9, or 10) amino acid modifications (e.g., substitutions) relative to SEQ ID NO: 161. The aforementioned loss-of-function modifications in a BHLH05 encoding gene or a transgene or genome rearrangement that suppresses expression of the BHLH05 gene thus include loss-of-function modifications in a gene encoding a BHLH05 allelic variant gene, or a transgene or genome rearrangement that suppresses expression of a BHLH05 allelic variant gene.

In certain embodiments, pennycress seed lots, plants, seeds, as well as the seed meals and compositions obtained therefrom, all having reduced sinigrin content, can include a loss-of-function modification in a IMD1 encoding gene or a transgene or genome rearrangement that suppresses expression of the IMD1 gene. As used herein, a loss-of-function mutation in a IMD1 gene can be any modification that is effective to reduce IMD1 polypeptide expression or IMD1 polypeptide function. In certain embodiments, suppressed IMD1 polypeptide expression and/or IMD1 polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress IMD1 coding sequence can have a sequence that deviates from the coding sequence set forth above (e.g., SEQ ID NO: 162 or 163), and is referred to as an allelic variant sequence. In certain embodiments, a IMD1 coding sequence allelic variant can have at least 80, at least 85, at least 90, at least 95, at least 98, or at least 99 percent sequence identity to SEQ ID NO: 162 or 163. In certain embodiments, a WT pennycress IMD1 polypeptide can have a sequence that deviates from the polypeptide sequence set forth above (SEQ ID NO:164), and is referred to as an allelic variant sequence provided the polypeptide maintains its wild-type function. For example, a IMD1 polypeptide allelic variant can have at least 80, at least 85, at least 90, at least 95, at least 98, or at least 99 percent sequence identity to SEQ ID NO: 164. An IMD1 polypeptide allelic variant can have one or more (e.g., 2, 3, 4, 5, 6, 7, 8, 9, or 10) amino acid modifications (e.g., substitutions) relative to SEQ ID NO: 164. The aforementioned loss-of-function modifications in a IMD1 encoding gene or a transgene or genome rearrangement that suppresses expression of the IMD1 gene thus include loss-of-function modifications in a gene encoding a IMD1 allelic variant gene, or a transgene or genome rearrangement that suppresses expression of a IMD1 allelic variant gene.

In certain embodiments, pennycress seed lots, plants, seeds, as well as the seed meals and compositions obtained therefrom, all having reduced sinigrin content, can include a loss-of-function modification in a CYP79B3 encoding gene or a transgene or genome rearrangement that suppresses expression of the CYP79B3 gene. As used herein, a loss-of-function mutation in a CYP79B3 gene can be any modification that is effective to reduce CYP79B3 polypeptide expression or CYP79B3 polypeptide function. In certain embodiments, suppressed CYP79B3 polypeptide expression and/or CYP79B3 polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress CYP79B3 coding sequence can have a sequence that deviates from the coding sequence set forth above (e.g., SEQ ID NO: 165 or 166), and is referred to as an allelic variant sequence. In certain embodiments, a CYP79B3 coding sequence allelic variant can have at least 80, at least 85, at least 90, at least 95, at least 98, or at least 99 percent sequence identity to SEQ ID NO: 165 or 166. In certain embodiments, a WT pennycress CYP79B3 polypeptide can have a sequence that deviates from the polypeptide sequence set forth above (SEQ ID NO:167), and is referred to as an allelic variant sequence provided the polypeptide maintains its wild-type function. For example, a CYP79B3 polypeptide allelic variant can have at least 80, at least 85, at least 90, at least 95, at least 98, or at least 99 percent sequence identity to SEQ ID NO: 167. A CYP79B3 polypeptide allelic variant can have one or more (e.g., 2, 3, 4, 5, 6, 7, 8, 9, or 10) amino acid modifications (e.g., substitutions) relative to SEQ ID NO:167. The aforementioned loss-of-function modifications in a CYP79B3 encoding gene or a transgene or genome rearrangement that suppresses expression of the CYP79B3 gene thus include loss-of-function modifications in a gene encoding a CYP79B3 allelic variant gene, or a transgene or genome rearrangement that suppresses expression of a CYP79B3 allelic variant gene.

In certain embodiments, pennycress seed lots, plants, seeds, as well as the seed meals and compositions obtained therefrom, all having reduced sinigrin content, can include a loss-of-function modification in a MAM1 encoding gene or a transgene or genome rearrangement that suppresses expression of the MAM1 gene. As used herein, a loss-of-function mutation in a MAM1 gene can be any modification that is effective to reduce MAM1 polypeptide expression or MAM1 polypeptide function. In certain embodiments, suppressed MAM1 polypeptide expression and/or MAM1 polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress MAM1 coding sequence can have a sequence that deviates from the coding sequence set forth above (e.g., SEQ ID NO: 168 or 169), and is referred to as an allelic variant sequence. In certain embodiments, a MAM1 coding sequence allelic variant can have at least 80, at least 85, at least 90, at least 95, at least 98, or at least 99 percent sequence identity to SEQ ID NO: 168 or 169. In certain embodiments, a WT pennycress MAM1 polypeptide can have a sequence that deviates from the polypeptide sequence set forth above (SEQ ID NO:170), and is referred to as an allelic variant sequence provided the polypeptide maintains its wild-type function. For example, a MAM1 polypeptide allelic variant can have at least 80, at least 85, at least 90, at least 95, at least 98, or at least 99 percent sequence identity to SEQ ID NO: 170. A MAM1 polypeptide allelic variant can have one or more (e.g., 2, 3, 4, 5, 6, 7, 8, 9, or 10) amino acid modifications (e.g., substitutions) relative to SEQ ID NO: 170. The aforementioned loss-of-function modifications in a MAM1 encoding gene or a transgene or genome rearrangement that suppresses expression of the MAM1 gene thus include loss-of-function modifications in a gene encoding a MAM1 allelic variant gene, or a transgene or genome rearrangement that suppresses expression of a MAM1 allelic variant gene.

In certain embodiments, pennycress seed lots, plants, seeds, as well as the seed meals and compositions obtained therefrom, all having reduced sinigrin content, can include a loss-of-function modification in an FMO-GS-Ox1 encoding gene or a transgene or genome rearrangement that suppresses expression of the FMO-GS-Ox1 gene. As used herein, a loss-of-function mutation in an FMO-GS-Ox1 gene can be any modification that is effective to reduce FMO-GS-Ox1 polypeptide expression or FMO-GS-Ox1 polypeptide function. In certain embodiments, suppressed FMO-GS-Ox1 polypeptide expression and/or FMO-GS-Ox1 polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress FMO-GS-Ox1 coding sequence can have a sequence that deviates from the coding sequence set forth above (e.g., SEQ ID NO: 171 or 172), and is referred to as an allelic variant sequence. In certain embodiments, an FMO-GS-Ox1 coding sequence allelic variant can have at least 80, at least 85, at least 90, at least 95, at least 98, or at least 99 percent sequence identity to SEQ ID NO: 171 or 172. In certain embodiments, a WT pennycress FMO-GS-Ox1 polypeptide can have a sequence that deviates from the polypeptide sequence set forth above (SEQ ID NO:173), and is referred to as an allelic variant sequence provided the polypeptide maintains its wild-type function. For example, an FMO-GS-Ox1 polypeptide allelic variant can have at least 80, at least 85, at least 90, at least 95, at least 98, or at least 99 percent sequence identity to SEQ ID NO: 173. An FMO-GS-Ox1 polypeptide allelic variant can have one or more (e.g., 2, 3, 4, 5, 6, 7, 8, 9, or 10) amino acid modifications (e.g., substitutions) relative to SEQ ID NO: 173. The aforementioned loss-of-function modifications in an FMO-GS-Ox1 encoding gene or a transgene or genome rearrangement that suppresses expression of the FMO-GS-Ox1 gene thus include loss-of-function modifications in a gene encoding an FMO-GS-Ox1 allelic variant gene, or a transgene or genome rearrangement that suppresses expression of a FMO-GS-Ox1 allelic variant gene.

In certain embodiments, pennycress seed lots, plants, seeds, as well as the seed meals and compositions obtained therefrom, all having reduced sinigrin content, can include a loss-of-function modification in a UGT74B1 encoding gene or a transgene or genome rearrangement that suppresses expression of the UGT74B1 gene. As used herein, a loss-of-function mutation in a UGT74B1 gene can be any modification that is effective to reduce UGT74B1 polypeptide expression or UGT74B1 polypeptide function. In certain embodiments, suppressed UGT74B1 polypeptide expression and/or UGT74B1 polypeptide function can comprise elimination or a reduction in such expression or function (i.e., can be complete or partial). Examples of genetic modifications include, without limitation, deletions, insertions, substitutions, translocations, inversions, duplications, and any combination thereof.

In certain embodiments, a WT pennycress UGT74B1 coding sequence can have a sequence that deviates from the coding sequence set forth above (e.g., SEQ ID NO: 174 or 175), and is referred to as an allelic variant sequence. In certain embodiments, a UGT74B1 coding sequence allelic variant can have at least 80, at least 85, at least 90, at least 95, at least 98, or at least 99 percent sequence identity to SEQ ID NO: 174 or 175. In certain embodiments, a WT pennycress UGT74B1 polypeptide can have a sequence that deviates from the polypeptide sequence set forth above (SEQ ID NO:176), and is referred to as an allelic variant sequence provided the polypeptide maintains its wild-type function. For example, a UGT74B1 polypeptide allelic variant can have at least 80, at least 85, at least 90, at least 95, at least 98, or at least 99 percent sequence identity to SEQ ID NO: 176. An UGT74B1 polypeptide allelic variant can have one or more (e.g., 2, 3, 4, 5, 6, 7, 8, 9, or 10) amino acid modifications (e.g., substitutions) relative to SEQ ID NO: 176. The aforementioned loss-of-function modifications in a UGT74B1 encoding gene or a transgene or genome rearrangement that suppresses expression of the UGT74B1 gene thus include loss-of-function modifications in a gene encoding a UGT74B1 allelic variant gene, or a transgene or genome rearrangement that suppresses expression of a UGT74B1 allelic variant gene.

In certain embodiments, the pennycress seeds, seed lots, seed meals (defatted and non-defatted), compositions comprising those seed meals, and pennycress plants provided herein can comprise loss-of-function mutation(s), transgene(s), and/or genomic rearrangement(s) that suppress expression and/or activity of at least two of any of the aforementioned endogenous pennycress genes or allelic variants thereof (e.g., MYB28, MYB29, MYB76, CYP83A1, AOP2, BCAT4, BCAT6, CYP79F1, GTR1, GTR2, TFP, BHLH05 IMD1, CYP79B3, MAM1, FMO-GS-Ox1, and/or UGT74B1) or encoded polypeptides). In one embodiment, the loss-of-function mutation(s), genomic rearrangement(s), and/or transgene(s) can suppress expression of both a GTR1 gene (e.g., of SEQ ID NO:15 or an allelic variant thereof) and a GTR2 gene (e.g., of SEQ ID NO: 17 or an allelic variant thereof). In one embodiment, the loss-of-function mutation(s), genomic rearrangement(s), and/or transgene(s) can suppress expression and/or activity of both a MYB28 gene (e.g., of SEQ ID NO:21 or an allelic variant thereof) and a MYB29 gene (e.g., of SEQ ID NO:24 or an allelic variant thereof). In one embodiment, the loss-of-function mutation(s), transgene(s), and/or genomic rearrangement(s) can suppress expression and/or activity of both a GTR1 gene (e.g., of SEQ ID NO: 15 or an allelic variant thereof) and a MYB29 gene (e.g., of SEQ ID NO:24 or an allelic variant thereof). In certain embodiments, suppression of gene expression and/or activity provided by the loss-of-function mutation(s), transgene(s), and/or genomic rearrangement(s) is partial. In certain embodiments, such partial suppression in the any of the aforementioned embodiments can comprise a reduction of at least 20%, 30%, 40%, 50%, 60%, 70%, 75%, 80%, 85%, 90%, or 95% of activity and/or transcript levels of the endogenous pennycress gene (e.g., MYB28, MYB29, MYB76, CYP83A1, AOP2, BCAT4, BCAT6, CYP79F1, GTR1, GTR2, TFP, BHLH05 IMD1, CYP79B3, MAM1, FMO-GS-Ox1, and/or UGT74B1) in the plant or a part of the plant (e.g., seed) comprising the loss-of-function mutation(s), transgene(s), and/or genomic rearrangement(s) in comparison to the activity and/or transcript levels in a wild-type control plant lacking the loss-of-function mutation(s), transgene(s), and/or genomic rearrangement(s).

In certain embodiments, a genome editing system such as a CRISPR-Cas9 system can be used to introduce one or more loss-of-function mutations into genes such as the glucosinolate biosynthesis, transporters and related regulatory genes (i.e., transcription factors) provided herewith in Table 1 and the sequence listing to obtain pennycress plants, seeds, seed lots, and compositions with reduced seed sinigrin content. For example, a CRISPR-Cas9 vector can include at least one guide sequence specific to a pennycress GTR2 sequence (see, e.g., SEQ ID NO: 16) and/or at least one guide sequence specific to a pennycress GTR2 sequence (see, e.g., SEQ ID NO:17). A Cas9 enzyme will bind to and cleave within the gene when the target site is followed by a PAM sequence. For example, the canonical SpCAS9 PAM site is the sequence 5′-NGG-3′, where N is any nucleotide followed by two guanine (G) nucleotides. The Cas9 component of a CRISPR-Cas9 system designed to introduce one or more loss-of-function modifications described herein can be any appropriate Cas9. In certain embodiments, the Cas9 of a CRISPR-Cas9 system described herein can be a Streptococcus pyogenes Cas9 (SpCas9). One example of a SpCas9 is described in Fauser et al., 2014.

The LOF mutations in any of the genes of coding sequences of Table 1 can be introduced by a variety of methods. Methods for introduction of the LOF mutations include, but are not limited to, traditional mutagenesis (e.g., Ethyl Methane Sulfonate (EMS), fast neutrons (FN), or gamma rays), TILLING, meganucleases, zinc finger nucleases, transcription activator-like effector nucleases, clustered regularly interspaced short palindromic repeat (CRISPR)-associated nuclease (e.g., Cas9, Cpf1, Cms1, S. aureus Cas9 variants, eSpCas9), targetrons, and the like. Various tools that can be used to introduce mutations into genes have been disclosed in Guha et al., 2017. Methods for modifying genomes by use of Cpf1 or Csm1 nucleases are disclosed in US Patent Application Publication 20180148735, which is incorporated herein by reference in its entirety, can be adapted for introduction of the LOF mutations disclosed herein. Methods for modifying genomes by use of CRISPR-CAS systems are disclosed in US Patent Application Publication 20180179547, which is incorporated herein by reference in its entirety, can be adapted for introduction of the LOF mutations disclosed herein. The genome editing reagents described herein can be introduced into a pennycress plant by any appropriate method. In certain embodiments, nucleic acids encoding the genome editing reagents can be introduced into a plant cell using Agrobacterium- or Ensifer mediated transformation, particle bombardment, liposome delivery, nanoparticle delivery, electroporation, polyethylene glycol (PEG) transformation, or any other method suitable for introducing a nucleic acid into a plant cell. In certain embodiments, the Site-Specific Nuclease (SSN) or other expressed gene editing reagents can be delivered as RNAs or as proteins to a plant cell and the RT, if one is used, can be delivered as DNA.

Also provided herein are defatted pennycress seed meal with reduced sinigrin content in comparison to defatted pennycress seed meal obtained from wild-type pennycress seed lots. Defatted-pennycress seed meal is a product obtained from high-pressure crushing of seed, or from a pre-press solvent extraction process, which removes oil from the whole seed. Solvents used in such extractions include, but are not limited to, hexane or mixed hexanes. The meal is the material that remains after most of the oil has been removed. The typical range of sinigrin in meal made from wild-type pennycress seed is greater than 190 micromoles sinigrin per gram meal by dry weight (μmol/gm dw). To be useful as a high protein animal feed, and competitive with other protein feedstuffs, the level of sinigrin level in meal should be less than 30 micromoles sinigrin per gram by dry weight of the meal. In certain embodiments, defatted pennycress seed meal having a sinigrin content of less than 30, 28, 25, or 15 μmol sinigrin/gm dw are provided. In certain embodiments, defatted pennycress seed meal having a sinigrin content of about 1, 2.5, 5, or 10 to about 15, 16, 18, 20, 25, 28, or 30 μmol sinigrin/gm dw is provided herein. Compositions comprising such defatted pennycress seed meal are also provided herein. Such seed meal or compositions can comprise polynucleotides encoding any of the aforementioned LOF mutations. Such seed meal or compositions can also comprise any marker that is characteristic of the pennycress mutant E3 196, E5 444P1, E5 356P5, I87113, E5 543, or I87207 germplasm. In certain embodiments, such biomarkers include a polynucleotide comprising at least one loss-of-function mutation in pennycress mutant E3 196, E5 444P1 E5 356P5, I87113, E5 543, or I87207. Mutations in the pennycress mutant E3 196, E5 444P1, E5 356P5, I87113, E5 543, or I87207 can be identified by sequencing the genomic DNA or pertinent genes (e.g., genes of Table 1) and comparing those sequences to the corresponding sequences of the parent pennycress lines from which they were obtained.

Non-defatted pennycress seed meal having less sinigrin than non-defatted control pennycress seed meal obtained from wild-type pennycress seed is provided herein. In certain embodiments, the sinigrin content of non-defatted pennycress seed meal and compositions comprising the same that are provided herein is reduced from about 1.25-, 1.5-, 2-, or 3-fold to about 4-, 5-, 6-, 7-, 10-, 20-, 40-, 50-, 60-, 70-, 80-, 100-, 120-, 140-, -160-, 180-, or 200-fold in comparison to control non-defatted pennycress seed meal and compositions comprising the same obtained from control wild-type pennycress seeds. In certain embodiments, the non-defatted pennycress seed meal is obtained from pennycress seeds that have been crushed, ground, macerated, expelled, extruded, or any combination thereof. Typically, the level of sinigrin in wild-type pennycress seed and non-defatted seed meal obtained therefrom varies from about 70 to about 150 μmol sinigrin/gm dw. To be useful as a high protein animal feed, and competitive with other protein feedstuffs, the sinigrin level in non-defatted meal should be less than 30 μmol sinigrin/gm dw of the meal. In certain embodiments, non-defatted pennycress seed meal having a sinigrin content of less than 30, 28, 25, 16, or 15 μmol sinigrin/gm dw are provided herein. In certain embodiments, non-defatted pennycress seed meal having a sinigrin content of about less than 15, 14, or 12 μmol sinigrin/gm dw is provided herein. In certain embodiments, non-defatted pennycress seed meal having a sinigrin content of 1, 2.5, 5, or 10 to about 15, 16, 18, 20, 25, 28, or 30 μmol sinigrin/gm dw are provided herein. Compositions comprising such non-defatted pennycress seed meal are also provided herein. Such seed meal or compositions can comprise polynucleotides encoding any of the aforementioned LOF mutations.

The disclosure will be further described in the following examples, which do not limit the scope of the disclosure described in the claims.

### EXAMPLES

### Example 1: Generation of Transgenic Pennycress Lines Harboring the CRISPR-Cas9 or CRISPR-Cpf1 or CRISPR-Cms1 Constructs

### Materials and Methods

Construction of the Thlaspi arvense (Pennycress) AOP2, BCAT4, BCAT6, CYP79F1, CYP83A1, GTR1, GTR2, MYB28 (HAG1), HAG3 (MYB29), MYB76 And TFP Gene-Specific CRISPR Genome-Editing Vectors.

The constructs and cloning procedures for generation of the Thlaspi arvense (pennycress) AOP2, BCAT4, BCAT6, CYP79F1, CYP83A1, GTR1, GTR2, MYB28 (HAG1), HAG3 (MYB29), MYB76 and TFP-specific CRISPR-SpCas9 and CRISPR-SaCas9 constructs were adapted in part from the following two publications that describe general procedures for use of SaCas9 in plants: Steinert J, et. al. (2015) and Fauser F, et. al. (2014).

The plant selectable markers (formerly NPT) in the original pDe-SpCas9 and pDe-SaCas9 binary vectors were swapped for hygromycin resistance (Hygromycin phosphotransferase, or HPT) or fluorescent protein marker (FP) gene.

Vector Transformation into Agrobacterium

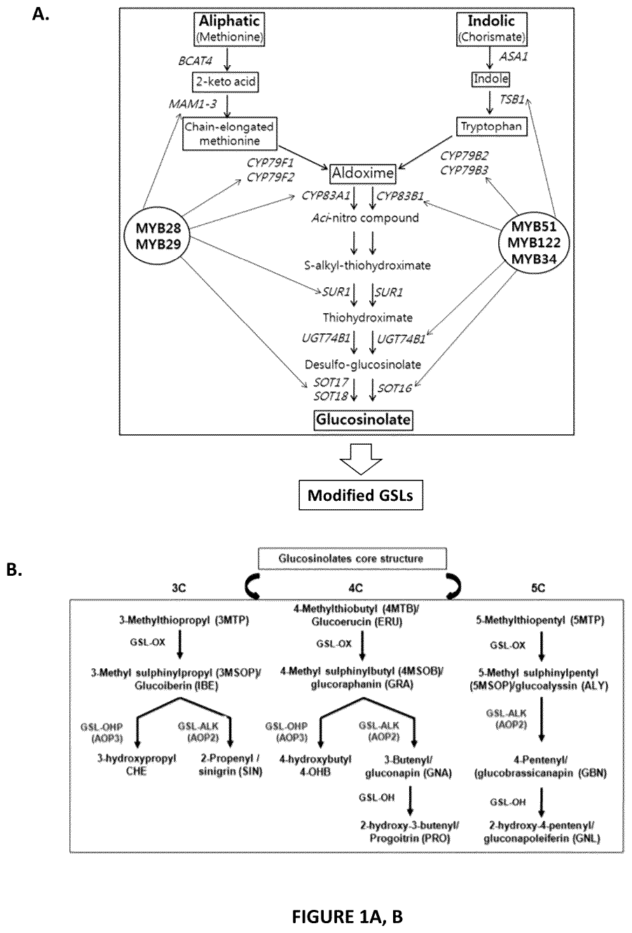

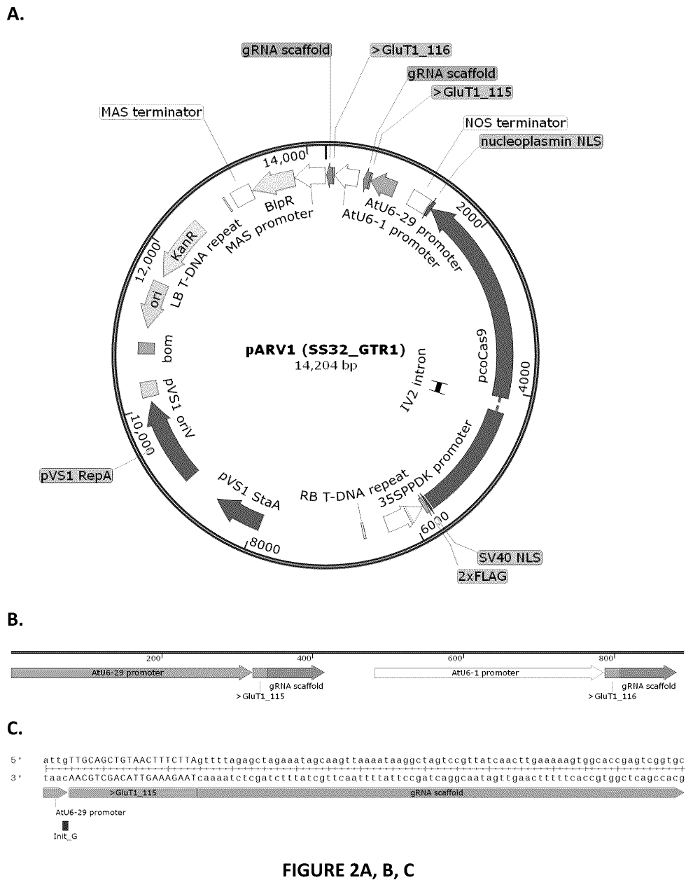

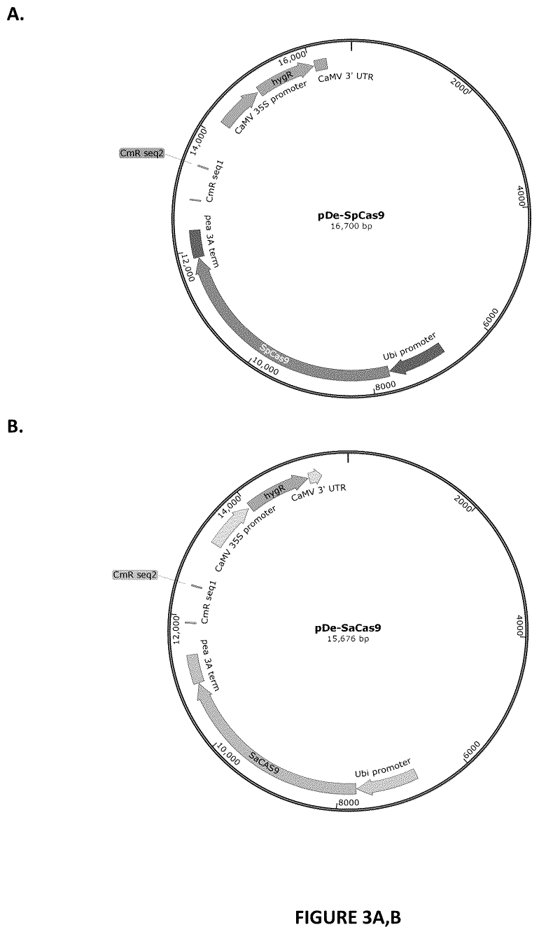

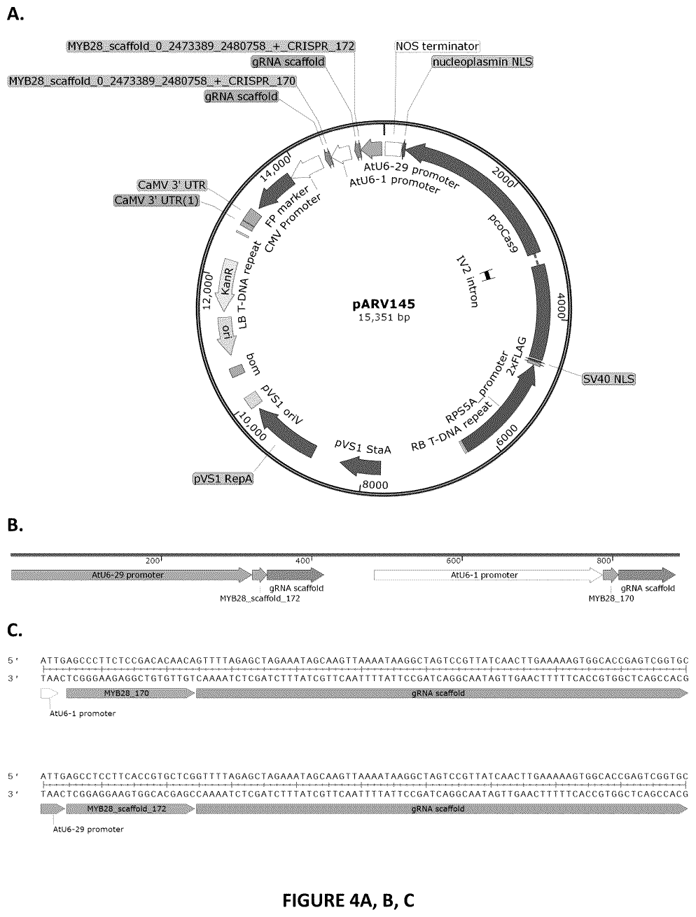

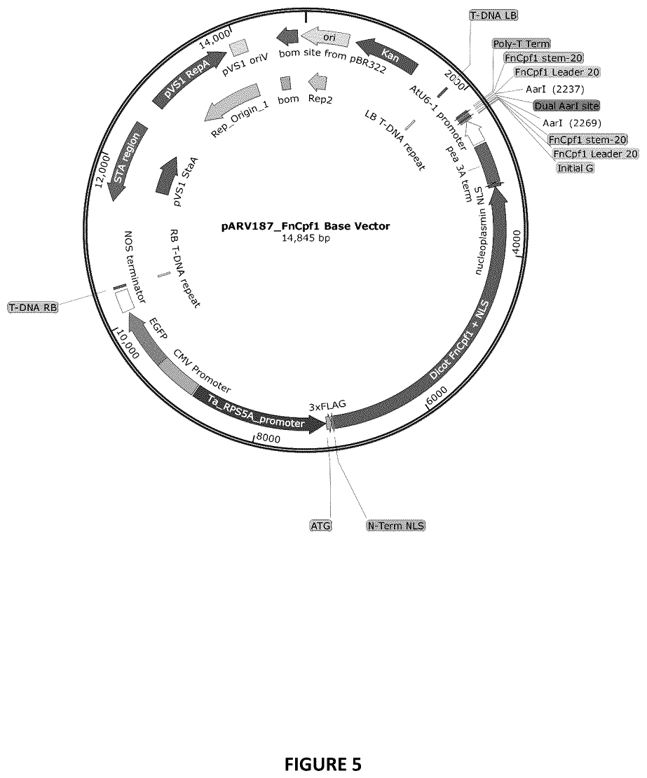

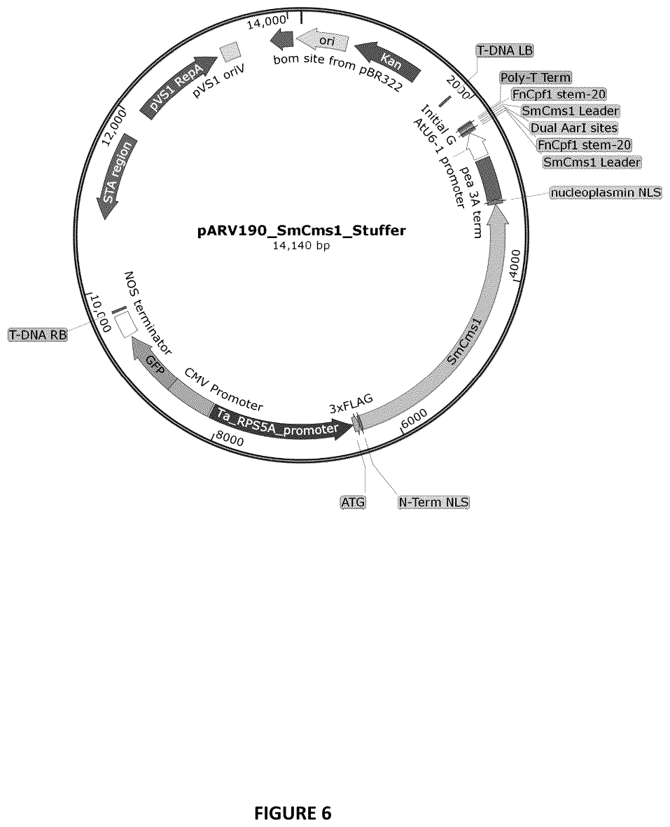

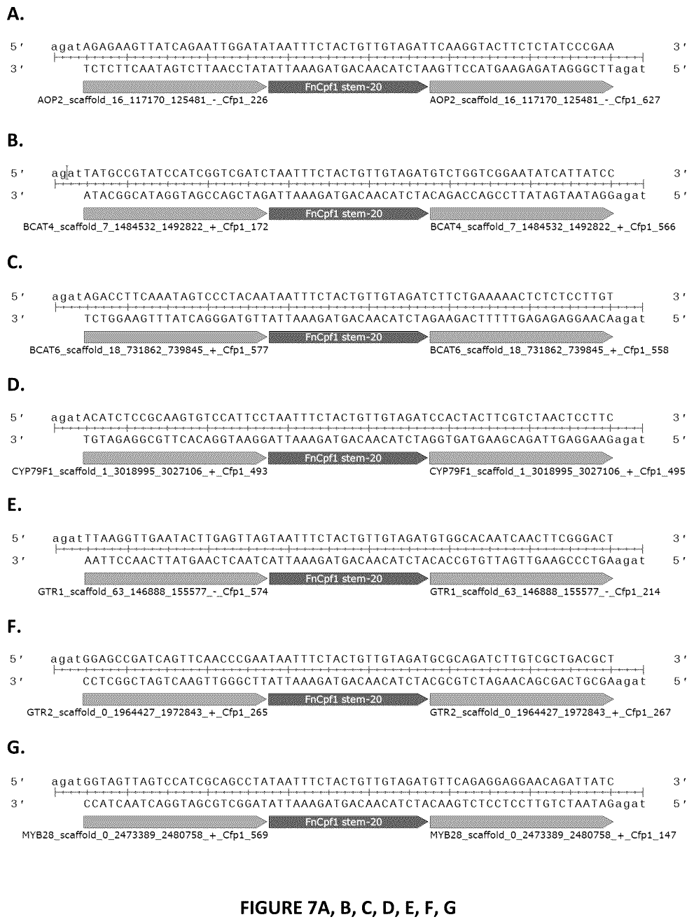

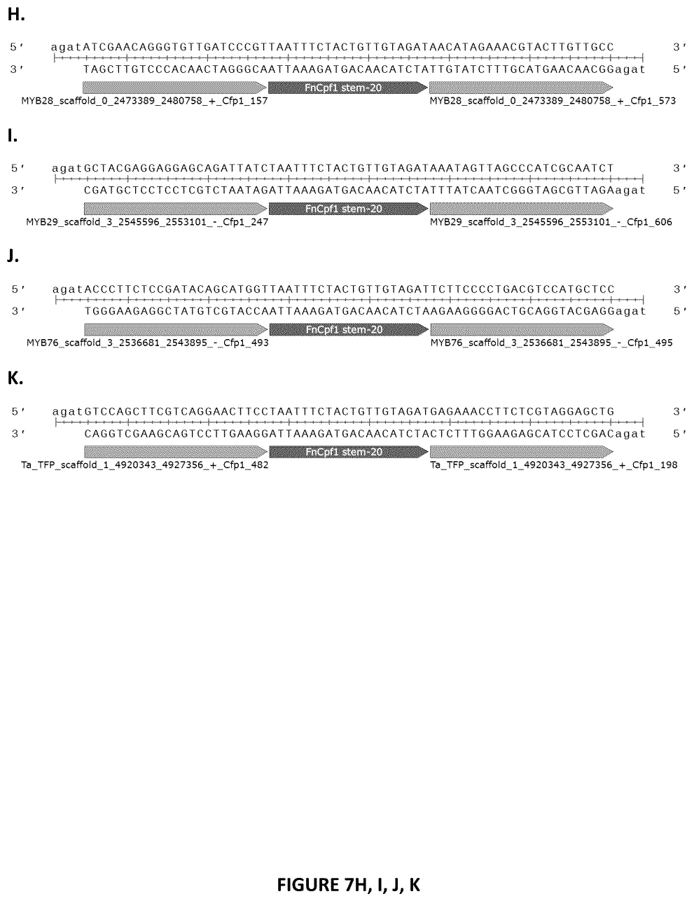

The pDe-SpCas9_Hyg, pDe-SaCas9_Hyg, pARV145, containing the Streptococcus pyogenes Cas9 (SpCas9) and the Staphylococcus aureus Cas9 (SaCas9) cassettes, or related vectors represented in FIGS. 1-7, with the corresponding sequence-specific protospacers were transformed into Agrobacterium tumefaciens strain GV3101 using the freeze/thaw method (Holsters et al, 1978).

The transformation product was plated on 1% agar Luria Broth (LB) plates with gentamycin (50 μg/ml) rifampicin (50 μg/ml) and spectinomycin (75 μg/ml). Single colonies were selected after two days of growth at 28° C.

### Plant Transformation—Pennycress Floral Dip

### Day One:

5 mL of LB+5 uL with appropriate antibiotics (Rifampin (50), Spectinomycin (75), and/or Gentamycin (50)) were inoculated with Agrobacterium. The cultures were allowed to grow, with shaking, overnight at 28° C.

### Day Two (Early Morning):

25 mL of Luria Broth+25 uL appropriate antibiotics (Rifampin (50), Spectinomycin (75), and/or Gentamycin (50)) were inoculated with the initial culture from day one. The cultures were allowed to grow, with shaking, overnight at 28° C.

### Day Two (Late Afternoon):

250 mL of Luria Broth+250 uL appropriate antibiotic (Rifampin (50), Spectinomycin (75), and/or Gentamycin (50)) were inoculated with 25 mL culture. The cultures were allowed to grow, with shaking, overnight at 28° C.

### Day Three:

When the culture had grown to an OD₆₀₀ of ˜1 (or looks thick and silky), the culture was decanted into large centrifuge tubes (all evenly weighted with analytical balance), and spun at 3,500 RPM at room temperature for 10 minutes to pellet cells. The supernatant was decanted off. The pelleted cells were resuspended in a solution of 5% sucrose and 0.02% Silwet L-77. The suspension was poured into clean beakers and placed in a vacuum chamber.

Newly flowering inflorescences of pennycress were fully submerged into the beakers, and subjected to a vacuum pressure of ˜30 inches mercury (˜14.7 psi) for 5 to 10 minutes.

After racemes of pennycress plants (W0011 variety; these plants were 5 generations removed from seeds) were dipped, they were covered loosely with Saran wrap to maintain humidity and kept in the dark overnight before being uncovered and placed back in the environmental growth chamber.

### Screening Transgenic Plants and Growth Condition

Pennycress seeds were surface sterilized by first rinsing in 70% ethanol then incubating 10 minutes in a 30% bleach, 0.05% SDS solution before being rinsed two times with sterile water and plated on selective plates (0.8% agar/one half-strength Murashige and Skoog salts with hygromycin B selection (40 U/ml) or glufosinate (18 μg/ml). Plates were wrapped in parafilm and kept in an environmental growth chamber at 21° C., 16:8 day/night for 8 days until antibiotic or herbicide selection was apparent.

Surviving hygromycin or glufosinate-resistant T1-generation seedlings were transplanted into autoclaved Redi-Earth soil mix and grown in an environmental growth chamber set to 16-hour days/8-hour nights at 21° C. and 50% humidity. T2-generation seeds were planted, and ˜1.5 mg of leaf tissue from each T2-generation plant was harvested with a 3-mm hole punch, then processed using the Thermo Scientific™ Phire™ Plant Direct PCR Kit (Catalog #F130WH) as per manufacturer's instructions. PCR (20 μl volume) was performed.

### Example 2: Generation and Characterization of EMS-Mutagenized Low Sinigrin Mutant Lines E3 196, E5 444P1, I87113 and I87207.

Mutants carrying domestication enabling low glucosinolate trait were isolated from two mutant populations independently created using chemical mutagenesis (ethyl methanesulfonate, EMS) protocol essentially as described in the Materials and Methods section below.

In other embodiments, pennycress plants exhibiting domestication enabling traits such as reduced seed glucosinolate content and loss-of-function mutations in domestication genes can be identified in mutant populations created using fast neutrons (FN), gamma rays (y rays) or other methods of introducing genetic diversity into genomic DNA.

### Materials and Methods

### Solutions

| | | |
|---|---|---|
| A) | 0.2 M sodium phosphate monobasic | 6.9 g/250 mL |
| | (NaH₂PO₄*H₂O) | |
| B) | 0.2 M sodium phosphate dibasic | 7.1 g/250 mL |
| | (NaH₂PO₄ anhydrous) | |

For 50 mL of 0.1 M sodium phosphate buffer at pH 7:

| | |
|---|---|
| 9.75 mL | A |
| 15.25 mL | B |
| 25.0 mL | dH₂O |

0.2% EMS in buffer:
20 mL 0.1M Sodium Phosphate Buffer, pH 7
40 μL EMS liquid (Sigma #M0880-5G)

0.1 M sodium thiosulfate at pH 7.3:
12.4 g sodium thiosulfate in 500 mL

### Primary Seed Surface Sterilization

In the Set #1 experiments, wild-type pennycress (Thlaspi arvense) seeds (W0011 ecotype) were surface sterilized for 10 minutes in a 30% bleach, 0.05% SDS solution before being rinsed 3× with sterile water. Sterilized seeds were immediately subjected to EMS treatment.

### Ethyl Methane Sulfonate (EMS) Treatment of Pennycress Seeds

In the Set #1 experiments, sterilized pennycress seeds (41 g) were agitated in distilled water overnight. Four 250 mL Erlenmeyer flasks with 10 g seed each, and 1 g in a separate small flask as a control, were agitated. The water was decanted.

25 mLs of 0.2% EMS in 0.1M sodium phosphate buffer (pH 7) was added. The control received only phosphate buffer with no EMS. The flasks were shaken in fume hood for 18 hours. The EMS solution was decanted off into an EMS waste bottle.

To rinse the seeds, 25 ml of dH₂O was added to each flask, and the flasks were shaken for 20 minutes. The rinse water was decanted into the EMS waste bottle.

To deactivate the EMS, seeds were washed for 20 minutes in 0.1M sodium thiosulfate (pH 7.3). The sodium thiosulfate solution was decanted into the EMS waste bottle. The seeds were rinsed 4 times with dH2O for 15 minutes.

The seeds were suspended in 0.1% agarose, and germinated directly in autoclaved Redi-Earth soil mix at a density of approximately 10 seeds per 4-inch pot.

In the Set #2 experiments, 42 grams of seeds derived from pennycress accession MN106 were collected as described elsewhere (Dorn et al., 2013), and were treated with 180 ml 0.2% ethyl methanesulfonate (EMS) in a chemical flow hood. The solution and seeds were kept mixed on a rotating platform for 14 hours at room temperature. The seeds were thereafter extensively rinsed with distilled water to remove all traces of the EMS. The seeds were then dried for 24 hours on filter paper in a chemical flow hood. These seeds were considered to be the progenitors of the M1-generation of plants.

### Plant Growth Conditions

In the Set #1 experiments, EMS-treated pennycress seeds were germinated and grown in an environmental growth chamber at 21° C., 16:8 6400K fluorescent light/dark, 50% humidity. Approximately 14 days after planting, plants were thinned and transplanted to a density of 4 plants per 4-inch pot. These M1-generation plants showed telltale chlorotic leaf sectors that are indicative of a successful mutagenesis.

After dry-down, these M1-generation W0011 plants were catalogued and harvested. The M2- and M3-generation seeds were surface sterilized, planted and grown according to the protocols previously described.

In the Set #2 experiments, the MN106 mutagenized seeds were sowed into two small field plots. These plots were allowed to grow over the winter. The following spring abundant albino sectors were noted on the flowering plants as an indication of a successful mutagenesis.

### Identification and Characterization of Low Seed Sinigrin Mutant Lines

In the Set #1 experiments, seeds (M3-generation) from putative M2-generation mutants were planted and grown in potting soil-containing 4-inch pots in a growth chamber, harvested and the sinigrin content in the seed was assessed upon its desiccation to a moisture level of 7-9%. EMS mutagenesis typically introduces single-nucleotide transition mutations (e.g., G to A, or C to T) into plant genomes.

In the Set #2 experiments, seeds were collected from mature M1-generation MN106 plants. M2-generation seeds from batches of 10 M1-generation plants were pooled together. In all, 500 pools representing 5000 mutagenized M1-generation plants were collected. In August, each pool was sowed in a field into an individual row. Robust growth was noted in October. During the following June, M3-generation seeds were collected from approximately 8,000 mature M2-generation individual plants and stored in individual packets.

In both Sets #1 and #2 experiments, NIR spectral analysis was used to make preliminary identification of lines with reduced glucosinolate in M3-generation seeds from each packet. These seeds were scanned using a Metrohm NIRS XDS Multi Vial Analyzer or a Perten DA7250 NIR Spectroscopy Analyzer to assess the amount of sinigrin as described elsewhere (Sidhu et. al., 2014; Golebiowski et. al, 2005; Riu et. al., 2006; Xin et. al., 2014). These analyses captured information related to the approximate levels of total glucosinolate and were used to identify low sinigrin candidates. Seeds showing a significant predicted reduction were used in a wet lab analysis to confirm or determine the sinigrin amount with better accuracy.

Near infrared (NIR) spectroscopic analysis was used to determine the sinigrin content of selected seed lines E3 196, E5 444P1, I87113 and I87207 and to compare the obtained values to the range of sinigrin in corresponding wild type seeds. These mutant lines showed sinigrin content significantly below population average and along with some other representative lines and controls were further analyzed using a method adapted from (Kliebenstein et. al., 2001). Results presented in Table 2 indicate that sinigrin levels in the seeds of these mutant lines are significantly lower and are outside of the corresponding ranges found in control parental seeds.

TABLE 2
Sinigrin content in seeds from selected pennycress lines with low glucosinolates
content was measured using high throughput ion-exchange chromatography-based
method. A minimum of three biological replicates each consisting of 20 mg (~20
seeds) per replicate was used. Each biological replicate was split into two technical
replicates that were loaded on the mini-column and treated independently after
seed extraction process. Last column represents standard error of the mean for
glucosinolates (primarily sinigrin) content in each line.

| | | | | | Sinigrin, | |
|---|---|---|---|---|---|---|
| | | | | | Mean | Std Error, |
| | | | Biological | Technical | μmoles/g | Mean |
| | Line ID | Origin | Reps | Reps | seed | μmoles/g |
| 1 | E3 196 | MN106-derived | 6 | 2 | 15 | 1.6 |
| 2 | E5 444P1 | MN106-derived | 6 | 2 | 23 | 3.5 |
| 3 | I87207 | W0011-derived | 3 | 2 | 25 | 4.1 |
| 4 | I87113 | W0011-derived | 6 | 2 | 30 | 4.5 |
| 5 | I87102 | W0011-derived | 3 | 2 | 94 | 8.0 |
| 6 | I87383 | W0011-derived | 3 | 2 | 96 | 10.7 |
| 7 | E5 051 P1 | MN106-derived | 3 | 2 | 99 | 8.9 |
| 8 | I87256 | W0011 wild type | 3 | 2 | 110 | 9.2 |
| 9 | E5 101 P1 | MN106-derived | 3 | 2 | 102 | 10.1 |
| 10 | E5 484P6 | MN106-derived | 3 | 2 | 106 | 10.4 |
| 11 | 1120/1062 1-13 | ARV breeding | 3 | 2 | 101 | 12.1 |
| 12 | 1082/1008 3-12-1 | ARV breeding | 3 | 2 | 106 | 12.2 |
| 13 | 1053/1023 2-5-1 | ARV breeding | 3 | 2 | 112 | 5.9 |
| 14 | Y1067 | ARV low fiber | 3 | 2 | 129 | 9.4 |
| 15 | Y1126 | ARV low fiber | 3 | 2 | 128 | 10.2 |
| 16 | Beecher | USDA | 120 | 2 | 103 | 2.5 |
| | (WT parent) | | | | | |
| 17 | W0011 | WIU/ISU | 6 | 2 | 102 | 6.4 |
| | (WT parent) | | | | | |
| 18 | MN106 | UMN | 6 | 2 | 116 | 8.5 |
| | (WT parent) | | | | | |

### Example 3. Identification of Underlying Gene Mutations in EMS-Generated Low Seed Sinigrin Mutant Lines.

Genomic DNA was extracted from each mutant, and each sample was subjected to whole-genome sequencing (adapted from Zhang, X., et al., 2018) and extensive bioinformatic analysis to identify induced mutations resulting in amino acid substitutions. For every gene target described in Table 1, a sequence from the mutant line was compared to a WT sequence from the parental line. If the EMS-induced change resulted in a non-silent mutation (amino acid change or a stop codon), the mutation was considered to be a candidate for the low sinigrin phenotype. Once the mutation was identified, a co-segregation analysis in the F2 single seeds or F3 families derived from each of these mutants was performed. This whole-genome sequencing (WGS) revealed that E3 196 (Nutty) line contains a mutation in a predicted pennycress ALKNYL HYDROXALKYL PRODUCING (AOP) polypeptide involved in the last step of sinigrin biosynthesis, while the I87113 line carries a homozygous mutation in the GTR1 gene which encodes a glucosinolate transporter.

Mutation in the AOP2-Like Gene Co-Segregates with Low Glucosinolate Phenotype in Seeds and Vegetative Tissues of Mutant E3 196 (Nutty) Pennycress Line.

In order to demonstrate that the mutation in the AOP2 gene discovered in the E3 196 (Nutty) mutant is responsible for the low sinigrin phenotype, a segregating F2 population from the cross of homozygous Nutty mutant with WT MN106 parental line was performed. The results are presented in Table 3.

TABLE 3
Glucosinolates content in seeds and vegetative tissues from
the segregating population created using mutant pennycress
line E3 196 (Nutty). Each line was genotyped for the presence
of G97R mutation found in AOP2 gene variant in E3 196 (Nutty)
using standard sequencing. Moisture and sinigrin content
in seeds were measured using NIRS, whereas total glucosinolates
content in fresh tissue was determined using a wet-lab method
described in Chopra et al. (2019).

| | NIR | | | Sinigrin, | Glucosinolates |
|---|---|---|---|---|---|
| | sample | Genotype, | Moisture, | μmoles/g | μmoles/g |
| | # | G97R | % | seed | tissue |
| 1 | 15 | wt | 7.3 | 115.4 | 26.1 |
| 2 | 23 | wt | 7.6 | 98.3 | 23.9 |
| 3 | 29 | wt | 7.1 | 101.1 | 20.7 |
| 4 | 34 | wt | 7.1 | 108.1 | 9.7 |
| 5 | 35 | wt | 7.3 | 111.3 | 24.6 |
| 6 | 37 | wt | 7.4 | 115.1 | 13.8 |
| 7 | 38 | wt | 7.3 | 106.0 | 7.8 |
| 8 | 8 | homo | 7.5 | 4.9 | 0.7 |
| 9 | 12 | homo | 7.5 | 9.2 | 0.5 |
| 10 | 17 | homo | 7.0 | 6.9 | 1.4 |
| 11 | 24 | homo | 7.7 | 13.7 | 0.4 |
| 12 | 28 | homo | 7.4 | 7.4 | 0.3 |
| 13 | 41 | homo | 6.9 | 2.1 | 2.1 |
| 14 | 1 | het | 6.9 | 107.7 | 19.0 |
| 15 | 6 | het | 7.1 | 106.3 | 21.5 |
| 16 | 7 | het | 7.1 | 102.0 | 23.9 |
| 17 | 10 | het | 7.7 | 110.0 | 25.6 |
| 18 | 13 | het | 7.3 | 95.4 | 28.6 |
| 19 | 14 | het | 7.5 | 100.4 | 17.2 |
| 20 | 19 | het | 7.4 | 89.8 | 17.7 |
| 21 | 22 | het | 7.4 | 108.1 | 24.4 |
| 22 | 26 | het | 7.4 | 103.5 | 23.3 |
| 23 | 27 | het | 7.6 | 99.6 | 23.7 |
| 24 | 32 | het | 7.0 | 114.3 | n/a |
| 25 | 33 | het | 7.2 | 103.6 | 23.6 |
| | Average | WT | | 107.9 | 18.1 |
| | Average | HET | | 103.4 | 22.6 |
| | Average | HOMO | | 7.4 | 0.9 |

The results presented in Table 3 strongly indicate that the G97R mutation present in the AOP2 gene variant in mutant line E3 196 (Nutty) mutant line results in ˜20-fold reduction of total glucosinolates content in dry seeds and vegetative tissues of the mutant plant.

### Mutation in the Homolog of GTR1 Gene Results in Low Glucosinolate Phenotype in Seeds and Vegetative Tissues of Mutant I87113 Pennycress Line.

Using a WGS approach, the I87113 line was found to carry a homozygous mutation believed to confer a L491F amino acid change in GTR1, a glucosinolate transporter and a member of a major facilitator superfamily. In 98 Embryophyta sequences this position is in a conserved transmembrane helical region and is populated exclusively with small hydrophobic AAs, suggesting that the L491F-causing mutation results in at least a partial loss of function. Indeed, in a separate set of NIRS and wet-lab experiments, the progeny of the I87113 mutant has consistently demonstrated a significant reduction in glucosinolates levels in dry seeds (˜30% of the WT level).

TABLE 4
Sinigrin content in seeds of gtr1-1 mutant I87113 as determined
using a wet-lab method described in Chopra et. al. (2019).

| | | Sinigrin, | |
|---|---|---|---|
| | | Mean | Std Error, |
| | Generation/ | μmoles/g | Mean |
| Line ID | Type | seed | μmoles/g |
| I87113 | M3 | 25 | 4 |
| I87113 | M3 | 30 | 4 |
| I87113 | M4 | 33 | 2 |
| W0011 | Control | 98 | 4 |
| Beecher | Control | 101.4 | 7 |

Example 4: Discovery and Characterization of Other Mutant Lines with Low Sinigrin Content in Seeds.

In the process of whole genome sequencing (WGS) of multiple EMS-mutagenized lines segregating for useful traits (flowering, pod-shattering, oil, protein and fiber content, etc.) mutations in other genes described as potential targets for suppression in Table 1 were identified. In these cases, mutations were present almost exclusively in a heterozygous form, consistent with the fact that they were not selected based on a low glucosinolate phenotype which typically requires a homozygous LOF mutation. Instead, they were identified using this opportunistic approach because the original seed stock was very heavily mutagenized (with an estimated 1,000-2,000 mutations per haploid genome), which makes the presence of more than one potentially useful mutation in the same line relatively likely. Because these lines were selected exclusively based on presence of non-silent mutations, most are expected to be in non-conserved regions and have little or no impact on corresponding gene functions. Nevertheless, these lines were subjected to NIRS and wet-lab assays in order to determine the effects of the identified mutations on glucosinolate content in seeds. The results are summarized in Table 5.

TABLE 5
Sinigrin content in seeds of the segregating populations created using mutant
pennycress lines identified via WGS. The genotypes of each mother line were
determined using standard sequencing. Moisture and sinigrin content in seeds
were measured using NIRS whereas, total glucosinolates content in dry seeds
was determined using a wet-lab procedure described in (Chopra et. al., 2019).

| | | | | NIRS | Wet-Lab | Genotype |
|---|---|---|---|---|---|---|
| | | | | Sinigrin, | Glucosinolate | of the |
| | | Gene | Moisture | μmoles/g | μmoles/g | mother |
| | Line Name | Affected | % | seed | seed | line |
| 1 | A7 11 | FMO_GS-OX1 | 7.6 | 103.3 | 113.5 | HET |
| 2 | A7 66 - CYP83A1 Mut | CYP83A1 | 7.3 | 101.0 | 123.5 | HOM |
| 3 | A7 66 - CYP83A1 WT | CYP83A1 | 8.1 | 85.3 | 119.2 | WT |
| 4 | A7 95 | IMD1 | 4.9 | 115.5 | 115.1 | HOM |
| 5 | D3 22 | IMD1 | 8.3 | 96.6 | 120.0 | HET |
| 6 | D3 N13P3 - F2 | bHLH05 | 17.1 | 81.1 | 66.0 | HOM |
| | (Mut) - 16 | (MYC3) | | | | |
| 7 | D3 N13P3 - F2 | bHLH05 | 12.3 | 80.2 | 91.4 | HOM |
| | (Mut) - 22 | (MYC3) | | | | |
| 8 | D3 N13P3 - F2 | bHLH05 | 13.7 | 119.1 | 125.1 | WT |
| | (Wt) -11 | (MYC3) | | | | |
| 9 | D3 N13P3 - F2 | bHLH05 | 16.8 | 122.8 | 118.8 | WT |
| | (Wt) -12 | (MYC3) | | | | |
| 10 | E5 133P2-1 | bHLH05 | 7.5 | 63.6 | 86.0 | unknown |
| | | (MYC3) | | | | |
| 11 | E5 133P2-2 | bHLH05 | 8.1 | 90.3 | 107.9 | unknown |
| | | (MYC3) | | | | |
| 12 | E5 133P2-3 | bHLH05 | 7.9 | 57.9 | 94.9 | unknown |
| | | (MYC3) | | | | |
| 13 | E5 356P5 | FMO_GS-OX1 | 7.5 | 92.5 | 96.7 | HET |
| 14 | E5 519 - CYP79B3 | CYP79B3 | 7.9 | 91.8 | 110.2 | HOM |
| | Mut 309 | | | | | |
| 15 | E5 519 - CYP79B3 | CYP79B3 | 8.1 | 80.9 | 108.4 | HOM |
| | Mut 311 | | | | | |
| 16 | E5 543 | MAM1 | 6.3 | 57.9 | 89.7 | HET |
| 17 | MN106 #33 | Wt | 8.2 | 91.2 | 106.6 | WT |
| 18 | A7 137 | Wt | 7.7 | 99.8 | 104.0 | WT |
| 19 | E5 301P1 | Wt | 8.3 | 94.5 | 99.0 | WT |

This analysis suggested that some of the mutations (such as in FMO-GS-Ox1 and MAM1 genes) may have at least a partial impact on corresponding protein function. To test this hypothesis, the seeds from the progeny of the original heterozygous lines (segregating in a typical 1:2:1 Mendelian ratio) were subjected to a single-seed wet-lab assay and PCR-based genotyping. The results summarized in Table 6 suggest that mutations in FMO-GS-Ox1 and MAM1 may result in reduction of glucosinolates in dry seeds of homozygous mutant lines (40-60% of WT level).

TABLE 6
Glucosinolates content in seeds of the segregating populations
created using mutant pennycress lines. Total glucosinolates
content (μmoles/g) in single seeds was determined using
a wet-lab method described in Chopra et al. (2019).

| | Gene | WT | Mutant |
|---|---|---|---|
| 1 | CYP83A1 | 138 (±12.22) | 113 (±19.72) |
| 2 | FMO-GS-Ox1 | 106 (±10.4) | 64 (±3.98) |
| 3 | MAM1 | 127 (±8.82) | 51 (±4.67) |

### Example 5. Identification and Characterization of CRISPR-Induced Mutations in Target Genes Related to Glucosinolate Pathway and Seed Accumulation.

Gene editing using Cas9, Cpf1 and Cms1 nucleases typically introduces a double-stranded break into a targeted genome area in close proximity to the nuclease's PAM site. During non-homologous end-joining process (NHEJ) double-stranded breaks are repaired, at times resulting in the introduction of INDELS-type mutations at the repair location in targeted genomes. To identify plants with small INDELS in targeted genes of interest, standard Sanger sequencing and/or T7 endonuclease assays (Guschin et. al., 2010) were employed. Standard PCR protocols followed by Sanger sequencing were used to identify and characterize larger (several hundred base pairs) deletions. Sequence analyses revealed that multiple guide RNAs/CRISPR nuclease combinations were effective in generating loss-of-function (LOF) mutations in gene targets described in Table 1. Plants carrying LOF mutations were grown to the next generation and the phenotypes in seeds or vegetative tissues were confirmed using analytical methods.

Multiple mutations in the MYB28 (HAG1) gene were identified, including frameshift mutations likely conferring complete loss of gene function, but no reduction in sinigrin was observed in any of the corresponding homozygous mutant lines (Table 7). On the other hand, mutations in another MYB family member, MYB29 (HAG3), did result in sinigrin reduction, on average, by 35-50% (Tables 7-9). However, in vegetative tissues of myb28/myb29 (hag1/hag3) mutations stack, a dramatic reductions in glucosinolate content relative to WT controls were observed, suggesting a redundancy in the MYB28 and MYB29 gene functions.

TABLE 7
Sinigrin levels as determined using a wet-lab method described in
Chopra et al. (2019), in homozygous lines generated using CRISPR-
induced mutagenesis in selected gene targets described in Table 1.

| | | | Gene | Sinigrin, | Glucosinolates | |
|---|---|---|---|---|---|---|
| | | Line Name | ratio | μmoles/g | μmoles/g fresh | % |
| | Gene Name(s) | Genotype | n | seed | tissue | Control |
| 1 | WT Control | WT-Beecher | | 105 | 46.1 | n/a |
| | (Beecher) | | | | | |
| 2 | WT Control | WT-W0011 | | 94.3 | 40.1 ± 5.7 | n/a |
| | (W0011) | | | | | |
| 3 | MYB28 (HAG1) | hag1-1 | T2 | 98.1 | | 98% |
| | | (homozygous − G | | | | |
| | | deletion) | | | | |
| 4 | MYB28 (HAG1) | hag1-2 | T3 | 100.7 | | 101% |
| | | (homozygous + A | | | | |
| | | insertion) | | | | |
| 5 | MYB28 (HAG1) | 2180A (hag1 | T1 | | 26.0 ± 3.1 | 65% |
| | MYB29 (HAG3) | het − 2bp; hag3-2 | | | | |
| | Stack | homo − 6 bp) | | | | |
| 6 | MYB28 (HAG1)/ | 2172A (hag1 | T1 | | 1.1 ± 0.3 | 3% |
| | MYB29 (HAG3) | biallelic − 2bp, +A; | | | | |
| | stack | hag3-1 | | | | |
| | | homo − 13 bp) | | | | |
| 7 | GTR1/GTR2 | 3A5K (gtr1-2 | T2 | 20.6 | | 21% |
| | stack | homo + G, gtr 2-3 | | | | |
| | | chimeric + G, +A, | | | | |
| | | WT) | | | | |
| 8 | GTR1/GTR2 | 3A5C (gtr1-3 | T2 | 48.9 | | 49% |
| | stack | het − T, gtr2-2 | | | | |
| | | homo + A) | | | | |

TABLE 8
Sinigrin levels in single T2-generation seeds obtained
from selected biallelic/homozygous MYB29 (HAG3)-edited
lines (wet-lab method, normalized to μmoles/g seed).

| | WT | Line | Line | Line |
|---|---|---|---|---|
| Seed # | Control | A263A | A264A | A269A |
| 1 | 117.9 | 70.8 | 121.5 | 77.4 |
| 2 | 106.7 | 58.8 | 103.5 | 84.1 |
| 3 | 119.6 | 46.5 | 94.5 | 60.6 |
| 4 | 124.6 | 42.1 | 70.5 | 56.3 |
| 5 | 130.3 | 64.5 | 84.7 | 56.2 |
| 6 | 123.9 | 51.3 | 86.1 | 54.6 |
| 7 | 111.7 | 56.4 | 94.1 | 62.1 |
| 8 | 126.5 | 45.4 | 89.6 | 41.9 |
| 9 | 127.5 | 52.1 | 114.0 | 57.1 |
| 10 | 125.1 | 45.9 | 83.5 | 51.5 |
| 11 | 124.5 | 44.0 | 71.3 | 63.3 |
| 12 | 116.1 | 49.8 | 68.7 | 57.2 |
| 13 | 126.1 | 75.7 | 113.4 | 85.7 |
| 14 | 128.1 | 53.0 | 73.2 | 61.5 |
| 15 | 115.9 | 46.7 | 84.9 | 87.3 |
| 16 | 114.4 | 46.2 | 74.6 | 69.2 |
| 17 | 103.2 | 55.3 | 101.6 | 86.8 |
| 18 | 114.5 | 54.3 | 99.6 | 81.5 |
| 19 | 101.4 | 47.6 | 116.8 | 56.2 |
| 20 | 150.0 | 98.4 | 62.6 | 41.5 |
| 21 | 127.1 | 48.1 | 71.1 | 63.7 |
| 22 | 135.0 | 58.6 | 101.3 | 60.8 |
| 23 | 133.7 | 70.3 | 78.3 | 48.1 |
| 24 | 126.9 | 51.5 | 83.3 | 65.7 |
| AVE, μmoles/g | 122.1 | 55.5 | 89.3 | 63.8 |
| STDEV | 10.8 | 12.8 | 16.8 | 13.6 |
| % Control | 100% | 45% | 73% | 52% |

TABLE 9
Sinigrin levels in vegetative tissues from selected 4-weeks old T2-generation
plants grown from biallelically modified MYB29 (HAG3) CRISPR-mutated line
A269A (wet-lab method, normalized to μmoles/g fresh tissue punch).

| | WT | A269A, line # | | | | | | | |
|---|---|---|---|---|---|---|---|---|---|
| BioRep # | Control | 13 | 16 | 21 | 22 | 11 | 14 | 19 | 24 |
| 1 | 19.8 | 4.0 | 7.5 | 3.0 | 3.4 | 3.9 | 8.3 | 7.9 | 8.2 |
| 2 | 19.9 | 4.1 | 5.6 | 3.1 | 5.7 | 2.7 | 6.9 | 5.6 | 8.4 |
| 3 | 16.4 | 3.4 | 5.0 | 3.6 | 5.8 | 2.8 | 7.0 | 6.4 | 7.8 |
| AVERAGE | 18.7 | 3.8 | 6.0 | 3.2 | 5.0 | 3.1 | 7.4 | 6.6 | 8.1 |
| STDEV | 2.0 | 0.4 | 1.3 | 0.3 | 1.4 | 0.7 | 0.8 | 1.2 | 0.3 |
| % Control | 100% | 20% | 32% | 17% | 27% | 17% | 40% | 35% | 43% |

TABLE 10
Sinigrin levels in vegetative tissues from selected T1-
generation seedlings grown from biallelically modified
AOP2 lines (wet-lab method, normalized to μmoles/4.3 mg
fresh tissue punch). Tissue samples were harvested from
cauline leaves when plants were setting pods (wet-lab
method, normalized to μmoles/g fresh tissue punch). T1
plants are generally chimeric for the edits, resulting in
overestimated sinigrin numbers and increased variability.

| | 2032 | | | | |
|---|---|---|---|---|---|
| | WT | | | | |
| BioRep # | control | A370A | A379A | A381A | A380A |
| 1 | 7.9 | 0.2 | 1.4 | −0.3 | 0.3 |
| 2 | 4.6 | 2.1 | 0.6 | 0.1 | 0.3 |
| 3 | 4.1 | 0.4 | 0.1 | 6.9 | 0.4 |
| 4 | 4.0 | −0.4 | 2.9 | 7.2 | −0.6 |
| 5 | 4.2 | 3.5 | 0.5 | 0.1 | −0.4 |
| 6 | 1.6 | 4.1 | 0.9 | 0.1 | −0.4 |
| AVERAGE | 4.4 | 1.7 | 1.1 | 2.3 | −0.1 |
| STDEV | 2.0 | 1.9 | 1.0 | 3.6 | 0.4 |
| % Control | 100% | 38% | 24% | 53% | −1% |

### REFERENCES

Tripathi, M. K., & Mishra, A. S. (2007). Glucosinolates in animal nutrition: A review. Animal Feed Science and Technology, 132 (1-2), 1-27.
EFSA Panel on Contaminants in the Food Chain. (2008). Glucosinolates as undesirable substances in animal feed-scientific opinion of the panel on contaminants in the food chain. EFSA Journal, 590, 1-76.
Fauser F., Schiml S., & Puchta H. (2014). Both CRISPR/Cas-based nucleases and nickases can be used efficiently for genome engineering in Arabidopsis thaliana. Plant J 79:348-359.
Guha T. K., Wai A, & Hausner G. (2017). Programmable Genome Editing Tools and their Regulation for Efficient Genome Engineering, Computational and Structural Biotechnology Journal, 15, 146-160.
Guschin D Y, Waite A J, Katibah G E, Miller J C, Holmes M C, & Rebar E J. (2010) A rapid and general assay for monitoring endogenous gene modification. In: Engineered zinc finger proteins: 247-256. Humana Press, Totowa, NJ.
Holsters, M., De Waele, D., Depicker, A., Messens, E., Van Montagu, M., & Schell, J. (1978). Transfection and transformation of Agrobacterium tumefaciens. Molecular and General Genetics 163 (2), 181-187.
Steinert J., Schiml S., Fauser F., & Puchta H. (2015). Highly efficient heritable plant genome engineering using Cas9 orthologues from Streptococcus thermophilus and Staphylococcus aureus. The Plant Journal 84:1295-305.
Kliebenstein, D. J., Lambrix, V. M., Reichelt, M., Gershenzon, J., & Mitchell-Olds, T. (2001). Gene duplication in the diversification of secondary metabolism: tandem 2-oxoglutarate-dependent dioxygenases control glucosinolate biosynthesis in Arabidopsis. The Plant Cell, 13 (3), 681-693.
Chopra, R., Folstad, N., Lyons, J., Ulmasov, T., Gallaher, C., Sullivan, L., McGovern, A., Mitacek, R., Frels, K., Altendorf, K. Killam, A. Ismail, B., Anderson, J. A., Wyse, D. L. & Marks, M. D. (2019). The adaptable use of Brassica NIRS calibration equations to identify pennycress variants to facilitate the rapid domestication of a new winter oilseed crop. Industrial Crops and Products, 128, 55-61.
Sidhu, H. K., Haagenson, D. M., Rahman, M., & Wiesenborn, D. P. (2014). Diode Array Near Infrared Spectrometer Calibrations for Composition Analysis of Single Plant Canola (Brassica napus) Seed. Applied Engineering in Agriculture, 30 (1), 69-76.
Golebiowski, T., Leong, A. S., & Panozzo, J. F. (2005). Near infrared reflectance spectroscopy of oil in intact canola seed (Brassica napus L.). II. Association between principal components and oil content. Journal of Near Infrared Spectroscopy, 13 (5), 255-264.
Riu, Y. K., Huang, K. L., Wang, W. M., Guo, J., Jin, Y. H., & Luo, Y. B. (2006). Detection of erucic acid and glucosinolate in intact rapeseed by near-infrared diffuse reflectance spectroscopy. Guang pu xue yu guang pu fen xi=Guang pu, 26 (12), 2190-2192.
Xin, H., Khan, N. A., Falk, K. C., & Yu, P. (2014). Mid-infrared spectral characteristics of lipid molecular structures in Brassica carinata seeds: relationship to oil content, fatty acid and glucosinolate profiles, polyphenols, and condensed tannins. Journal of Agricultural and Food Chemistry, 62 (32), 7977-7988.
Dorn, K. M., Fankhauser, J. D., Wyse, D. L., & Marks, M. D. (2013). De novo assembly of the pennycress (Thlaspi arvense) transcriptome provides tools for the development of a winter cover crop and biodiesel feedstock. The Plant Journal, 75 (6), 1028-1038.
Zhang, X., Li, R., Chen, L., Niu, S., Chen, L., Gao, J., Wen, J., Yi, B., Ma, C., Tu, J. and Fu, T., (2018). Fine-mapping and candidate gene analysis of the Brassica juncea white-flowered mutant Bjpc2 using the whole-genome resequencing. Molecular Genetics and Genomics, 293 (2), pp. 359-370.

### OTHER EMBODIMENTS

It is to be understood that while certain embodiments have been described in conjunction with the detailed description thereof and examples, the foregoing description is intended to illustrate and not limit the scope of the disclosure. Other aspects, advantages, and modifications are within the scope of the following embodiments and claims.
Embodiment 1. A composition comprising non-defatted pennycress seed meal that comprises less than 30, 28, 25, 16, or 15 micromoles sinigrin per gram by dry weight.
Embodiment 2. The composition of embodiment 1, wherein said seed meal comprises about 1, 2.5, 5, or 10 to about 15, 16, 18, 20, or 25 micromoles sinigrin per gram by dry weight.
Embodiment 3. The composition of any one of embodiments 1 or 2, wherein said composition has an oil content of about 30% or 35% to about 40% or 50% by dry weight.
Embodiment 4. The composition of any one of embodiments 1 to 3, wherein said composition further comprises a preservative, a dust preventing agent, a bulking agent, a flowing agent, or any combination thereof.
Embodiment 5. The composition of any one of embodiments 1 to 4, wherein said pennycress seed meal is obtained from pennycress seeds that have been crushed, ground, macerated, expelled, extruded, or any combination thereof.
Embodiment 6. The composition of any one of embodiments 1 to 5, wherein said composition comprises: (i) a detectable amount of a polynucleotide comprising at least one loss-of-function mutation in at least one endogenous pennycress coding sequence or gene comprising a polynucleotide sequence selected from the group consisting of SEQ ID NO: 1, 2, 4, 5, 7, 8, 10, 11, 13, 14, 16, 18, 19, 20, 22, 23, 25, 26, 28, 29, 92, 93, 159, 160, 162, 163, 165, 166, 168, 169, 171, 172, 174, 175, and allelic variants thereof; (ii) a detectable amount of a polynucleotide comprising at least one loss-of-function mutation in pennycress mutant E3 196, E5 444P1, E5 356P5, I87113, E5 543, or I87207; or (iii) crushed, ground, and/or macerated seed of pennycress mutant lines E3 196, E5 444P1, E5 356P5, I87113, I87207, E5 543, or germplasm therefrom.
Embodiment 7. A non-defatted pennycress seed meal that comprises less than 30, 28, 25, 16, or 15 micromoles sinigrin per gram by dry weight.
Embodiment 8. The seed meal of embodiment 7, wherein said seed meal comprises about 1, 2.5, 5, or 10 to about 15, 16, 18, 20, or 25 micromoles sinigrin per gram by dry weight.
Embodiment 9. The seed meal of embodiment 7 or 8, wherein said composition has an oil content of 30% or 35% to 40% or 50% by dry weight.
Embodiment 10. The seed meal of any one of embodiments 7 to 9, wherein said seed meal comprises: (i) a detectable amount of a polynucleotide comprising at least one loss-of-function mutation in at least one endogenous pennycress coding sequence or gene comprising a polynucleotide sequence selected from the group consisting of SEQ ID NO: 1, 2, 4, 5, 7, 8, 10, 11, 13, 14, 16, 18, 19, 20, 22, 23, 25, 26, 28, 29, 92, 93, 159, 160, 162, 163, 165, 166, 168, 169, 171, 172, 174, 175, and allelic variants thereof; (ii) a detectable amount of a polynucleotide comprising at least one loss-of-function mutation in pennycress mutant E3 196, E5 356P5, I87113, or E5 543; or (ii) crushed, ground, and/or macerated seed of pennycress mutant lines E3 196, E5 356P5, I87113, E5 543, or germplasm therefrom.
Embodiment 11. A pennycress seed comprising less than 30, 28, 25, 16, or 15 micromoles sinigrin per gram by dry weight.
Embodiment 12. The pennycress seed of embodiment 11, wherein the seed comprises about 1, 2.5, 5, or 10 to about 15, 16, 18, 20, or 25 micromoles sinigrin per gram by dry weight.
Embodiment 13. The pennycress seed of embodiment 11 or 12, wherein the seed comprises: (i) at least one loss-of-function mutation in at least one endogenous pennycress gene encoding a polypeptide selected from the group consisting of SEQ ID NO: 3, 6, 9, 12, 15, 17, 21, 24, 27, 30, 94, 161, 164, 167, 170, 173, 176, and allelic variants thereof; (ii) at least one transgene or genome rearrangement that suppresses expression of at least one endogenous pennycress gene encoding a polypeptide selected from the group consisting of SEQ ID NO: 3, 6, 9, 12, 15, 17, 21, 24, 27, 30, 94, 161, 164, 167, 170, 173, 176, and allelic variants thereof; or (iii) seed of pennycress mutant lines E3 196, E5 444P1, E5 356P5, I87113, E5 543, I87207, or germplasm therefrom.
Embodiment 14. The pennycress seed of any one of embodiments 11 to 13, wherein the seed comprises at least one loss-of-function mutation in at least one endogenous pennycress coding sequence or gene comprising a polynucleotide sequence selected from the group consisting of SEQ ID NO: 1, 2, 4, 5, 7, 8, 10, 11, 13, 14, 16, 18, 19, 20, 22, 23, 25, 26, 28, 29, 92, 93, 159, 160, 162, 163, 165, 166, 168, 169, 171, 172, 174, 175, and allelic variants thereof.
Embodiment 15. The pennycress seed of any one of embodiments 11 to 14, wherein the seed comprises at least one loss-of-function mutation in at least one endogenous pennycress gene encoding a sinigrin biosynthetic enzyme and/or at least one loss-of-function mutation in at least one endogenous pennycress gene encoding a sinigrin transporter.
Embodiment 16. The pennycress seed of embodiment 15, wherein: (i) the sinigrin biosynthetic enzyme comprises a polypeptide selected from the group consisting of SEQ ID NO: 3, 6, 9, 12, 21, 24, 27, 94 162, 163, 165, 166, 168, 169, 171, 172, 174, 175, and allelic variants thereof; or (ii) the pennycress seed comprises a loss-of-function mutation in an endogenous pennycress gene encoding the polypeptide of SEQ ID NO: 21 or an allelic variant thereof and a loss-of-function mutation in an endogenous pennycress gene encoding the polypeptide of a SEQ ID NO: 24 or an allelic variant thereof.
Embodiment 17. The pennycress seed of embodiment 15 or 16, wherein: (i) the sinigrin transporter comprises a polypeptide selected from the group consisting of SEQ ID NO: 15, 17 and allelic variants thereof; (ii) the pennycress seed comprises a loss-of-function mutation in an endogenous pennycress gene encoding the polypeptide of SEQ ID NO: 15 or an allelic variant thereof and a loss-of-function mutation in an endogenous pennycress gene encoding the polypeptide of a SEQ ID NO: 17 or an allelic variant thereof; or (iii) the pennycress seed comprises a loss-of-function mutation in an endogenous pennycress gene encoding the polypeptide of SEQ ID NO: 15 or an allelic variant thereof and a loss-of-function mutation in an endogenous pennycress gene encoding the polypeptide of a SEQ ID NO: 24 or an allelic variant thereof.
Embodiment 18. A seed lot comprising a population of pennycress seeds comprising less than 30, 28, 25, 16, or 15 micromoles sinigrin per gram by dry weight.
Embodiment 19. The seed lot of embodiment 18, wherein the pennycress seeds comprise 1, 2.5, 5, or 10 to 15, 16, 18, 20, or 25 micromoles sinigrin per gram by dry weight.
Embodiment 20. The seed lot of embodiment 18 or 19, wherein the seed comprises: (i) at least one loss-of-function mutation in at least one endogenous pennycress gene encoding a polypeptide selected from the group consisting of SEQ ID NO: 3, 6, 9, 12, 15, 17, 21, 24, 27, 30, 94, 161, 164, 167, 170, 173, 176, and allelic variants thereof; or (ii) at least one transgene or genome rearrangement that suppresses expression of at least one endogenous pennycress gene encoding a polypeptide selected from the group consisting of SEQ ID NO: 3, 6, 9, 12, 15, 17, 21, 24, 27, 30, 94, 161, 164, 167, 170, 173, 176, and allelic variants thereof; or (ii) seed of pennycress mutant lines E3 196, E5 356P5, I87113, E5 543, or germplasm therefrom.
Embodiment 21. The seed lot of any one of embodiments 18 to 20, wherein the seed comprises at least one loss-of-function mutation in at least one endogenous pennycress coding sequence or gene comprising a polynucleotide sequence selected from the group consisting of SEQ ID NO: 1, 2, 4, 5, 7, 8, 10, 11, 13, 14, 16, 18, 19, 20, 22, 23, 25, 26, 28, 29, 92, 93, 159, 160, 162, 163, 165, 166, 168, 169, 171, 172, 174, 175, and allelic variants thereof.
Embodiment 22. The seed lot of any one of embodiments 18 to 20, wherein the seed comprises at least one loss-of-function mutation in at least one endogenous pennycress gene encoding a sinigrin biosynthetic enzyme and/or at least one loss-of-function mutation in at least one endogenous pennycress gene encoding a sinigrin transporter.
Embodiment 23. The seed lot of embodiment 22, wherein: (i) the sinigrin biosynthetic enzyme comprises a polypeptide selected from the group consisting of SEQ ID NO: 3, 6, 9, 12, 21, 24, 27, 94, 164, 167, 170, 173, 176, and allelic variants thereof; or (ii) the pennycress seed lot comprises a loss-of-function mutation in an endogenous pennycress gene encoding the polypeptide of SEQ ID NO: 21 or an allelic variant thereof and a loss-of-function mutation in an endogenous pennycress gene encoding the polypeptide of a SEQ ID NO: 24 or an allelic variant thereof.
Embodiment 24. The seed lot of embodiment 22 or 23, wherein: (i) the sinigrin transporter comprises a polypeptide selected from the group consisting of SEQ ID NO: 15, 17 and allelic variants thereof; (ii) the pennycress seed lot comprises a loss-of-function mutation in an endogenous pennycress gene encoding the polypeptide of SEQ ID NO: 15 or an allelic variant thereof and a loss-of-function mutation in an endogenous pennycress gene encoding the polypeptide of a SEQ ID NO: 17 or an allelic variant thereof; or (iii) the pennycress seed lot comprises a loss-of-function mutation in an endogenous pennycress gene encoding the polypeptide of SEQ ID NO: 15 or an allelic variant thereof and a loss-of-function mutation in an endogenous pennycress gene encoding the polypeptide of a SEQ ID NO: 24 or an allelic variant thereof.
Embodiment 25. The seed lot of any one of embodiments 18 to 24, wherein said population of pennycress seeds comprise seeds having at least one loss-of-function mutation in an endogenous pennycress gene that encodes SEQ ID NO:2 or an allelic variant thereof.
Embodiment 26. The seed lot of any one of embodiments 18 to 25, wherein the loss-of-function mutation in the gene encoding SEQ ID NO:2 or the allelic variant thereof comprises an insertion, deletion, or substitution of one or more nucleotides.
Embodiment 27. The seed lot of embodiment 26, wherein the loss-of-function mutation in the gene encoding SEQ ID NO:2 or the allelic variant thereof comprises a mutation that introduces a pre-mature stop codon or frameshift mutation at codon positions 1-108 of SEQ ID NO: 1 or an allelic variant thereof.
Embodiment 28. The seed lot of embodiment 26, wherein the loss-of-function mutation is in a polynucleotide encoding MYB28, MYB29, MYB76, or any combination thereof.
Embodiment 29. The seed lot of any one of embodiments 18 to 28, wherein the population comprises at least 10 seeds comprising less than 25 micromoles sinigrin per gram by dry weight or 1, 2.5, 5, or 10 to 15, 16, 18, 20, or 25 micromoles sinigrin per gram by dry weight.
Embodiment 30. The seed lot of any one of embodiments 18 to 29, wherein at least 95% of the pennycress seeds in the seed lot are seeds comprising less than 30, 28, 25, 16, or 15 micromoles sinigrin per gram by dry weight or 1, 2.5, 5, or 10 to 15, 16, 18, 20, or 25 micromoles sinigrin per gram by dry weight.
Embodiment 31. The seed lot of any one of embodiments 18 to 30, wherein less than 5% of the seeds in said seed lot have greater than 25 or 30 micromoles sinigrin per gram by dry weight.
Embodiment 32. The seed lot of any one of embodiments 18 to 31, wherein said seeds further comprise an agriculturally acceptable excipient or adjuvant.
Embodiment 33. The seed lot of any one of embodiments 18 to 32, wherein said seeds further comprise a fungicide, a safener, or any combination thereof.
Embodiment 34. A method of making non-defatted pennycress seed meal comprising less than 30, 28, 25, 16, or 15 micromoles sinigrin per gram by dry weight or 1, 2.5, 5, or 10 to 15, 16, 18, 20, or 25 micromoles sinigrin per gram by dry weight, comprising the step of grinding, macerating, extruding, and/or crushing the seed lot of any one of embodiments 18 to 32 thereby obtaining the non-defatted seed meal.
Embodiment 35. A method of making defatted pennycress seed meal comprising less than 30 micromoles sinigrin per gram by dry weight or about 1, 2.5, 5, or 10 to about 15, 16, 18, 20, 25, 28, or 30 micromoles sinigrin per gram by dry weight, comprising the steps of solvent extracting the seed lot of any one of embodiments 18 to 32, and separating the extracted seed meal from the solvent, thereby obtaining the defatted seed meal.
Embodiment 36. Pennycress seed meal comprising less than 30, 28, or micromoles sinigrin per gram by dry weight or about 1, 2.5, 5, or 10 to about 15, 16, 18, 20, 25, 28, or 30 micromoles sinigrin per gram by dry weight, wherein the seed meal is defatted.
Embodiment 37. The seed meal of embodiment 36, wherein said seed meal has an oil content of about 0% or 0.5% to about 12% or 15% by dry weight.
Embodiment 38. The pennycress seed meal of embodiments 36 or 37, wherein said meal comprises: (i) a detectable amount of a polynucleotide comprising at least one loss-of-function mutation in at least one endogenous pennycress coding sequence or gene comprising a polynucleotide sequence selected from the group consisting of SEQ ID NO: 1, 2, 4, 5, 7, 8, 10, 11, 13, 14, 16, 18, 19, 20, 22, 23, 25, 26, 28, 29, 92, 93, 159, 160, 162, 163, 165, 166, 168, 169, 171, 172, 174, 175, and allelic variants thereof; (ii) a detectable amount of a polynucleotide comprising at least one loss-of-function mutation in pennycress mutant E3 196, E5 444P1, E5 356P5, I87113, E5 543, or I87207; or (iii) crushed, ground, and/or macerated seed of pennycress mutant lines E3 196, E5 444P1, E5 356P5, I87113, I87207, E5 543, or germplasm therefrom.
Embodiment 39. The pennycress seed meal of any one of embodiments 36 to 38, wherein said meal comprises ground and/or macerated seed of a population of pennycress seeds comprising seeds having at least one loss-of-function mutation in at least one endogenous pennycress coding sequence or gene comprising a polynucleotide sequence selected from the group consisting of SEQ ID NO: 1, 2, 4, 5, 7, 8, 10, 11, 13, 14, 16, 18, 19, 20, 22, 23, 25, 26, 28, 29, 92, 93, 159, 160, 162, 163, 165, 166, 168, 169, 171, 172, 174, 175, and allelic variants thereof.
Embodiment 40. The pennycress seed meal of any one of embodiments 36 to 39, wherein said meal comprises ground and/or macerated seed of a population of pennycress seeds comprising seeds having at least one loss-of-function mutation in at least one endogenous pennycress gene encoding a polypeptide selected from the group consisting of SEQ ID NO: 3, 6, 9, 12, 15, 17, 21, 24, 27, 30, 94, 161, 164, 167, 170, 173, 176, and allelic variants thereof.
Embodiment 41. The pennycress seed meal of any one of embodiments 36 to 40, wherein said meal comprises ground and/or macerated seed of a population of pennycress seeds comprising seeds having at least one transgene or genome rearrangement that suppresses expression of at least one endogenous pennycress gene encoding a polypeptide selected from the group consisting of SEQ ID NO: 3, 6, 9, 12, 15, 17, 21, 24, 27, 30, 94, 161, 164, 167, 170, 173, 176, and allelic variants thereof.
Embodiment 42. A composition comprising defatted pennycress seed meal that comprises less than 30, 28, 25, 16, or 15 micromoles sinigrin per gram by dry weight.
Embodiment 43. The composition of embodiment 42, wherein said seed meal comprises about 1, 2.5, 5, or 10 to about 15, 16, 18, 20, 25, 28, or 30 micromoles sinigrin per gram by dry weight.
Embodiment 44. The composition of embodiments 42 or 43, wherein said composition has an oil content of about 0% or 0.5% to about 12% or 15% by dry weight.
Embodiment 45. The composition of any one of embodiments 42 to 44, wherein said composition further comprises a preservative, a dust preventing agent, a bulking agent, a flowing agent, or any combination thereof.
Embodiment 46. The composition of any one of embodiments 42 to 45, wherein said composition comprises: (i) a detectable amount of a polynucleotide comprising at least one loss-of-function mutation in at least one endogenous pennycress coding sequence or gene comprising a polynucleotide sequence selected from the group consisting of SEQ ID NO: 1, 2, 4, 5, 7, 8, 10, 11, 13, 14, 16, 18, 19, 20, 22, 23, 25, 26, 28, 29, 92, 93, 159, 160, 162, 163, 165, 166, 168, 169, 171, 172, 174, 175, and allelic variants thereof; (ii) a detectable amount of a polynucleotide comprising at least one loss-of-function mutation in pennycress mutant E3 196, E5 444P1, E5 356P5, I87113, E5 543, or I87207; or (iii) crushed, ground, and/or macerated seed of pennycress mutant lines E3 196, E5 444P1, E5 356P5, I87113, I87207, E5 543, or germplasm therefrom.
Embodiment 47. Pennycress seed cake comprising 30 micromoles sinigrin per gram by dry weight or about 1, 2.5, 5, or 10 to about 15, 16, 18, 20, 25, 28, or 30 micromoles sinigrin per gram by dry weight.
Embodiment 48. The seed cake of embodiment 47, wherein said seed cake has an oil content of about 0% or 0.5% to about 12% or 15% by dry weight.
Embodiment 49. The pennycress seed cake of embodiment 47, wherein the cake comprises crushed or expelled seed of the seed lot of any one of embodiments 18 to 33.
Embodiment 50. The pennycress seed cake of any one of embodiments 47 to 49, wherein the cake comprises: (i) a detectable amount of a polynucleotide comprising at least one loss-of-function mutation in at least one endogenous pennycress coding sequence or gene comprising a polynucleotide sequence selected from the group consisting of SEQ ID NO: 1, 2, 4, 5, 7, 8, 10, 11, 13, 14, 16, 18, 19, 20, 22, 23, 25, 26, 28, 29, 92, 93, 159, 160, 162, 163, 165, 166, 168, 169, 171, 172, 174, 175, and allelic variants thereof; (ii) a detectable amount of a polynucleotide comprising at least one loss-of-function mutation in pennycress mutant Æ3 196, E5 444P1, E5 356P5, I87113, E5 543, or I87207; or (iii) seed cake obtained from seed of pennycress mutant lines E3 196, E5 444P1, E5 356P5, I87113, I87207, E5 543, or germplasm therefrom.
Embodiment 51. A method of making a pennycress seed lot comprising the steps of:
(a) introducing at least one loss-of-function mutation in at least one endogenous pennycress gene encoding a polypeptide selected from the group consisting of SEQ ID NO: 3, 6, 9, 12, 15, 17, 21, 24, 27, 30, 92, 93, 159, 160, 162, 163, 165, 166, 168, 169, 171, 172, 174, 175, and allelic variants thereof;
(b) selecting germplasm that is homozygous for said loss-of-function mutation; and,
(c) harvesting seed from the homozygous germplasm, thereby obtaining a seed lot, wherein said seed lot comprises a population of pennycress seed having less than 30, 28, 25, 16, or 15 micromoles sinigrin per gram by dry weight.
Embodiment 52. The method of embodiment 51, wherein the harvested seed of the seed lot comprise 1, 2.5, 5, or 10 to 15, 16, 18, 20, or 25 micromoles sinigrin per gram by dry weight.
Embodiment 53. The method of embodiment 51 or 52, wherein said harvested seed of the seed lot comprises the seed lot of any one of embodiments 18 to 33.
Embodiment 54. A method of making a pennycress seed lot comprising the steps of:
(a) introducing at least one transgene or genome rearrangement that suppresses expression of at least one endogenous pennycress gene encoding a polypeptide selected from the group consisting of SEQ ID NO: 3, 6, 9, 12, 15, 17, 21, 24, 27, 30, 94, 161, 164, 167, 170, 173, 176, and allelic variants thereof into a pennycress plant genome;
(b) selecting a transgenic plant line that comprises said transgene or genome rearrangement; and,
(c) harvesting seed from the transgenic plant line, thereby obtaining a seed lot, wherein said seed lot comprises a population of pennycress seed having less than 30, 28, 25, 16, or 15 micromoles sinigrin per gram by dry weight.
Embodiment 55. The method of embodiment 54, wherein the harvested seed of the seed lot comprise 1, 2.5, 5, or 10 to 15, 16, 18, 20, or 25 micromoles sinigrin per gram by dry weight.
Embodiment 56. The method of embodiment 54 or 55, wherein said harvested seed comprise a seed lot of any one of embodiments 18 to 33.

1.-56. (canceled)

57. A pennycress seed comprising a loss-of-function mutation in at least one endogenous pennycress gene encoding the polypeptide of SEQ ID NO: 21 or an allelic variant thereof, wherein said loss-of-function mutation reduces expression of said polypeptide and/or reduces transcription factor activity of the polypeptide of SEQ ID NO: 21 or said allelic variant thereof, wherein said allelic variant of SEQ ID NO: 21 has at least 99% sequence identity to SEQ ID NO: 21, and wherein said seed exhibits a reduction in sinigrin content in comparison to the sinigrin content of a control seed which lacks said loss-of-function mutation.

58. The pennycress seed of claim 57, wherein the seed comprises the loss-of-function mutation in the endogenous pennycress gene encoding the polypeptide of SEQ ID NO: 21.

59. The pennycress seed of claim 57, wherein the seed comprises the loss-of-function mutation in the endogenous pennycress gene comprising the polynucleotide sequence of SEQ ID NO: 20 which encodes the polypeptide of SEQ ID NO: 21 or an allelic variant thereof having at least 99% sequence identity to SEQ ID NO: 20 which encodes the allelic variant of the polypeptide of SEQ ID NO: 21, respectively.

60. The pennycress seed of claim 57, wherein the seed comprises the loss-of-function mutation in the endogenous pennycress gene comprising the polynucleotide sequence of SEQ ID NO: 20.

61. The pennycress seed of claim 57, wherein the seed further comprises a loss-of-function mutation in the endogenous pennycress gene encoding the polypeptide of SEQ ID NO: 6 or an allelic variant thereof, wherein said loss-of-function mutation reduces expression of said polypeptide and/or reduces enzymatic activity of the polypeptide of SEQ ID NO: 6 or said allelic variant thereof, wherein said allelic variant of SEQ ID NO: 6 has at least 99% sequence identity to SEQ ID NO: 6.

62. A pennycress seed lot comprising a population of the pennycress seed of claim 57.

63. The pennycress seed lot of claim 62, wherein said seed lot further comprises an agriculturally acceptable excipient or adjuvant.

64. The pennycress seed lot of claim 62, wherein said seed lot further comprises a fungicide, a safener, or any combination thereof.

65. The pennycress seed lot of claim 62, wherein the pennycress seed further comprises a loss-of-function mutation in the endogenous pennycress gene encoding the polypeptide of SEQ ID NO: 6 or an allelic variant thereof, wherein said loss-of-function mutation reduces expression of said polypeptide and/or reduces enzymatic activity of the polypeptide of SEQ ID NO: 6 or said allelic variant thereof, wherein said allelic variant of SEQ ID NO: 6 has at least 99% sequence identity to SEQ ID NO: 6.

66. Pennycress plants comprising a loss-of-function mutation in at least one endogenous pennycress gene encoding the polypeptide of SEQ ID NO: 21 or an allelic variant thereof, wherein said loss-of-function mutation reduces expression of said polypeptide and/or reduces transcription factor activity of the polypeptide of SEQ ID NO: 21 or said allelic variant thereof, wherein said allelic variant of SEQ ID NO: 21 has at least 99% sequence identity to SEQ ID NO: 21, and wherein a seed lot obtained from the plants exhibits a reduction in sinigrin content in comparison to the sinigrin content of a control seed lot which lacks said loss-of-function mutation.

67. The pennycress plants of claim 66, wherein the pennycress plants further comprise a loss-of-function mutation in the endogenous pennycress gene encoding the polypeptide of SEQ ID NO: 6 or an allelic variant thereof, wherein said loss-of-function mutation reduces expression of said polypeptide and/or reduces enzymatic activity of the polypeptide of SEQ ID NO: 6 or said allelic variant thereof, wherein said allelic variant of SEQ ID NO: 6 has at least 99% sequence identity to SEQ ID NO: 6.

68. A method for obtaining a progeny plant from a pennycress plant, wherein said pennycress plant comprises a loss-of-function mutation in at least one endogenous pennycress gene encoding the polypeptide of SEQ ID NO: 21 or an allelic variant thereof, wherein said loss-of-function mutation reduces expression of said polypeptide and/or reduces transcription factor activity of the polypeptide of SEQ ID NO: 21 or said allelic variant thereof, wherein said allelic variant of SEQ ID NO: 21 has at least 99% sequence identity to SEQ ID NO: 21, and wherein a seed lot obtained from said pennycress plant exhibits a reduction in sinigrin content in comparison to the sinigrin content of a control seed lot which lacks said loss-of-function mutation, said method comprising crossing said pennycress plant to a pennycress plant with wild-type sinigrin levels, selfing a progeny plant of the cross, and selecting a progeny plant of the selfing that provides a seed lot that exhibits a reduction in sinigrin content in comparison to the sinigrin content of a control seed lot which lacks said loss-of-function mutation.

69. The method of claim 68, wherein the pennycress plant that is crossed to the pennycress plant with wild-type sinigrin levels further comprises a loss-of-function mutation in the endogenous pennycress gene encoding the polypeptide of SEQ ID NO: 6 or an allelic variant thereof, wherein said loss-of-function mutation reduces expression of said polypeptide and/or reduces enzymatic activity of the polypeptide of SEQ ID NO: 6 or said allelic variant thereof, wherein said allelic variant of SEQ ID NO: 6 has at least 99% sequence identity to SEQ ID NO: 6 and wherein the selected progeny plant comprises the loss-of-function mutations in both the endogenous pennycress gene encoding the polypeptide of SEQ ID NO: 21 or an allelic variant thereof and in the endogenous pennycress gene encoding the polypeptide of SEQ ID NO: 6 or the allelic variant thereof.

70. A method for making a pennycress seed lot comprising harvesting seeds from the pennycress plants of claim 66.

71. The method of claim 70, wherein the pennycress plants further comprise a loss-of-function mutation in the endogenous pennycress gene encoding the polypeptide of SEQ ID NO: 6 or an allelic variant thereof, wherein said loss-of-function mutation reduces expression of said polypeptide and/or reduces enzymatic activity of the polypeptide of SEQ ID NO: 6 or said allelic variant thereof, wherein said allelic variant of SEQ ID NO: 6 has at least 99% sequence identity to SEQ ID NO: 6.


SEQUENCE LISTING (261 sequences)

SEQ ID NO: 1 - DNA, 1365 bp, Thlaspi arvense
  source 1..1365: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagtatggggatccaggatcctcatgttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 2 - DNA, 5220 bp, Thlaspi arvense
  source 1..5220: mol_type = genomic DNA
gaacactcgtgaagtaaaacggctcacgggccgcatcgccgcccttaacagattcatttttcgatcaaccgacaagtgtctccccttctaccaacttctctgtggtaaaaagcacttcaagtgggacaaaaaggctttcggccaactcaaggagtatctatcaacgccaccagtcctagcgaagcccgagctcggcaaaatgctttacctctacgtcgccgtatctcactcggccgtcagcggcgtactcgtcaaagaaaaccggggcgagcagaagccgatcttttatgtgagcaagtcattggacggacctgagagtagatattctacgatggaaaagctcgcactcgcagtagtaatctcagcccggaagttgcgtccctatttccaatcacactcaatcacggttctcacaaatcaaccgctgcgaacagtcctgcacagcccaagtcaatcggggcaaatgacgatgtgggccgttgagttaagcgaatacgacatcaactacaagaacaggacgtgcgccaagtctcaggtctttgccgatttcctcatagagctaccactcgactctactcccgtggactctcccaccgtcgtgcaatggtcgctatatgtcgacggctcgtcatctcgaaatggatcgggtatcggagtcagattaatatcgccaattggaaaaatcctcgagcagtcattttgccttgagtttgcggcgtccaacaacgaagcagaatacgaagctcttatcgccggcctgtgcctggcaaaagcaataggattaaggcaaatccaagcattctgtgattcccagctagtagctagtcaattcaatggtgactacgaagcaaaaaatgaacgaatggatgcctacttaaaagtaatccaaaacctttcaaaggacttcgacgatctcaccctcaccaaaatcctgcacagtgataattctccagtcgatgcgctggccgccctcgcgtcaatctccgacccaaacctttaccgtatgatacctgtaaagagcatcaaaactccgaatgggagacttggtaaaaagtaaagataaccacgaaaaaagaaaggataattatgaataataatattcataattcatttaggaaaatgttcattaatattacattaaaaatataatatttatctaaactaaaaattaattatttctcgaaagaaaaataaaatatcgagagatagaaaatagatttaaaccatcttagttagaattaaattttccaaacaggcttagtttgtaaaaatcgagtcgttataataataatcaaaatctaatacaccaaaaaacaaaggaagaaaacaactgactcgtcatagcctatagccaatttgattttggcattttgcgacccaacaaatccaaaggtaagtgggtttacctctatatataaatttgcatgaaagtaattactgaaaatagttattatataattggtgccgacaacgtcaacaaacacaattattcattacattattgtacacgttcatgctcatgcacatgtactttattgattttcacacaatacataaacttaattacttcttctcccctccgaagttattaatattacaatatcaaatacttatcacacaaattaaacagaagtcaactaatccaaaaccctttatttgatactacatagttttgcaaattagttgactcttttgaaaaccaccaaaactcgtaacgtaacgcacacgtatgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagtatggggatccaggatcctcatgttttggacaaagtttacgagtttactcaacttctacgtcctgatcattgtgacggtaacaagagcatcaggtaatttgtgaaaaatactcaatattgcttcataatataaaaatactcaatattgcttcctaatctttttggcagtttatttcactacataaaataaacccgcttttacatttttattgtttgtggtataagaatattagttcactcaaacagcatgaaactaataattgaaattttgtatttgtgtgaaaaactttagttcacttaaataacatttttgttgtttgtgttgtaaacagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgtaagtttccaacttcttcttcttcttctttttctttttttaagttgacactcacacgtactgacgtacacgttggtggatttaaaagtaaccctagtggagaagaagatgaattttcatttacattatatcataaacctactttttaaattagaataagaataattaaaactaaacccattttttattggctcactatggcctaaagaatataattaaaatattatataggctcaataagtcataatattctttagcctatagaatatttttaaagtattcaataattaaaattattttatagcatatagaatattttatgggctcaataattacaagtattctacaggatcaccatggcctaaagaataatcaaaagtaaaccgaattttaaaattacaggtatagagaaagaaaaaagaaagctaaaattaaaaccaagaataagttaaaaatgtatgagaagtaacaaacttagtggcgaaaaagagaaaaagattattattcagtcacgttcacgctcactatggaccaacttatcctatagaaactattaatattttcttgattttattcgctcttatcactttcacaagtgcatgtttgactaaaagcgttataacatgatgttttgttttcttctgatcatgatctcttgctgttacttacaaacaaaacaaatggtgattttgtttttgtttttttttgcaggcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataagttcttatttcctttgctgtgcaaatccgaaacacgtttacaaaatttacatcagttctttagttattataccaacgacaaaaaaaaaagaaaacattcaaaaatcataaggagagtaccgatggagtttctcgtgatccaaagacaccaacttggtttttgcttccgtacgtgctgactgctgagaaagtaagtgttggagtgtcccatttatacaaaaatttcataatactttcaacacatgttgcttttcagttcttttggctttttctgtagagaaacacatataatgaatatacaaagggaggaaagttggtgaatttatcacacacaaaataaaaacattaagttggtgaatcgtttccttccttttattgagtctcaatacattgtattattacctttagagtataagatgaacaccaacttaacgactaacctaaccttccttttttttttttttgaaaaacaaacttaggctatgaatatcaatagtttttaatatgagtttaatagtttttggttaaaaattttaaagttgaaaaagaagttttgaggaagaaacctaatatgagttttatagtttttgtttttgtttttaaagaactctatatagagaagttttcgaagatcgtagtggccaataaaatagttttctcaaaaactaaaacaaaaagtacattataaactacaaaatattctcagtcttatccttaattagctaactaactaaacactataaccctagttacattctaaccattgtataaccattgtataagatatacatttagaattctcagctagtgaaatgatttttgtaaagtaaagcaataatgaagagaaaaaggagaacagcacatctttaaagtaatctctttttgattgatctcggttggatccaactgtgcatttgagtactatggagcaaaaagctgacgttttaactgcatccacacatcacctcgggaatgtttctggaaaaccttctcgagataagattgaaagtagaagatgatcattgatgaataatcttcaacattgttacaaaatataagtaaagtatttacgaggtgaaattttgagtgataactagttctagtttctctttaactagagctttatctcatgcataataccaaataacttcttttttttttataagatccgtatgcc

SEQ ID NO: 3 - protein, 454 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSESMGIQDPHVLDKVYEFTQLLRPDHCDGNKSISETIQTFSEKLSELDIMVRRMVMESFGIEKYLDKHLNSTNYRLRLMKYIAPPDADATNVAADAKDADDNAKTITNDKVDAAGANDVDAGDIANGIANLHIGDDANAGANGAGVDANDGGEDAKTGEDAKTGECASVKSNAEDGTDVNASADAGVTVGSNADANANANANTSTDAGVGDSVKANGGADDVEKKLGLPSHTDKNLITVLYQYEIEGLEVLTKDDKWIRLKPSHNSFVVMAGDSLYALMNGRLTRPFHRVRVTEKKKTRYSIALFSAPTADYIIDTPKELVDEKHPRIFEPFNYNDLMSFYHSEAGRKARSTLDAFCAVSRA

SEQ ID NO: 4 - DNA, 1071 bp, Thlaspi arvense
  source 1..1071: mol_type = genomic DNA
atggctccttctgcgcaaccagttcctacaagtgtttcggatgagaaatacgcaaatgtgaagtgggaagagttagggttcgggtttgttcgtacggacaatatgtatgttgccaagtgcaaacatggagagagtttccaagagggaactgttgttccttatgctgatttccaaatcagcccttgctctgcagttcttaattatggccagggtttatatgaagggctgaaggcttacaggacagaagatggccggataatgatattccgaccagaccaaaacggtctccgccttcaagccggagccaagagactttgtatgccgtatccatcggtcgatcaatttgtctccgccgtcaaacaagttgttcttgccaacaagaaatggattcctcctccggggaaaggaacattgtatatcagacctattttgttcggaagtggtcctatacttggctcacttccggtccctgagtacaccttctcagtatttgcttgtcccgttggacgttttcacaaggataactctggcttgaacctgaaaattgaagataagtttcgccgcgcttttcctagtggaaccggtggtgtgaagagtatcacaaactattctcctgtttggataacattggcagaggcgaaagctcagggtttctctgatgttttgtttttggatgctgcaactggcaaaaacgtcgaagagcttttcgcttccaacattttcatagtcaagggaaatgttgtgtcgactccagaaatttcaggaactattttgcccggagtcacacgaaaaagtatcattgaattaactcgtgatttcggctacaaggttgaggaacgtgttgttccccttgaggatcttctcgactcggaagaagttttctgcactggcactgctgcgattgtgacaactattgcgtccgtaaccttcaaagacaaaaagaccggattcaaaacaggagaaaaaacattggccgcgaagctctttgcgacgttaatggatatccagatgggtcgggtcgaggataagaagggatggacggtggaggttgaccggtgccaccagggttga

SEQ ID NO: 5 - DNA, 6184 bp, Thlaspi arvense
  source 1..6184: mol_type = genomic DNA
ggaagttgcaacgaaagcttcagaagctcaacgggaatgttaagtgacagagacaacaacagcaacaacaataaccgagagttcgaggcggaagttgcaacattaagcaatgtaaaacacataaacgttgtgaagttgttgtgtagcataacgagtgaagacagcaagttgcttgtgtatgagtttatgcctaatggaagcttgtgggaacagttgcacgagcgtcgtggtgagcaagagattggatggcgtgtgagacaaacgatagctttaggagctgctaaagggctggagtatctgcaccatgggttagatcggcctgtgattcatcgtgacgtcaagtccagcaatatcttgcttgatgaggagtggagaccaaggattgctgattttggattggctaaaatcattcagtctgattcggttcaacgagatttctctgctcctctcgttgaaggaactctcggttacattgcccctggtacgtcgtcttttctttggtttaatgaaactatataaaccctaaaccagaacatgaactatccgaattttacaaccttataaaattatctccaagtttcttgcttctcaagcttcaaattccttgcgtttctatatacagtttcttaacttctctcagttgcaaataatttctatgacgtacacagaatacgcctacactacgagcgtgaacgagaagagcgacgtatacagctttggggtggttctaatggagttggtgacggggaagaagccggtggaggcagagttcggagaggacagagacatcgtcatgtgggtttggaacaggagcaaggagatgaacagagagaagatgatggagatcatcgatccgattatacaaaatgaatacaaagaggatgctcttaaagtgttgacaattgctttgttatgtactgccaagtctcctcaggttagaccgtttatgaaatcagtggtttgtatgctggagaaaacagagccttcttgcaacaacaaaagtggagaagcaagttacgttgtgagtgatgatgaggagattactgatgtttaatagctcatagattgaacaaactcacagaagggttacttgtcaatctagctcatgtctaaggaccaagaatataattttttttccaaatagttataaaattatacagaacttttaggaattgttctaagattatacaagagtttttttttttttttttttgccagcggaaagtttcaatatttccaagttcttagaaatataatccagttatccaaataattagtatctatataaaacaaatttgagttgttcacaaagatagtttggccgagtggtctaaggcgccagatttaggctctggtccgaaagggcgtgggttcaaatcccacagctgtcagaattttatttcatttgacttataaatttttcgggtgctcttccttcaacttttcaatgttcacgtagttcgtcattttcctcatcggcggtatggatgctggacggtggagattcgtcttcctcctataattagttgcctgtatttggaaactaattgaaagaaataacatgaagaagaaaaaaagaaagaaagagtggtggatatacatgtacatgagaaagaaatagaaagtcaatgaaaaatcgaggaccgaaggggaataatataattcgaaaaaagtatatgtattatgtatagacatgtaaggtgtaacgtactaacatgttggtgcatgaataaaattttatttagtaaaaatggatacaacttaaaaaaaacagaaatttgggggaaaaagttatcacaattatgaagaacaagttactagtatatatatattaatgtttttcttttagaaaacaaaagtattcttatttttaagacttttaatttagaaaaatgattaccttaatacaactggatgaattcgtattatttattcaaatggaaaactacacaagaacaaatactatagttttgaggaagaataaaagaaaactgaaaaggcatgaatgtttatttatattattattaccaacaactttttaaaattattacatcacaagggcatgacataatatgataaacacagagatccagagacatcttcagatgttttcttagctatactatagtatgaaaacacagagatccagagacatcttcagatgttttcttagctatagtatttatataattgtcaagttatacggcttcaagttatggctccttctgcgcaaccagttcctacaaggtaaaatatatcattgcaaatattataatagtctcgcatttgcatgtttaaatctgttactatgatctatgatttgctgatgatagtttcttttacagtgtttcggatgagaaatacgcaaatgtgaagtgggaagagttagggttcgggtttgttcgtacggacaatatgtatgttgccaagtgcaaacatggagagagtttccaagagggaactgttgttccttatgctgatttccaaatcagcccttgctctgcagttcttaattatggccaggtttgttattaatcaatagagatcaaacttattaagaaaaatttatatcatcagaaagaagatataaccatcaccatatatagaactaatatcgttttagcattgcatgcatatttgagctattagacatttaaaattttgaaaagataatagaatataaatcaaacgaaatgatttaaaagtgttaatatgatgatattcttatcaagtaaattgtttcatcaaataattactataatatgtttcaattcaaagaaaatctagatatgtataacaacataagtagtctaactgcccccattaaaatgaactaatgagctttcttaaaatatttatgaagaattaaattattagatgctagattgagtgatgcggaagcaaagtagtctgaaatatttgtaatttggaaataaatagggtttatatgaagggctgaaggcttacaggacagaagatggccggataatgatattccgaccagaccaaaacggtctccgccttcaagccggagccaagagactttgtatgccgtatccatcggtcgatcaatttgtctccgccgtcaaacaagttgttcttgccaacaagaaatgggtatgtacaggctcggttccaaaagaaaaaaatctggaccgctcaaaaaaaccaaactgtgttctacataaattctaaattttgaaattatggtcgtgttaggcataattgtaaaagaaaaaaaaatgctttagaatgcttcaaaaggcaaataaatgtaatttctatgcatttttcaagatattaaatcttaacaaatgtatatattttaaaaattatattaatgttcttctttgaattttatatatatacagattcctcctccggggaaaggaacattgtatatcagacctattttgttcggaagtggtcctatacttggctcacttccggtccctgagtacaccttctcagtatttgcttgtcccgttggacgttttcacaaggtgagtgttcttgtcaatatatattatttttattttagtttaaacagtaatagactagagcaattttttggaatgattttgattataatatgttttctggtataggataactctggcttgaacctgaaaattgaagataagtttcgccgcgcttttcctagtggaaccggtggtgtgaagagtatcacaaactattctcctgtaagtttgaaaacgtacatcaaatttgatattaacatgattagctgtgttttagatttttgtatgaacgattaatagttaattaatataacggataattttttcttaatgaaggtttggataacattggcagaggcgaaagctcagggtttctctgatgttttgtttttggatgctgcaactggcaaaaacgtcgaagagcttttcgcttccaacattttcatagtcaaggtaattatatatataaatatatatatatataatacgttttgaaagagttttaaagaatattaagatcgatgtgatatggtctgtatagggaaatgttgtgtcgactccagaaatttcaggaactattttgcccggagtcacacgaaaaagtatcattgaattaactcgtgatttcggctacaaggtttgaatatttcaataccatgatattataattttttttttttttgaaaaagggtttcatgatattataattttatccatgttgattttgtaaataatctagttttttttccttggattcttattataataggttgaggaacgtgttgttccccttgaggatcttctcgactcggaagaagttttctgcactggcactgctgcgattgtgacaactattgcgtccgtaaccttcaaagacaaaaagtaatgttttattccacatctaatcatttttctcaacaaaataagaagaaaaataaaacaaatacagaataactataccatgatttaaatctggattttgctttattctcatatataagtttgactaaaaattgggtgtacatattcaatgcgtacaggaccggattcaaaacaggagaaaaaacattggccgcgaagctctttgcgacgttaatggatatccagatgggtcgggtcgaggataagaagggatggacggtggaggttgaccggtgccaccagggttgaggatttgcggtttgagtagagttgctttttgtgtaagaaactttgagttgtctataaagctttggactgcttctctttatatattcgttcacaacaatgtttattatattaaaaatagtaataatttaggcgaaaataataaggatattttctatccatctttttttgttgttaaagcttttttatccattgttttaagcaattaacttttattctattctattatatcagttcaaagaataggctttttcttggcacaaaataataggtttcaaatgtacatatatacaacatatatttgttaattgataatcaaatgtatctcagtgatccgattaagtttatatatgcctaatgtttttttatctatacgtttatttaatattaaccttatagctctgtatttttttttttgatgtatttatatatgactaacttttggttggttatttcaccaacttatttgtttagagaaaaataaattgggaactctagttagatcacagaataatcatcacgtggagaaacccatttgtttctcgtcacgtggagaaaacgttaagctttaaaattttctttttaattattattatctcgcgggtatggctggaagaaagagagtgtttaaaatgtgaatgcgctcttagttaggtgaaggttaataggtaggagggtaggtcaaatgtgtatcagtagtgatggataaaaaatttaaactgtagataatttctaacaaaaaaacatataataaattacattttcacttttcagtatgccacaaacctatatatgattcaaaaagcatttttctactcagaaagctgagagaagtaacaattttggttattagcaaaaaaattcgttcatgttttttcttttcttctgtcagtgtatttgtatgctcaacttctagtcttgattataccgtagtatgccactactaattgttgttttcttcatattgcaaaaacacttaaaattgcaaatatcggaccaataagcaaaccccaaagtactttaaacgaccactttctttgtttttttattattagacattcaaagttgattgtttcttacttaacctaaacttaggcagataaaatattcttgaatatagatccatgacttgagtcactactgcaacgaaggcgtctttagtttttgagcgaagtcgtgagagtttagcttctcattcatcactctgaatttctcttttatcctctttatctgttcaaaacattaaaaacaaaagtatgttattagcataaagctgtctcatacttggttatacgtagaccatatttagtttttcaatagcaaatacaaaagtaaagcatgatcataagattcagggtcaaggtttggtttacccttctcagctcgatctccgtgcttcgtttcttttgatcaagtgattgccggagattcgtgatgtcgaaaatactatcaaggtcgtcttcaaatgcgttttccaactcttcccggagaagagcaggtaacttatcgacaataggcatcaggagaaaacagtt

SEQ ID NO: 6 - protein, 356 aa, Thlaspi arvense
MAPSAQPVPTSVSDEKYANVKWEELGFGFVRTDNMYVAKCKHGESFQEGTVVPYADFQISPCSAVLNYGQGLYEGLKAYRTEDGRIMIFRPDQNGLRLQAGAKRLCMPYPSVDQFVSAVKQVVLANKKWIPPPGKGTLYIRPILFGSGPILGSLPVPEYTFSVFACPVGRFHKDNSGLNLKIEDKFRRAFPSGTGGVKSITNYSPVWITLAEAKAQGFSDVLFLDAATGKNVEELFASNIFIVKGNVVSTPEISGTILPGVTRKSIIELTRDFGYKVEERVVPLEDLLDSEEVFCTGTAAIVTTIASVTFKDKKTGFKTGEKTLAAKLFATLMDIQMGRVEDKKGWTVEVDRCHQG

SEQ ID NO: 7 - DNA, 1080 bp, Thlaspi arvense
  source 1..1080: mol_type = genomic DNA
atggctccatctgtgcagccttcttcatcacctcttcgtacaagtgaaggagatgaaaaatacgcgaatgtgaaatgggaagagcttggattcactctgactccgacagattacatgtatgtggcgaaatgcagacaaggagagagtttttcagaaggaaagattgttccttatggggacatttcaatcagcccttgttctccgattctcaattatggccagggactatttgaaggtctcaaagcttacaggacagaagacgaccggatcaggcttttcagacctgaagaaaacgctcgtcgtatgcaaacaggtgcggataggctttgtatgacaccaccttctctggagcaattcgtcgactcagttaagcaaaccgtgcttgccaacaagaaatgggttcctcctccgggtaaaggagctttgtatataaggcctttgctaatagggagtggcgctatacttggagttgctccatcacctgagtacactttcctcatttacgcatctcccgtaggagattaccacaaggtaagctcaggcttgaacctaaaagttgatcataagtatcaccgagcccattcgggtggaacgggcggtgtcaagagctgcactaactattctccagttgtgaaatcgatggtcgaagcaaagtcgtcgggtttctctgatgtcttgttcctggattcggcaactggtagaaacatcgaagaggtttctgcttgtaacatctttattgtcaaggggaacattgtgtccacaccaccaacttcaggaaccattttaccaggaatcacaaggaaaagcataatcgagctagctcgtgatctcagctaccaggttcaagaacgtgatgtttctgtggaggagctcctagaagcagaggaagctttctgcacgggaactgcagtggtcgtgaaagctgttgaaactgtgaccttccatgacaagaaggtaaagttcaggacaggagaagcagcgttgtgcacgaagcttcactcgatgctgacgagtattcaaacaggtcttgttgaagataccaaaggttggatggtggagatcgatccttgtcaaggttga

SEQ ID NO: 8 - DNA, 5021 bp, Thlaspi arvense
  source 1..5021: mol_type = genomic DNA
atgtccaatttctctactcctttcacctcaaaactaagcttttcaaccaaaaccgagggattagacccgttcccatgatcatttacaggaggataaccaacctcaatagttacaaaaatggaatctccaggcctgtacacatctttatccgtttgcacacttagagtcggtttgagattgcttccaggaacacccgaagagtcattaacgtcgctagtactgccgatccccaaaaatgaaaaccttgatgataacatagcttcaacggctgtgttccttcacagatataactagcaaagcagagaagctgactagactaaatccctgagaacagtttgattaggcataaagcctgcgaaattgacgaaagaagagtttagagaacagatgagcaaccctttgaaggttatagaaaatgcctcaaacgtagaactgagagcaagtaaaaagagtcataaggtacaaaagaaagactcgcaatccagcgtgtttcgaaatatcgatcatagatccagagaatgtgcagaggcccagaaattagggtttctagggagaagaaggtgacttacacgtgatgacgagaatgatcacgtgagttggaggagcaagaaaaccagattcaaggttcgtcaaattggcagcaagtagcttaaagaatggcaatcaaatccagagagaagacgacgcggatcgctaagatcataaaaccaattccgaacagatgtcgctcacagtcagattcacagagactgagcttctaaaaaaaaacagagacaccggaaaaaagtcttcaaagccgagagtccggcaacagaatcctccgccgtaagaaacggggaaatgtccagaaaagcttaaaggttaagttttctgcagtaaaccggaaaagatgaattcagcttctcgagagcaaaataacgaagcagctagtgaggtgatccagctctatatttcccctctcctaagcttctctggtgcttcaaaattgccagtggtgtccttcttttggattttatcaggttgaaattagatggatctggttgccgttgaaggtttctctcgtcgggaaatctggatcgaagggacgattgaaacaaaatgcttccccatctggtttcggcaaatcttcctcagcttcttcttcactcggcaaaaattatggtcctcttatatgttattgggcttctacttgggttcaatggttctgttgggtcctttgaaaatttaatgttcaaaaaaaaaacacaagtatatataacgtcgaaaaaaacaaatagagccatggtttagtttcaacaatgtctcataaattaaagaccggtcacatcttttttttttttttgaaaataagaccggtcacatcttttcaacaaagaacggcaactttagtttatttagtctttagccatcagcctttttatagtttgaagattctacgagtcatgtgtgtgtaaagggaatataaacaaaatacgaaagacaaaaggaatcaaagggtttgtttgttgtcgctataaaacgacgcgtgataagccgctcctaaagcacgtgaccgctctaagcctctgcttcgcttcacttcacttctctctctctctttgtcttcctctttccatggctccatctgtgcagccttcttcatcacctcttcgtacaaggttaataaaatcatcaacctttcatgaaaatttattggaaaaaaaagatttctttttttcattaaagtcgtgaatctcctgatctgggtatctcgtgtttgttcctgtcgtttatttataactattttattcatttgtgtgatttttataacttttggtggcatgcattgaaatttaaaacagtgaaggagatgaaaaatacgcgaatgtgaaatgggaagagcttggattcactctgactccgacagattacatgtatgtggcgaaatgcagacaaggagagagtttttcagaaggaaagattgttccttatggggacatttcaatcagcccttgttctccgattctcaattatggccaggttttgttcttacaatcacttatatggttgtgcaagcttgctgaatctcactggattataggacttttgggtttcattagagcatgattcattaactagtgagggcgcaaaatgatccgcagattgcgtaaagctgttttatttgagtgtatgagcgagcaaaagtttagaactttagtgtttatgtacatgcatgtgaggatttgagagaagaccttgcgatgattcattagttagacgctttcatcaaaaactattgaggggcgaaaaatgaagattgtgtaaagttgtttgctttgagtgtataaaagagcaaaagctttgaactttagtgtttgagagaaaactttcctctatgcgtttggctttgtagggactatttgaaggtctcaaagcttacaggacagaagacgaccggatcaggcttttcagacctgaagaaaacgctcgtcgtatgcaaacaggtgcggataggctttgtatgacaccaccttctctggagcaattcgtcgactcagttaagcaaaccgtgcttgccaacaagaaatgggtatggactatgaactatggaccaatgtctctctattcagatatgattcttgatctctacaagtttctaaaagtgtgttgttatgtttttttggtttttttatttggttttaggttcctcctccgggtaaaggagctttgtatataaggcctttgctaatagggagtggcgctatacttggagttgctccatcacctgagtacactttcctcatttacgcatctcccgtaggagattaccacaaggttagattttttgtgctgctgtttttgaagcaaactctgccaaaaagactcgttaaatggttaagccggttttttctcaggtaagctcaggcttgaacctaaaagttgatcataagtatcaccgagcccattcgggtggaacgggcggtgtcaagagctgcactaactattctccagtaagtaaagagacaccagaaacatttcatccatggcaacaggtttagggaatatatatatgtttttctctgatttcttcttgtataaattttgaaaaggttgtgaaatcgatggtcgaagcaaagtcgtcgggtttctctgatgtcttgttcctggattcggcaactggtagaaacatcgaagaggtttctgcttgtaacatctttattgtcaaggtaaataatgagattcaaaggcttatcacatctaatactgaagatttttatacacttcgtagtaatcattcaaatttttaggggaacattgtgtccacaccaccaacttcaggaaccattttaccaggaatcacaaggaaaagcataatcgagctagctcgtgatctcagctaccaggttgcaacaatttttaattcactctatatagttataccctttcatatatatatatatatatagactctgaggaggtagttcacaatgtgatcacaggttcaagaacgtgatgtttctgtggaggagctcctagaagcagaggaagctttctgcacgggaactgcagtggtcgtgaaagctgttgaaactgtgaccttccatgacaagaagtaattagtttcttctattttgcagatataaagaccctaacaatcaaatctttcttgggttacatagtgctgatacctcgggtcatgtctgcctttgaccacagggtaaagttcaggacaggagaagcagcgttgtgcacgaagcttcactcgatgctgacgagtattcaaacaggtcttgttgaagataccaaaggttggatggtggagatcgatccttgtcaaggttgaaaagctgttgcaaaattcgcttactcgttctgtatcgtttcttttctcttagtgttttcttttatgagacttgtaatgaagaactcttaaaagataaggtacaacaaggtcgttcaataagtgactaggattcgtcccggtccatatcatttgctttgttgttctccactgtagcctagtagttaaaatgcataaaacccatgatccaaaaaatgattcaatgaaccaatctaaatttgtaagtttcatatattggttgagtgatgtagcagaaatctacaaagtttcattcgtacaaatacaaacttaaaggacgggagaatgaaagatgtttgggcaccgaccaatcggttcaattactgttttgactcgaatgaaaaaagatatattttttaatttttcacttttttagaaatcgaaaactaaaaaaaaaaaaatcattgacagctgtgggatttgaacccacgccctttcggaccagagcctaaatctggcgccttagaccactcggccaaactatcattgttgacaagtttttagtaatgtttaacttaactaaacatgtactatcttcggtatttttgcttttattattattagggaagattacacatatagcacaaatttattattttattactagattagcttttaattttttttttactgactaaatttttgtgtccgtaatatccattaatcatttgttatgaaaaatcatatttacaagaatgccattattatttttttctaactcagcaaaaaatagttaataattacgaaaaatgccattatattcgaaaaaatctattgtaccagaactgctaatgtatgatgacatacttaattttataaacatacttatttttagcagtacacttttgtcacagatttattcagaataatagacttatatcaataagtctctcatatgaagcaatatacttttttcagagaaaaggtatttttgctgataaacttatttcttagacttgttatgttttgaccatagatttacttatttttaataaactaattttttgtaatagacttatttcacatacttatattttgcacgatagactaatatgtatatgagtgataactgataacagatttaaattttacgttctgaatgatgatagacttatcgttagttttgtattagtctgctatcattaaaagacttattttattt

SEQ ID NO: 9 - protein, 359 aa, Thlaspi arvense
MAPSVQPSSSPLRTSEGDEKYANVKWEELGFTLTPTDYMYVAKCRQGESFSEGKIVPYGDISISPCSPILNYGQGLFEGLKAYRTEDDRIRLFRPEENARRMQTGADRLCMTPPSLEQFVDSVKQTVLANKKWVPPPGKGALYIRPLLIGSGAILGVAPSPEYTFLIYASPVGDYHKVSSGLNLKVDHKYHRAHSGGTGGVKSCTNYSPVVKSMVEAKSSGFSDVLFLDSATGRNIEEVSACNIFIVKGNIVSTPPTSGTILPGITRKSIIELARDLSYQVQERDVSVEELLEAEEAFCTGTAVVVKAVETVTFHDKKVKFRTGEAALCTKLHSMLTSIQTGLVEDTKGWMVEIDPCQG

SEQ ID NO: 10 - DNA, 1551 bp, Thlaspi arvense
  source 1..1551: mol_type = genomic DNA
atggcatcaatcactttactgggccgaatactagcaagacccatcaaaaccaaagaccggtctcgccagcttcctcctggtccacgaggatggcccatcctcggcaatctacccgaactcatcatgactcgtcctaggcacaagtatttcgcccttgccatgaaagagctcaaatccgagatcggatgtttcaacttagccggagtccacgccatcatcataaactccgacgagatcgctagagaagcgtttagagagcgagacgccgacttcgcggacaggcctgatcttttcaacatgaggaccatcggagacaatcacaaatcaatggggatttcaccttacggtgaacaattcatgaagatgaaaagagtgatcacaacggagattatgtccgttaaaacattgaacatgttgatggctgcgagaaccatcgaagcggataatctcattgcttaccttctatcgatgtatcaacgaggcgagacctgcgacgttagagaattctcgagggtttatggttacacggtgaccatgagaatgttgtttggaagaagacatgtcacgagagaaaacattttttccgatgaaggaagactaggaaaagctgaaaaacatcatcttgaggcgattttcgaaaccctaaactgtttgccgagcttttctcctgcggattacgtagaaaaatggttgcaaggatggaacgtcgatggtcaagaggagagggtgatagtgaatagtaatattgttcgtagttacaacaatccgataatcgacgagagggtcgcgttatggagggagaaaggtggtaaggctgctgttgaagattggattgatacgttcattacgctaaaagatgaaaacggaaagtacttggtcacgccagacgaaatcaaagctcaatgcgttgaattttgtatagcagcgatcgataatccggcaaataacatggaatggacacttgcggagatgttaaagaacccggagattctgaggaaagctgtgaaggagttagacgaagtagtgggaaaagagaggcttgttcaagaatcagacataccgaatctaaactacttaaaagcttgttgccgagaaacattcaggattcacccaagtgctcattatgtcccttctcatgcggctcgtcaagataccactctcgggggatatttcattcccaaaggtagccacattcatgtaagccgtccgggactaggccggaaccctaaaatatggaaagatccattggtattcaaaccggagcgccacctcgaaggagacggaatcacaaaagaggtttctctggtcgagacagagttgcgtttcatctcgtttggcaccggtcgacgtggctgcatcggtgttaaagtcgggacgatcatgatggttataatgttggctaggtttcttcaagggtttaactggaaactccatcctgcttatggaccgttaagtctagaggaagatgatgcattgcttatggctaagcctctgctcttgtccattgagccacgcttggcaccaaacctttacccaaaattccgcccttga

SEQ ID NO: 11 - DNA, 4847 bp, Thlaspi arvense
  source 1..4847: mol_type = genomic DNA
gatattatccctaatataaattgcttcccgctttcctgaaatatgttgatattcgattgcaaacgtttgttgtcgattatccatatatagcgtaattgctttatcgttgtactaatagttatttttgagtatttggtatataaaaaaaaaaattgtgtaagaaaataataaaaatggtttcttatagccgcttcggatttccaaaaaggcaagaagggctctttgtatgtatcatatatgcaaagatggttacaacctaaatcagtgtaagttaggtggatattgatttactgcatgtgatgcactaattctatctgttaattgttcagaataagatgtgtttttaacgtatactatatagttggacttggatcgaatcttcttttttttttttcctttttggatttcttgttgttcagttttggtttgtttagaatcgtaaatttgactcagtttgacttttgattttctcaaaccacggacacagccacaggacgaaaaacagaaagaaataaaactacatcatgaatccaaatgatgaaagtaatttccattgaagctgacgacactcatggaaattcgatagagaacatctaaacgtggttacctacccaagagctcccatctagtttctattccaaaaattcctttattctgaatcactttttgctttctctctctctttcaagtgttgcacattttacatttattctttttttttcattttcttttaataagaaactactactataatattaatgtagacaaattcttaactaatatcatttatttatctttttttcctttcaaaacaaacatatatcatttagtacatttatgaaaacaccattagattgatgtactttatgaaaatatattaaactatcatattaaaacatatcctacttaagagttttaggaatgattttcaatcatcagtctcaccaacataatttgcgagtagggatcggattaacctatgggacctcctcgatccacctcatatgggaatgggagagcctccagtctccacccaaggactcaagtagctagatagtaagttagatagtgtgatacaagactgtagtactgtacgctaggcacgtttgttacgtgtgagtctgtgacagtgctctagttttgctacccattctacgtacctcctacgtaatggcgctaagtggcagaactatctgcctcagaaactagttatcattatttttcttctattttgctaaacatgcttagaaattttgattttatttagttaacaaatgtacaaattgaaatgaaacaataaagcttttcctattaaacatgattagccgctgaaaattagatacaaaatatataaacgtgatttccttacagctagtacctacccataagcccaaaccccacacgtgagcatcgcatgcgcatgaaatcgaatctataaatacttgaaacacaagacaattccccaatatcaaattatcgctagtgttcattgctaacgagcacacagctctctctacacagacacacaaacatgatgacgatgctgaggagccttaccacatcattaccatacccttttcaaatcctactagtctttatcatctccatggcatcaatcactttactgggccgaatactagcaagacccatcaaaaccaaagaccggtctcgccagcttcctcctggtccacgaggatggcccatcctcggcaatctacccgaactcatcatgactcgtcctaggcacaagtatttcgcccttgccatgaaagagctcaaatccgagatcggatgtttcaacttagccggagtccacgccatcatcataaactccgacgagatcgctagagaagcgtttagagagcgagacgccgacttcgcggacaggcctgatcttttcaacatgaggaccatcggagacaatcacaaatcaatggggatttcaccttacggtgaacaattcatgaagatgaaaagagtgatcacaacggagattatgtccgttaaaacattgaacatgttgatggctgcgagaaccatcgaagcggataatctcattgcttaccttctatcgatgtatcaacgaggcgagacctgcgacgttagagaattctcgagggtttatggttacacggtgaccatgagaatgttgtttggaagaagacatgtcacgagagaaaacattttttccgatgaaggaagactaggaaaagctgaaaaacatcatcttgaggcgattttcgaaaccctaaactgtttgccgagcttttctcctgcggattacgtagaaaaatggttgcaaggatggaacgtcgatggtcaagaggagagggtgatagtgaatagtaatattgttcgtagttacaacaatccgataatcgacgagagggtcgcgttatggagggagaaaggtggtaaggctgctgttgaagattggattgatacgttcattacgctaaaagatgaaaacggaaagtacttggtcacgccagacgaaatcaaagctcaatgcgttgtaagtaaaaaaaaaatttatttattgttgatcatcatcactaattagatctatatttactgaataattaataatttaatatgacgatcatcgaaatatatattactgatgattctactgaaaatggttatgacttatgagtcatgagaactttaaaagatctgttgcagccatatttttacagatgattgtttggatactacttacaaagttataattaagtaacttaaaaatcattttaaaagtacaaatatttagtaatacaaaaaatggattagaaacaaaaaaaatggcatctattttgatatttttacgtgacgaatcaacttttgatcattgtttggtgttttgttacaggaattttgtatagcagcgatcgataatccggcaaataacatggaatggacacttgcggagatgttaaagaacccggagattctgaggaaagctgtgaaggagttagacgaagtagtgggaaaagagaggcttgttcaagaatcagacataccgaatctaaactacttaaaagcttgttgccgagaaacattcaggattcacccaagtgctcattatgtcccttctcatgcggctcgtcaagataccactctcgggggatatttcattcccaaaggtaaaacaaactgtgttttttcgtaggattttgcatttttatcactagcaagtcaatacatgcatagtttagcttaacatgtagttgagccaccaataattcgatatgatacttatattaaaaaagtatcggttttggacaggtagccacattcatgtaagccgtccgggactaggccggaaccctaaaatatggaaagatccattggtattcaaaccggagcgccacctcgaaggagacggaatcacaaaagaggtttctctggtcgagacagagttgcgtttcatctcgtttggcaccggtcgacgtggctgcatcggtgttaaagtcgggacgatcatgatggttataatgttggctaggtttcttcaagggtttaactggaaactccatcctgcttatggaccgttaagtctagaggaagatgatgcattgcttatggctaagcctctgctcttgtccattgagccacgcttggcaccaaacctttacccaaaattccgcccttgagaaaagaacaagactccttgttgtttctctgttctgttctacaacgctttgtcattactatgttatgttactttacttggcacccagtcgtttgttgttgttttagactatccttggctaaatatcgccatcgtttcatttgtgtattttttttttctttttctatcactgtaacttaataacaatcaaacattccgagtcattatacttatagccaataagtgaggatcagtttaataaagttgagattgttatagattataatattaaatggtgaatccttacgtagacaatattaaaagtcgatggctcgtgccatggttataaatattaacaatactttaagctattaaatcagttagtacgtagacaaataaaaattaaaccgtttaatagagaaggtcggtgcatggccggatgtgatggtgcggctcattttcttataaatatcattaatattattctgttcatcccgcatttgttttacccgttttacaccatcttcattcgaaacctcaactcttcagtcttcaatatggagtataatataacgtaaaactatctaccataattattttcaaaaaaaaaaaaaactatctaccataatcatcctatattaaatttttatatgttaacattatataataatgcattaaaccattaactctctaatatgtgtttgccaaattaagttcactttcgaacagattaaattatcttttcatttgaccctcttatactgaaaattaattattttggttatatttctgtaaattttgtaagacgttaatgcaccgtaagtctctcaaggagaatgaaggatatgcaatgtatattttattaatataaagatattgaactaatcacataatgatacatggtcaaatacgtttttttaagatttcttaaacaagaattttgtctctcttctccttttttattaatttatatttttaattgttagaaattacgttagatgcaactgatggaaatattcttagattctagagatgaatttgcttctgactaccgaaatggaaagatagaggcaggaaagccagtaagaaaatttcattggaattggtctgtcagatgagttaataaagcaatattgttgacttttttttttttttttgaaagggtatagttctgagttgtaaaatggattgaatcaaaagtttgatgatagac

SEQ ID NO: 12 - protein, 516 aa, Thlaspi arvense
MASITLLGRILARPIKTKDRSRQLPPGPRGWPILGNLPELIMTRPRHKYFALAMKELKSEIGCFNLAGVHAIIINSDEIAREAFRERDADFADRPDLFNMRTIGDNHKSMGISPYGEQFMKMKRVITTEIMSVKTLNMLMAARTIEADNLIAYLLSMYQRGETCDVREFSRVYGYTVTMRMLFGRRHVTRENIFSDEGRLGKAEKHHLEAIFETLNCLPSFSPADYVEKWLQGWNVDGQEERVIVNSNIVRSYNNPIIDERVALWREKGGKAAVEDWIDTFITLKDENGKYLVTPDEIKAQCVEFCIAAIDNPANNMEWTLAEMLKNPEILRKAVKELDEVVGKERLVQESDIPNLNYLKACCRETFRIHPSAHYVPSHAARQDTTLGGYFIPKGSHIHVSRPGLGRNPKIWKDPLVFKPERHLEGDGITKEVSLVETELRFISFGTGRRGCIGVKVGTIMMVIMLARFLQGFNWKLHPAYGPLSLEEDDALLMAKPLLLSIEPRLAPNLYPKFRP

SEQ ID NO: 13 - DNA, 1854 bp, Thlaspi arvense
  source 1..1854: mol_type = genomic DNA
atggagagaaagccctttgaggttgagacgacggagaatcacaaaccctactccaccgtcgatggcggtggcgttggttctgatttgagatcgccggtcgattcatttgatgacgagcagaaaaagctcgtttacagaggctggaaagtcatgccttttatcattggtaatgagacatttgagaagattgggatcatagggacattatcaaaccttcttttgtacctaactcaagtattcaaccttaagaaagttacagctgcaacaatcatcaatgcctttagtggcacaatcaacttcgggactttcatcgctgctttcctctgcgacacttactttggtcgctacaagactctcagtgtagctgtcatcgcttgtttcctgggatcgcttgtgatattactgacggctgcagttcctgcattgcacccgactccatgtggaacacatagctggtgccaagggccaagcccgggccagatcgcgttcttgctgctgggtttagcgtttcttgtggtcggtgcgggtgggatcaggccgtgtaacttggcttttggagctgatcagttcaaccccaaatccgaatccgggaagaaaggaatcaacagcttctttaactggtatttcttcaccttcacgtttgcgcagatcgtctcgctcacgctggtcgtgtatatccagtcgaacgtgagctggacgatcggtttgctcatccctgtggctctgatgttcttggcctgcgtcatcttctttgctggacataaactgtatgtgaaagtgaaagcctcgggtagtcccttggctagtatcggtcacgttatcacggcagcgatcaagaaacgagggttgaagcaagttaagcagccttggctcgatctttacaaccacattccaactaactatccaaactccaccttgaaatacaccgaccagtttaggtttcttgacaaagcagcgattatgacccctgaggacaagctgaattccgatggagctgctttcgatccatggaccctatgtacattgcagaaagtggaagaagtgaaatgcattgtgagagtgattccgatctggtttgcttgcgcgatttactacctcactgtaactatacagatgacttatccggtcttccaagcgcagcagagcgaccggagattgggttctggtggcttcaagatccccgcagccacctatgtggtgttcttgatgtcgggtatgactgttttcatcgtgttctacgaccgtgtccttgtcccgttgctcagaagagtgaccgggttagaaaccggtttgaccctcttgcagagagtcggatcagggatcttctttgccatgttgagtttgttggtctccgggttcgtagaggaacggagaagaaccttcgccctgacgaaaccgactctcgggatggagccacgagcgggagagatctcctccatgtcggccatgtggctgattccgcagctcttgcttgcaggcgtaggagaggcttttacagccattggacagatggagttttattacaagcagttccctgagaacatgaagagcttcgctggctctatcttctatgtcggtgcaggtgtttcgagctatcttgctagcttcttgatctcgactgttcatcgaagaactgaacattcaccctccgggaactggttagctgaggatctgaacaaagggagactcgattacttctacttcatgctcaccggaatcatggtcgttaacatggtttacttcttgataatgtctaaatggtatagatacaaaggcattaacgatgaagcgaattctttggtcgagaccaatgaagaagagaccaagcagaaacaagtcaagaattctgtctga

SEQ ID NO: 14 - DNA, 5463 bp, Thlaspi arvense
  source 1..5463: mol_type = genomic DNA
actccgggcgcgcatccggactctggagattatggcggcaccacgagtaagaccggatctgctatcgtgctagggttcgtgatctcgtggtttccgagtcccaagagacagaggaggtggctgagattggattctctcccataatctttctccttagcggctcatttcctgtgtcccagacgcctttgcgatcctccgtgcgatggctcttcttctgatgcgcctatttggtcaaactcatggtttttgcccttttcttattttttacactcgcagtttgtggtgggtgcatgctttcaaacctacttgtagagaccttgttgtaccccttgttctttcaggatttattatataaataaacaatgactttttcatgttttatttttattttttagtcctagaactttgacatgcaccttaatcgtacatatacgactaaactcgtgcttcaatcgaacagactttctaatctggagtgagcaccataaatctttgctataccttatgaatgaactgaacttctgtgcgtatttttagtgtgctgcggttttccagaaaatgggtcaatgtatgactcgactactgggatccgcatgctcttgttgtctgttttatctgcaggaaacaagcgtttggcctgacacatacgttcctgtgacagcgataatcgttgctctgaataacaggcacacagtatgagtgctcttgactgggtggagctttggatgtcttcaagggctggctttgcttcgcttaagtccctcgaaatgtgcatcttcgggatggcgaccttctagggaaagttgcttctgagcctggtggccgagcccatctccgggagcgaggtttcctctccgctctactgctcctttgttcttctgccatgtggcaccccagaacgactgttgttgcctctgaccgccgtggattttaacattttcggcgattaattgcttgactacttcttttgtggtgttgtatatcaatatttctcttttcttcttctaacgataattattaagacataaattataaaatccgtacagaagtatgtgtttgtctaaatgttagaaattacgttgttttcaagtttcatcttctctaacttctcatatctaaacaaatatatatctatatatattctgttatgccaaataaatatatatttagtttgtaataaaattatatttatttatattatttattcatacggtatatatgataattatgttaaaatattatatcatattataaaaagttttaaaataatattaaaattaaattgacttacattattttaatattatatatgatttattaatattatctgttaataaattcatataatttaatttatttttttaaaaataaatttagtaatatatttaataaacatcatggtagattttagacattttataaagtagggtttgtcttttaatagtaaaaatcagttaaacagatcggaaaaatgagtcattcacacatgtctctatcctccacagccaaattgaccattgtttgattttccccttttgtccagattcggacaattcccactggatccatccactatataacaagacgatagagcgaagcttgagatgaagagtagagtcatcctcagccatagagagagaagagataagaagaataataacattaacaacaaagacatctcctgtaatttcacacagattgaaaccatggagagaaagccctttgaggttgagacgacggagaatcacaaaccctactccaccgtcgatggcggtggcgttggttctgatttgagatcgccggtcgattcatttgatgacgagcagaaaaagctcgtttacagaggctggaaagtcatgccttttatcattggtaacgctttttttctctcttcagacatcgtgtttttttttttttttttttttaattacactctgttttgtgtccctttgtcggattttaaacacctcccggagaaaaagaaatgagaaagtaaaattcaacattttgtccgtgatttgtttaatttcattctctttttctcttctctttgatttgacctgactgtgtcaaaaatcgtcgtgctttgagatttagaaatgaaattgtttctaaaaaaacgagatttgattgaaacctagatcaaaaccagaggaaatttcactagatctagagaaagaaatattacaattgtttttcttttcaaatatttttaattttccgccgaaagagactccgaatcatgcaagataagtggataagatttgagaattttgtacggttgaagacaaagtttctgggagttagatcagttattagatgaattgtgtgtttccgtttcaaagctgtgaattaaattttaaaagttttattaccttatttttggtttaactaatcatttatttttgtttttttcttgacaggtaatgagacatttgagaagattgggatcatagggacattatcaaaccttcttttgtacctaactcaagtattcaaccttaagaaagttacagctgcaacaatcatcaatgcctttagtggcacaatcaacttcgggactttcatcgctgctttcctctgcgacacttactttggtcgctacaagactctcagtgtagctgtcatcgcttgtttcctggtactaacaattgatcttgaaatggaatattataaacgtcaagattcagtctttttctttactaaaaaaccatttttttacgtctttgtagggatcgcttgtgatattactgacggctgcagttcctgcattgcacccgactccatgtggaacacatagctggtgccaagggccaagcccgggccagatcgcgttcttgctgctgggtttagcgtttcttgtggtcggtgcgggtgggatcaggccgtgtaacttggcttttggagctgatcagttcaaccccaaatccgaatccgggaagaaaggaatcaacagcttctttaactggtatttcttcaccttcacgtttgcgcagatcgtctcgctcacgctggtcgtgtatatccagtcgaacgtgagctggacgatcggtttgctcatccctgtggctctgatgttcttggcctgcgtcatcttctttgctggacataaactgtatgtgaaagtgaaagcctcgggtagtcccttggctagtatcggtcacgttatcacggcagcgatcaagaaacgagggttgaagcaagttaagcagccttggctcgatctttacaaccacattccaactaactatccaaactccaccttgaaatacaccgaccagtttaggtaaacccaacatttctctctctctctgtttctatgttccctttgcttttatattaatctcttgctgagcaatactggtttgttatgtttgctaggtttcttgacaaagcagcgattatgacccctgaggacaagctgaattccgatggagctgctttcgatccatggaccctatgtacattgcagaaagtggaagaagtgaaatgcattgtgagagtgattccgatctggtttgcttgcgcgatttactacctcactgtaactatacagatgacttatccggtcttccaagcgcagcagagcgaccggagattgggttctggtggcttcaagatccccgcagccacctatgtggtgttcttgatgtcgggtatgactgttttcatcgtgttctacgaccgtgtccttgtcccgttgctcagaagagtgaccgggttagaaaccggtttgaccctcttgcagagagtcggatcagggatcttctttgccatgttgagtttgttggtctccgggttcgtagaggaacggagaagaaccttcgccctgacgaaaccgactctcgggatggagccacgagcgggagagatctcctccatgtcggccatgtggctgattccgcagctcttgcttgcaggcgtaggagaggcttttacagccattggacagatggagttttattacaagcagttccctgagaacatgaagagcttcgctggctctatcttctatgtcggtgcaggtgtttcgagctatcttgctagcttcttgatctcgactgttcatcgaagaactgaacattcaccctccgggaactggttagctgaggatctgaacaaagggagactcgattacttctacttcatgctcaccggaatcatggtcgttaacatggtttacttcttgataatgtctaaatggtatagatacaaaggcattaacgatgaagcgaattctttggtcgagaccaatgaagaagagaccaagcagaaacaagtcaagaattctgtctgatctgtaaaacttttattatttttggtcttctaattcagtctctactctttgttgtttgtactccattagagaaaatgaatgaaggagaaatataataataccttagcgtttttgtcgaataagatttttctgctaaattcaaactgtgtatgaagtttgttgtttcgtcacgaaccactaaaagaagactacaaggattgtgattttttttatgtattaaagccaaaaagagaggttcttttatgtatctttgtccaagaatgtaaaagaaagaagaagaaaaaaaatggccaaatagttcggaggacctctgcaaaggtgtgaagaagaagatgaggttatccagaaatgttttcttcttctcttctccgtctttataaattacattggaaaattattcaggacgaatcgagaggtagaagctttccttcaggcattgctatatacaaaatatttgggttattttcgtgtgggagattgatttagctgaggaaagctttatacttttattatcttctggtctctaaagttgcttagaaggtcttagagattggggaaatcacaattaccaatcaagtgccaagctcgaaagtagcaaggattgtgataaagtcgttggataatagaccgttgcttctgtattgcctagtggatatttcctgagttaaagatgggtacacaataaagggtggacaaaaagtaggcggggtcttagacttgatttggcaaatgacaatcacttatataagctaatggcaacataaccgtctctctcaccaatacatgccgaatttgaagcactctgatttgagccagtactaaaaacatggagcttgaacctgagcttgaaactgatggaaaagagcttatagccgatgttggacagtttaaagccgaggtaaaacagtttgaggccgaggtgaaagagttagtagccgaggtaagacagctggaggccgagctgacgcaacttgaagccgaggtgaagcaagatacttcggatgagcttgtagcagaggataagcttgtagctgaggaagcagttcgtcttggagttaatcgtgagacaaaatagagaagacatgatcctaaacctccctggtttttgatccaaagaatatcaacaaatggacaagtggttcataagcactctttttcccttactt

SEQ ID NO: 15 - protein, 617 aa, Thlaspi arvense
MERKPFEVETTENHKPYSTVDGGGVGSDLRSPVDSFDDEQKKLVYRGWKVMPFIIGNETFEKIGIIGTLSNLLLYLTQVFNLKKVTAATIINAFSGTINFGTFIAAFLCDTYFGRYKTLSVAVIACFLGSLVILLTAAVPALHPTPCGTHSWCQGPSPGQIAFLLLGLAFLVVGAGGIRPCNLAFGADQFNPKSESGKKGINSFFNWYFFTFTFAQIVSLTLVVYIQSNVSWTIGLLIPVALMFLACVIFFAGHKLYVKVKASGSPLASIGHVITAAIKKRGLKQVKQPWLDLYNHIPTNYPNSTLKYTDQFRFLDKAAIMTPEDKLNSDGAAFDPWTLCTLQKVEEVKCIVRVIPIWFACAIYYLTVTIQMTYPVFQAQQSDRRLGSGGFKIPAATYVVFLMSGMTVFIVFYDRVLVPLLRRVTGLETGLTLLQRVGSGIFFAMLSLLVSGFVEERRRTFALTKPTLGMEPRAGEISSMSAMWLIPQLLLAGVGEAFTAIGQMEFYYKQFPENMKSFAGSIFYVGAGVSSYLASFLISTVHRRTEHSPSGNWLAEDLNKGRLDYFYFMLTGIMVVNMVYFLIMSKWYRYKGINDEANSLVETNEEETKQKQVKNSV

SEQ ID NO: 16 - DNA, 1842 bp, Thlaspi arvense
  source 1..1842: mol_type = genomic DNA
atggagagacagcctctcgaactcgagtctacggatcaccaaaaaccttcccccgccgtctacggtggctctgttacgacggttgattctgttgaggaagaagttcaggagaaaaaagtcgtatatagaggctggaaagtcatgccctttatcattggaaatgagacattcgagaagcttgggatcattggaacactatcaaaccttctggtttttttaacagctgtcttcaacatgaagagtatcacagctgcaacaatcattaacgcattcagtggcacaataaatttcggaactttcgttgctgctttcctctgcgacacttacttcggtcgatacaagacactaaccgtcgcggtcatcgcctgttttcttggatcacttgtgatactattgacagctgcagtgccacaactgcacccagctccatgtggaacagcgagctcgtgcagcggtccaagcggtgggcaaatcgcgtttcttctgatgggtctcgggtttcttgtagttggagcgggcgggatcagaccgtgcaatctagctttcggagccgatcagttcaacccgaagagcgagtcagggagaagagggactgatagtttcttcaattggtacttcttcagcttcactttcgcgcagatcttgtcgctgacgctagtggtctacatccagtctaacgtcagctggacgatcggtctaaccataccggtggttctaatgttcttggcctccgtgatcttctttgcgggagataagctgtatgtgaaagtcaaggcctcgggtagtccattggctggtatagctcaagtgatcattgttgcaatcaagaaacgcggattaaagcccgtgaaacagccttggcttaacctctacaattactgccctccaaaacacgcaaactccattctcaaatacaccgaccaattcagatttcttgataaggcggcgatcttggctcccgaggacaagttggaggcggatggtaagcctgcggatccatggaagctgtgtacgatgcaacaggtggaagaagtgaagtgcattgtgagagtgcttcctatatggttcgctgcatcaatctactacgtaaccataacccagcaaatgacatatccggtcttccaagccctgcagagcgatcgtcgcttaggctcgggagggttcgtgatccccgcagccacctacgtggtcttcttgatgacagggatgacggttttcatcatcatctacgaccgtctcctcgtgcctaccttgagaagaataaccggtctagacaccgggatcacgctcctgcagagaatcggaaccgggatcttcttcgcctttgcaagcttagtagtctccggtttcgtcgaggagcggaggagacacattgcgctgactaaaccaactcttggcatggcgccaagaaaaggagaaatctcctcaatgtcagctatgtggctcatcccgcagctcactctctcgggtgtagccgaggcgtttggagccatcggacagatggagttttactacaagcagttcccagaaaacatgaggagtttcgcgggttccatcttttatgtaggaataggggtttcgagttacctcggcagcttcttgattgcaacggttcaccggacgacgcagaactcggcgggtggtaactggttggctgaggatttgaacaaaggcagattggattacttctatttcatgatcgctggaatcttggctgttaatttcgcctacttcttggtcgtgtcaagatggtataggtacaaagaaagtgatgatgatcaaaagacagcttctgaaaccaatggagatgtcatcaaacaacaagacaagaacactgcctga

SEQ ID NO: 17 - protein, 613 aa, Thlaspi arvense
MERQPLELESTDHQKPSPAVYGGSVTTVDSVEEEVQEKKVVYRGWKVMPFIIGNETFEKLGIIGTLSNLLVFLTAVFNMKSITAATIINAFSGTINFGTFVAAFLCDTYFGRYKTLTVAVIACFLGSLVILLTAAVPQLHPAPCGTASSCSGPSGGQIAFLLMGLGFLVVGAGGIRPCNLAFGADQFNPKSESGRRGTDSFFNWYFFSFTFAQILSLTLVVYIQSNVSWTIGLTIPVVLMFLASVIFFAGDKLYVKVKASGSPLAGIAQVIIVAIKKRGLKPVKQPWLNLYNYCPPKHANSILKYTDQFRFLDKAAILAPEDKLEADGKPADPWKLCTMQQVEEVKCIVRVLPIWFAASIYYVTITQQMTYPVFQALQSDRRLGSGGFVIPAATYVVFLMTGMTVFIIIYDRLLVPTLRRITGLDTGITLLQRIGTGIFFAFASLVVSGFVEERRRHIALTKPTLGMAPRKGEISSMSAMWLIPQLTLSGVAEAFGAIGQMEFYYKQFPENMRSFAGSIFYVGIGVSSYLGSFLIATVHRTTQNSAGGNWLAEDLNKGRLDYFYFMIAGILAVNFAYFLVVSRWYRYKESDDDQKTASETNGDVIKQQDKNTA

SEQ ID NO: 18 - DNA, 5002 bp, Thlaspi arvense
  source 1..5002: mol_type = genomic DNA
tgccatgaatctctgaactggagtgaacaaaacctttttgtatatcgttgatcgcatttctttgacaactacaagcaactaaatatgaaagcatatgcttgaggattttatttgaggatcaaattggtaaaatgaaagagattaaagttgaattgaagcccaagttgaagagattgaaataatggcacacatcccttattggtgtcatgtctgactaattttaccaacatggcaacatctcataagtaatattgaatatcaggctttgtgatttctacaattaattttaacagttttaactgatatggtccaagactcgctattgaatagaaatcaaaaagccatatccaacatctctgttaaatgtagtcttaggtcggttacttgtatacgttttaaatgattcaaaaaattggcgcctcatcatttatccgattgtagagtaaaattaaaataatatcataagaaattataattttctctaatccacaaagattgtcttacatcttatgtactttatattacaatatagagcaatatatctgaattagtctttggaaaaaaaatctgaatcaaataaaatcgaacgcaagtaaaattcttaaaataacttctagtatgtaaaaaatattcttattttctggttaaagtacacgtaaaaattatataaattactagaatatatattcatgacaaaagagtataagtatagtatataacctcgaaatcataacaagaaaatttaatgaagatggatatatatatagtatacagtcgaaaaataaagagaagaagaagaagaagaagataaataaataaataagaaaatggatgatggaaaacaatttgaagaagaagaagataaataaataaataagaaaatggatgatggaaaacaatttgactgaaagtggacggagagcttaaagaacgtgccgttattaagagggagtgttatgatggggccaagggcacttgaaatggaatctacattttcatgtattttttttctgtttcttttgaagagaagtttattttttttgtaagaaaaggagaaaagataattaataaaaaatgtaatattttaatctctaacacagctctctttcacggccaaattcacaaattaccactttgtctagattccggcatctccaccatcctcttccttcttaccactatatagtctataaatagagacggctctaagataaagaacagagtcatcattagcctttgagacaaatacaacagacaaaacaaggaagtacaatttaaaaaaaatggagagacagcctctcgaactcgagtctacggatcaccaaaaaccttcccccgccgtctacggtggctctgttacgacggttgattctgttgaggaagaagttcaggagaaaaaagtcgtatatagaggctggaaagtcatgccctttatcattggtaaatatttaccgtttttaacacttttttttcttggtcctggaaactatttttttcagttggattcatgtagttaggaattacccataaaatcataagaaaaagatcgaaaacatcagtcttgctcgaagacttgcttggtttgaattttttttttttttttaaatttgaaagattctctgtgttgtttgtcttaagctttgacttagtcaaacagtaacttaagacaaaaagaaggacatcttgaaattgaggagattttataacataactaatcaagaattaattttaaaacacagatttcatctaactgtttgattcttggtttggtttctgatacaatcaaatgcaaagcttgaaacttaatcttcatttttgttgttgttatcaggaaatgagacattcgagaagcttgggatcattggaacactatcaaaccttctggtttttttaacagctgtcttcaacatgaagagtatcacagctgcaacaatcattaacgcattcagtggcacaataaatttcggaactttcgttgctgctttcctctgcgacacttacttcggtcgatacaagacactaaccgtcgcggtcatcgcctgttttcttgtaaaaacccatcctttcttgtcttgtctttcttcctccacaagcatatatatacaagcggaaaaaacttatcggtgaaatggtttctttttgtagggatcacttgtgatactattgacagctgcagtgccacaactgcacccagctccatgtggaacagcgagctcgtgcagcggtccaagcggtgggcaaatcgcgtttcttctgatgggtctcgggtttcttgtagttggagcgggcgggatcagaccgtgcaatctagctttcggagccgatcagttcaacccgaagagcgagtcagggagaagagggactgatagtttcttcaattggtacttcttcagcttcactttcgcgcagatcttgtcgctgacgctagtggtctacatccagtctaacgtcagctggacgatcggtctaaccataccggtggttctaatgttcttggcctccgtgatcttctttgcgggagataagctgtatgtgaaagtcaaggcctcgggtagtccattggctggtatagctcaagtgatcattgttgcaatcaagaaacgcggattaaagcccgtgaaacagccttggcttaacctctacaattactgccctccaaaacacgcaaactccattctcaaatacaccgaccaattcaggtaaccaaaaaacttaaacttgttttcctttaagagtcttttttattgtcaggatgttctttactcagagaaaggttcctgttttgggcagatttcttgataaggcggcgatcttggctcccgaggacaagttggaggcggatggtaagcctgcggatccatggaagctgtgtacgatgcaacaggtggaagaagtgaagtgcattgtgagagtgcttcctatatggttcgctgcatcaatctactacgtaaccataacccagcaaatgacatatccggtcttccaagccctgcagagcgatcgtcgcttaggctcgggagggttcgtgatccccgcagccacctacgtggtcttcttgatgacagggatgacggttttcatcatcatctacgaccgtctcctcgtgcctaccttgagaagaataaccggtctagacaccgggatcacgctcctgcagagaatcggaaccgggatcttcttcgcctttgcaagcttagtagtctccggtttcgtcgaggagcggaggagacacattgcgctgactaaaccaactcttggcatggcgccaagaaaaggagaaatctcctcaatgtcagctatgtggctcatcccgcagctcactctctcgggtgtagccgaggcgtttggagccatcggacagatggagttttactacaagcagttcccagaaaacatgaggagtttcgcgggttccatcttttatgtaggaataggggtttcgagttacctcggcagcttcttgattgcaacggttcaccggacgacgcagaactcggcgggtggtaactggttggctgaggatttgaacaaaggcagattggattacttctatttcatgatcgctggaatcttggctgttaatttcgcctacttcttggtcgtgtcaagatggtataggtacaaagaaagtgatgatgatcaaaagacagcttctgaaaccaatggagatgtcatcaaacaacaagacaagaacactgcctgatcattatgggtttctcatcttctcgacttagtttttggtcagcttcgatcccctttggattttcttggaataagcccatagagaaaccaagtaatcatcatatacttgttctcgctctttttttttcttttcctcgtttatatttaaatcacttctctttctttctatcattgtggacaaaaagggaaattataacatcaaatgtaatgcaaaaataaaaaaaaatttgtcttacactttgaggatgctgtttctatttctgccattcttcatcatacaagagaacaaacttacaagaaggaaacaaaccgaccaaataatactactaaggcctttgatacttatatgcataatttgtatcgaagtttcaaacagagatattgagacgagtcgtgggctttctctctacgattttgtcagactgtgtaagattgttgaatggtttcaatgtcgagaccttttagcctcacgacagattcaacgtgacccttgagtgtgtgaagctcccgcaacctatcaaagtgtaagagagaatgagacaaatcacaaatgttccggttcgaccaaaacaaagcaaaaacaaacgttccggttttgtgttccttctctgaccctcacggctcacaggaagaaactaggcttacctagcgatctcggctcttctctttgcttcttcagccatttgggaaagctcgttgaaatgggttcgctctgggaacattttagcatctggtgcttgcaggccatgtagcgttctctgtgcgtgcgcccattgtagctccctttgttctttcccaaaatccttctgcctcgtgaatgcaatctatggaaacgagtttacatgaaagatcaagtacagataaagaaaatggaaagaggtgaaatgagaagaaaatatgtttgctttttgatacttaccctttgctcaataacaaggtcccaggctctgccgctaagggcgtaccgaatcaagaacttgatgatatcaagaggaatatagaacacaatgttgtagagccaaatgaccccggcccatccccacccgatcccttcgatcgcagcaaagctccaattcgcatagacggcaattagagtagccacctgtttggaagaaaatggtcaagaaaacagtgatttcaaaacttcttacttgagtgctgaccacagatagtgtgtagtgtcacactaaccaattgtgcgacgataaaggctatcacaagcaacatcccaggacgttccacataagaccaactccgtgaccgggtcacaaagattagcgcctgactaataatgctgact

SEQ ID NO: 19 - DNA, 1110 bp, Thlaspi arvense
  source 1..1110: mol_type = genomic DNA
atgtcaagaaagccgtgttgtgtcggagaagggctgaagaaaggggcatggaccaccgaagaagacaagaaactcatctcttacatccacgagcacggtgaaggaggctggcgcgacattccccaaaaagctgggttgaaacggtgtggaaagagttgtaggctgcgatggactaactacctaaaacctgagatcaaaagaggcgagtttagttcagaggaggaacagattatcattatgcttcatgcttctcgtggcaacaagtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtga

SEQ ID NO: 20 - DNA, 2745 bp, Thlaspi arvense
  source 1..2745: mol_type = genomic DNA
ccacattggatccatgttatttattgtgtgtgcaaactcattcaattccatgtataatactcataaattcatttaatttgatactccaatttttttattctatgaaaattgaatatttaataatttagggttattaatttaatataatttatctagtaagaaagcgcattttcctaaccaaaataaatatatatatatatataccaaaatatagtaatatacaacatcgctttgcaaacattgttcttataaaaaaagaataacgaactagctataactaattaatctatttaaaataagaataatttacatactctgtgtatatatatagaggagcatagctcgtaagctcaccaccatcacacaatccatgcctcttggtttttcttcttcagcttcttgattctaatcttagtgtcgtaatattataagaataggacaaaaaaaaagatttggggctctacgtgaaaaacatgaaaacgcctagcagctctgtgggaaagacccaagagcgtttctcgattagtctcatattcagatgcatcagagttctcataaacagatctatttcttacttatctttttagaaaaattcctttcaaattttactttccttaagaatagtgttctctacattttttttcttgggtgttcgtgagaggttatatgaattttttgtcattagtgttcatatcggaagaagatgtcaagaaagccgtgttgtgtcggagaagggctgaagaaaggggcatggaccaccgaagaagacaagaaactcatctcttacatccacgagcacggtgaaggaggctggcgcgacattccccaaaaagctggttaatatctctattaaaatctatacatgttcaattagtatttcttgtatgaaatttttataatcaatatggtgttaactaaatagggttgaaacggtgtggaaagagttgtaggctgcgatggactaactacctaaaacctgagatcaaaagaggcgagtttagttcagaggaggaacagattatcattatgcttcatgcttctcgtggcaacaagtacgtttctatgtttaaatgtgtgtgtatatatgtatcctcgaataaacaatgaaatgcatgaaaaggtttcatatatattactttaattaaagatacaattattattctaatatcgtgtcttccatattatattttaatcgcctttgatctttgaatctctcttcttatcgttaggtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtgaatcctcgtatccaaacggaaaggtttcaaactatttaaaactttcctgaaccacaatttatgtatgtctttcttatttggaacttttagtatatgtccaagtctccaagatctcatggttatttaatcccaggtttagggtttgtgtgatgttaagtaagggtgaatctttatatatgaattagggtttctctgacattgagaaccatgcattgcggatcaattggtaattgattgcacgagccacgatgtttcttttataatgtttattactaataaagcttgttttgttatcgtatttcataatagcatccattatcatcattttttttgggtttatacttaaagtccacattccatcataaacatcaatcatgcacttttttggttttcatactcttctttatgattaaataattgtttccaactttccatggcatgaaggtggttgtatctttcgttggatctgaaccgaccacaatataataaataagtaagtttttaaaataaatgataaaaaaaggctcaaacgacagagacgttccaagaaaaagtgtaaacgtgtggtccataaacaattgagacgaaagctaaagatcaagaggtgaattaagcagataactggaattttgtgacccgacaaatattatcttaaaacgaaatctaatgt

SEQ ID NO: 21 - protein, 369 aa, Thlaspi arvense
MSRKPCCVGEGLKKGAWTTEEDKKLISYIHEHGEGGWRDIPQKAGLKRCGKSCRLRWTNYLKPEIKRGEFSSEEEQIIIMLHASRGNKWSVIARHLPRRTDNEIKNYWNTHLKKRLIEQGVDPVTHKPLASNSGPTATTPPENLHFLDESSSDKQYSRSSSMPSLSRLPSSGFNTVSEIASNVGTPVQVGSLSCKKRFKKSSSTSRLLNKFAAKATSIKDILSASMEGSSSAATTISHASFLNGFSEQSRNEEDSSNASLTNTLAEFDPFSQSSLYPEHEINVTSDIGMDQVYDFSQFLEKLGSEGHNELNVEYGHDLLMSDVSQEVSSPSVDDQDNMIGSFEGWSNYLLDHADFIYDTDSDSLEKHFM

SEQ ID NO: 22 - DNA, 1080 bp, Thlaspi arvense
  source 1..1080: mol_type = genomic DNA
atgtcaagaaagccatgttgtgtgggagaagggctgaagaaaggagcatggaccgccgaagaagacaagaaactcatctcttacatccatgaacatggcgaaggaggctggcgtgacattccccaaaaagctggactaaaacgatgtggaaaaagttgtagattgcgatgggctaactatttgaaaccggatatcaagagaggagaatttagctacgaggaggagcagattatcatcatgcttcacgcttcccgtggcaacaagtggtcggtcatagcgagacatttgcccaaaaggacagacaacgagatcaagaactattggaacacacaccttaaaaaacgccttatcgatcaaggtattgatcccgtgacccacaagccacttgcccctagccctagtccggccacgctcaagccttctgatttccaagatgactcatcaaacctgggaaactcggatgagcattcacattcgggttctatgtctccaaaatctcttcctccgtcttcaagctcctgcaatctagcggagataagcagcagtgatgagacaccgaaaaatgatggttccttgaaatccaagaaacgttcttttaagagatcaagttctacatcaaagctgttaaacaaagttgcatctagggctgcttccattggaaatatcttatcagcgtccatggaaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatga

SEQ ID NO: 23 - DNA, 5588 bp, Thlaspi arvense
  source 1..5588: mol_type = genomic DNA
aaattcaagtttgataatatatgttgttaagttaattttttactaagtaagtgtacattcattatgacgattccaaaatctaattttctcaattaagtccaaaacaaaacagaaattataaaatttccaattcgtttcaaccctcgtgaccaggtccgtatgataacaattaaaaaggatcagtgtcaacatatttgtttctattcatcacaagtctcctccaaacatatttgaaacactggcaagggacattttataatgtttgtgtccacggtctgaatatccctttttattcatttgaatattaccctaattaaagaagccacctccgagaaaaagttcaggtgaactctgctctggtacatccatataagtgggcgattctaatgccatattaattaacacttgtcactttttcttggtgcatagacacaaaatttaccaattcagctagttaagctataattaactaattcagattcatactgtcatcataatcagttaaaatccatgcaaagcgatactaatcttatacagtgcatgatgatagtctatgttgttttttgttttttaattaacgcttaattatccaaagatcaaattaatcaggcgccctttaaatcgtctctccatggcgttcatgttttatttgtatcagtcatcacagtttattcaagttgcatttatgatcatgaaagtgacagcctaagcaactaacataccaaaaaccatgaatagttttgttcaaagtttcgctaaacgatggttgaactgggtctaatgtcctattttttcccaaattgattatgccttcttagccctttttataaacaaaaactgggtctaatgtcgggactactatttatatattttttaaaaaataattctaacattttaatgcattataatttataattatatgttcagtattgcataaagcatataaaaaaccaaattaagacataatatatttttattaaaattaataaattataaatttaagttgaatgaagtcgtcaaattttacacttaactaatcatttttcttttgagttaactctgcaagttccgaagaggcataactattagaaacaatagtgaagtccaagtgagatgtgagatgtgagatgagaagaaacaaacattgataagtaagcaaaattactatttaatattttgaatacagctcctagtgtttcagggatagccaaagtggcaaacaaataggaccgaggctgaaaacgaacacgtgatgattaatcgcatatatatatatatataataaaatagtaataaatacgctaataattatgaaaaatgataatggtttttatctgtgcaataaaaaagaatttatcaatatgttttctttaaacatatttattaaacacgttatagatggttttatccgtatcattttactgctacaacatacatgttttttttttaatatataaacatatatatatatatatattttctaatatggtcttataatttgtctattagaaaccatagacgatgttactctttacctaataatctctaatgaaaatgatatgatgataaccatatttgtgacctttttcgggtcggcactgcaattatcaattcaattactatcagaaaactattgcaattatgaaaaccatattttttaagtctctctcaaactatttaacagtttttaaaataaagaaactatttgaacagttccacgtccaaaagaagatgatgaacgctcactacccttttaatgaggttttttttttgttatttcatctgaaatcacaatgtaaacctttacaaaagagccatgtagctaccaaaaagagccttgatatgtctaggaaccacacattgatgagttaatattgtatttatttttttagatttatttattttattttattttacgtaggcttttaaaagtattctgacacgagtgcgcactataaggcttataaatagatgctcatatacatacataattaactcattacagagaagtatctcacaatgtcttagatctttgatacatctacaactttttttttactagcgttttcaaatgttactgaaaatgaaaacacaaaatcactcaacgatctaccatagtttttcagttagtttcatattcagatgcatcagagttctcatcaacagatctattggtctcttgccttaatttagacgacatttctgagttcttttcccctttgctactcatcttctttaggttgcgagatttttgtgtgtgttaaacttataggtcgtatgtaaatctatagatattgtatatatatccgaaggaaaaaaaggagaaaaatgtcaagaaagccatgttgtgtgggagaagggctgaagaaaggagcatggaccgccgaagaagacaagaaactcatctcttacatccatgaacatggcgaaggaggctggcgtgacattccccaaaaagctggtatattacacatatatagcttatagttaaaccaagcataatctatgatctaattgatgtttatatggctgtggtgtttaattaggactaaaacgatgtggaaaaagttgtagattgcgatgggctaactatttgaaaccggatatcaagagaggagaatttagctacgaggaggagcagattatcatcatgcttcacgcttcccgtggcaacaagtaatacatatatacttaagtcaaaattcatatattaattctcaatcttaaattaagcattaataatttgttttctcttgttttaacgttattttcatttttttaaaaaccaatgtcctgttgaattttatttataaaaataaaatattcaacctcaagtcaaagttgatttaaaaatagttataagttgggataaagataccaggaacaacttatttttatataaaaaatgaactttctaacttcatcttaatttgtgtaaaaactgtcaaaaagtacgtattatatattgcgaaacaggggagtgcactgaaagttttgatatattctacttttctttgacgtccttactataggtggtcggtcatagcgagacatttgcccaaaaggacagacaacgagatcaagaactattggaacacacaccttaaaaaacgccttatcgatcaaggtattgatcccgtgacccacaagccacttgcccctagccctagtccggccacgctcaagccttctgatttccaagatgactcatcaaacctgggaaactcggatgagcattcacattcgggttctatgtctccaaaatctcttcctccgtcttcaagctcctgcaatctagcggagataagcagcagtgatgagacaccgaaaaatgatggttccttgaaatccaagaaacgttcttttaagagatcaagttctacatcaaagctgttaaacaaagttgcatctagggctgcttccattggaaatatcttatcagcgtccatggaaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatgaccagttgattgctacccggacttgagttgagtggttaattaagggtttcactatattaatttttctaagatgttttcgtaagttattaattaagggtcactatagggttatccaataagggattctcttagttagagaaccatgtgttcccctggatcaattagtatttgatttgcgggagacacgagttatttttttttgctttaagaactgttaagagtaaatattaaataaagcttgcattttcataattccattatgtataattattctgtatatttaaatatcaatcatgctaagagtaattattctggatagatttagaggaacttacgaacgtaacaaggattaaaaagtgactatttttagctgacataaacttttaacatgccttcatgacccaaaatctaaactactttttggagctttcgcatgcgttcactgtactacaatcatttgatttaaagtttgagcgttagattagaacagatgagccgtgttaccaaaaatatatggaagaaaatgtaagacattttagaattttggggatgaaactcccattaaaaaaaaaccccctacctacgaataaagtcattgaagtttttttagagatgaatttttttttgggggttgcataaacatgattttttacacttgtatattagaagtatatataactggaaacattaaataaatgagatagttaaaattaaatgaattgaatactaaactagtggagcagagttaaaaaagagaataatttgcttttctcgtaaactaatttgcagagagatatatacttgatatactttaacgttacagcaaaatatcttaagaaaaagttaagaaaatttagtcaaaaataaaaaatttcagtaaatgtatttagtaagacgatacactctagattttgttcatttaaaactaataatctggcgatcataaacaacttctcggaaaaccaaaagcaatatattgataaagttttttgtgtggtaaatattcataaagtctgtagatattgtttttttaatttagatataaagtatttcgatctttcgtaatcgaacgtggggcagaaacgcaccatcctttttcttttcgacataacatggaaaatagcaaaaatctatattagtacgacgaataccagtaaataaataaaaacaaacaagaaataaaaatgctgttcccaatctgtgtatatatatggtccacaaatacataattattttcagaagctaaagaacaataagtgagcaaacataattggattagtgacccgacatatacagtattgtgtgcaaagttaataaacattcaatgatataaatatcatcataattcatagtatgtgtgatacaaggagctttggtttttatgtggctacacgtcaacgactcaattcctgtttttatatcttagaaacttggtttgctaccgacaatctgtagctaccgctgattcctaatcgatttagatcttgaacacacataatcaattcacacgcagtatcttgtcttctaaacttgcagcccataaacctttttgcctgataaaactctcaatatgcagcacacaggttacaggtattgagtgtcttgaatcatctccaacatcttcagactcgactgtacttttctttgacttaggcgatagttacgtatattgatacgtaatagtgacttatatatctatctttcaatctcatttggattcaagtgcgtataacttaaaca

SEQ ID NO: 24 - protein, 359 aa, Thlaspi arvense
MSRKPCCVGEGLKKGAWTAEEDKKLISYIHEHGEGGWRDIPQKAGLKRCGKSCRLRWANYLKPDIKRGEFSYEEEQIIIMLHASRGNKWSVIARHLPKRTDNEIKNYWNTHLKKRLIDQGIDPVTHKPLAPSPSPATLKPSDFQDDSSNLGNSDEHSHSGSMSPKSLPPSSSSCNLAEISSSDETPKNDGSLKSKKRSFKRSSSTSKLLNKVASRAASIGNILSASMEGTLVSSTALSPCLNDDFSEASQFQMDEYDPFPQSSEHITDHMKEDTGMIFDLNNSEYDFSQFLEQFSNEGEETENIGGYNQDLLSSDVSSPSVDEDNMMGNITGSGWSSYLVDHSDFVYDKIQDNDDRNFI

SEQ ID NO: 25 - DNA, 1017 bp, Thlaspi arvense
  source 1..1017: mol_type = genomic DNA
atgtcgaaaagaccatgctgtatcggagaagggttaaagaaaggagcatggacgtcaggggaagacaaaaaactcatctcttatatccatgaacatggcgaaggaggttggcgtgacattcccgaaaaagctgggctaaaacggtgtggaaagagttgcagactgcgatgggcgaactatttgaaccccgatatcaagagaggaggatttagctacgaggaagaacagatcatcatcatgcttcatgcttctcgtggcaacaagtggtcagtcatagcaagacatttgccgcaaagaacagacaacgagatcaaaaactattggaacacacatctcaagaaacgcctgatcaataagagcactgattccgtgacccacaagcctctagcttcctctaaccctagtcctaccgagcgtaagaagctcgattcccaagaagaatccaatcccaaggagcagtcgttacagccgggttcgaagtctccagtatctctttccctttcttcgagtttcaacgacactgtacccgagatcatgaccagtgatgagacgcctctagaaagtggtttcttgagttgcaaaaaaagtgtcgagagatcgagctcaacatcaaggcttttaaacaaagttgcagctagagcttcttccatcgggagtatcttatcaacctccatagaaggaactttgagatctcctgcatcgtcctcatgtctcccaaactcattgtgtcaatcatctgaacacaacaaggatcaagatctcggtacgagcattgatcttagcatccccgattacgattactcccactttctcgagcacttcatcaatagcgaagacgaagccgaaaacattggtggctgcaatcaagatctccttatgtccgatttcccatcaacattagtggataaagaaaatatgaattttgaagacataaccggttggtcaagttatcttctcgaccatcccagttttacgtatgaatcggaacaagattccgacgacaacaacttgttatga

SEQ ID NO: 26 - DNA, 3950 bp, Thlaspi arvense
  source 1..3950: mol_type = genomic DNA
ggtaatccatcggaaagtttatacggattatcgacgacttgctttcgtcggaaatttaagacgaattttcgatgacttgcaacattccgtccatttgtaatcgacgaatcatttttgtcgaaaatttccgacggatttttgacaaaaatggccgtcgcttctttcttgtagtgtataataaatgcgctttaatttagatatttcggtattgtgcatttagtaggtgaagtatattaattatatatcctagaaacaaagattagtagaattttgatccagtggcttgctaataaataggactcaagagcggttttgatacatcaaatctatgattcaaacttcaaaatatgtgatttcttgcatattatgaatacaaagatactcagggatggatacgtacgtgctctgaattaatcgaatcttgtgccacaatttatggtgtgcaaattggattattaatttgtaatagacatgaatattaaaaataaaacatatttaatctgaaaaatttcagaacatcgtagatcagataacatctggattcgagatggagagaaaaaaaaaaaagtagattgtggccttgtgggcgattctagtgggacatctgctgaattagagaaagaaatggttaaatgttaaaatattttagattggatagtatctgaagtagagaccacagaaaaagaaaaaaagtgaattgtgggtgagcctagaggaacaaggctgtgacatacgtgggaagacagacgaaagagcccaaaaacgtgcaccgtccaagattctctatacgtacctaacctccttccagggactcttcacttgacacttacacttacacttgcacttgcactcactcacactcacactcacactcacactcacacttacactcaaacttagccagctaattttgttttaatttgtattggtgatttgactgatgaataatataacaacggcaacatcaacggttatagagtggaaaaaaaaaaaaaccgcgaactacacatcttgtaaatattagtttagatgtcattgtctcccatatcgagacgtagacacgtcgtaagcgatttttattttttttgtcactcttcaaatgatatatagaaaacttggttttcttcacatgtttatcaacggtaaaattgtagatgtaatctaaattaataaattaatatttacaagatgtgtcattcattctaatcacgttcattttatatttatgtataacactacagtagcggataagcttagtatgtgcagaaagaatgggtagaatcgtacgtgggtttacaagtttattgtcatgagtaatatatactagatctaagctaaaaaaaaacaaatattagacccttaaaatgaacacatacggtattacgatcttatacacataatctcgcggttaatctcaacggaccatgcatcatatgtaattatattaaataaacatcagtctctctatatatccttgatttacaatatcttttctttctgcacattttttcgaaatgcaaagaaggaatgtcgaaaagaccatgctgtatcggagaagggttaaagaaaggagcatggacgtcaggggaagacaaaaaactcatctcttatatccatgaacatggcgaaggaggttggcgtgacattcccgaaaaagctggtacatgcatttcatttacagattttgtaatatttatttatcgaattgatctttatatgcttttcattttctgagtttaattagggctaaaacggtgtggaaagagttgcagactgcgatgggcgaactatttgaaccccgatatcaagagaggaggatttagctacgaggaagaacagatcatcatcatgcttcatgcttctcgtggcaacaagtactacaacgtttgcctatatatttgtttttgtatatgcatgtaatacaatcatcaacgtatacataactttacgacacgatcaatgtaattggactgtactatattttttattaggtggtcagtcatagcaagacatttgccgcaaagaacagacaacgagatcaaaaactattggaacacacatctcaagaaacgcctgatcaataagagcactgattccgtgacccacaagcctctagcttcctctaaccctagtcctaccgagcgtaagaagctcgattcccaagaagaatccaatcccaaggagcagtcgttacagccgggttcgaagtctccagtatctctttccctttcttcgagtttcaacgacactgtacccgagatcatgaccagtgatgagacgcctctagaaagtggtttcttgagttgcaaaaaaagtgtcgagagatcgagctcaacatcaaggcttttaaacaaagttgcagctagagcttcttccatcgggagtatcttatcaacctccatagaaggaactttgagatctcctgcatcgtcctcatgtctcccaaactcattgtgtcaatcatctgaacacaacaaggatcaagatctcggtacgagcattgatcttagcatccccgattacgattactcccactttctcgagcacttcatcaatagcgaagacgaagccgaaaacattggtggctgcaatcaagatctccttatgtccgatttcccatcaacattagtggataaagaaaatatgaattttgaagacataaccggttggtcaagttatcttctcgaccatcccagttttacgtatgaatcggaacaagattccgacgacaacaacttgttatgatctatctgtctgtggctcgtcacctgctcattgaaccaggggctgaccagttaatctcatgttggtatgtccaagataaggggttcaatatcttagataaatttcaacttgttaaattgcgttactagattatttagtaagaggcacttatgtaataaaaaaaaaaccatgtactcccgtgtatcgaatagtattgatttgatctgcgtaagagtttttcttataactattttgtaaaatatatgttgaataaattaagctttatgcatgtgtacctttttttaataaaaaaaaaactaagctttatggtccacaaacactaccaaaaggtaaagagcaatgattgagcaacaaaattggatgggtgacctggcgaatctttattaatgttcaatgaaatgatcatacatttatgggaagcttttgtttctatgtgcatggctgtacatgaataatttaatttttttattggtcgtgttacatcattatcaaaaacattccatcaaattcatttacatatatgcgaatatgcgattatgatgaacaactccacgatacagttttccacatgtgacacacacggatctgtaacagatagagacatagagttgcacatcaaaatctttcgataagatttgtatctatcaatatacagaaacttgtctgcatttgttaggaatcgaacctcaaatatatggtgtagtaggtcaagggctttcacaattttactgttgttgtgtgtaaaactgatcgtgtgaagtgaacgttcagaaaaaagaatacttcctctctatgtttttctacacttaatttagttttgtgcctaaaaatataatttcaatagtaaataatacttgacgtggtttattacagaaaaagttacatacagtataaatgtaagaattacatacagaaagaatcatgttagcattcattcaaagaaaaaatggaattatgttagcaaatcaagtcgatctaagttttattacatgttggcattcattaaacccttgcagtatctgaaaagaattgctctccacaccagaccaagtccggttcgataataccatgattgctattggaaaacgtcgtaccgagacataagaaccggtagtaagttaattacgaaagtagccttacagctaaacaatcggtagtctaaactctgaagcgatcgcgattagccccttgggagtgattgggag

SEQ ID NO: 27 - protein, 338 aa, Thlaspi arvense
MSKRPCCIGEGLKKGAWTSGEDKKLISYIHEHGEGGWRDIPEKAGLKRCGKSCRLRWANYLNPDIKRGGFSYEEEQIIIMLHASRGNKWSVIARHLPQRTDNEIKNYWNTHLKKRLINKSTDSVTHKPLASSNPSPTERKKLDSQEESNPKEQSLQPGSKSPVSLSLSSSFNDTVPEIMTSDETPLESGFLSCKKSVERSSSTSRLLNKVAARASSIGSILSTSIEGTLRSPASSSCLPNSLCQSSEHNKDQDLGTSIDLSIPDYDYSHFLEHFINSEDEAENIGGCNQDLLMSDFPSTLVDKENMNFEDITGWSSYLLDHPSFTYESEQDSDDNNLL

SEQ ID NO: 28 - DNA, 1047 bp, Thlaspi arvense
  source 1..1047: mol_type = genomic DNA
atggctcggactttgcaaggcgagtggatgaaggtggagcagaaaggaggacaagtaccagcaccgagaagctcacacggcatagccgtgatcggagacaagctctattgtttcggcggcgaggatccgccatatgagtccatcgacaacgacctttatgtctttgacttcaacacccacacttggtcaatcgctccggccaacggagacgttccaaagaccagagtcttaggcacccgcatggtggccgtgggaacgaagctctatgtgttcggaggccgcaataaacagttagagttcgaggacttttactcgtacgatacggtgaaagaggagtggaagttcctgacgaagctggacgaaaagggaggacccgaagctcgtactttccattcgatgacttcagatgaaaaccacgtgtacgtgttcggtggggtgagcaaaggagggctgaatgcaaccccctttcggttcaggacgatcgaggcctataacattgctgaagggaaatgggctcagctccctgaccctggagaggatttcgagaaaagaggaatggccggattccttgtggtgcaaggcaagctttgggtgttttacggattcgcgactgcgaatgatcctaagatacccacactctacgggtcccaggactacgagtctaatcgtgtgcactgctatgatcccgctactcaaaaatggaccgaagtggagaccacaggtttcgagaaaccttctcgtaggagctgttttgcgcatgcggctgtggggaaatatataataatattcggaggtgagattgagcgggacccagaagcacatcaaggtccggggacgttgtcccgtgagggttttgcgttggacactgagacattggtgtgggagaggtatgaaggaggaccaatcaaaccgagcaaccgcggttgggtggcctccacgacgaccaccatcaacggaaagaaaggtctgctcgtgcatggagggaaacttatgaccaacgagcgcaccgatgagatgtacttcttcgcagtcaattcctccacgtaa

SEQ ID NO: 29 - DNA, 4094 bp, Thlaspi arvense
  source 1..4094: mol_type = genomic DNA
cgaacattttttttttttggcaaacacacttagaacgtgatcacaatccaaggaaacccagcactcgatgcctaaatatctgaagttgatgcaaaaactgaaatataaagtttagtatagatagataactggttatatttatagaagacaaccgaaaggttctgcaaaaggggtagtttttatactagttcaatctaacaaatgatcagtttttttttgggcaaatataatcagtttgtttatatatatatatatatatttttggtcaattgtttatttatatataacacactattttatatcatatcattctaaatttctaatgggtttaaattttttattactatgcaattaaaatacacagctcatatattgaactaaatagttgtaaataattttaggtgaattacaaattgttatcacctatatctatattattaatttcgaatcaggaaagtaacattttgttcactaaagctaaaagcctaaaagtatgataaaatgttcaaaatggtgaataaagaatgatatggggtttaagtgtttaactggtaatattacagcgatacacaaaataggaaaaaaaaacaataatttttgtatctcgtgttaaaagtcaaacaaatgtacaattatgctaagatagtttgataacgctgctttagatatttttgtggtgtttgctgttttaaaaattgttgtatttttgaaaattatatggtagaaaaaaaagaaaagaaatgttccacaacgcaataaaaatcaagtttgaagatatcgatcatgttttatagtaataagcaaaaaaaaacaaacacatgcatcatcatcatcaagctatcaagtaacaatcatgtttcatagtaataagcaatatcatcatcatgctgtcaagtatcaatcatgtttcatagttttaagcaatatgaattaacacatgcatcatcatcttcatgctttaatttagactaacatgtggcgcagaaaccgagaagctcacacggcatatatacccgtcgcctaacagctatgttagcttttgttaatcttcatgactgtggcacatctctttgagcactgcgcaccatccatgcatgttttctaactgctccaagaccttttatcctaaacaaacacaattattcattctccttttctgatttattttctgttttttttttgccaactgattttctgatttatcgctcactcgcgaagatatttttgtaagggagatttaccaactaagcttaaagaaattaatatgcttctcacattttttagataataatttaacactttcacttcctttttgggggtacaaaatacccacaagttcacagaaattcataagcaccacacagttggaaatatcttcttcttttttttcttttcttttttgtttgttaaacaatcaataattggagataccttgacacgcagaattgtttagcctatatatagaagagcagcgaaagaagaggagacacacaaacaaatagaaaagaaaagaaaaaaagagttgaatattgcagctatggctcggactttgcaaggcgagtggatgaaggtattgcctacttagtcaaggatttttatttaagatattaattttgcacgagagaagcataagccataggctatgtattatgtatatatgtataatattggttattagtaaaaagttatgtatgacaacacccttaattcccatgttctcgacacgttataccccattcgtatccctcctgaaaagaggggatgttgatggtttacttgtaattaacctgtcatgtattcgaatgattaaccggctcatcagtatccgttttaaccatcaattcattttggtcactggttgacaggtggagcagaaaggaggacaagtaccagcaccgagaagctcacacggcatagccgtgatcggagacaagctctattgtttcggcggcgaggatccgccatatgagtccatcgacaacgacctttatgtctttgacttcaacacccacacttggtcaatcgctccggccaacggagacgttccaaagaccagagtcttaggcacccgcatggtggccgtgggaacgaagctctatgtgttcggaggccgcaataaacagttagagttcgaggacttttactcgtacgatacggtgaaagaggagtggaagttcctgacgaagctggacgaaaagggaggacccgaagctcgtactttccattcgatgacttcagatgaaaaccacgtgtacgtgttcggtggggtgagcaaaggagggctgaatgcaaccccctttcggttcaggacgatcgaggcctataacattgctgaagggaaatgggctcagctccctgaccctggagaggatttcgagaaaagaggaatggccggattccttgtggtgcaaggcaagctttgggtgttttacggattcgcgactgcgaatgatcctaagatacccacactctacgggtcccaggactacgagtctaatcgtgtgcactgctatgatcccgctactcaaaaatggaccgaagtggagaccacaggtttcgagaaaccttctcgtaggagctgttttgcgcatgcggctgtggggaaatatataataatattcggaggtgagattgagcgggacccagaagcacatcaaggtccggggacgttgtcccgtgagggttttgcgttggacactgagacattggtgtgggagaggtatgaaggaggaccaatcaaaccgagcaaccgcggttgggtggcctccacgacgaccaccatcaacggaaagaaaggtctgctcgtgcatggagggaaacttatgaccaacgagcgcaccgatgagatgtacttcttcgcagtcaattcctccacgtaatcaatgctctctcaccctccaaggtctgtttgtgtgtgtggggggtcaaggttttatatgattcaataaaggtttgtgagaccttcagaaactcaccctatcgtcgctgttttcctttttaaatattattatcaaataaaataaatgtgtgtgctgtgtgactgtgtgtggggtttggaaactattatcattggaataatatgtgtgttttgtgtacgtgtgagtataataaaatcataaattatactatgaaattatctacttatttgtcgtatcatttaacttccctcaatcgccataacatttattcatgtgtatccaagtggatgacgaaactatatctaccaattctaatttggaagcattatgattttttttgcgattttgaaatttaaaagtgggatagagtaaatatttggaaagcatgggaatatttattttcctaactggattataaaaaaaactgtatttatagttatttcatgggtataaaaaaagatatgtttttagttaaataattgatattttatattatagattaaatttaagtgtatagtatatacaccttttaatattatttgatgtcgggtgttttatcatataaatataaatataaacaatactaataatatgtatactgtatatacctttttaacactttttttttaatactaacatataacacattgaaatagtcatacgcagcgccaatattttagcttgaaggttaataatttgatatgttgagctcttgcatcattcgagatccaaaaagcatccacaaaaatacaaaacatcagtaattaattagcttttgcttggagagtttgagggcagtcatcattcattttcagcttaatctggaaagcattatagttcaaacattattttcagattcttaacttccttttttagctgactttactttcccttgcgtatagttttggattaaagattatcagagaaatccacatgagatttcagatttgatgaaaatgaagaaaaactttttaagtttggaaaatgataaatgtatcagctctggtcaaagatgagatcgaaataggcccactggactatcttggggaacaagctttaagccttcgaaaaaaaggctgtttccgcttgttttattattaccaaacagatagaaagatgtcttaatagtttcttgttctataaaagtcttattacagacactcttaaaactctctt

SEQ ID NO: 30 - protein, 348 aa, Thlaspi arvense
MARTLQGEWMKVEQKGGQVPAPRSSHGIAVIGDKLYCFGGEDPPYESIDNDLYVFDFNTHTWSIAPANGDVPKTRVLGTRMVAVGTKLYVFGGRNKQLEFEDFYSYDTVKEEWKFLTKLDEKGGPEARTFHSMTSDENHVYVFGGVSKGGLNATPFRFRTIEAYNIAEGKWAQLPDPGEDFEKRGMAGFLVVQGKLWVFYGFATANDPKIPTLYGSQDYESNRVHCYDPATQKWTEVETTGFEKPSRRSCFAHAAVGKYIIIFGGEIERDPEAHQGPGTLSREGFALDTETLVWERYEGGPIKPSNRGWVASTTTTINGKKGLLVHGGKLMTNERTDEMYFFAVNSST

SEQ ID NO: 31 - DNA, 19 bp, Thlaspi arvense
  source 1..19: mol_type = genomic DNA
acgatcttcgaaaacttct

SEQ ID NO: 32 - DNA, 22 bp, Thlaspi arvense
  source 1..22: mol_type = genomic DNA
atcttcgaaaacttctctatat

SEQ ID NO: 33 - DNA, 22 bp, Thlaspi arvense
  source 1..22: mol_type = genomic DNA
aagaaacctaatatgagtttta

SEQ ID NO: 34 - DNA, 19 bp, Thlaspi arvense
  source 1..19: mol_type = genomic DNA
tttttcaactttaaaattt

SEQ ID NO: 35 - DNA, 22 bp, Thlaspi arvense
  source 1..22: mol_type = genomic DNA
aaacactgtaaaagaaactatc

SEQ ID NO: 36 - DNA, 19 bp, Thlaspi arvense
  source 1..19: mol_type = genomic DNA
acatttgcgtatttctcat

SEQ ID NO: 37 - DNA, 19 bp, Thlaspi arvense
  source 1..19: mol_type = genomic DNA
cgaaccctaactcttccca

SEQ ID NO: 38 - DNA, 22 bp, Thlaspi arvense
  source 1..22: mol_type = genomic DNA
tcgggtttgttcgtacggacaa

SEQ ID NO: 39 - DNA, 19 bp, Thlaspi arvense
  source 1..19: mol_type = genomic DNA
tcgctaagatcataaaacc

SEQ ID NO: 40 - DNA, 19 bp, Thlaspi arvense
  source 1..19: mol_type = genomic DNA
agattcacagagactgagc

SEQ ID NO: 41 - DNA, 22 bp, Thlaspi arvense
  source 1..22: mol_type = genomic DNA
aagacttttttccggtgtctct

SEQ ID NO: 42 - DNA, 22 bp, Thlaspi arvense
  source 1..22: mol_type = genomic DNA
gtgttttttttttgaacattaa

SEQ ID NO: 43 - DNA, 19 bp, Thlaspi arvense
  source 1..19: mol_type = genomic DNA
agacaattccccaatatca

SEQ ID NO: 44 - DNA, 22 bp, Thlaspi arvense
  source 1..22: mol_type = genomic DNA
gtgataatttgatattggggaa

SEQ ID NO: 45 - DNA, 19 bp, Thlaspi arvense
  source 1..19: mol_type = genomic DNA
agtgttcattgctaacgag

SEQ ID NO: 46 - DNA, 22 bp, Thlaspi arvense
  source 1..22: mol_type = genomic DNA
catgtttgtgtgtctgtgtaga

SEQ ID NO: 47 - DNA, 22 bp, Thlaspi arvense
  source 1..22: mol_type = genomic DNA
gtttctccgggactcccttatg

SEQ ID NO: 48 - DNA, 22 bp, Thlaspi arvense
  source 1..22: mol_type = genomic DNA
ggggttcctcaagcagaggttc

SEQ ID NO: 49 - DNA, 22 bp, Thlaspi arvense
  source 1..22: mol_type = genomic DNA
gtgcaagcgctctacaaggaaa

SEQ ID NO: 50 - DNA, 22 bp, Thlaspi arvense
  source 1..22: mol_type = genomic DNA
gagtccggatgcgcgcccggag

SEQ ID NO: 51 - DNA, 22 bp, Thlaspi arvense
  source 1..22: mol_type = genomic DNA
gaagttagagaagatgaaactt

SEQ ID NO: 52 - DNA, 22 bp, Thlaspi arvense
  source 1..22: mol_type = genomic DNA
ttatgccaaataaatatatatt

SEQ ID NO: 53 - DNA, 22 bp, Thlaspi arvense
  source 1..22: mol_type = genomic DNA
aaacgtgcttttcgagaaaaaa

SEQ ID NO: 54 - DNA, 22 bp, Thlaspi arvense
  source 1..22: mol_type = genomic DNA
atattaacaatggaaatttaca

SEQ ID NO: 55 - DNA, 22 bp, Thlaspi arvense
  source 1..22: mol_type = genomic DNA
tttgcttcttattccctttttc

SEQ ID NO: 56 - DNA, 22 bp, Thlaspi arvense
  source 1..22: mol_type = genomic DNA
aaaaattatataaattactaga

SEQ ID NO: 57 - DNA, 22 bp, Thlaspi arvense
  source 1..22: mol_type = genomic DNA
aatgaagatggatatatatata

SEQ ID NO: 58 - DNA, 19 bp, Thlaspi arvense
  source 1..19: mol_type = genomic DNA
accgattggtgcaagcaga

SEQ ID NO: 59 - DNA, 19 bp, Thlaspi arvense
  source 1..19: mol_type = genomic DNA
tcaactaacttagatggcc

SEQ ID NO: 60 - DNA, 22 bp, Thlaspi arvense
  source 1..22: mol_type = genomic DNA
aataagctgcacacctacagtt

SEQ ID NO: 61 - DNA, 22 bp, Thlaspi arvense
  source 1..22: mol_type = genomic DNA
attttaattgaagagtggggaa

SEQ ID NO: 62 - DNA, 22 bp, Thlaspi arvense
  source 1..22: mol_type = genomic DNA
tgaaaagaaatctcaaaccacc

SEQ ID NO: 63 - DNA, 22 bp, Thlaspi arvense
  source 1..22: mol_type = genomic DNA
ctactcgttaactagcacaaac

SEQ ID NO: 64 - DNA, 19 bp, Thlaspi arvense
  source 1..19: mol_type = genomic DNA
agtttctttattttaaaaa

SEQ ID NO: 65 - DNA, 22 bp, Thlaspi arvense
  source 1..22: mol_type = genomic DNA
atacttttaaaagcctacgtaa

SEQ ID NO: 66 - DNA, 19 bp, Thlaspi arvense
  source 1..19: mol_type = genomic DNA
agccttatagtgcgcactc

SEQ ID NO: 67 - DNA, 22 bp, Thlaspi arvense
  source 1..22: mol_type = genomic DNA
aaatagatgctcatatacatac

SEQ ID NO: 68 - DNA, 19 bp, Thlaspi arvense
  source 1..19: mol_type = genomic DNA
aagacgaattttcaatgac

SEQ ID NO: 69 - DNA, 22 bp, Thlaspi arvense
  source 1..22: mol_type = genomic DNA
gaatgttgcaagtcattgaaaa

SEQ ID NO: 70 - DNA, 19 bp, Thlaspi arvense
  source 1..19: mol_type = genomic DNA
cattccgtccatttgtaac

SEQ ID NO: 71 - DNA, 22 bp, Thlaspi arvense
  source 1..22: mol_type = genomic DNA
acaaaaatgattcgtcggttac

SEQ ID NO: 72 - DNA, 19 bp, Thlaspi arvense
  source 1..19: mol_type = genomic DNA
aaactatgaaacatgattg

SEQ ID NO: 73 - DNA, 22 bp, Thlaspi arvense
  source 1..22: mol_type = genomic DNA
tcatgaagattaacaaaagcta

SEQ ID NO: 74 - DNA, 19 bp, Thlaspi arvense
  source 1..19: mol_type = genomic DNA
gccacagtcatgaagatta

SEQ ID NO: 75 - DNA, 22 bp, Thlaspi arvense
  source 1..22: mol_type = genomic DNA
tggagataccttgacacgcaga

SEQ ID NO: 76 - DNA, 20 bp, Thlaspi arvense
  source 1..20: mol_type = genomic DNA
ttgcagctgtaactttctta

SEQ ID NO: 77 - DNA, 20 bp, Thlaspi arvense
  source 1..20: mol_type = genomic DNA
gaagttgattgtgccactaa

SEQ ID NO: 78 - DNA, 20 bp, Thlaspi arvense
  source 1..20: mol_type = genomic DNA
gatcggtctaaccataccgg

SEQ ID NO: 79 - DNA, 20 bp, Thlaspi arvense
  source 1..20: mol_type = genomic DNA
attgttgcaatcaagaaacg

SEQ ID NO: 80 - DNA, 1109 bp, Thlaspi arvense
  source 1..1109: mol_type = genomic DNA
atgtcaagaaagccgtgtttgtcggagaagggctgaagaaaggggcatggaccaccgaagaagacaagaaactcatctcttacatccacgagcacggtgaaggaggctggcgcgacattccccaaaaagctgggttgaaacggtgtggaaagagttgtaggctgcgatggactaactacctaaaacctgagatcaaaagaggcgagtttagttcagaggaggaacagattatcattatgcttcatgcttctcgtggcaacaagtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtga

SEQ ID NO: 81 - protein, 11 aa, Thlaspi arvense
MSRKPCLSEKG

SEQ ID NO: 82 - DNA, 1111 bp, Thlaspi arvense
  source 1..1111: mol_type = genomic DNA
atgtcaagaaagccgtgttgatgtcggagaagggctgaagaaaggggcatggaccaccgaagaagacaagaaactcatctcttacatccacgagcacggtgaaggaggctggcgcgacattccccaaaaagctgggttgaaacggtgtggaaagagttgtaggctgcgatggactaactacctaaaacctgagatcaaaagaggcgagtttagttcagaggaggaacagattatcattatgcttcatgcttctcgtggcaacaagtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtga

SEQ ID NO: 83 - protein, 6 aa, Thlaspi arvense
MSRKPC

SEQ ID NO: 84 - DNA, 1111 bp, Thlaspi arvense
  source 1..1111: mol_type = genomic DNA
atgtcaagaaagccgtgttgtgtcggagaagggctgaagaaaggggcatggaccaccgaagaagacaagaaactcatctcttacatccacgaggcacggtgaaggaggctggcgcgacattccccaaaaagctgggttgaaacggtgtggaaagagttgtaggctgcgatggactaactacctaaaacctgagatcaaaagaggcgagtttagttcagaggaggaacagattatcattatgcttcatgcttctcgtggcaacaagtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtga

SEQ ID NO: 85 - protein, 33 aa, Thlaspi arvense
MSRKPCCVGEGLKKGAWTTEEDKKLISYIHEAR

SEQ ID NO: 86 - DNA, 1110 bp, Thlaspi arvense
  source 1..1110: mol_type = genomic DNA
atgtcaagaaagccgtgttgtgtcggagaagggctgaagaaaggggcatggaccaccgaagaagacaagaaactcatctcttacatccacgggcacggtgaaggaggctggcgcgacattccccaaaaagctgggttgaaacggtgtggaaagagttgtaggctgcgatggactaactacctaaaacctgagatcaaaagaggcgagtttagttcagaggaggaacagattatcattatgcttcatgcttctcgtggcaacaagtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtga

SEQ ID NO: 87 - protein, 369 aa, Thlaspi arvense
MSRKPCCVGEGLKKGAWTTEEDKKLISYIHGHGEGGWRDIPQKAGLKRCGKSCRLRWTNYLKPEIKRGEFSSEEEQIIIMLHASRGNKWSVIARHLPRRTDNEIKNYWNTHLKKRLIEQGVDPVTHKPLASNSGPTATTPPENLHFLDESSSDKQYSRSSSMPSLSRLPSSGFNTVSEIASNVGTPVQVGSLSCKKRFKKSSSTSRLLNKFAAKATSIKDILSASMEGSSSAATTISHASFLNGFSEQSRNEEDSSNASLTNTLAEFDPFSQSSLYPEHEINVTSDIGMDQVYDFSQFLEKLGSEGHNELNVEYGHDLLMSDVSQEVSSPSVDDQDNMIGSFEGWSNYLLDHADFIYDTDSDSLEKHFM

SEQ ID NO: 88 - DNA, 1111 bp, Thlaspi arvense
  source 1..1111: mol_type = genomic DNA
atgtcaagaaagccgtgttgtgtcggagaagggctgaagaaaggggcatggaccaccgaagaagacaagaaactcatctcttacatccacgaagcacggtgaaggaggctggcgcgacattccccaaaaagctgggttgaaacggtgtggaaagagttgtaggctgcgatggactaactacctaaaacctgagatcaaaagaggcgagtttagttcagaggaggaacagattatcattatgcttcatgcttctcgtggcaacaagtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtga

SEQ ID NO: 89 - protein, 33 aa, Thlaspi arvense; identical to SEQ ID NO: 85

SEQ ID NO: 90 - DNA, 1108 bp, Thlaspi arvense
  source 1..1108: mol_type = genomic DNA
atgtcaagaaagccgtgttgtgtcggagaagggctgaagaaaggggcatggaccaccgaagaagacaagaaactcatctcttacatccacgcacggtgaaggaggctggcgcgacattccccaaaaagctgggttgaaacggtgtggaaagagttgtaggctgcgatggactaactacctaaaacctgagatcaaaagaggcgagtttagttcagaggaggaacagattatcattatgcttcatgcttctcgtggcaacaagtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtga

SEQ ID NO: 91 - protein, 32 aa, Thlaspi arvense
MSRKPCCVGEGLKKGAWTTEEDKKLISYIHAR

SEQ ID NO: 92 - DNA, 1509 bp, Thlaspi arvense
  source 1..1509: mol_type = genomic DNA
atggaagatatcatcatcggcgttgtggctctcgccgccgttctcctcttcttcctctaccaaagcccaaaaaccaaacggtataagctgcctcccggcccacggccgcttccggtgatcggaaacctccaccagcttagccaggttaacccacaacggttcttctatggatgggccaaaaaatatggaccaatcttatcatacaagataggaaacaaaacaatgatggtaatttcttcggctgagctaaccaaagagcttctcaagacgcaagatgtcaacttcgccaaccggcctcctcaccgtggtcacgagctcatgacctacggccgaagtgacatggcgatgaaccactacacaccgttgtaccgggagatgaggaagatgggcatgaaccacttgttctcccccactcgtgtagccacctttaagcacgtacgggaggaggaggctaggaggatgatgtttaagatcgagaaggctgcggagagatctgaaccggtcgatataagcgagcttatgttgaccttcacgaactcggttgtgtgtaggcaagctttcgggaagaagtacaatgaagatggggaagagatgaagagattcatcaggattctttatgggactcagagcgtattggggaagatttttttctctgattttttcccgtttactcgctacgttcttgataattggaccggcctcacgaaatatatgatggactgttttgaaagacaagacacttacatacaagagattatcgatgagacacttgatcccaacaaggtaaagccagaaacggagagcatgatcgatctcttgatggaggtctacaaagaacaaccattcgcctccaagttcacaattgggaatgtcaaaggcgttatcttgaatatagtggttgcgggaaccgacacggcggctgcggcggttgtgtgggggatgacgtatctaatgaagtaccctcaagttatgaagaaagctcaagcagaagtgagagagtatgcaaaagagaaagatctaacgtttattactgaagacgacgtcaagaaccttccttacttcagagctttagttaaagaaaccttaaggatcgaaccagtgattcctctccttatccctcgttgttgcattcaagacaccaagatcgccggttacgatgtccccgcggggaccacggtcaacgtaaacgcgtgggcggtgtcacgcgacgagaaggagtggggcccaaaccctgatgaattcaggcccgagaggtttcttgagaaggacgtggacttcaaaggcacggactatgagtttataccgtttgggtcaggccggagaatgtgccctggaatgcgtcttggcgcggcgatgatcgaggttccgtatgcgaaccttttgctcaactttgacttcaaacttgctgatggactgaaaccagaagagatcaacatggatgttatgacaggtcttgctatgcacaaggcggttcatctcaggcttgttcccgagaaagtgaggaagtga

SEQ ID NO: 93 - DNA, 4468 bp, Thlaspi arvense
  source 1..4468: mol_type = genomic DNA
atggcaagacttgatattcgagtcggtaaaatcgtcaaggctcggaaacatcctaacgcagattcattgtatttggaagagatcgatgttggaggaggtgaagttcgcaccgttgtgagtggactggtcaaatacatacctcttgaggagatgcaggtttgttaaatctccacgaatcaaacctaacctagtcatctgctttcatcccaatatatctgactgttctttttggacagaaccgtatggtttgtgttctttgcaacttgaagccggcgaaaatgagggatgttatgtcacaagcgatggttcttgccgcttctagtagtgatggcagcaaggtaaaagtccacaagaatgagtctctgacctgttcatcgttacgctatatccatttcgtttatagataatttgttttttgggaactttcaggttgagttagtcgagcctcctgaatctgctaagatcggagagagggttaggtttgccgggtttgaaggcgagccagacgatgttttaaaccctaagaagaaaatatgggagacacttgtggtggatctgcacacaaatgagaatctagttgcttgctacaaagatttgcctttcactacagattcaggtgtatgcaaggtttcatccatcagcaatggtacaatccggtaaaaaagctttaagttggtctttatctttcttcatttctctctctctcttttgaggggaaacacatttttctgagatttcatttgaagaattgattctattaacaaaccttgtcttgtaaactcaaactcgtcacttttttggttcttaatttggttttgagaaacaatatgttaattttttctttagacaaacttggtaggccttttggtatatcaatatcagctttttatactggacccggacggaaagcaaccacacaagttgttgaggtttcgatgatagtgtataaataattatcgtatttatcaaaaggtcaatcacatcatttatttattttcagtaacaactttgtaataaagtcttccgcaagcaaacttttgtgcaaatttcttggaaactcagtttgcgacaaactacaaagtaaatgttggctatgtatgacatcgttatgattagttcaaagtaccttaggtaaatttggttttacttttgtatttataaaattgagtatgttttagaattctagctatatatgtcagcgtatgttacatttttctgtataagattggattgtactatagtgcataggcccggggttggtttagaccactcagtgaagcaagttaaataaagatctgtgtaaactacaaatagatatggacaaatatgactgcgactgcatgaaagtatataaccatatatgttaattaaaagcataggacattagagggaccttgggtaggtgaaggtgtttctcaactctaacacgtgaggttttcataagtaggtagtaaaaaaggagtaagcgttttgattataaaattagatttcggtttaattcggttatggtatttggtttggtaaagaagaaaaggaatgcaattaataatttctctatatattaaggagctgtgtgctgtttatgtaccactcaaaagtagtaagtaaacaactaagaatggaagatatcatcatcggcgttgtggctctcgccgccgttctcctcttcttcctctaccaaagcccaaaaaccaaacggtataagctgcctcccggcccacggccgcttccggtgatcggaaacctccaccagcttagccaggttaacccacaacggttcttctatggatgggccaaaaaatatggaccaatcttatcatacaagataggaaacaaaacaatgatggtaatttcttcggctgagctaaccaaagagcttctcaagacgcaagatgtcaacttcgccaaccggcctcctcaccgtggtcacgagctcatgacctacggccgaagtgacatggcgatgaaccactacacaccgttgtaccgggagatgaggaagatgggcatgaaccacttgttctcccccactcgtgtagccacctttaagcacgtacgggaggaggaggctaggaggatgatgtttaagatcgagaaggctgcggagagatctgaaccggtcgatataagcgagcttatgttgaccttcacgaactcggttgtgtgtaggcaagctttcgggaagaagtacaatgaagatggggaagagatgaagagattcatcaggattctttatgggactcagagcgtattggggaagatttttttctctgattttttcccgtttactcgctacgttcttgataattggaccggcctcacgaaatatatgatggactgttttgaaagacaagacacttacatacaagagattatcgatgagacacttgatcccaacaaggtaaagccagaaacggagagcatgatcgatctcttgatggaggtctacaaagaacaaccattcgcctccaagttcacaattgggaatgtcaaaggcgttatcttggtacgtataaaaccacttctcaattttcttttgttttgaatcgttgcttgtgctacaagcacccactcgcttgcgtttcactcttcttttcgttgttgtgtttcaaaagaatatagtggttgcgggaaccgacacggcggctgcggcggttgtgtgggggatgacgtatctaatgaagtaccctcaagttatgaagaaagctcaagcagaagtgagagagtatgcaaaagagaaagatctaacgtttattactgaagacgacgtcaagaaccttccttacttcagagctttagttaaagaaaccttaaggatcgaaccagtgattcctctccttatccctcgttgttgcattcaagacaccaagatcgccggttacgatgtccccgcggggaccacggtcaacgtaaacgcgtgggcggtgtcacgcgacgagaaggagtggggcccaaaccctgatgaattcaggcccgagaggtttcttgagaaggacgtggacttcaaaggcacggactatgagtttataccgtttgggtcaggccggagaatgtgccctggaatgcgtcttggcgcggcgatgatcgaggttccgtatgcgaaccttttgctcaactttgacttcaaacttgctgatggactgaaaccagaagagatcaacatggatgttatgacaggtcttgctatgcacaaggcggttcatctcaggcttgttcccgagaaagtgaggaagtgacagtttctagtagtaaaataaatctctcactcttgttatataatatattgttccctaaagcgtactatgctttaaggtttggtttctgtattaagtggcaatcgtctctcttataatttttccttttacatcaaacgacttacgcaaaaaagctagatagccgagccaaaaggtcgtcttggtacataagaaaaaaaaaccaaagcaaaagagaacataaatgattaaattaggctttttttttttcttgtgattcatttaagttgattcctcgaaacatgttgaccactcatggggttcattaataatttccaaaacaaagcttctaagtcatagtgaagtgacaagagcctatcctaagccactaaacgcggctcaagtcttctatgagccatcacactagtgactggacaaatgttagctgttttgatatccagtggactcgcgggtctttggtcctgatgaaccacgtaatctattagaatatatactagctcaaagggtacttgtgttcaatcaatagatttcctctttagatagtcttttataggtaatattagatcaacgtaatattagtttaagggaaattcttatcgaagttcattataaaatgaaaaaaatattattagtttttttttgtaatacaaacaaatattaatttttttttctccaaaatatcactttcagaatatcaattttcaaaatactaatatttattttaaaaatatttataaaacttaacaaaggtctcttaacaatataataataataattttaatatagtataattgtatggtttaattttaaggtttcttaaaaaagttaaattaaattatctcaaagtttgatttgagagacccttttcattcttttcttcccaacttttattatttttaatattagaaacttttttaaatacttctaatgaagatgttctaataatttatacaaaataataaactatgcaaaccttataaaatccttttgcaagaaatataatatacatatctacaaaagaattggttctgcatctgtattttccttctccatgtagacattctttctagtttttctacccacatgacttgtattttcttattcttagcattttgtaattatgtataacaatgccttctcatatttttatgtgttttctcatcttgttatatggacgtcgaattatcattgcccatattattttatccatgattttttgttttatgttttcagaa

SEQ ID NO: 94 - protein, 502 aa, Thlaspi arvense
MEDIIIGVVALAAVLLFFLYQSPKTKRYKLPPGPRPLPVIGNLHQLSQVNPQRFFYGWAKKYGPILSYKIGNKTMMVISSAELTKELLKTQDVNFANRPPHRGHELMTYGRSDMAMNHYTPLYREMRKMGMNHLFSPTRVATFKHVREEEARRMMFKIEKAAERSEPVDISELMLTFTNSVVCRQAFGKKYNEDGEEMKRFIRILYGTQSVLGKIFFSDFFPFTRYVLDNWTGLTKYMMDCFERQDTYIQEIIDETLDPNKVKPETESMIDLLMEVYKEQPFASKFTIGNVKGVILNIVVAGTDTAAAAVVWGMTYLMKYPQVMKKAQAEVREYAKEKDLTFITEDDVKNLPYFRALVKETLRIEPVIPLLIPRCCIQDTKIAGYDVPAGTTVNVNAWAVSRDEKEWGPNPDEFRPERFLEKDVDFKGTDYEFIPFGSGRRMCPGMRLGAAMIEVPYANLLLNFDFKLADGLKPEEINMDVMTGLAMHKAVHLRLVPEKVRK

SEQ ID NO: 95 - DNA, 1510 bp, Thlaspi arvense
  source 1..1510: mol_type = genomic DNA
atggaagatatcatcatcggcgttgtggctctcgccgccgttctcctcttcttcctctaccaaagcccaaaaaccaaacggtataagctgcctcccggcccacggccgcttccggtgatcggaaacctccaccagcttagccaggttaacccacaacggttcttctatggatgggccaaaaaatatggaccaatcttatcataccaagataggaaacaaaacaatgatggtaatttcttcggctgagctaaccaaagagcttctcaagacgcaagatgtcaacttcgccaaccggcctcctcaccgtggtcacgagctcatgacctacggccgaagtgacatggcgatgaaccactacacaccgttgtaccgggagatgaggaagatgggcatgaaccacttgttctcccccactcgtgtagccacctttaagcacgtacgggaggaggaggctaggaggatgatgtttaagatcgagaaggctgcggagagatctgaaccggtcgatataagcgagcttatgttgaccttcacgaactcggttgtgtgtaggcaagctttcgggaagaagtacaatgaagatggggaagagatgaagagattcatcaggattctttatgggactcagagcgtattggggaagatttttttctctgattttttcccgtttactcgctacgttcttgataattggaccggcctcacgaaatatatgatggactgttttgaaagacaagacacttacatacaagagattatcgatgagacacttgatcccaacaaggtaaagccagaaacggagagcatgatcgatctcttgatggaggtctacaaagaacaaccattcgcctccaagttcacaattgggaatgtcaaaggcgttatcttgaatatagtggttgcgggaaccgacacggcggctgcggcggttgtgtgggggatgacgtatctaatgaagtaccctcaagttatgaagaaagctcaagcagaagtgagagagtatgcaaaagagaaagatctaacgtttattactgaagacgacgtcaagaaccttccttacttcagagctttagttaaagaaaccttaaggatcgaaccagtgattcctctccttatccctcgttgttgcattcaagacaccaagatcgccggttacgatgtccccgcggggaccacggtcaacgtaaacgcgtgggcggtgtcacgcgacgagaaggagtggggcccaaaccctgatgaattcaggcccgagaggtttcttgagaaggacgtggacttcaaaggcacggactatgagtttataccgtttgggtcaggccggagaatgtgccctggaatgcgtcttggcgcggcgatgatcgaggttccgtatgcgaaccttttgctcaactttgacttcaaacttgctgatggactgaaaccagaagagatcaacatggatgttatgacaggtcttgctatgcacaaggcggttcatctcaggcttgttcccgagaaagtgaggaagtga

SEQ ID NO: 96 - protein, 81 aa, Thlaspi arvense
MEDIIIGVVALAAVLLFFLYQSPKTKRYKLPPGPRPLPVIGNLHQLSQVNPQRFFYGWAKKYGPILSYQDRKQNNDGNFFG

SEQ ID NO: 97 - DNA, 1509 bp, Thlaspi arvense
  source 1..1509: mol_type = genomic DNA
atggaagatatcatcatcggcgttgtggctctcgccgccgttctcctcttcttcctctaccaaagcccaaaaaccaaacggtataagctgcctcccggcccacggccgcttccggtgatcggaaacctccaccagcttagccaggttaacccacaacggttcttctatggatgggccaaaaaatatggaccaatcttatcatccaagataggaaacaaaacaatgatggtaatttcttcggctgagctaaccaaagagcttctcaagacgcaagatgtcaacttcgccaaccggcctcctcaccgtggtcacgagctcatgacctacggccgaagtgacatggcgatgaaccactacacaccgttgtaccgggagatgaggaagatgggcatgaaccacttgttctcccccactcgtgtagccacctttaagcacgtacgggaggaggaggctaggaggatgatgtttaagatcgagaaggctgcggagagatctgaaccggtcgatataagcgagcttatgttgaccttcacgaactcggttgtgtgtaggcaagctttcgggaagaagtacaatgaagatggggaagagatgaagagattcatcaggattctttatgggactcagagcgtattggggaagatttttttctctgattttttcccgtttactcgctacgttcttgataattggaccggcctcacgaaatatatgatggactgttttgaaagacaagacacttacatacaagagattatcgatgagacacttgatcccaacaaggtaaagccagaaacggagagcatgatcgatctcttgatggaggtctacaaagaacaaccattcgcctccaagttcacaattgggaatgtcaaaggcgttatcttgaatatagtggttgcgggaaccgacacggcggctgcggcggttgtgtgggggatgacgtatctaatgaagtaccctcaagttatgaagaaagctcaagcagaagtgagagagtatgcaaaagagaaagatctaacgtttattactgaagacgacgtcaagaaccttccttacttcagagctttagttaaagaaaccttaaggatcgaaccagtgattcctctccttatccctcgttgttgcattcaagacaccaagatcgccggttacgatgtccccgcggggaccacggtcaacgtaaacgcgtgggcggtgtcacgcgacgagaaggagtggggcccaaaccctgatgaattcaggcccgagaggtttcttgagaaggacgtggacttcaaaggcacggactatgagtttataccgtttgggtcaggccggagaatgtgccctggaatgcgtcttggcgcggcgatgatcgaggttccgtatgcgaaccttttgctcaactttgacttcaaacttgctgatggactgaaaccagaagagatcaacatggatgttatgacaggtcttgctatgcacaaggcggttcatctcaggcttgttcccgagaaagtgaggaagtga

SEQ ID NO: 98 - protein, 502 aa, Thlaspi arvense
MEDIIIGVVALAAVLLFFLYQSPKTKRYKLPPGPRPLPVIGNLHQLSQVNPQRFFYGWAKKYGPILSSKIGNKTMMVISSAELTKELLKTQDVNFANRPPHRGHELMTYGRSDMAMNHYTPLYREMRKMGMNHLFSPTRVATFKHVREEEARRMMFKIEKAAERSEPVDISELMLTFTNSVVCRQAFGKKYNEDGEEMKRFIRILYGTQSVLGKIFFSDFFPFTRYVLDNWTGLTKYMMDCFERQDTYIQEIIDETLDPNKVKPETESMIDLLMEVYKEQPFASKFTIGNVKGVILNIVVAGTDTAAAAVVWGMTYLMKYPQVMKKAQAEVREYAKEKDLTFITEDDVKNLPYFRALVKETLRIEPVIPLLIPRCCIQDTKIAGYDVPAGTTVNVNAWAVSRDEKEWGPNPDEFRPERFLEKDVDFKGTDYEFIPFGSGRRMCPGMRLGAAMIEVPYANLLLNFDFKLADGLKPEEINMDVMTGLAMHKAVHLRLVPEKVRK

SEQ ID NO: 99 - DNA, 20 bp, Thlaspi arvense
  source 1..20: mol_type = genomic DNA
ggtgtcgtacgatagagtgt

SEQ ID NO: 100 - DNA, 20 bp, Thlaspi arvense
  source 1..20: mol_type = genomic DNA
acggtctttccgagagtatg

SEQ ID NO: 101 - DNA, 19 bp, Thlaspi arvense
  source 1..19: mol_type = genomic DNA
atccaggatcctcatgttt

SEQ ID NO: 102 - DNA, 20 bp, Thlaspi arvense
  source 1..20: mol_type = genomic DNA
actcaacgaattaccgtctg

SEQ ID NO: 103 - DNA, 20 bp, Thlaspi arvense
  source 1..20: mol_type = genomic DNA
gttaagtctaatgccgaaga

SEQ ID NO: 104 - DNA, 20 bp, Thlaspi arvense
  source 1..20: mol_type = genomic DNA
ccactcataacggtctttcc

SEQ ID NO: 105 - DNA, 20 bp, Thlaspi arvense
  source 1..20: mol_type = genomic DNA
ccactcataacggtctttcc

SEQ ID NO: 106 - DNA, 20 bp, Thlaspi arvense
  source 1..20: mol_type = genomic DNA
agcctccttcaccgtgctcg

SEQ ID NO: 107 - DNA, 20 bp, Thlaspi arvense
  source 1..20: mol_type = genomic DNA
tcaagaaagccgtgttgtgt

SEQ ID NO: 108 - DNA, 20 bp, Thlaspi arvense
  source 1..20: mol_type = genomic DNA
aagaagacaagaaactcatc

SEQ ID NO: 109 - DNA, 19 bp, Thlaspi arvense
  source 1..19: mol_type = genomic DNA
agggttgaaacggtgtgga

SEQ ID NO: 110 - DNA, 20 bp, Thlaspi arvense
  source 1..20: mol_type = genomic DNA
gaccatgctgtatcggagaa

SEQ ID NO: 111 - DNA, 20 bp, Thlaspi arvense
  source 1..20: mol_type = genomic DNA
aaggagcagtcgttacagcc

SEQ ID NO: 112 - DNA, 20 bp, Thlaspi arvense
  source 1..20: mol_type = genomic DNA
gacgaagccgaaaacattgg

SEQ ID NO: 113 - DNA, 20 bp, Thlaspi arvense
  source 1..20: mol_type = genomic DNA
acttgcccctagccctagtc

SEQ ID NO: 114 - DNA, 20 bp, Thlaspi arvense
  source 1..20: mol_type = genomic DNA
actcggatgagcattcacat

SEQ ID NO: 115 - DNA, 20 bp, Thlaspi arvense
  source 1..20: mol_type = genomic DNA
tgaacatggcgaaggaggct

SEQ ID NO: 116 - DNA, 20 bp, Thlaspi arvense
  source 1..20: mol_type = genomic DNA
tggaaggaaccttggttagc

SEQ ID NO: 117 - DNA, 20 bp, Thlaspi arvense
  source 1..20: mol_type = genomic DNA
ccaatcttatcatacaagat

SEQ ID NO: 118 - DNA, 20 bp, Thlaspi arvense
  source 1..20: mol_type = genomic DNA
aaaccctactccaccgtcga

SEQ ID NO: 119 - DNA, 20 bp, Thlaspi arvense
  source 1..20: mol_type = genomic DNA
gaagaagtgaaatgcattgt

SEQ ID NO: 120 - DNA, 20 bp, Thlaspi arvense
  source 1..20: mol_type = genomic DNA
ttcctctgcgacacttactt

SEQ ID NO: 121 - DNA, 20 bp, Thlaspi arvense
  source 1..20: mol_type = genomic DNA
gaagaagtgaaatgcattgt

SEQ ID NO: 122 - DNA, 20 bp, Thlaspi arvense
  source 1..20: mol_type = genomic DNA
tgtatgtgaaagtcaaagcc

SEQ ID NO: 123 - DNA, 20 bp, Thlaspi arvense
  source 1..20: mol_type = genomic DNA
tccaagcgcagcagagcgac

SEQ ID NO: 124 - DNA, 20 bp, Thlaspi arvense
  source 1..20: mol_type = genomic DNA
tctcctccatgtcggccatg

SEQ ID NO: 125 - DNA, 20 bp, Thlaspi arvense
  source 1..20: mol_type = genomic DNA
ccctactccaccgtcgatgg

SEQ ID NO: 126 - DNA, 20 bp, Thlaspi arvense
  source 1..20: mol_type = genomic DNA
actctcgggatggagccacg

SEQ ID NO: 127 - DNA, 1016 bp, Thlaspi arvense
  source 1..1016: mol_type = genomic DNA
atgtcgaaaagaccatgctgtatcggagaagggttaaagaaagggcatggacgtcaggggaagacaaaaaactcatctcttatatccatgaacatggcgaaggaggttggcgtgacattcccgaaaaagctgggctaaaacggtgtggaaagagttgcagactgcgatgggcgaactatttgaaccccgatatcaagagaggaggatttagctacgaggaagaacagatcatcatcatgcttcatgcttctcgtggcaacaagtggtcagtcatagcaagacatttgccgcaaagaacagacaacgagatcaaaaactattggaacacacatctcaagaaacgcctgatcaataagagcactgattccgtgacccacaagcctctagcttcctctaaccctagtcctaccgagcgtaagaagctcgattcccaagaagaatccaatcccaaggagcagtcgttacagccgggttcgaagtctccagtatctctttccctttcttcgagtttcaacgacactgtacccgagatcatgaccagtgatgagacgcctctagaaagtggtttcttgagttgcaaaaaaagtgtcgagagatcgagctcaacatcaaggcttttaaacaaagttgcagctagagcttcttccatcgggagtatcttatcaacctccatagaaggaactttgagatctcctgcatcgtcctcatgtctcccaaactcattgtgtcaatcatctgaacacaacaaggatcaagatctcggtacgagcattgatcttagcatccccgattacgattactcccactttctcgagcacttcatcaatagcgaagacgaagccgaaaacattggtggctgcaatcaagatctccttatgtccgatttcccatcaacattagtggataaagaaaatatgaattttgaagacataaccggttggtcaagttatcttctcgaccatcccagttttacgtatgaatcggaacaagattccgacgacaacaacttgttatga

SEQ ID NO: 128 - protein, 45 aa, Thlaspi arvense
MSKRPCCIGEGLKKGHGRQGKTKNSSLISMNMAKEVGVTFPKKLG

SEQ ID NO: 129 - DNA, 989 bp, Thlaspi arvense
  source 1..989: mol_type = genomic DNA
atgtcgaaaagaccatgctatggacgtcaggggaagacaaaaaactcatctcttatatccatgaacatggcgaaggaggttggcgtgacattcccgaaaaagctgggctaaaacggtgtggaaagagttgcagactgcgatgggcgaactatttgaaccccgatatcaagagaggaggatttagctacgaggaagaacagatcatcatcatgcttcatgcttctcgtggcaacaagtggtcagtcatagcaagacatttgccgcaaagaacagacaacgagatcaaaaactattggaacacacatctcaagaaacgcctgatcaataagagcactgattccgtgacccacaagcctctagcttcctctaaccctagtcctaccgagcgtaagaagctcgattcccaagaagaatccaatcccaaggagcagtcgttacagccgggttcgaagtctccagtatctctttccctttcttcgagtttcaacgacactgtacccgagatcatgaccagtgatgagacgcctctagaaagtggtttcttgagttgcaaaaaaagtgtcgagagatcgagctcaacatcaaggcttttaaacaaagttgcagctagagcttcttccatcgggagtatcttatcaacctccatagaaggaactttgagatctcctgcatcgtcctcatgtctcccaaactcattgtgtcaatcatctgaacacaacaaggatcaagatctcggtacgagcattgatcttagcatccccgattacgattactcccactttctcgagcacttcatcaatagcgaagacgaagccgaaaacattggtggctgcaatcaagatctccttatgtccgatttcccatcaacattagtggataaagaaaatatgaattttgaagacataaccggttggtcaagttatcttctcgaccatcccagttttacgtatgaatcggaacaagattccgacgacaacaacttgttatga

SEQ ID NO: 130 - protein, 36 aa, Thlaspi arvense
MSKRPCYGRQGKTKNSSLISMNMAKEVGVTFPKKLG

SEQ ID NO: 131 - DNA, 491 bp, Thlaspi arvense
  source 1..491: mol_type = genomic DNA
atgtcaagaaagccatgttgtgtgggagaagggctgaagaaaggagcatggaccgccgaagaagacaagaaactcatctcttacatccatgaaaaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatga

SEQ ID NO: 132 - protein, 69 aa, Thlaspi arvense
MSRKPCCVGEGLKKGAWTAEEDKKLISYIHEKEPWLALPHCLHVSMMTFPKLANSRWTNMIHSLSRLNT

SEQ ID NO: 133 - DNA, 477 bp, Thlaspi arvense
  source 1..477: mol_type = genomic DNA
atgtcaagaaagccatgttgtgtgggagaagggctgaagaaaggagcatggaccgccgaagaagacaagaaactcatctaaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatga

SEQ ID NO: 134 - protein, 26 aa, Thlaspi arvense
MSRKPCCVGEGLKKGAWTAEEDKKLI

SEQ ID NO: 135 - DNA, 1082 bp, Thlaspi arvense
  source 1..1082: mol_type = genomic DNA
atgtcaagaaagccatgttgtgtgggagaagggctgaagaaaggagcatggaccgccgaagaagacaagaaactcatctcttacatccatgaaacatggcgaaggaggctggcgtgacattccccaaaaagctggactaaaacgatgtggaaaaagttgtagattgcgatgggctaactatttgaaaccggatatcaagagaggagaatttagctacgaggaggagcagattatcatcatgcttcacgcttcccgtggcaacaagtggtcggtcatagcgagacatttgcccaaaaggacagacaacgagatcaagaactattggaacacacaccttaaaaaacgccttatcgatcaaggtattgatcccgtgacccacaagccacttgcccctagccctagtccggccacgctcaagccttctgatttccaagatgactcatcaaacctgggaaactcggatgagcattcacattcgggttctatgtctccaaaatctcttcctccgtcttcaagctcctgcaatctagcggagataagcagcagtgatgagacaccgaaaaatgatggttccttgaaatccaagaaacgttcttttaagagatcaagttctacatcaaagctgttaaacaaagttgcatctagggctgcttccattggaaatatcttatcagcgtccatggaaaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatga

SEQ ID NO: 136 - protein, 38 aa, Thlaspi arvense
MSRKPCCVGEGLKKGAWTAEEDKKLISYIHETWRRRLA

SEQ ID NO: 137 - DNA, 1077 bp, Thlaspi arvense
  source 1..1077: mol_type = genomic DNA
atgtcaagaaagccatgttgtgtgggagaagggctgaagaaaggagcatggaccgccgaagaagacaagaaactcatctcttacatccatgaacatggcgaaggaggctggcgtgacattccccaaaaagctggactaaaacgatgtggaaaaagttgtagattgcgatgggctaactatttgaaaccggatatcaagagaggagaatttagctacgaggaggagcagattatcatcatgcttcacgcttcccgtggcaacaagtggtcggtcatagcgagacatttgcccaaaaggacagacaacgagatcaagaactattggaacacacaccttaaaaaacgccttatcgatcaaggtattgatcccgtgacccacaagccacttgcccctagccctagtccggccacgctcaagccttctgatttccaagatgactcatcaaacctgggaaactcggatgagcattcacattcgggttctatgtctccaaaatctcttcctccgtcttcaagctcctgcaatctagcggagataagcagcagtgatgagacaccgaaaaatgatggttccttgaaatccaagaaacgttcttttaagagatcaagttctacatcaaagctgttaaacaaagttgcatctagggctgcttccattggaaatatcttatcagcgtccaaaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatga

SEQ ID NO: 138 - protein, 358 aa, Thlaspi arvense
MSRKPCCVGEGLKKGAWTAEEDKKLISYIHEHGEGGWRDIPQKAGLKRCGKSCRLRWANYLKPDIKRGEFSYEEEQIIIMLHASRGNKWSVIARHLPKRTDNEIKNYWNTHLKKRLIDQGIDPVTHKPLAPSPSPATLKPSDFQDDSSNLGNSDEHSHSGSMSPKSLPPSSSSCNLAEISSSDETPKNDGSLKSKKRSFKRSSSTSKLLNKVASRAASIGNILSASKGTLVSSTALSPCLNDDFSEASQFQMDEYDPFPQSSEHITDHMKEDTGMIFDLNNSEYDFSQFLEQFSNEGEETENIGGYNQDLLSSDVSSPSVDEDNMMGNITGSGWSSYLVDHSDFVYDKIQDNDDRNFI

SEQ ID NO: 139 - DNA, 1076 bp, Thlaspi arvense
  source 1..1076: mol_type = genomic DNA
atgtcaagaaagccatgttgtgtgggagaagggctgaagaaaggagcatggaccgccgaagaagacaagaaactcatctcttacatccatgaacatggcgaaggaggctggcgtgacattccccaaaaagctggactaaaacgatgtggaaaaagttgtagattgcgatgggctaactatttgaaaccggatatcaagagaggagaatttagctacgaggaggagcagattatcatcatgcttcacgcttcccgtggcaacaagtggtcggtcatagcgagacatttgcccaaaaggacagacaacgagatcaagaactattggaacacacaccttaaaaaacgccttatcgatcaaggtattgatcccgtgacccacaagccacttgcccctagccctagtccggccacgctcaagccttctgatttccaagatgactcatcaaacctgggaaactcggatgagcattcacattcgggttctatgtctccaaaatctcttcctccgtcttcaagctcctgcaatctagcggagataagcagcagtgatgagacaccgaaaaatgatggttccttgaaatccaagaaacgttcttttaagagatcaagttctacatcaaagctgttaaacaaagttgcatctagggctgcttccattggaaatatcttatcagcgtccaaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatga

SEQ ID NO: 140 - protein, 264 aa, Thlaspi arvense
MSRKPCCVGEGLKKGAWTAEEDKKLISYIHEHGEGGWRDIPQKAGLKRCGKSCRLRWANYLKPDIKRGEFSYEEEQIIIMLHASRGNKWSVIARHLPKRTDNEIKNYWNTHLKKRLIDQGIDPVTHKPLAPSPSPATLKPSDFQDDSSNLGNSDEHSHSGSMSPKSLPPSSSSCNLAEISSSDETPKNDGSLKSKKRSFKRSSSTSKLLNKVASRAASIGNILSASKEPWLALPHCLHVSMMTFPKLANSRWTNMIHSLSRLNT

SEQ ID NO: 141 - DNA, 1079 bp, Thlaspi arvense
  source 1..1079: mol_type = genomic DNA
atgtcaagaaagccatgttgtgtgggagaagggctgaagaaaggagcatggaccgccgaagaagacaagaaactcatctcttacatccatgaacatggcgaaggaggctggcgtgacattccccaaaaagctggactaaaacgatgtggaaaaagttgtagattgcgatgggctaactatttgaaaccggatatcaagagaggagaatttagctacgaggaggagcagattatcatcatgcttcacgcttcccgtggcaacaagtggtcggtcatagcgagacatttgcccaaaaggacagacaacgagatcaagaactattggaacacacaccttaaaaaacgccttatcgatcaaggtattgatcccgtgacccacaagccacttgcccctagccctagtccggccacgctcaagccttctgatttccaagatgactcatcaaacctgggaaactcggatgagcattcacattcgggttctatgtctccaaaatctcttcctccgtcttcaagctcctgcaatctagcggagataagcagcagtgatgagacaccgaaaaatgatggttccttgaaatccaagaaacgttcttttaagagatcaagttctacatcaaagctgttaaacaaagttgcatctagggctgcttccattggaaatatcttatcagcgtccatggaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatga

SEQ ID NO: 142 - protein, 265 aa, Thlaspi arvense
MSRKPCCVGEGLKKGAWTAEEDKKLISYIHEHGEGGWRDIPQKAGLKRCGKSCRLRWANYLKPDIKRGEFSYEEEQIIIMLHASRGNKWSVIARHLPKRTDNEIKNYWNTHLKKRLIDQGIDPVTHKPLAPSPSPATLKPSDFQDDSSNLGNSDEHSHSGSMSPKSLPPSSSSCNLAEISSSDETPKNDGSLKSKKRSFKRSSSTSKLLNKVASRAASIGNILSASMEEPWLALPHCLHVSMMTFPKLANSRWTNMIHSLSRLNT

SEQ ID NO: 143 - DNA, 1841 bp, Thlaspi arvense
  source 1..1841: mol_type = genomic DNA
atggagagaaagccctttgaggttgagacgacggagaatcacaaaccctactccaccgtcgatggcggtggcgttggttctgatttgagatcgccggtcgattcatttgatgacgagcagaaaaagctcgtttacagaggctggaaagtcatgccttttatcattggtaatgagacatttgagaagattgggatcatagggacattatcaaaccttcttttgtacctaactcaagtattcaaccttaagaaagttacagctgcaacaatcatcaatgcctttagtggcacaatcaacttcgggactttcatcgctgctttccttttggtcgctacaagactctcagtgtagctgtcatcgcttgtttcctgggatcgcttgtgatattactgacggctgcagttcctgcattgcacccgactccatgtggaacacatagctggtgccaagggccaagcccgggccagatcgcgttcttgctgctgggtttagcgtttcttgtggtcggtgcgggtgggatcaggccgtgtaacttggcttttggagctgatcagttcaaccccaaatccgaatccgggaagaaaggaatcaacagcttctttaactggtatttcttcaccttcacgtttgcgcagatcgtctcgctcacgctggtcgtgtatatccagtcgaacgtgagctggacgatcggtttgctcatccctgtggctctgatgttcttggcctgcgtcatcttctttgctggacataaactgtatgtgaaagtgaaagcctcgggtagtcccttggctagtatcggtcacgttatcacggcagcgatcaagaaacgagggttgaagcaagttaagcagccttggctcgatctttacaaccacattccaactaactatccaaactccaccttgaaatacaccgaccagtttaggtttcttgacaaagcagcgattatgacccctgaggacaagctgaattccgatggagctgctttcgatccatggaccctatgtacattgcagaaagtggaagaagtgaaatgcattgtgagagtgattccgatctggtttgcttgcgcgatttactacctcactgtaactatacagatgacttatccggtcttccaagcgcagcagagcgaccggagattgggttctggtggcttcaagatccccgcagccacctatgtggtgttcttgatgtcgggtatgactgttttcatcgtgttctacgaccgtgtccttgtcccgttgctcagaagagtgaccgggttagaaaccggtttgaccctcttgcagagagtcggatcagggatcttctttgccatgttgagtttgttggtctccgggttcgtagaggaacggagaagaaccttcgccctgacgaaaccgactctcgggatggagccacgagcgggagagatctcctccatgtcggccatgtggctgattccgcagctcttgcttgcaggcgtaggagaggcttttacagccattggacagatggagttttattacaagcagttccctgagaacatgaagagcttcgctggctctatcttctatgtcggtgcaggtgtttcgagctatcttgctagcttcttgatctcgactgttcatcgaagaactgaacattcaccctccgggaactggttagctgaggatctgaacaaagggagactcgattacttctacttcatgctcaccggaatcatggtcgttaacatggtttacttcttgataatgtctaaatggtatagatacaaaggcattaacgatgaagcgaattctttggtcgagaccaatgaagaagagaccaagcagaaacaagtcaagaattctgtctga

SEQ ID NO: 144 - protein, 116 aa, Thlaspi arvense
MERKPFEVETTENHKPYSTVDGGGVGSDLRSPVDSFDDEQKKLVYRGWKVMPFIIGNETFEKIGIIGTLSNLLLYLTQVFNLKKVTAATIINAFSGTINFGTFIAAFLLVATRLSV

SEQ ID NO: 145 - DNA, 1843 bp, Thlaspi arvense
  source 1..1843: mol_type = genomic DNA
atggagagacagcctctcgaactcgagtctacggatcaccaaaaaccttcccccgccgtctacggtggctctgttacgacggttgattctgttgaggaagaagttcaggagaaaaaagtcgtatatagaggctggaaagtcatgccctttatcattggaaatgagacattcgagaagcttgggatcattggaacactatcaaaccttctggtttttttaacagctgtcttcaacatgaagagtatcacagctgcaacaatcattaacgcattcagtggcacaataaatttcggaactttcgttgctgctttcctctgcgacacttacttcggtcgatacaagacactaaccgtcgcggtcatcgcctgttttcttggatcacttgtgatactattgacagctgcagtgccacaactgcacccagctccatgtggaacagcgagctcgtgcagcggtccaagcggtgggcaaatcgcgtttcttctgatgggtctcgggtttcttgtagttggagcgggcgggatcagaccgtgcaatctagctttcggagccgatcagttcaacccgaagagcgagtcagggagaagagggactgatagtttcttcaattggtacttcttcagcttcactttcgcgcagatcttgtcgctgacgctagtggtctacatccagtctaacgtcagctggacgatcggtctaaccataccggtggttctaatgttcttggcctccgtgatcttctttgcgggagataagctgtatgtgaaagtcaaggcctcgggtagtccattggctggtatagctcaagtgatcattgttgcaatcaagaaacgcggattaaagcccgtgaaacagccttggcttaacctctacaattactgccctccaaaacacgcaaactccattctcaaatacaccgaccaattcagatttcttgataaggcggcgatcttggctcccgaggacaagttggaggcggatggtaagcctgcggatccatggaagctgtgtacgatgcaacaggtggaagaagtgaagtgcatttgtgagagtgcttcctatatggttcgctgcatcaatctactacgtaaccataacccagcaaatgacatatccggtcttccaagccctgcagagcgatcgtcgcttaggctcgggagggttcgtgatccccgcagccacctacgtggtcttcttgatgacagggatgacggttttcatcatcatctacgaccgtctcctcgtgcctaccttgagaagaataaccggtctagacaccgggatcacgctcctgcagagaatcggaaccgggatcttcttcgcctttgcaagcttagtagtctccggtttcgtcgaggagcggaggagacacattgcgctgactaaaccaactcttggcatggcgccaagaaaaggagaaatctcctcaatgtcagctatgtggctcatcccgcagctcactctctcgggtgtagccgaggcgtttggagccatcggacagatggagttttactacaagcagttcccagaaaacatgaggagtttcgcgggttccatcttttatgtaggaataggggtttcgagttacctcggcagcttcttgattgcaacggttcaccggacgacgcagaactcggcgggtggtaactggttggctgaggatttgaacaaaggcagattggattacttctatttcatgatcgctggaatcttggctgttaatttcgcctacttcttggtcgtgtcaagatggtataggtacaaagaaagtgatgatgatcaaaagacagcttctgaaaccaatggagatgtcatcaaacaacaagacaagaacactgcctga

SEQ ID NO: 146 - protein, 461 aa, Thlaspi arvense
MERQPLELESTDHQKPSPAVYGGSVTTVDSVEEEVQEKKVVYRGWKVMPFIIGNETFEKLGIIGTLSNLLVFLTAVFNMKSITAATIINAFSGTINFGTFVAAFLCDTYFGRYKTLTVAVIACFLGSLVILLTAAVPQLHPAPCGTASSCSGPSGGQIAFLLMGLGFLVVGAGGIRPCNLAFGADQFNPKSESGRRGTDSFFNWYFFSFTFAQILSLTLVVYIQSNVSWTIGLTIPVVLMFLASVIFFAGDKLYVKVKASGSPLAGIAQVIIVAIKKRGLKPVKQPWLNLYNYCPPKHANSILKYTDQFRFLDKAAILAPEDKLEADGKPADPWKLCTMQQVEEVKCICESASYMVRCINLLRNHNPANDISGLPSPAERSSLRLGRVRDPRSHLRGLLDDRDDGFHHHLRPSPRAYLEKNNRSRHRDHAPAENRNRDLLRLCKLSSLRFRRGAEETHCAD

SEQ ID NO: 147 - DNA, 1366 bp, Thlaspi arvense
  source 1..1366: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagtaatggggatccaggatcctcatgttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 148 - protein, 116 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSERYGDPGSSCFGQSLRVYSTSTS

SEQ ID NO: 149 - DNA, 1363 bp, Thlaspi arvense
  source 1..1363: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagaatggggatccaggatcctcatgttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 150 - protein, 115 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSENGDPGSSCFGQSLRVYSTSTS

SEQ ID NO: 151 - DNA, 1363 bp, Thlaspi arvense
  source 1..1363: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagtggggatccaggatcctcatgttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 152 - protein, 115 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSESGDPGSSCFGQSLRVYSTSTS

SEQ ID NO: 153 - DNA, 1360 bp, Thlaspi arvense
  source 1..1360: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagggatccaggatcctcatgttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 154 - protein, 114 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSERDPGSSCFGQSLRVYSTSTS

SEQ ID NO: 155 - DNA, 1366 bp, Thlaspi arvense
  source 1..1366: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagttatggggatccaggatcctcatgttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 156 - protein, 116 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSESYGDPGSSCFGQSLRVYSTSTS

SEQ ID NO: 157 - DNA, 1357 bp, Thlaspi arvense
  source 1..1357: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttctggggatccaggatcctcatgttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 158 - protein, 39 aa, Thlaspi arvense
MFWTKFTSLLNFYVLIIVTVTRASAKRSRRFQRSYQNWI

SEQ ID NO: 159 - DNA, 1818 bp, Thlaspi arvense
  source 1..1818: mol_type = genomic DNA
atgaacgactacttcctcaaccagtcaacagccaccgacgataacgcgtcggcggcgatggaagctttcatcggaacaaatcactcaactctctggccccaaccatctcttcctcctcctcctccactatctcaattcaacgaagacactcttcaacaacgtctccaagctttgatcgaatcagcaggagaaggctggacctacgcgattttctggcagatctcacacgatttcgattcttccaccggagacaacacagtgattctcggatggggagatgggtactacaaaggggaggaagataaagagaagaagaagaatagctcaagttcgaattcggcggagcaagagcatcggaaaagagtaatccgtgagctgaattcgttgatctccggcggaaccggagtttccgatgaatcgaacgacgaagaagtcaccgatactgagtggttcttcttggtctcgatgacacagagcttcatgaacggcgttgggcttcccggggagtcgtacttgaactctcgcgtgatttggttatccgggccgggtgctttaatcgggtcgggttgcgaacgagcgggtcaaggtcagatatacgggttacagacgatggtgtgtatcgcggcggagaacggcgtcgttgagcttggttcatcggaggtgttaagccatagctcagatctgatggataaagtcaacagtctgttcaattccaacaacggtaatggagaagcttcttcttgggggtttaatctgaatccggatcaaggagagaacgatccggctttgtggataagcgaaccgaccaccaccggaatcgaatcgggtcaggtaatcccggcgataaacaacagtaattccaattcaaattcaaaatccgattctcatcaaatctccaagctcgagaagaacgagagctcgattgaaaaccctagacaacaacaaaatccgtcgcttgtagagcgagatttgaatttctcgagttctgggttgaatcaaaacgggaactttcaagatgggtcgtcgcggatgatgaaatcgaacgaaacactgagctttacggcggaggagagcaacaagaggaggtctccggtttcgaaagggagtaacaacgacgaagggatgctttctttcagcaccgtggttcgttccgcggcgaaatccgtcgattcggatcattccgatctcgaagcgtcggtggttaaggaagcaattgtcgttgaaccggagaagaaaccgaggaaacggggaagaaaaccggcgaacggaagagaggagccgttgaatcacgtggaagcagagagacagagaagggagaagctaaaccagagattctactctttgagagctgtggttcccaacgtttcgaaaatggacaaagcgtctcttctcggagacgccatttcgtatatcaacgagcttaaatcgaagctgcagcaagcggaatccgataaggaagagattcagaagcagctcgacgggatgagcaaggaaggaaacagggaaggcggcggcggaacgaaggcgaaagaacgaaaatgctcgaatcaagattcggctagctcgatagaaatggagatcgacgtgaagatcataggttgggatgtgatgatacgtgtacaatgcagcaagaagaatcatcccggggcaagattcatggaagcgctcaaggaattggatctggaagtgaatcacgcaagtttgtcagtggtgaatgatttgatgattcaacaagccactgtgaagatgggaagccagtttttcaatcatgaccagctcaaggttgctttgatgtcgaaagtcggagaagacaattga

SEQ ID NO: 160 - DNA, 1818 bp, Thlaspi arvense; identical to SEQ ID NO: 159
  source 1..1818: mol_type = genomic DNA

SEQ ID NO: 161 - protein, 605 aa, Thlaspi arvense
MNDYFLNQSTATDDNASAAMEAFIGTNHSTLWPQPSLPPPPPLSQFNEDTLQQRLQALIESAGEGWTYAIFWQISHDFDSSTGDNTVILGWGDGYYKGEEDKEKKKNSSSSNSAEQEHRKRVIRELNSLISGGTGVSDESNDEEVTDTEWFFLVSMTQSFMNGVGLPGESYLNSRVIWLSGPGALIGSGCERAGQGQIYGLQTMVCIAAENGVVELGSSEVLSHSSDLMDKVNSLFNSNNGNGEASSWGFNLNPDQGENDPALWISEPTTTGIESGQVIPAINNSNSNSNSKSDSHQISKLEKNESSIENPRQQQNPSLVERDLNFSSSGLNQNGNFQDGSSRMMKSNETLSFTAEESNKRRSPVSKGSNNDEGMLSFSTVVRSAAKSVDSDHSDLEASVVKEAIVVEPEKKPRKRGRKPANGREEPLNHVEAERQRREKLNQRFYSLRAVVPNVSKMDKASLLGDAISYINELKSKLQQAESDKEEIQKQLDGMSKEGNREGGGGTKAKERKCSNQDSASSIEMEIDVKIIGWDVMIRVQCSKKNHPGARFMEALKELDLEVNHASLSVVNDLMIQQATVKMGSQFFNHDQLKVALMSKVGEDN

SEQ ID NO: 162 - DNA, 1332 bp, Thlaspi arvense
  source 1..1332: mol_type = genomic DNA
atggcggcgtgtttacaatcgaacatccggctgaatctgaacaatatcgtcggaggaaaatgcagatccctcaccgatcagtctcgcacgccgtgtcgaataaggtgcgccgccgcttcacctgggaaaaaacggttcaacatcgctctccttcctggcgacggtatcggtcctgaagtcatctccgttgccaagaatgtgcttcagcaagctggatctctcgaaggttcttctatctctcgctcagtttatttactaattttgacaaaattagtaatcagcgaatgtgttgcttacccagaagagtgtgcgtatttgatgtgtatcacaggactggagttcaatttccaggagatgcccatcggaggagcagccttggatttggtcggagtggccttgccggaggaaaccttatcggctgcaaaacagtctgatgccatacttcttggagctatcggagggtacaaatgggacaacaatgagaaacatctgagacctgagatggctctgatttaccttcggagagatctcaaagtctttgctaatctgagacctgcttcagttttgccacagctagttgatgcatccaccttgaagagagaagtagcagaaggtgttgatatgatgattgtaagggagcttacaggaggaatttactttggagagcccagaggcattaagaccaacgaaaacggagaagaagtcggttttaatacagagttttacgctgctcacgagattgacagaattgctcgtgttgcatttgagactgctaggaaacggcgtggcaagctgtgttctgtcgacaaagccaatgtgttggatgcatcagtattgtggaggagaagagtaacagcgttagcctctgagtatccagatgttgagctctcacatatgtatgtagacaacgctgcaatgcagcttattcgtgacccgaaacagtttgacacaatcgtcaccaataacattttcggtgacatattatccgatgaggcctcaatgatcactggaagcattgggatgcttccatctgctagtctcggtgtatcgggacctggactctttgagccgatacatggttctgcgccagatatagctggtcaggacaaggcaaacccattggccaccattctcagcgcagcgatgcttctgaaatatggacttggagaagagaaggccgcaaagaggatcgaggacgcggtcttggatgctctcaacaaagggtttagaaccggagacatctactcccccggaaataaactggtgggatgcaaggagatgggtgaggaggtgcttaaatcagtggactccagagttacagctactgtttaa

SEQ ID NO: 163 - DNA, 1936 bp, Thlaspi arvense
  source 1..1936: mol_type = genomic DNA
atggcggcgtgtttacaatcgaacatccggctgaatctgaacaatatcgtcggaggaaaatgcagatccctcaccgatcagtctcgcacgccgtgtcgaataaggtgcgccgccgcttcacctgggaaaaaacggttcaacatcgctctccttcctggcgacggtatcggtcctgaagtcatctccgttgccaagaatgtgcttcagcaagctggatctctcgaaggttcttctatctctcgctcagtttatttactaattttgacaaaattagtaatcagcgaatgtgttgcttacccagaagagtgtgcgtatttgatgtgtatcacaggactggagttcaatttccaggagatgcccatcggaggagcagccttggatttggtcggagtggccttgccggaggaaaccttatcggctgcaaaacagtctgatgccatacttcttggagctatcggagggtgtgtgtttgtgtgtcttgtttctttttttaattggtctcttggttcagaaatctgtctgcttcgagtcttcttctcagcttgagttttttggatgattcatggttggttggattatattcaggtacaaatgggacaacaatgagaaacatctgagacctgagatggctctgatttaccttcggagagatctcaaagtctttgctaatctgagacctgcttcagttttgccacaggtatatatatatatatatatatatataaacgtttatctaattcggtttgatctgcttaccacttgcaatttttacaatcacttgcttgttgttctcagctagttgatgcatccaccttgaagagagaagtagcagaaggtgttgatatgatgattgtaagggagcttacaggaggaatttactttggagagcccagaggcattaagaccaacgaaaacggagaagaagtcggttttaatacagagttttacgctgctcacgaggtcacatactcttcacagtgtggtctatgatatgatgggtctaaggctatatatgtataatataatgcattaattgctcatgttacttccagattgacagaattgctcgtgttgcatttgagactgctaggaaacggcgtggcaagctgtgttctgtcgacaaagccaatgtgttggatgtacgtttgatcaacaattttttccatttctgtgtgtgtgtgtttttttttctcgtaatgcagaattatttttttcatataggcatcagtattgtggaggagaagagtaacagcgttagcctctgagtatccagatgttgagctctcacatatgtatgtagacaacgctgcaatgcagcttattcgtgacccgaaacagtttgacacaatcgtcaccaataacattttcggtgacatattatccgatgaggcctcaatgatcactggaagcattgggatgcttccatctgctagtctcggtgtatcggtaaccaacaaatttaaactgaaaatctttcaaggttcctccctgtttattaccatatatacacatggaatgttgaaacttgtgtttggatcagggacctggactctttgagccgatacatggttctgcgccagatatagctggtcaggacaaggcaaacccattggccaccattctcagcgcagcgatgcttctgaaatatggacttggagaagagaaggccgcaaagaggatcgaggacgcggtcttggatgctctcaacaaagggtttagaaccggagacatctactcccccggaaatgtatgttttgatggtatttttatcatttgcttatataataagttaaacaacaaacagttagtagtatattatatacttttgtgttattttaatgaaatttggaatgtgaagcagaaactggtgggatgcaaggagatgggtgaggaggtgcttaaatcagtggactccagagttacagctactgtttaa

SEQ ID NO: 164 - protein, 443 aa, Thlaspi arvense
MAACLQSNIRLNLNNIVGGKCRSLTDQSRTPCRIRCAAASPGKKRFNIALLPGDGIGPEVISVAKNVLQQAGSLEGSSISRSVYLLILTKLVISECVAYPEECAYLMCITGLEFNFQEMPIGGAALDLVGVALPEETLSAAKQSDAILLGAIGGYKWDNNEKHLRPEMALIYLRRDLKVFANLRPASVLPQLVDASTLKREVAEGVDMMIVRELTGGIYFGEPRGIKTNENGEEVGFNTEFYAAHEIDRIARVAFETARKRRGKLCSVDKANVLDASVLWRRRVTALASEYPDVELSHMYVDNAAMQLIRDPKQFDTIVTNNIFGDILSDEASMITGSIGMLPSASLGVSGPGLFEPIHGSAPDIAGQDKANPLATILSAAMLLKYGLGEEKAAKRIEDAVLDALNKGFRTGDIYSPGNKLVGCKEMGEEVLKSVDSRVTATV

SEQ ID NO: 165 - DNA, 1635 bp, Thlaspi arvense
  source 1..1635: mol_type = genomic DNA
atggatactttagcttcaaactcttcgggtctcaggaccaagtcgagtcccgagacgtcgccgtttagcaacatgtatctcctcacaacacttcaagcccttgcggttatttctctcttgatgatattcaagaaaataaagtattcctcttcacaaaaaaaaaagtttcatcctctcccgccgggccccagcgggtttccagtcgtcggaatgattccggcgatgcttaaaaaccgtccggttttccggtggcttcacagcctcatgaaagagcttaacacggagatagcttgtgttcgtctaggaaagactcacgtgatccccgtcacatgtcctaagatcgcacgtgagattttcaagcaacaagacgcactcttcgcgtcgagaccactcacttatgctcaaaagatactttccaacggctacaaaacctgcgtgatcacaccgttcggagaacaattcaagaagatgaggaaagtaatcatgacggagattgtttgtccagcaagacaccgatggttacacgacaacagagccgaggaaaccgatcatttaactgcttggctttacaacatggttaagaaatccgaaccggtcgatctccggtttgttacaaggcactactgcggaaatgcgattaagaggcttatgttcggaacgaggacgttctcggcgaaaaccgaaaccgacggtggaccaaccgtggaagatatcgagcatatggatgctatgtttgaagggttagggtttacgtttgcgttctgtgtatcggattatctaccgatgcttacgggattggatctaaacggacatgagaagatcatgagagaagctagtgcgattatggacaaatatcacgatcccattattgatgagaggattaagatgtggagagaaggaaagagaactcagattgaagattttctagacattttcatctctatcaaggacgaagctggccagcctttgcttaccgctgatgaaatcaaaccaaccattaaggaacttgtaatggcggcgccggacaacccatcaaacgccgtggaatgggccatggcggagatgataaacaaacctgagattctccacaaagcaatggaagagattgaaagagtcgttggcaaagaaagactcgtccaagaatccgatatcccaaaacttaactatctcaaagctattatccgagaagctttccgtctgcatcccgtcgccgccttcaacctcccacacgtggcactttccgacacaaccgtcgctggttatcacatccctaaggggagtcaagttttacttagccgttacggtcttggtcgtaacccaaaggtttggtctgatccacttagctttaaaccggagagacacctcaatgagtgcttggaagtaacgttgactgagaatgatctccggtttatctcgtttagtaccggaaaaagaggatgtgctgctccggcgttaggtacggcgataaccgtcatgatgctcgccaggcttttgcaagggtttaagtggaagttagctggaggtgagacacgtgttgagttgatggaatcgagtcatgatatgtttcttgcgacgcctttggttatggtcggagaattgagattgtcggaggatctttaccccacggtgaagtga

SEQ ID NO: 166 - DNA, 2050 bp, Thlaspi arvense
  source 1..2050: mol_type = genomic DNA
atggatactttagcttcaaactcttcgggtctcaggaccaagtcgagtcccgagacgtcgccgtttagcaacatgtatctcctcacaacacttcaagcccttgcggttatttctctcttgatgatattcaagaaaataaagtattcctcttcacaaaaaaaaaagtttcatcctctcccgccgggccccagcgggtttccagtcgtcggaatgattccggcgatgcttaaaaaccgtccggttttccggtggcttcacagcctcatgaaagagcttaacacggagatagcttgtgttcgtctaggaaagactcacgtgatccccgtcacatgtcctaagatcgcacgtgagattttcaagcaacaagacgcactcttcgcgtcgagaccactcacttatgctcaaaagatactttccaacggctacaaaacctgcgtgatcacaccgttcggagaacaattcaagaagatgaggaaagtaatcatgacggagattgtttgtccagcaagacaccgatggttacacgacaacagagccgaggaaaccgatcatttaactgcttggctttacaacatggttaagaaatccgaaccggtcgatctccggtttgttacaaggcactactgcggaaatgcgattaagaggcttatgttcggaacgaggacgttctcggcgaaaaccgaaaccgacggtggaccaaccgtggaagatatcgagcatatggatgctatgtttgaagggttagggtttacgtttgcgttctgtgtatcggattatctaccgatgcttacgggattggatctaaacggacatgagaagatcatgagagaagctagtgcgattatggacaaatatcacgatcccattattgatgagaggattaagatgtggagagaaggaaagagaactcagattgaagattttctagacattttcatctctatcaaggacgaagctggccagcctttgcttaccgctgatgaaatcaaaccaaccattaaggtaactaattagatatattgaattatatttattagatttcttgtcgactaattacgcatattacttatatgtttggatttaatatagcagaactaagtttattattagtattagcaatttttaagattaagtggtaaaaaagtggatacaaatgtttttgggggggggttctagcgacaacctaagattttaagttgtcgttaaaactttttttttttctgattttttaaaaagggtaaaacgccaaaacatccatagtcagaaactttatgctaatccatagtcagaaactttatgctaatccatagtcagaaactttatgtatagcataaagtttatatttatgttgacccgtgagtccatgcatagaatagtaaaacaataattaatcatttatatattttatattgaataggaacttgtaatggcggcgccggacaacccatcaaacgccgtggaatgggccatggcggagatgataaacaaacctgagattctccacaaagcaatggaagagattgaaagagtcgttggcaaagaaagactcgtccaagaatccgatatcccaaaacttaactatctcaaagctattatccgagaagctttccgtctgcatcccgtcgccgccttcaacctcccacacgtggcactttccgacacaaccgtcgctggttatcacatccctaaggggagtcaagttttacttagccgttacggtcttggtcgtaacccaaaggtttggtctgatccacttagctttaaaccggagagacacctcaatgagtgcttggaagtaacgttgactgagaatgatctccggtttatctcgtttagtaccggaaaaagaggatgtgctgctccggcgttaggtacggcgataaccgtcatgatgctcgccaggcttttgcaagggtttaagtggaagttagctggaggtgagacacgtgttgagttgatggaatcgagtcatgatatgtttcttgcgacgcctttggttatggtcggagaattgagattgtcggaggatctttaccccacggtgaagtga

SEQ ID NO: 167 - protein, 544 aa, Thlaspi arvense
MDTLASNSSGLRTKSSPETSPFSNMYLLTTLQALAVISLLMIFKKIKYSSSQKKKFHPLPPGPSGFPVVGMIPAMLKNRPVFRWLHSLMKELNTEIACVRLGKTHVIPVTCPKIAREIFKQQDALFASRPLTYAQKILSNGYKTCVITPFGEQFKKMRKVIMTEIVCPARHRWLHDNRAEETDHLTAWLYNMVKKSEPVDLRFVTRHYCGNAIKRLMFGTRTFSAKTETDGGPTVEDIEHMDAMFEGLGFTFAFCVSDYLPMLTGLDLNGHEKIMREASAIMDKYHDPIIDERIKMWREGKRTQIEDFLDIFISIKDEAGQPLLTADEIKPTIKELVMAAPDNPSNAVEWAMAEMINKPEILHKAMEEIERVVGKERLVQESDIPKLNYLKAIIREAFRLHPVAAFNLPHVALSDTTVAGYHIPKGSQVLLSRYGLGRNPKVWSDPLSFKPERHLNECLEVTLTENDLRFISFSTGKRGCAAPALGTAITVMMLARLLQGFKWKLAGGETRVELMESSHDMFLATPLVMVGELRLSEDLYPTVK

SEQ ID NO: 168 - DNA, 1188 bp, Thlaspi arvense
  source 1..1188: mol_type = genomic DNA
atgatcaccaccacctgttgcactgtggtcgcccagttaggcttgccctttggatcttccttgtcctctctctgcctgacccgtccgaacaaaaagccgtccttgttcacctcatgttgctcctctgtgtccaaaaatgttggcgctagtgctgctgacagcaaacacgtcgtggaacggtggccagagtacattccgaataagctccctgacaagaactatgtgcgtatttttgacacgacgctccgtgacggcgaacaagctcctggtgcagcccttactccaccgcagaagctagagattgcccggcagctcgctaagctccgagtagacatcatggaagttggtttccctgggtcctctgaggaagagttcgaaaccgtcaagaccatcgccaagaccgtagggaacgaggtggatgaggaaacaggctacgtcccagtgatatgcgccctcgcacgatgcaaacatagagacattgaggcggtttgggaggcggtgaagtacgcaaagaggccttcgatactcatattcatatctactagtgacattcatatgaaacataagttgaaaaagactaaagaagaagtcatagagatggcagcaagtagtattaggtttgctaagaacttaggcttcaatgacatccaattgggttgcgaagatgccggcaggtcggataaggagtttctatgcaagattctaggagaagcgatcaaagcgggtgcaaccacgatgaacatggcagacacggtagggatcaacatgccggaagaatttggagaactcgtgagctaccttaaagccaacactcctggaattgacgatgttgtcttaagcgttcattgtcacaacgaccttggtgttgctaccgccaacgcaatcgccggtgtgtgtgctggagcaagacaagttgacgttacggtcaatggaataggtgaaagatgtgggaacgcgccacttgaagaggtcgtgatggctttgaaatgccgaggagcatacctgatgaatggggtttacacaagaacagacatacgccaaattatggctactagcaagatggttcaggaatatactggcttttatgttcaaccacataagcccatagttggagccaacagtttttttcatgagagcagcgatgaatatgatggttgttcaaacattttgcaggatatggattattga

SEQ ID NO: 169 - DNA, 1820 bp, Thlaspi arvense
  source 1..1820: mol_type = genomic DNA
atgatcaccaccacctgttgcactgtggtcgcccagttaggcttgccctttggatcttccttgtcctctctctgcctgacccgtccgaacaaaaagccgtccttgttcacctcatgttgctcctctgtgtccaaaaatgttggcgctagtgctgctgacagcaaacacgtcgtggaacggtggccagagtacattccgaataagctccctgacaagaactatgtgcgtatttttgacacgacgctccgtgacggcgaacaagctcctggtgcagcccttactccaccgcagaagctagagattgcccggcagctcgctaagctccgagtagacatcatggaagttggtttccctgggtcctctgaggaagagttcgaaaccgtcaagaccatcgccaagaccgtagggaacgaggttgtttctctgtttccttcattttctagtataaaaatttctacgttcctactgtatattcagtcaaccttgtcgctttggggacatatgataatatttgaacgaatattaacatagttttgacgcaaatagtacaaacacaaattgagtaaatctccaaattttaccatagttatatagtaaacgattgatttgattaggtggatgaggaaacaggctacgtcccagtgatatgcgccctcgcacgatgcaaacatagagacattgaggcggtttgggaggcggtgaagtacgcaaagaggccttcgatactcatattcatatctactagtgacattcatatgaaacataagttgaaaaagactaaagaagaagtcatagagatggcagcaagtagtattaggtttgctaagaacttaggcttcaatgacatccaattgggttgcgaagatgccggcaggtccatatcttaaaaccttaatatacatatatagccttttgttagataatgttaaggatttttgtgaataatgaccatgtcacaaaaattctagcgtgtgattaaaactaagcaggtcggataaggagtttctatgcaagattctaggagaagcgatcaaagcgggtgcaaccacgatgaacatggcagacacggtagggatcaacatgccggaagaatttggagaactcgtgagctaccttaaagccaacactcctggaattgacgatgttgtcttaagcgttcattgtcacaacgaccttggtgttgctaccgccaacgcaatcgccgtatttttctaaccctctccttcatctcatatatttaaaatatctatataaatacatatagatcattattgcggttatattatatacgtagggtgtgtgtgctggagcaagacaagttgacgttacggtcaatggaataggtgaaagatgtgggaacgcgccacttgaagaggtaaaagatcatgattatcatcaagaccgtcttacactatttgtaggatcggttccatatttttatatacggccctgatccatcatcatcaattcatcaacaacataaatatttttttggtatgtataggtcgtgatggctttgaaatgccgaggagcatacctgatgaatggggtttacacaagaacagacatacgccaaattatggctactagcaagatggtattttttacattaatttacacatggaaaaacgatcgtgctagttttttttttatatatgttggcattatagtaatatatgtaaattcttgagcaggttcaggaatatactggcttttatgttcaaccacataagcccatagttggagccaacagtttttttcatgagagcagcgatgaatatgatggttgttcaaacattttgcaggatatggattattga

SEQ ID NO: 170 - protein, 395 aa, Thlaspi arvense
MITTTCCTVVAQLGLPFGSSLSSLCLTRPNKKPSLFTSCCSSVSKNVGASAADSKHVVERWPEYIPNKLPDKNYVRIFDTTLRDGEQAPGAALTPPQKLEIARQLAKLRVDIMEVGFPGSSEEEFETVKTIAKTVGNEVDEETGYVPVICALARCKHRDIEAVWEAVKYAKRPSILIFISTSDIHMKHKLKKTKEEVIEMAASSIRFAKNLGFNDIQLGCEDAGRSDKEFLCKILGEAIKAGATTMNMADTVGINMPEEFGELVSYLKANTPGIDDVVLSVHCHNDLGVATANAIAGVCAGARQVDVTVNGIGERCGNAPLEEVVMALKCRGAYLMNGVYTRTDIRQIMATSKMVQEYTGFYVQPHKPIVGANSFFHESSDEYDGCSNILQDMDY

SEQ ID NO: 171 - DNA, 798 bp, Thlaspi arvense
  source 1..798: mol_type = genomic DNA
atgttgtatcgaaaagagtacttgtggttcatgatgattttgtgggccagatctttatcagagggcctaaaatattctccgtctcttaggaatcttgtaaacacgatgacaccttccttatcaccaattagatctcaccacgtggcagttatcggagctggagccgccggtttagtagccgcgcgagagcttcgacgggaaggtcactcggtggtcgttttcgagaggcagaatcaggtcggaggaacatggatctacaccgatcatgtcgagccggatccgttaagcgtcgacccgacccgacccgttgttcactcgagcgtctatggctctctccggaccaaccttccgcgtgagtgtatgggatacagagacttcccgttcgtgatccgatccggcgtctcggaatcgagagacccgaggaggtttccgagtcatagtgaagttctggcgtatctgcaggatttcgcaaaggagtttcggatcgaggagatgatacggttcgagacggcggttgagcgtgttgctccggcggaggaaagcgacggcgaagcaggaaaaatgaaatggaggattgaatctacagagaaagaggagaaacttcgtcgcgatgagatttacgatgccgttgtcgtctgtaacggacattacattgagcctcgtctagctgaaattcctggtgattcccctttgattctcctcttgaagtttcaacatcttgactttcgatggattcgtgtgttaataagattactcttttgcttcattcaagaaaatttagatagaatttggaaatag

SEQ ID NO: 172 - DNA, 798 bp, Thlaspi arvense; identical to SEQ ID NO: 171
  source 1..798: mol_type = genomic DNA

SEQ ID NO: 173 - protein, 265 aa, Thlaspi arvense
MLYRKEYLWFMMILWARSLSEGLKYSPSLRNLVNTMTPSLSPIRSHHVAVIGAGAAGLVAARELRREGHSVVVFERQNQVGGTWIYTDHVEPDPLSVDPTRPVVHSSVYGSLRTNLPRECMGYRDFPFVIRSGVSESRDPRRFPSHSEVLAYLQDFAKEFRIEEMIRFETAVERVAPAEESDGEAGKMKWRIESTEKEEKLRRDEIYDAVVVCNGHYIEPRLAEIPGDSPLILLLKFQHLDFRWIRVLIRLLFCFIQENLDRIWK

SEQ ID NO: 174 - DNA, 1392 bp, Thlaspi arvense
  source 1..1392: mol_type = genomic DNA
atggtggaaatagtcaacaaatgcaaaggccacgtcatcattttaccttacccagttcaaggccaccttaaccccatggttcagttcgctaaacgtctcgtctccaaaggcgtcaaagtcaccatcgcaaccactacctacaccgccttctccatctccactccatcagtctccgtcgagccaatctccgacggatacgacttcatccccatagctattcccggtttcagcgtcgacacctactcggaatccttcaagctccacggctccgaaaccctaacccgcgtgatcgagaaattcaaatccaccgattccccgatcgattctttagtctacgactcctttcttccctggggactcgacgtcgccagatccaactcgatttcagctgccgctttcttcaccaacaacctcacggtttgttccgtgctgcgtaatttctccaccggggagtttcctctccccgccgatccagattctgcgccgtacctaatccgtggcttgccggcgttgagctacgacgagctgccttccttcgtcggacgccactggttgagccacccggagcacgggaaggttcttctgaatcagttccataaccatgaaaacgccgattggcttttcgtcaatggattcgaaggcttagaaacacaagattgtgaaactggagaatcagaggcgatgaaggcgacgttgatcggcccgttgatcccatcggcgtacctcgacgaccggataaaagacgataaagactatggctcgagcctaatgaaaccgctctcagaggaatgtatggagtggctcgggactaagccagcaaagtcagtagttttcgtctcgttcggttcctttgggatcctctttgacaagcagctcgctgaggtagcaaaggctctgcaagaatcgaacttgaacttcttgtgggtgatcaaagaagctcatatcgcgaaactgcctgaagggtttgtggaatcgaccaaagacagagccttgctggtttcttggtgtaaccagcttgaggttttagcacacgagtccatcggttgctttttgactcactgcggttggaactcgacgcttgaagggttgagtttgggagtgccgatggtgggtgtgccgcagtggagtgatcagatgaacgatgctaagtttgtggaggaagtttggagagttgggtacagagcgaaagaggaagctgaggaaggagtcgtgaagagcgaagaagtggtgaggtgtttgaaaggagtgatggaaggagagagtagtgtgaagattagagagagttcgaagaagtggaaagatttggctgtgaaggcaatgactgaaggaggaagctctgatcggagcattgacgagtttgtggagagtttaaggaaggaaacgttgaggtag

SEQ ID NO: 175 - DNA, 1477 bp, Thlaspi arvense
  source 1..1477: mol_type = genomic DNA
atggtggaaatagtcaacaaatgcaaaggccacgtcatcattttaccttacccagttcaaggccaccttaaccccatggttcagttcgctaaacgtctcgtctccaaaggcgtcaaagtcaccatcgcaaccactacctacaccgccttctccatctccactccatcagtctccgtcgagccaatctccgacggatacgacttcatccccatagctattcccggtttcagcgtcgacacctactcggaatccttcaagctccacggctccgaaaccctaacccgcgtgatcgagaaattcaaatccaccgattccccgatcgattctttagtctacgactcctttcttccctggggactcgacgtcgccagatccaactcgatttcagctgccgctttcttcaccaacaacctcacggtttgttccgtgctgcgtaatttctccaccggggagtttcctctccccgccgatccagattctgcgccgtacctaatccgtggcttgccggcgttgagctacgacgagctgccttccttcgtcggacgccactggttgagccacccggagcacgggaaggttcttctgaatcagttccataaccatgaaaacgccgattggcttttcgtcaatggattcgaaggcttagaaacacaagtaagaaagagtccaagatttacgcgatattgttcaattgaaacttggaattgatgttttttggcttggatttgtgttcgattaggattgtgaaactggagaatcagaggcgatgaaggcgacgttgatcggcccgttgatcccatcggcgtacctcgacgaccggataaaagacgataaagactatggctcgagcctaatgaaaccgctctcagaggaatgtatggagtggctcgggactaagccagcaaagtcagtagttttcgtctcgttcggttcctttgggatcctctttgacaagcagctcgctgaggtagcaaaggctctgcaagaatcgaacttgaacttcttgtgggtgatcaaagaagctcatatcgcgaaactgcctgaagggtttgtggaatcgaccaaagacagagccttgctggtttcttggtgtaaccagcttgaggttttagcacacgagtccatcggttgctttttgactcactgcggttggaactcgacgcttgaagggttgagtttgggagtgccgatggtgggtgtgccgcagtggagtgatcagatgaacgatgctaagtttgtggaggaagtttggagagttgggtacagagcgaaagaggaagctgaggaaggagtcgtgaagagcgaagaagtggtgaggtgtttgaaaggagtgatggaaggagagagtagtgtgaagattagagagagttcgaagaagtggaaagatttggctgtgaaggcaatgactgaaggaggaagctctgatcggagcattgacgagtttgtggagagtttaaggaaggaaacgttgaggtag

SEQ ID NO: 176 - protein, 463 aa, Thlaspi arvense
MVEIVNKCKGHVIILPYPVQGHLNPMVQFAKRLVSKGVKVTIATTTYTAFSISTPSVSVEPISDGYDFIPIAIPGFSVDTYSESFKLHGSETLTRVIEKFKSTDSPIDSLVYDSFLPWGLDVARSNSISAAAFFTNNLTVCSVLRNFSTGEFPLPADPDSAPYLIRGLPALSYDELPSFVGRHWLSHPEHGKVLLNQFHNHENADWLFVNGFEGLETQDCETGESEAMKATLIGPLIPSAYLDDRIKDDKDYGSSLMKPLSEECMEWLGTKPAKSVVFVSFGSFGILFDKQLAEVAKALQESNLNFLWVIKEAHIAKLPEGFVESTKDRALLVSWCNQLEVLAHESIGCFLTHCGWNSTLEGLSLGVPMVGVPQWSDQMNDAKFVEEVWRVGYRAKEEAEEGVVKSEEVVRCLKGVMEGESSVKIRESSKKWKDLAVKAMTEGGSSDRSIDEFVESLRKETLR

SEQ ID NO: 177 - DNA, 798 bp, Thlaspi arvense
  source 1..798: mol_type = genomic DNA
atgttgtatcgaaaagagtacttgtggttcatgatgattttgtgggccagatctttatcagagggcctaaaatattctccgtctcttaggaatcttgtaaacacgatgacaccttccttatcaccaattagatctcaccacgtggcagttatcggagctggagccgccggtttagtagccgcgcgagagcttcgacgggaaggtcactcggtggtcgttttcgagaggcagaatcaggtcggaggaacatggatctacaccgatcatgtcgagccggatccgttaagcgtcgacccgacccgacccgttgttcactcgagcgtctatggctctctccggaccaaccttccgcgtgagtgtatgggatacagagacttcccgttcgtgatccgatccggcgtctcggaatcgagagacccgaggaggtttccgagtcatagtgaagttctggcgtatctgcaggatttcgcaaaggagtttcggatcgaggagatgatacggttcgagacggcggttgagcgtgttgctccggcggaggaaagcgacggcgaagcaggaaaaatgaaatggaggattgaatctacagagaaagaggagaaacttcgtcgcgatgagatttacgatgccgttgtcgtctgtaacggacattacattgagcctcatctagctgaaattcctggtgattcccctttgattctcctcttgaagtttcaacatcttgactttcgatggattcgtgtgttaataagattactcttttgcttcattcaagaaaatttagatagaatttggaaatag

SEQ ID NO: 178 - protein, 265 aa, Thlaspi arvense
MLYRKEYLWFMMILWARSLSEGLKYSPSLRNLVNTMTPSLSPIRSHHVAVIGAGAAGLVAARELRREGHSVVVFERQNQVGGTWIYTDHVEPDPLSVDPTRPVVHSSVYGSLRTNLPRECMGYRDFPFVIRSGVSESRDPRRFPSHSEVLAYLQDFAKEFRIEEMIRFETAVERVAPAEESDGEAGKMKWRIESTEKEEKLRRDEIYDAVVVCNGHYIEPHLAEIPGDSPLILLLKFQHLDFRWIRVLIRLLFCFIQENLDRIWK

SEQ ID NO: 179 - DNA, 1188 bp, Thlaspi arvense
  source 1..1188: mol_type = genomic DNA
atgatcaccaccacctgttgcactgtggtcgcccagttaggcttgccctttggatcttccttgtcctctctctgcctgacccgtccgaacaaaaagccgtccttgttcacctcatgttgctcctctgtgtccaaaaatgttggcgctagtgctgctgacagcaaacacgtcgtggaacggtggccagagtacattccgaataagctccctgacaagaactatgtgcgtatttttgacacgacgctccgtgacggcgaacaagctcctggtgcagcccttactccaccgcagaagctagagattgcccggcagctcgctaagctccgagtagacatcatggaagttggtttccctgggtcctctgaggaagagttcgaaaccgtcaagaccatcgccaagaccgtagggaacgaggtggatgaggaaacaggctacgtcccagtgatatgcgccctcgcacgatgcaaacataaagacattgaggcggtttgggaggcggtgaagtacgcaaagaggccttcgatactcatattcatatctactagtgacattcatatgaaacataagttgaaaaagactaaagaagaagtcatagagatggcagcaagtagtattaggtttgctaagaacttaggcttcaatgacatccaattgggttgcgaagatgccggcaggtcggataaggagtttctatgcaagattctaggagaagcgatcaaagcgggtgcaaccacgatgaacatggcagacacggtagggatcaacatgccggaagaatttggagaactcgtgagctaccttaaagccaacactcctggaattgacgatgttgtcttaagcgttcattgtcacaacgaccttggtgttgctaccgccaacgcaatcgccggtgtgtgtgctggagcaagacaagttgacgttacggtcaatggaataggtgaaagatgtgggaacgcgccacttgaagaggtcgtgatggctttgaaatgccgaggagcatacctgatgaatggggtttacacaagaacagacatacgccaaattatggctactagcaagatggttcaggaatatactggcttttatgttcaaccacataagcccatagttggagccaacagtttttttcatgagagcagcgatgaatatgatggttgttcaaacattttgcaggatatggattattga

SEQ ID NO: 180 - protein, 395 aa, Thlaspi arvense
MITTTCCTVVAQLGLPFGSSLSSLCLTRPNKKPSLFTSCCSSVSKNVGASAADSKHVVERWPEYIPNKLPDKNYVRIFDTTLRDGEQAPGAALTPPQKLEIARQLAKLRVDIMEVGFPGSSEEEFETVKTIAKTVGNEVDEETGYVPVICALARCKHKDIEAVWEAVKYAKRPSILIFISTSDIHMKHKLKKTKEEVIEMAASSIRFAKNLGFNDIQLGCEDAGRSDKEFLCKILGEAIKAGATTMNMADTVGINMPEEFGELVSYLKANTPGIDDVVLSVHCHNDLGVATANAIAGVCAGARQVDVTVNGIGERCGNAPLEEVVMALKCRGAYLMNGVYTRTDIRQIMATSKMVQEYTGFYVQPHKPIVGANSFFHESSDEYDGCSNILQDMDY

SEQ ID NO: 181 - DNA, 1188 bp, Thlaspi arvense
  source 1..1188: mol_type = genomic DNA
atgatcaccaccacctgttgcactgtggtcgcccagttaggcttgccctttggatcttccttgtcctctctctgcctgacccgtccgaacaaaaagccgtccttgttcacctcatgttgctcctctgtgtccaaaaatgttggcgctagtgctgctgacagcaaacacgtcgtggaacggtggccagagtacattccgaataagctccctgacaagaactatgtgcgtatttttgacacgacgctccgtgacggcgaacaagctcctggtgcagcccttactccaccgcagaagctagagattgcccggcagctcgctaagctccgagtagacatcatggaagttggtttccctgggtcctctgaggaagagttcgaaaccgtcaagaccatcgccaagaccgtagggaacgaggtggatgaggaaacaggctacgtcccagtgatatgcgccctcgcacgatgcaaacatagagacattgaggcggtttgggaggcggtgaagtacgcaaagaggccttcgatactcatattcatatctactagtgacattcatatgaaacataagttgaaaaagactaaagaagaagtcatagagatggcagcaagtagtattaggtttgctaagaacttaggcttcaatgacatccaattgggttgcgaagatgccggcaggtcggataaggagtttctatgcaagattctaggagaagcgatcaaagcgggtgcaaccacgatgaacatggcagacacggtagggatcaacatgccggaagaatttggagaactcgtgagctaccttaaagccaacactcctggaattgacgatgttgtcttaagcgttcattgtcacaacgaccttggtgttgctaccgccaacgcaatcgccggtgtgtgtgctggaacaagacaagttgacgttacggtcaatggaataggtgaaagatgtgggaacgcgccacttgaagaggtcgtgatggctttgaaatgccgaggagcatacctgatgaatggggtttacacaagaacagacatacgccaaattatggctactagcaagatggttcaggaatatactggcttttatgttcaaccacataagcccatagttggagccaacagtttttttcatgagagcagcgatgaatatgatggttgttcaaacattttgcaggatatggattattga

SEQ ID NO: 182 - protein, 395 aa, Thlaspi arvense
MITTTCCTVVAQLGLPFGSSLSSLCLTRPNKKPSLFTSCCSSVSKNVGASAADSKHVVERWPEYIPNKLPDKNYVRIFDTTLRDGEQAPGAALTPPQKLEIARQLAKLRVDIMEVGFPGSSEEEFETVKTIAKTVGNEVDEETGYVPVICALARCKHRDIEAVWEAVKYAKRPSILIFISTSDIHMKHKLKKTKEEVIEMAASSIRFAKNLGFNDIQLGCEDAGRSDKEFLCKILGEAIKAGATTMNMADTVGINMPEEFGELVSYLKANTPGIDDVVLSVHCHNDLGVATANAIAGVCAGTRQVDVTVNGIGERCGNAPLEEVVMALKCRGAYLMNGVYTRTDIRQIMATSKMVQEYTGFYVQPHKPIVGANSFFHESSDEYDGCSNILQDMDY

SEQ ID NO: 183 - DNA, 1365 bp, Thlaspi arvense
  source 1..1365: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagtatgaggatccaggatcctcatgttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 184 - protein, 454 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSESMRIQDPHVLDKVYEFTQLLRPDHCDGNKSISETIQTFSEKLSELDIMVRRMVMESFGIEKYLDKHLNSTNYRLRLMKYIAPPDADATNVAADAKDADDNAKTITNDKVDAAGANDVDAGDIANGIANLHIGDDANAGANGAGVDANDGGEDAKTGEDAKTGECASVKSNAEDGTDVNASADAGVTVGSNADANANANANTSTDAGVGDSVKANGGADDVEKKLGLPSHTDKNLITVLYQYEIEGLEVLTKDDKWIRLKPSHNSFVVMAGDSLYALMNGRLTRPFHRVRVTEKKKTRYSIALFSAPTADYIIDTPKELVDEKHPRIFEPFNYNDLMSFYHSEAGRKARSTLDAFCAVSRA

SEQ ID NO: 185 - DNA, 1818 bp, Thlaspi arvense
  source 1..1818: mol_type = genomic DNA
atgaacgactacttcctcaaccagtcaacagccaccgacgataacgcgtcggcggcgatggaagctttcatcggaacaaatcactcaactctctggccccaaccatctcttcctcctcctcctccactatctcaattcaacgaagacactcttcaacaacgtctccaagctttgatcgaatcagcaggagaaggctgaacctacgcgattttctggcagatctcacacgatttcgattcttccaccggagacaacacagtgattctcggatggggagatgggtactacaaaggggaggaagataaagagaagaagaagaatagctcaagttcgaattcggcggagcaagagcatcggaaaagagtaatccgtgagctgaattcgttgatctccggcggaaccggagtttccgatgaatcgaacgacgaagaagtcaccgatactgagtggttcttcttggtctcgatgacacagagcttcatgaacggcgttgggcttcccggggagtcgtacttgaactctcgcgtgatttggttatccgggccgggtgctttaatcgggtcgggttgcgaacgagcgggtcaaggtcagatatacgggttacagacgatggtgtgtatcgcggcggagaacggcgtcgttgagcttggttcatcggaggtgttaagccatagctcagatctgatggataaagtcaacagtctgttcaattccaacaacggtaatggagaagcttcttcttgggggtttaatctgaatccggatcaaggagagaacgatccggctttgtggataagcgaaccgaccaccaccggaatcgaatcgggtcaggtaatcccggcgataaacaacagtaattccaattcaaattcaaaatccgattctcatcaaatctccaagctcgagaagaacgagagctcgattgaaaaccctagacaacaacaaaatccgtcgcttgtagagcgagatttgaatttctcgagttctgggttgaatcaaaacgggaactttcaagatgggtcgtcgcggatgatgaaatcgaacgaaacactgagctttacggcggaggagagcaacaagaggaggtctccggtttcgaaagggagtaacaacgacgaagggatgctttctttcagcaccgtggttcgttccgcggcgaaatccgtcgattcggatcattccgatctcgaagcgtcggtggttaaggaagcaattgtcgttgaaccggagaagaaaccgaggaaacggggaagaaaaccggcgaacggaagagaggagccgttgaatcacgtggaagcagagagacagagaagggagaagctaaaccagagattctactctttgagagctgtggttcccaacgtttcgaaaatggacaaagcgtctcttctcggagacgccatttcgtatatcaacgagcttaaatcgaagctgcagcaagcggaatccgataaggaagagattcagaagcagctcgacgggatgagcaaggaaggaaacagggaaggcggcggcggaacgaaggcgaaagaacgaaaatgctcgaatcaagattcggctagctcgatagaaatggagatcgacgtgaagatcataggttgggatgtgatgatacgtgtacaatgcagcaagaagaatcatcccggggcaagattcatggaagcgctcaaggaattggatctggaagtgaatcacgcaagtttgtcagtggtgaatgatttgatgattcaacaagccactgtgaagatgggaagccagtttttcaatcatgaccagctcaaggttgctttgatgtcgaaagtcggagaagacaattga

SEQ ID NO: 186 - protein, 65 aa, Thlaspi arvense
MNDYFLNQSTATDDNASAAMEAFIGTNHSTLWPQPSLPPPPPLSQFNEDTLQQRLQALIESAGEG

SEQ ID NO: 187 - DNA, 1817 bp, Thlaspi arvense
  source 1..1817: mol_type = genomic DNA
atgaacgactacttcctcaaccagtcaacagccaccgacgataacgcgtcggcggcgatggaagctttcatcggaacaaatcactcaactctctggccccaaccatctcttcctcctcctcctccactatctcaattcaacgaagacactcttcaacaacgtctccaagctttgatcgaatcagcaggagaaggctggacctacgcgattttctggcagatctcacacgatttcgattcttccaccggagacaaccagtgattctcggatggggagatgggtactacaaaggggaggaagataaagagaagaagaagaatagctcaagttcgaattcggcggagcaagagcatcggaaaagagtaatccgtgagctgaattcgttgatctccggcggaaccggagtttccgatgaatcgaacgacgaagaagtcaccgatactgagtggttcttcttggtctcgatgacacagagcttcatgaacggcgttgggcttcccggggagtcgtacttgaactctcgcgtgatttggttatccgggccgggtgctttaatcgggtcgggttgcgaacgagcgggtcaaggtcagatatacgggttacagacgatggtgtgtatcgcggcggagaacggcgtcgttgagcttggttcatcggaggtgttaagccatagctcagatctgatggataaagtcaacagtctgttcaattccaacaacggtaatggagaagcttcttcttgggggtttaatctgaatccggatcaaggagagaacgatccggctttgtggataagcgaaccgaccaccaccggaatcgaatcgggtcaggtaatcccggcgataaacaacagtaattccaattcaaattcaaaatccgattctcatcaaatctccaagctcgagaagaacgagagctcgattgaaaaccctagacaacaacaaaatccgtcgcttgtagagcgagatttgaatttctcgagttctgggttgaatcaaaacgggaactttcaagatgggtcgtcgcggatgatgaaatcgaacgaaacactgagctttacggcggaggagagcaacaagaggaggtctccggtttcgaaagggagtaacaacgacgaagggatgctttctttcagcaccgtggttcgttccgcggcgaaatccgtcgattcggatcattccgatctcgaagcgtcggtggttaaggaagcaattgtcgttgaaccggagaagaaaccgaggaaacggggaagaaaaccggcgaacggaagagaggagccgttgaatcacgtggaagcagagagacagagaagggagaagctaaaccagagattctactctttgagagctgtggttcccaacgtttcgaaaatggacaaagcgtctcttctcggagacgccatttcgtatatcaacgagcttaaatcgaagctgcagcaagcggaatccgataaggaagagattcagaagcagctcgacgggatgagcaaggaaggaaacagggaaggcggcggcggaacgaaggcgaaagaacgaaaatgctcgaatcaagattcggctagctcgatagaaatggagatcgacgtgaagatcataggttgggatgtgatgatacgtgtacaatgcagcaagaagaatcatcccggggcaagattcatggaagcgctcaaggaattggatctggaagtgaatcacgcaagtttgtcagtggtgaatgatttgatgattcaacaagccactgtgaagatgggaagccagtttttcaatcatgaccagctcaaggttgctttgatgtcgaaagtcggagaagacaattga

SEQ ID NO: 188 - protein, 86 aa, Thlaspi arvense
MNDYFLNQSTATDDNASAAMEAFIGTNHSTLWPQPSLPPPPPLSQFNEDTLQQRLQALIESAGEGWTYAIFWQISHDFDSSTGDNQ

SEQ ID NO: 189 - DNA, 1818 bp, Thlaspi arvense
  source 1..1818: mol_type = genomic DNA
atgaacgactacttcctcaaccagtcaacagccaccgacgataacgcgtcggcggcgatggaagctttcatcggaacaaatcactcaactctctggccccaaccatctcttcctcctcctcctccactatctcaattcaacgaagacactcttcaacaacgtctccaagctttgatcgaatcagcaggagaaggctggacctacgcgattttctggcagatctcacacgatttcgattcttccaccggagacaacacagtgattctcggatggggagatgggtactacaaaggggaggaagataaagagaagaagaagaatagctcaagttcgaattcggcggagcaagagcatcggaaaagagtaatccgtgagctgaattcgttgatctccggcggaaccgaagtttccgatgaatcgaacgacgaagaagtcaccgatactgagtggttcttcttggtctcgatgacacagagcttcatgaacggcgttgggcttcccggggagtcgtacttgaactctcgcgtgatttggttatccgggccgggtgctttaatcgggtcgggttgcgaacgagcgggtcaaggtcagatatacgggttacagacgatggtgtgtatcgcggcggagaacggcgtcgttgagcttggttcatcggaggtgttaagccatagctcagatctgatggataaagtcaacagtctgttcaattccaacaacggtaatggagaagcttcttcttgggggtttaatctgaatccggatcaaggagagaacgatccggctttgtggataagcgaaccgaccaccaccggaatcgaatcgggtcaggtaatcccggcgataaacaacagtaattccaattcaaattcaaaatccgattctcatcaaatctccaagctcgagaagaacgagagctcgattgaaaaccctagacaacaacaaaatccgtcgcttgtagagcgagatttgaatttctcgagttctgggttgaatcaaaacgggaactttcaagatgggtcgtcgcggatgatgaaatcgaacgaaacactgagctttacggcggaggagagcaacaagaggaggtctccggtttcgaaagggagtaacaacgacgaagggatgctttctttcagcaccgtggttcgttccgcggcgaaatccgtcgattcggatcattccgatctcgaagcgtcggtggttaaggaagcaattgtcgttgaaccggagaagaaaccgaggaaacggggaagaaaaccggcgaacggaagagaggagccgttgaatcacgtggaagcagagagacagagaagggagaagctaaaccagagattctactctttgagagctgtggttcccaacgtttcgaaaatggacaaagcgtctcttctcggagacgccatttcgtatatcaacgagcttaaatcgaagctgcagcaagcggaatccgataaggaagagattcagaagcagctcgacgggatgagcaaggaaggaaacagggaaggcggcggcggaacgaaggcgaaagaacgaaaatgctcgaatcaagattcggctagctcgatagaaatggagatcgacgtgaagatcataggttgggatgtgatgatacgtgtacaatgcagcaagaagaatcatcccggggcaagattcatggaagcgctcaaggaattggatctggaagtgaatcacgcaagtttgtcagtggtgaatgatttgatgattcaacaagccactgtgaagatgggaagccagtttttcaatcatgaccagctcaaggttgctttgatgtcgaaagtcggagaagacaattga

SEQ ID NO: 190 - protein, 605 aa, Thlaspi arvense
MNDYFLNQSTATDDNASAAMEAFIGTNHSTLWPQPSLPPPPPLSQFNEDTLQQRLQALIESAGEGWTYAIFWQISHDFDSSTGDNTVILGWGDGYYKGEEDKEKKKNSSSSNSAEQEHRKRVIRELNSLISGGTEVSDESNDEEVTDTEWFFLVSMTQSFMNGVGLPGESYLNSRVIWLSGPGALIGSGCERAGQGQIYGLQTMVCIAAENGVVELGSSEVLSHSSDLMDKVNSLFNSNNGNGEASSWGFNLNPDQGENDPALWISEPTTTGIESGQVIPAINNSNSNSNSKSDSHQISKLEKNESSIENPRQQQNPSLVERDLNFSSSGLNQNGNFQDGSSRMMKSNETLSFTAEESNKRRSPVSKGSNNDEGMLSFSTVVRSAAKSVDSDHSDLEASVVKEAIVVEPEKKPRKRGRKPANGREEPLNHVEAERQRREKLNQRFYSLRAVVPNVSKMDKASLLGDAISYINELKSKLQQAESDKEEIQKQLDGMSKEGNREGGGGTKAKERKCSNQDSASSIEMEIDVKIIGWDVMIRVQCSKKNHPGARFMEALKELDLEVNHASLSVVNDLMIQQATVKMGSQFFNHDQLKVALMSKVGEDN

SEQ ID NO: 191 - DNA, 1109 bp, Thlaspi arvense
  source 1..1109: mol_type = genomic DNA
atgtcaagaaagccgtgttgtgtcggagaagggctgaagaaaggggcatggaccaccgaagaagacaagaaactcatctcttacatccacggcacggtgaaggaggctggcgcgacattccccaaaaagctgggttgaaacggtgtggaaagagttgtaggctgcgatggactaactacctaaaacctgagatcaaaagaggcgagtttagttcagaggaggaacagattatcattatgcttcatgcttctcgtggcaacaagtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtga

SEQ ID NO: 192 - protein, 45 aa, Thlaspi arvense
MSRKPCCVGEGLKKGAWTTEEDKKLISYIHGTVKEAGATFPKKLG

SEQ ID NO: 193 - DNA, 1018 bp, Thlaspi arvense
  source 1..1018: mol_type = genomic DNA
atgtcgaaaagaccatgctgtatcggagaagggttaaagaaaggagcatggacgtcaggggaagacaaaaaactcatctcttatatccatgaacatggcgaaggaggttggcgtgacattcccgaaaaagctgggctaaaacggttgtggaaagagttgcagactgcgatgggcgaactatttgaaccccgatatcaagagaggaggatttagctacgaggaagaacagatcatcatcatgcttcatgcttctcgtggcaacaagtggtcagtcatagcaagacatttgccgcaaagaacagacaacgagatcaaaaactattggaacacacatctcaagaaacgcctgatcaataagagcactgattccgtgacccacaagcctctagcttcctctaaccctagtcctaccgagcgtaagaagctcgattcccaagaagaatccaatcccaaggagcagtcgttacagccgggttcgaagtctccagtatctctttccctttcttcgagtttcaacgacactgtacccgagatcatgaccagtgatgagacgcctctagaaagtggtttcttgagttgcaaaaaaagtgtcgagagatcgagctcaacatcaaggcttttaaacaaagttgcagctagagcttcttccatcgggagtatcttatcaacctccatagaaggaactttgagatctcctgcatcgtcctcatgtctcccaaactcattgtgtcaatcatctgaacacaacaaggatcaagatctcggtacgagcattgatcttagcatccccgattacgattactcccactttctcgagcacttcatcaatagcgaagacgaagccgaaaacattggtggctgcaatcaagatctccttatgtccgatttcccatcaacattagtggataaagaaaatatgaattttgaagacataaccggttggtcaagttatcttctcgaccatcccagttttacgtatgaatcggaacaagattccgacgacaacaacttgttatga

SEQ ID NO: 194 - protein, 70 aa, Thlaspi arvense
MSKRPCCIGEGLKKGAWTSGEDKKLISYIHEHGEGGWRDIPEKAGLKRLWKELQTAMGELFEPRYQERRI

SEQ ID NO: 195 - DNA, 1067 bp, Thlaspi arvense
  source 1..1067: mol_type = genomic DNA
atgtcaagaaagccatgttgtgtgggagaagggctgaagaaaggagcatggaccgccgaagaagacaagaaactcatctcttacatccatgaacatggcgaaggaggctggcgtgacattccccaaaaagctggactaaaacgatgtggaaaaagttgtagattgcgatgggctaactatttgaaaccggatatcaagagaggagaatttagctacgaggaggagcagattatcatcatgcttcacgcttcccgtggcaacaagtggtcggtcatagcgagacatttgcccaaaaggacagacaacgagatcaagaactattggaacacacaccttaaaaaacgccttatcgatcaaggtattgatcccgtgacccacaagccacttgtccggccacgctcaagccttctgatttccaagatgactcatcaaacctgggaaactcggatgagcattcacattcgggttctatgtctccaaaatctcttcctccgtcttcaagctcctgcaatctagcggagataagcagcagtgatgagacaccgaaaaatgatggttccttgaaatccaagaaacgttcttttaagagatcaagttctacatcaaagctgttaaacaaagttgcatctagggctgcttccattggaaatatcttatcagcgtccatggaaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatga

SEQ ID NO: 196 - protein, 171 aa, Thlaspi arvense
MSRKPCCVGEGLKKGAWTAEEDKKLISYIHEHGEGGWRDIPQKAGLKRCGKSCRLRWANYLKPDIKRGEFSYEEEQIIIMLHASRGNKWSVIARHLPKRTDNEIKNYWNTHLKKRLIDQGIDPVTHKPLVRPRSSLLISKMTHQTWETRMSIHIRVLCLQNLFLRLQAPAI

SEQ ID NO: 197 - DNA, 1074 bp, Thlaspi arvense
  source 1..1074: mol_type = genomic DNA
atgtcaagaaagccatgttgtgtgggagaagggctgaagaaaggagcatggaccgccgaagaagacaagaaactcatctcttacatccatgaacatggcgaaggaggctggcgtgacattccccaaaaagctggactaaaacgatgtggaaaaagttgtagattgcgatgggctaactatttgaaaccggatatcaagagaggagaatttagctacgaggaggagcagattatcatcatgcttcacgcttcccgtggcaacaagtggtcggtcatagcgagacatttgcccaaaaggacagacaacgagatcaagaactattggaacacacaccttaaaaaacgccttatcgatcaaggtattgatcccgtgacccacaagccacttgcccctagccctaccacgctcaagccttctgatttccaagatgactcatcaaacctgggaaactcggatgagcattcacattcgggttctatgtctccaaaatctcttcctccgtcttcaagctcctgcaatctagcggagataagcagcagtgatgagacaccgaaaaatgatggttccttgaaatccaagaaacgttcttttaagagatcaagttctacatcaaagctgttaaacaaagttgcatctagggctgcttccattggaaatatcttatcagcgtccatggaaggaaccttggttagctctaccgcactgtctccatgtctcaatgatgacttttccgaagctagccaattccagatggacgaatatgatccattccctcagtcgtctgaacacataactgatcatatgaaggaggacaccggcatgatctttgatctcaacaactccgaatatgatttctcgcagtttctcgagcaatttagtaacgaaggcgaagaaaccgagaacattgggggatataatcaagatctcctttcgtctgacgtctcatcaccaagcgttgatgaagacaatatgatgggaaacataaccggttccggttggtccagttatcttgttgaccattccgattttgtttatgacaagatccaagataacgacgacaggaacttcatatga

SEQ ID NO: 198 - protein, 356 aa, Thlaspi arvense
MSRKPCCVGEGLKKGAWTAEEDKKLISYIHEHGEGGWRDIPQKAGLKRCGKSCRLRWANYLKPDIKRGEFSYEEEQIIIMLHASRGNKWSVIARHLPKRTDNEIKNYWNTHLKKRLIDQGIDPVTHKPLAPSPTLKPSDFQDDSSNLGNSDEHSHSGSMSPKSLPPSSSSCNLAEISSSDETPKNDGSLKSKKRSFKRSSSTSKLLNKVASRAASIGNILSASMEGTLVSSTALSPCLNDDFSEASQFQMDEYDPFPQSSEHITDHMKEDTGMIFDLNNSEYDFSQFLEQFSNEGEETENIGGYNQDLLSSDVSSPSVDEDNMMGNITGSGWSSYLVDHSDFVYDKIQDNDDRNFI

SEQ ID NO: 199 - DNA, 1332 bp, Thlaspi arvense
  source 1..1332: mol_type = genomic DNA
atggcggcgtgtttacaatcgaacatccggctgaatctgaacaatatcgtcggaggaaaatgcagatccctcaccgatcagtctcgcacgccgtgtcgaataaggtgcgccgccgcttcacctgggaaaaaacggttcaacatcgctctccttcctggcgacggtatcggtcctgaagtcatctccgttgccaagaatgtgcttcagcaagctggatctctcgaaggttcttctatctctcgctcagtttatttactaattttgacaaaattagtaatcagcgaatgtgttgcttacccagaagagtgtgcgtatttgatgtgtatcacaggactggagttcaatttccaggagatgcccatcggaggagcagccttggatttggtcggagtggccttgccggaggaaaccttatcggctgcaaaacagtctgatgccatacttcttggagctatcggagggtacaaatgggacaacaatgagaaacatctgagacctgagatggctctgatttaccttcggagagatctcaaagtctttgctaatctgagacctgcttcagttttgccacagctagttgatgcatccaccttgaagagagaagtagcagaaggtgttgatatgatgattgtaagggagcttacaggaggaatttactttggagagcccaaaggcattaagaccaacgaaaacggagaagaagtcggttttaatacagagttttacgctgctcacgagattgacagaattgctcgtgttgcatttgagactgctaggaaacggcgtggcaagctgtgttctgtcgacaaagccaatgtgttggatgcatcagtattgtggaggagaagagtaacagcgttagcctctgagtatccagatgttgagctctcacatatgtatgtagacaacgctgcaatgcagcttattcgtgacccgaaacagtttgacacaatcgtcaccaataacattttcggtgacatattatccgatgaggcctcaatgatcactggaagcattgggatgcttccatctgctagtctcggtgtatcgggacctggactctttgagccgatacatggttctgcgccagatatagctggtcaggacaaggcaaacccattggccaccattctcagcgcagcgatgcttctgaaatatggacttggagaagagaaggccgcaaagaggatcgaggacgcggtcttggatgctctcaacaaagggtttagaaccggagacatctactcccccggaaataaactggtgggatgcaaggagatgggtgaggaggtgcttaaatcagtggactccagagttacagctactgtttaa

SEQ ID NO: 200 - protein, 443 aa, Thlaspi arvense
MAACLQSNIRLNLNNIVGGKCRSLTDQSRTPCRIRCAAASPGKKRFNIALLPGDGIGPEVISVAKNVLQQAGSLEGSSISRSVYLLILTKLVISECVAYPEECAYLMCITGLEFNFQEMPIGGAALDLVGVALPEETLSAAKQSDAILLGAIGGYKWDNNEKHLRPEMALIYLRRDLKVFANLRPASVLPQLVDASTLKREVAEGVDMMIVRELTGGIYFGEPKGIKTNENGEEVGFNTEFYAAHEIDRIARVAFETARKRRGKLCSVDKANVLDASVLWRRRVTALASEYPDVELSHMYVDNAAMQLIRDPKQFDTIVTNNIFGDILSDEASMITGSIGMLPSASLGVSGPGLFEPIHGSAPDIAGQDKANPLATILSAAMLLKYGLGEEKAAKRIEDAVLDALNKGFRTGDIYSPGNKLVGCKEMGEEVLKSVDSRVTATV

SEQ ID NO: 201 - DNA, 1332 bp, Thlaspi arvense
  source 1..1332: mol_type = genomic DNA
atggcggcgtgtttacaatcgaacatccggctgaatctgaacaatatcgtcggaggaaaatgcagatccctcaccgatcagtctcgcacgccgtgtcgaataaggtgcgccgccgcttcacctgggaaaaaacggttcaacatcgctctccttcctggcgacggtatcggtcctgaagtcatctccgttgccaagaatgtgcttcagcaagctggatctctcgaaggttcttctatctctcgctcagtttatttactaattttgacaaaattagtaatcagcgaatgtgttgcttacccagaagagtgtgcgtatttgatgtgtatcacaggactggagttcaatttccaggagatgcccatcggaggagcagccttggatttggtcggagtggccttgccggaggaaaccttatcggctgcaaaacagtctgatgccatacttcttggagctatcggagggtacaaatgggacaacaatgagaaacatctgagacctgagatggctctgatttaccttcggagagatctcaaagtctttgctaatctgagacctgcttcagttttgccacagctagttgatgcatccaccttgaagagagaagtagcagaaggtgttgatatgatgattgtaagggagcttacaggaggaatttactttggagagcccagaggcattaagaccaacgaaaacggagaagaagtcggttttaatacagagttttacgctgctcacgagattgacagaattgctcgtgttgcatttgagactgctaggaaacggcgtggcaagctgtgttctgtcgacaaagccaatgtgttggatgcatcagtattgtggaggagaagagtaacagcgttagcctctgagtatccagatgttgagctctcacatatgtatgtagacaacgctgcaatgcagcttattcgtgacccgaaacagtttgacacaatcgtcaccaataacattttcggtgacatattatccgatgaggcctcaatgatcactggaagcattgggatgcttccatctgctagtctcggtgtattgggacctggactctttgagccgatacatggttctgcgccagatatagctggtcaggacaaggcaaacccattggccaccattctcagcgcagcgatgcttctgaaatatggacttggagaagagaaggccgcaaagaggatcgaggacgcggtcttggatgctctcaacaaagggtttagaaccggagacatctactcccccggaaataaactggtgggatgcaaggagatgggtgaggaggtgcttaaatcagtggactccagagttacagctactgtttaa

SEQ ID NO: 202 - protein, 443 aa, Thlaspi arvense
MAACLQSNIRLNLNNIVGGKCRSLTDQSRTPCRIRCAAASPGKKRFNIALLPGDGIGPEVISVAKNVLQQAGSLEGSSISRSVYLLILTKLVISECVAYPEECAYLMCITGLEFNFQEMPIGGAALDLVGVALPEETLSAAKQSDAILLGAIGGYKWDNNEKHLRPEMALIYLRRDLKVFANLRPASVLPQLVDASTLKREVAEGVDMMIVRELTGGIYFGEPRGIKTNENGEEVGFNTEFYAAHEIDRIARVAFETARKRRGKLCSVDKANVLDASVLWRRRVTALASEYPDVELSHMYVDNAAMQLIRDPKQFDTIVTNNIFGDILSDEASMITGSIGMLPSASLGVLGPGLFEPIHGSAPDIAGQDKANPLATILSAAMLLKYGLGEEKAAKRIEDAVLDALNKGFRTGDIYSPGNKLVGCKEMGEEVLKSVDSRVTATV

SEQ ID NO: 203 - DNA, 1635 bp, Thlaspi arvense
  source 1..1635: mol_type = genomic DNA
atggatactttagcttcaaactcttcgggtctcaggaccaagtcgagtcccgagacgtcgccgtttagcaacatgtatctcctcacaacacttcaagcccttgcggttatttctctcttgatgatattcaagaaaataaagtattcctcttcacaaaaaaaaaagtttcatcctctcccgccgggccccagcgggtttccagtcgtcggaatgattccggcgatgcttaaaaaccgtccggttttccggtggcttcacagcctcatgaaagagcttaacacggagatagcttgtgttcgtctaggaaagactcacgtgatccccgtcacatgtcctaagatcgcacgtgagattttcaagcaacaagacgcactcttcgcgtcgagaccactcacttatgctcaaaagatactttccaacagctacaaaacctgcgtgatcacaccgttcggagaacaattcaagaagatgaggaaagtaatcatgacggagattgtttgtccagcaagacaccgatggttacacgacaacagagccgaggaaaccgatcatttaactgcttggctttacaacatggttaagaaatccgaaccggtcgatctccggtttgttacaaggcactactgcggaaatgcgattaagaggcttatgttcggaacgaggacgttctcggcgaaaaccgaaaccgacggtggaccaaccgtggaagatatcgagcatatggatgctatgtttgaagggttagggtttacgtttgcgttctgtgtatcggattatctaccgatgcttacgggattggatctaaacggacatgagaagatcatgagagaagctagtgcgattatggacaaatatcacgatcccattattgatgagaggattaagatgtggagagaaggaaagagaactcagattgaagattttctagacattttcatctctatcaaggacgaagctggccagcctttgcttaccgctgatgaaatcaaaccaaccattaaggaacttgtaatggcggcgccggacaacccatcaaacgccgtggaatgggccatggcggagatgataaacaaacctgagattctccacaaagcaatggaagagattgaaagagtcgttggcaaagaaagactcgtccaagaatccgatatcccaaaacttaactatctcaaagctattatccgagaagctttccgtctgcatcccgtcgccgccttcaacctcccacacgtggcactttccgacacaaccgtcgctggttatcacatccctaaggggagtcaagttttacttagccgttacggtcttggtcgtaacccaaaggtttggtctgatccacttagctttaaaccggagagacacctcaatgagtgcttggaagtaacgttgactgagaatgatctccggtttatctcgtttagtaccggaaaaagaggatgtgctgctccggcgttaggtacggcgataaccgtcatgatgctcgccaggcttttgcaagggtttaagtggaagttagctggaggtgagacacgtgttgagttgatggaatcgagtcatgatatgtttcttgcgacgcctttggttatggtcggagaattgagattgtcggaggatctttaccccacggtgaagtga

SEQ ID NO: 204 - protein, 544 aa, Thlaspi arvense
MDTLASNSSGLRTKSSPETSPFSNMYLLTTLQALAVISLLMIFKKIKYSSSQKKKFHPLPPGPSGFPVVGMIPAMLKNRPVFRWLHSLMKELNTEIACVRLGKTHVIPVTCPKIAREIFKQQDALFASRPLTYAQKILSNSYKTCVITPFGEQFKKMRKVIMTEIVCPARHRWLHDNRAEETDHLTAWLYNMVKKSEPVDLRFVTRHYCGNAIKRLMFGTRTFSAKTETDGGPTVEDIEHMDAMFEGLGFTFAFCVSDYLPMLTGLDLNGHEKIMREASAIMDKYHDPIIDERIKMWREGKRTQIEDFLDIFISIKDEAGQPLLTADEIKPTIKELVMAAPDNPSNAVEWAMAEMINKPEILHKAMEEIERVVGKERLVQESDIPKLNYLKAIIREAFRLHPVAAFNLPHVALSDTTVAGYHIPKGSQVLLSRYGLGRNPKVWSDPLSFKPERHLNECLEVTLTENDLRFISFSTGKRGCAAPALGTAITVMMLARLLQGFKWKLAGGETRVELMESSHDMFLATPLVMVGELRLSEDLYPTVK

SEQ ID NO: 205 - DNA, 1635 bp, Thlaspi arvense
  source 1..1635: mol_type = genomic DNA
atggatactttagcttcaaactcttcgggtctcaggaccaagtcgagtcccgagacgtcgccgtttagcaacatgtatctcctcacaacacttcaagcccttgcggttatttctctcttgatgatattcaagaaaataaagtattcctcttcacaaaaaaaaaagtttcatcctctcccgccgggccccagcgggtttccagtcgtcggaatgattccggcgatgcttaaaaaccgtccggttttccggtggcttcacagcctcatgaaagagcttaacacggagatagcttgtgttcgtctaggaaagactcacgtgatccccgtcacatgtcctaagatcgcacgtgagattttcaagcaacaagacgcactcttcgcgtcgagaccactcacttatgctcaaaagatactttccaacggctacaaaacctgcgtgatcacaccgttcggagaacaattcaagaagatgaggaaagtaatcatgacggagattgtttgtccagcaagacaccgatggttacacgacaacagagccgaggaaaccgatcatttaactgcttggctttacaacatggttaagaaatccgaaccggtcgatctccggtttgttacaaggcactactgcggaaatgcgattaagaggcttatgttcggaacgaggacgttctcggcgaaaaccgaaaccgacggtggaccaaccgtggaagatatcgagcatatggatgctatgtttgaagggttagggtttacgtttgcgttctgtgtatcggattatctaccgatgcttacgggattggatctaaacggacatgagaagatcatgagagaagctagtgcgattatggacaaatatcacgatcccattattgatgagaggattaagatgtggagagaaggaaagagaactcagattgaagattttctagacattttcatctctatcaaggacgaagctggccagcctttgcttaccgctgatgaaatcaaaccaaccattaaggaacttgtaatggcggcgccggacaacccatcaaacgccgtggaatgggccatggcggagatgataaacaaacctgagattctccacaaagcaatggaagagattgaaagagtcgttggcaaagaaagactcgtccaagaatccgatatcccaaaacttaactatctcaaagctattatccgagaagctttccgtctgcatcccgtcgccgccttcaacctcccacacgtggcactttccgacacaaccgtcgctggttatcacatccctaaggggagtcaagttttacttagccgttacggtcttggtcgtaacccaaaggtttggtctgattcacttagctttaaaccggagagacacctcaatgagtgcttggaagtaacgttgactgagaatgatctccggtttatctcgtttagtaccggaaaaagaggatgtgctgctccggcgttaggtacggcgataaccgtcatgatgctcgccaggcttttgcaagggtttaagtggaagttagctggaggtgagacacgtgttgagttgatggaatcgagtcatgatatgtttcttgcgacgcctttggttatggtcggagaattgagattgtcggaggatctttaccccacggtgaagtga

SEQ ID NO: 206 - protein, 544 aa, Thlaspi arvense
MDTLASNSSGLRTKSSPETSPFSNMYLLTTLQALAVISLLMIFKKIKYSSSQKKKFHPLPPGPSGFPVVGMIPAMLKNRPVFRWLHSLMKELNTEIACVRLGKTHVIPVTCPKIAREIFKQQDALFASRPLTYAQKILSNGYKTCVITPFGEQFKKMRKVIMTEIVCPARHRWLHDNRAEETDHLTAWLYNMVKKSEPVDLRFVTRHYCGNAIKRLMFGTRTFSAKTETDGGPTVEDIEHMDAMFEGLGFTFAFCVSDYLPMLTGLDLNGHEKIMREASAIMDKYHDPIIDERIKMWREGKRTQIEDFLDIFISIKDEAGQPLLTADEIKPTIKELVMAAPDNPSNAVEWAMAEMINKPEILHKAMEEIERVVGKERLVQESDIPKLNYLKAIIREAFRLHPVAAFNLPHVALSDTTVAGYHIPKGSQVLLSRYGLGRNPKVWSDSLSFKPERHLNECLEVTLTENDLRFISFSTGKRGCAAPALGTAITVMMLARLLQGFKWKLAGGETRVELMESSHDMFLATPLVMVGELRLSEDLYPTVK

SEQ ID NO: 207 - DNA, 1509 bp, Thlaspi arvense
  source 1..1509: mol_type = genomic DNA
atggaagatatcatcatcggcgttgtggctctcgccgccgttctcctcttcttcctctaccaaagcccaaaaaccaaacggtataagctgcctcccggcccacggccgcttccggtgatcggaaacctccaccagcttagccaggttaacccacaacggttcttctatggatgggccaaaaaatatggaccaatcttatcatacaagataggaaacaaaacaatgatggtaatttcttcggctgagctaaccaaagagcttctcaagacgcaagatgtcaacttcgccaaccggcctcctcaccgtggtcacgagctcatgacctacggccgaagtgacatggcgatgaaccactacacaccgttgtaccgggagatgaggaagatgggcatgaaccacttgttctcccccactcgtgtagccacctttaagcacgtacgggaggaggaggctaggaggatgatgtttaagatcgagaaggctgcggagagatctgaaccggtcgatataagcgagcttatgttgaccttcacgaactcggttgtgtgtaggcaagctttcgggaagaagtacaatgaagatggggaagagatgaagagattcatcaggattctttatgggactcagagcgtattggggaagatttttttctctgattttttcccgtttactcgctacgttcttgataattggaccggcctcacgaaatatatgatggactgttttgaaagacaagacacttacatacaagagattatcgatgagacacttgatcccaacaaggtaaagccagaaacggagagcatgatcgatctcttgatggaggtctacaaagaacaaccattcgcctccaagttcacaattgggaatgtcaaaggcgttatcttgaatatagtggttgcgggaaccgacacggcggctgcggcggttgtgtgggggatgacgtatctaatgaagtaccctcaagttatgaagaaagctcaagcagaagtgagagagtatgcaaaagagaaagatctaacgtttattactgaagacgacgtcaagaaccttccttacttcagagctttagttaaagaaaccttaaggatcgaaccagtgattcctctccttatccctcgttgttgcattcaagacaccaagatcgccggttacgatgtccccgcggggaccacggtcaacgtaaacgcgtgggcggtgtcacgcgacgagaaggagtggggcccaaaccctgatgaattcaggcccaagaggtttcttgagaaggacgtggacttcaaaggcacggactatgagtttataccgtttgggtcaggccggagaatgtgccctggaatgcgtcttggcgcggcgatgatcgaggttccgtatgcgaaccttttgctcaactttgacttcaaacttgctgatggactgaaaccagaagagatcaacatggatgttatgacaggtcttgctatgcacaaggcggttcatctcaggcttgttcccgagaaagtgaggaagtga

SEQ ID NO: 208 - protein, 502 aa, Thlaspi arvense
MEDIIIGVVALAAVLLFFLYQSPKTKRYKLPPGPRPLPVIGNLHQLSQVNPQRFFYGWAKKYGPILSYKIGNKTMMVISSAELTKELLKTQDVNFANRPPHRGHELMTYGRSDMAMNHYTPLYREMRKMGMNHLFSPTRVATFKHVREEEARRMMFKIEKAAERSEPVDISELMLTFTNSVVCRQAFGKKYNEDGEEMKRFIRILYGTQSVLGKIFFSDFFPFTRYVLDNWTGLTKYMMDCFERQDTYIQEIIDETLDPNKVKPETESMIDLLMEVYKEQPFASKFTIGNVKGVILNIVVAGTDTAAAAVVWGMTYLMKYPQVMKKAQAEVREYAKEKDLTFITEDDVKNLPYFRALVKETLRIEPVIPLLIPRCCIQDTKIAGYDVPAGTTVNVNAWAVSRDEKEWGPNPDEFRPKRFLEKDVDFKGTDYEFIPFGSGRRMCPGMRLGAAMIEVPYANLLLNFDFKLADGLKPEEINMDVMTGLAMHKAVHLRLVPEKVRK

SEQ ID NO: 209 - DNA, 1510 bp, Thlaspi arvense
  source 1..1510: mol_type = genomic DNA
atggaagatatcatcatcggcgttgtggctctcgccgccgttctcctcttcttcctctaccaaagcccaaaaaccaaacggtataagctgcctcccggcccacggccgcttccggtgatcggaaacctccaccagcttagccaggttaacccacaacggttcttctatggatgggccaaaaaatatggaccaatcttatcatacaaagataggaaacaaaacaatgatggtaatttcttcggctgagctaaccaaagagcttctcaagacgcaagatgtcaacttcgccaaccggcctcctcaccgtggtcacgagctcatgacctacggccgaagtgacatggcgatgaaccactacacaccgttgtaccgggagatgaggaagatgggcatgaaccacttgttctcccccactcgtgtagccacctttaagcacgtacgggaggaggaggctaggaggatgatgtttaagatcgagaaggctgcggagagatctgaaccggtcgatataagcgagcttatgttgaccttcacgaactcggttgtgtgtaggcaagctttcgggaagaagtacaatgaagatggggaagagatgaagagattcatcaggattctttatgggactcagagcgtattggggaagatttttttctctgattttttcccgtttactcgctacgttcttgataattggaccggcctcacgaaatatatgatggactgttttgaaagacaagacacttacatacaagagattatcgatgagacacttgatcccaacaaggtaaagccagaaacggagagcatgatcgatctcttgatggaggtctacaaagaacaaccattcgcctccaagttcacaattgggaatgtcaaaggcgttatcttgaatatagtggttgcgggaaccgacacggcggctgcggcggttgtgtgggggatgacgtatctaatgaagtaccctcaagttatgaagaaagctcaagcagaagtgagagagtatgcaaaagagaaagatctaacgtttattactgaagacgacgtcaagaaccttccttacttcagagctttagttaaagaaaccttaaggatcgaaccagtgattcctctccttatccctcgttgttgcattcaagacaccaagatcgccggttacgatgtccccgcggggaccacggtcaacgtaaacgcgtgggcggtgtcacgcgacgagaaggagtggggcccaaaccctgatgaattcaggcccgagaggtttcttgagaaggacgtggacttcaaaggcacggactatgagtttataccgtttgggtcaggccggagaatgtgccctggaatgcgtcttggcgcggcgatgatcgaggttccgtatgcgaaccttttgctcaactttgacttcaaacttgctgatggactgaaaccagaagagatcaacatggatgttatgacaggtcttgctatgcacaaggcggttcatctcaggcttgttcccgagaaagtgaggaagtga

SEQ ID NO: 210 - protein, 81 aa, Thlaspi arvense
MEDIIIGVVALAAVLLFFLYQSPKTKRYKLPPGPRPLPVIGNLHQLSQVNPQRFFYGWAKKYGPILSYKDRKQNNDGNFFG

SEQ ID NO: 211 - DNA, 1510 bp, Thlaspi arvense
  source 1..1510: mol_type = genomic DNA
atggaagatatcatcatcggcgttgtggctctcgccgccgttctcctcttcttcctctaccaaagcccaaaaaccaaacggtataagctgcctcccggcccacggccgcttccggtgatcggaaacctccaccagcttagccaggttaacccacaacggttcttctatggatgggccaaaaaatatggaccaatcttatcatacaatgataggaaacaaaacaatgatggtaatttcttcggctgagctaaccaaagagcttctcaagacgcaagatgtcaacttcgccaaccggcctcctcaccgtggtcacgagctcatgacctacggccgaagtgacatggcgatgaaccactacacaccgttgtaccgggagatgaggaagatgggcatgaaccacttgttctcccccactcgtgtagccacctttaagcacgtacgggaggaggaggctaggaggatgatgtttaagatcgagaaggctgcggagagatctgaaccggtcgatataagcgagcttatgttgaccttcacgaactcggttgtgtgtaggcaagctttcgggaagaagtacaatgaagatggggaagagatgaagagattcatcaggattctttatgggactcagagcgtattggggaagatttttttctctgattttttcccgtttactcgctacgttcttgataattggaccggcctcacgaaatatatgatggactgttttgaaagacaagacacttacatacaagagattatcgatgagacacttgatcccaacaaggtaaagccagaaacggagagcatgatcgatctcttgatggaggtctacaaagaacaaccattcgcctccaagttcacaattgggaatgtcaaaggcgttatcttgaatatagtggttgcgggaaccgacacggcggctgcggcggttgtgtgggggatgacgtatctaatgaagtaccctcaagttatgaagaaagctcaagcagaagtgagagagtatgcaaaagagaaagatctaacgtttattactgaagacgacgtcaagaaccttccttacttcagagctttagttaaagaaaccttaaggatcgaaccagtgattcctctccttatccctcgttgttgcattcaagacaccaagatcgccggttacgatgtccccgcggggaccacggtcaacgtaaacgcgtgggcggtgtcacgcgacgagaaggagtggggcccaaaccctgatgaattcaggcccgagaggtttcttgagaaggacgtggacttcaaaggcacggactatgagtttataccgtttgggtcaggccggagaatgtgccctggaatgcgtcttggcgcggcgatgatcgaggttccgtatgcgaaccttttgctcaactttgacttcaaacttgctgatggactgaaaccagaagagatcaacatggatgttatgacaggtcttgctatgcacaaggcggttcatctcaggcttgttcccgagaaagtgaggaagtga

SEQ ID NO: 212 - protein, 81 aa, Thlaspi arvense
MEDIIIGVVALAAVLLFFLYQSPKTKRYKLPPGPRPLPVIGNLHQLSQVNPQRFFYGWAKKYGPILSYNDRKQNNDGNFFG

SEQ ID NO: 213 - DNA, 1332 bp, Thlaspi arvense
  source 1..1332: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtcttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 214 - protein, 443 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLLDKVYEFTQLLRPDHCDGNKSISETIQTFSEKLSELDIMVRRMVMESFGIEKYLDKHLNSTNYRLRLMKYIAPPDADATNVAADAKDADDNAKTITNDKVDAAGANDVDAGDIANGIANLHIGDDANAGANGAGVDANDGGEDAKTGEDAKTGECASVKSNAEDGTDVNASADAGVTVGSNADANANANANTSTDAGVGDSVKANGGADDVEKKLGLPSHTDKNLITVLYQYEIEGLEVLTKDDKWIRLKPSHNSFVVMAGDSLYALMNGRLTRPFHRVRVTEKKKTRYSIALFSAPTADYIIDTPKELVDEKHPRIFEPFNYNDLMSFYHSEAGRKARSTLDAFCAVSRA

SEQ ID NO: 215 - DNA, 1363 bp, Thlaspi arvense
  source 1..1363: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagtatggggatccaggatcctcattttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 216 - protein, 115 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSESMGIQDPHFGQSLRVYSTSTS

SEQ ID NO: 217 - DNA, 1364 bp, Thlaspi arvense
  source 1..1364: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagtatggggatccaggatcctcatgtttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 218 - protein, 140 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSESMGIQDPHVWTKFTSLLNFYVLIIVTVTRASAKRSRRFQRSYQNWI

SEQ ID NO: 219 - DNA, 1366 bp, Thlaspi arvense
  source 1..1366: mol_type = genomic DNA
atgggttcactttcaaacactcctcagcttccagtcatctacctctcggaccaaaccctaaaaccaggaagctcaaagtgggttgaagtcaggagtgatgtccgtaaagctcttgaagagtacggcggtttcgaggtgtcgtacgatagagtgtcggaggagcttaagaagtcggttttgcaagccatggaagagcttttcgcgttaccagttgaggctaaacagagaaacgtctctcctaaacccttcagcggttattccactcataacggtctttccgagagtatggggatccaggatcctcatgtttttggacaaagtttacgagtttactcaacttctacgtcctgatcattgtgacggtaacaagagcatcagcgaaacgatccagacgttttcagagaagttatcagaattggatataatggtgagaagaatggtaatggaaagcttcgggatagagaagtaccttgacaaacacctgaactcaacgaattaccgtctgcggctgatgaagtatatagcaccgcctgatgctgatgctactaatgttgcggctgatgccaaagatgctgatgataatgctaagacgattacaaatgataaagttgatgcggctggtgctaatgatgtagatgctggtgatatcgctaatggtattgctaatcttcatattggtgatgatgctaacgctggtgctaatggtgctggtgttgatgctaatgatggtggtgaggatgctaagactggtgaggatgctaagactggtgaatgtgctagtgttaagtctaatgccgaagatggtactgatgttaatgccagtgctgatgctggtgttactgttggctctaatgctgatgctaatgctaatgctaatgctaatactagtactgatgctggtgttggcgatagtgttaaagctaatggtggtgctgatgatgttgagaagaaattgggtctaccttctcacactgataagaaccttataacggtgctttatcaatacgagattgaaggcttggaggttctaaccaaagatgacaagtggatcagactcaaaccatctcataattctttcgttgttatggctggagattctctatacgcacttatgaatggtagactaactcgtccctttcatcgagtaagagtaacggagaaaaagaagacaagatattcaatagcattgttctcggctccaaccgcagattacatcatagacacaccaaaagaacttgtggacgagaagcatccacgtatcttcgaaccatttaactataacgacttgatgagtttctatcatagtgaagctggtcgtaaagctcgatctactcttgatgctttctgtgccgtctctcgagcataa

SEQ ID NO: 220 - protein, 116 aa, Thlaspi arvense
MGSLSNTPQLPVIYLSDQTLKPGSSKWVEVRSDVRKALEEYGGFEVSYDRVSEELKKSVLQAMEELFALPVEAKQRNVSPKPFSGYSTHNGLSESMGIQDPHVFGQSLRVYSTSTS

SEQ ID NO: 221 - DNA, 1956 bp, Thlaspi arvense
  source 1..1956: mol_type = genomic DNA
atgaagagtagagtcatcctcagccatagagagagaagagataagaagaataataacattaacaacaaagacatctcctgtaatttcacacagattgaaaccatggagagaaagccctttgaggttgagacgacggagaatcacaaaccctactccaccgtcgatggcggtggcgttggttctgatttgagatcgccggtcgattcatttgatgacgagcagaaaaagctcgtttacagaggctggaaagtcatgccttttatcattggtaatgagacatttgagaagattgggatcatagggacattatcaaaccttcttttgtacctaactcaagtattcaaccttaagaaagttacagctgcaacaatcatcaatgcctttagtggcacaatcaacttcgggactttcatcgctgctttcctctgcgacacttactttggtcgctacaagactctcagtgtagctgtcatcgcttgtttcctgggatcgcttgtgatattactgacggctgcagttcctgcattgcacccgactccatgtggaacacatagctggtgccaagggccaagcccgggccagatcgcgttcttgctgctgggtttagcgtttcttgtggtcggtgcgggtgggatcaggccgtgtaacttggcttttggagctgatcagttcaaccccaaatccgaatccgggaagaaaggaatcaacagcttctttaactggtatttcttcaccttcacgtttgcgcagatcgtctcgctcacgctggtcgtgtatatccagtcgaacgtgagctggacgatcggtttgctcatccctgtggctctgatgttcttggcctgcgtcatcttctttgctggacataaactgtatgtgaaagtgaaagcctcgggtagtcccttggctagtatcggtcacgttatcacggcagcgatcaagaaacgagggttgaagcaagttaagcagccttggctcgatctttacaaccacattccaactaactatccaaactccaccttgaaatacaccgaccagtttaggtttcttgacaaagcagcgattatgacccctgaggacaagctgaattccgatggagctgctttcgatccatggaccctatgtacattgcagaaagtggaagaagtgaaatgcattgtgagagtgattccgatctggtttgcttgcgcgatttactacctcactgtaactatacagatgacttatccggtcttccaagcgcagcagagcgaccggagattgggttctggtggcttcaagatccccgcagccacctatgtggtgttcttgatgtcgggtatgactgttttcatcgtgttctacgaccgtgtccttgtcccgttgctcagaagagtgaccgggttagaaaccggtttgaccctcttgcagagagtcggatcagggatcttctttgccatgttgagtttgttggtctccgggttcgtagaggaacggagaagaaccttcgccctgacgaaaccgactctcgggatggagccacgagcgggagagatctcctccatgtcggccatgtggctgattccgcagctcttgtttgcaggcgtaggagaggcttttacagccattggacagatggagttttattacaagcagttccctgagaacatgaagagcttcgctggctctatcttctatgtcggtgcaggtgtttcgagctatcttgctagcttcttgatctcgactgttcatcgaagaactgaacattcaccctccgggaactggttagctgaggatctgaacaaagggagactcgattacttctacttcatgctcaccggaatcatggtcgttaacatggtttacttcttgataatgtctaaatggtatagatacaaaggcattaacgatgaagcgaattctttggtcgagaccaatgaagaagagaccaagcagaaacaagtcaagaattctgtctga

SEQ ID NO: 222 - protein, 651 aa, Thlaspi arvense
MKSRVILSHRERRDKKNNNINNKDISCNFTQIETMERKPFEVETTENHKPYSTVDGGGVGSDLRSPVDSFDDEQKKLVYRGWKVMPFIIGNETFEKIGIIGTLSNLLLYLTQVFNLKKVTAATIINAFSGTINFGTFIAAFLCDTYFGRYKTLSVAVIACFLGSLVILLTAAVPALHPTPCGTHSWCQGPSPGQIAFLLLGLAFLVVGAGGIRPCNLAFGADQFNPKSESGKKGINSFFNWYFFTFTFAQIVSLTLVVYIQSNVSWTIGLLIPVALMFLACVIFFAGHKLYVKVKASGSPLASIGHVITAAIKKRGLKQVKQPWLDLYNHIPTNYPNSTLKYTDQFRFLDKAAIMTPEDKLNSDGAAFDPWTLCTLQKVEEVKCIVRVIPIWFACAIYYLTVTIQMTYPVFQAQQSDRRLGSGGFKIPAATYVVFLMSGMTVFIVFYDRVLVPLLRRVTGLETGLTLLQRVGSGIFFAMLSLLVSGFVEERRRTFALTKPTLGMEPRAGEISSMSAMWLIPQLLFAGVGEAFTAIGQMEFYYKQFPENMKSFAGSIFYVGAGVSSYLASFLISTVHRRTEHSPSGNWLAEDLNKGRLDYFYFMLTGIMVVNMVYFLIMSKWYRYKGINDEANSLVETNEEETKQKQVKNSV

SEQ ID NO: 223 - DNA, 1957 bp, Thlaspi arvense
  source 1..1957: mol_type = genomic DNA
atgaagagtagagtcatcctcagccatagagagagaagagataagaagaataataacattaacaacaaagacatctcctgtaatttcacacagattgaaaccatggagagaaagccctttgaggttgagacgacggagaatcacaaaccctactccaccgtcgatggcggtggcgttggttctgatttgagatcgccggtcgattcatttgatgacgagcagaaaaagctcgtttacagaggctggaaagtcatgccttttatcattggtaatgagacatttgagaagattgggatcatagggacattatcaaaccttcttttgtacctaactcaagtattcaaccttaagaaagttacagctgcaacaatcatcaatgcctttagtggcacaatcaacttcgggactttcatcgctgctttcctctgcgacacttactttggtcgctacaagactctcagtgtagctgtcatcgcttgtttcctgggatcgcttgtgatattactgacggctgcagttcctgcattgcacccgactccatgtggaacacatagctggtgccaagggccaagcccgggccagatcgcgttcttgctgctgggtttagcgtttcttgtggtcggtgcgggtgggatcaggccgtgtaacttggcttttggagctgatcagttcaaccccaaatccgaatccgggaagaaaggaatcaacagcttctttaactggtatttcttcaccttcacgtttgcgcagatcgtctcgctcacgctggtcgtgtatatccagtcgaacgtgagctggacgatcggtttgctcatccctgtggctctgatgttcttggcctgcgtcatcttctttgctggacataaactgtatgtgaaagtgaaagcctcgggtagtcccttggctagtatcggtcacgttatcacggcagcgatcaagaaacgagggttgaagcaagttaagcagccttggctcgatctttacaaccacattccaactaactatccaaactccaccttgaaatacaccgaccagtttaggtttcttgacaaagcagcgattatgacccctgaggacaagctgaattccgatggagctgctttcgatccatggaccctatgtacattgcagaaagtggaagaagtgaaatgcatgtgtgagagtgattccgatctggtttgcttgcgcgatttactacctcactgtaactatacagatgacttatccggtcttccaagcgcagcagagcgaccggagattgggttctggtggcttcaagatccccgcagccacctatgtggtgttcttgatgtcgggtatgactgttttcatcgtgttctacgaccgtgtccttgtcccgttgctcagaagagtgaccgggttagaaaccggtttgaccctcttgcagagagtcggatcagggatcttctttgccatgttgagtttgttggtctccgggttcgtagaggaacggagaagaaccttcgccctgacgaaaccgactctcgggatggagccacgagcgggagagatctcctccatgtcggccatgtggctgattccgcagctcttgcttgcaggcgtaggagaggcttttacagccattggacagatggagttttattacaagcagttccctgagaacatgaagagcttcgctggctctatcttctatgtcggtgcaggtgtttcgagctatcttgctagcttcttgatctcgactgttcatcgaagaactgaacattcaccctccgggaactggttagctgaggatctgaacaaagggagactcgattacttctacttcatgctcaccggaatcatggtcgttaacatggtttacttcttgataatgtctaaatggtatagatacaaaggcattaacgatgaagcgaattctttggtcgagaccaatgaagaagagaccaagcagaaacaagtcaagaattctgtctga

SEQ ID NO: 224 - protein, 546 aa, Thlaspi arvense
MKSRVILSHRERRDKKNNNINNKDISCNFTQIETMERKPFEVETTENHKPYSTVDGGGVGSDLRSPVDSFDDEQKKLVYRGWKVMPFIIGNETFEKIGIIGTLSNLLLYLTQVFNLKKVTAATIINAFSGTINFGTFIAAFLCDTYFGRYKTLSVAVIACFLGSLVILLTAAVPALHPTPCGTHSWCQGPSPGQIAFLLLGLAFLVVGAGGIRPCNLAFGADQFNPKSESGKKGINSFFNWYFFTFTFAQIVSLTLVVYIQSNVSWTIGLLIPVALMFLACVIFFAGHKLYVKVKASGSPLASIGHVITAAIKKRGLKQVKQPWLDLYNHIPTNYPNSTLKYTDQFRFLDKAAIMTPEDKLNSDGAAFDPWTLCTLQKVEEVKCMCESDSDLVCLRDLLPHCNYTDDLSGLPSAAERPEIGFWWLQDPRSHLCGVLDVGYDCFHRVLRPCPCPVAQKSDRVRNRFDPLAESRIRDLLCHVEFVGLRVRRGTEKNLRPDETDSRDGATSGRDLLHVGHVADSAALACRRRRGFYSHWTDGVLLQAVP

SEQ ID NO: 225 - DNA, 1842 bp, Thlaspi arvense
  source 1..1842: mol_type = genomic DNA
atggagagacagcctctcgaactcgagtctacggatcaccaaaaaccttcccccgccgtctacggtggctctgttacgacggttgattctgttgaggaagaagttcaggagaaaaaagtcgtatatagaggctggaaagtcatgccctttatcattggaaatgagacattcgagaagcttgggatcattggaacactatcaaaccttctggtttttttaacagctgtcttcaacatgaagagtatcacagctgcaacaatcattaacgcattcagtggcacaataaatttcggaactttcgttgctgctttcctctgcgacacttacttcggtcgatacaagacactaaccgtcgcggtcatcgcctgttttcttggatcacttgtgatactattgacagctgcagtgccacaactgcacccagctccatgtggaacagcgagctcgtgcagcggtccaagcggtgggcaaatcgcgtttcttctgatgggtctcgggtttcttgtagttggagcgggcgggatcagaccgtgcaatctagctttcggagccgatcagttcaacccgaagagcgagtcagggagaagagggactgatagtttcttcaattggtacttcttcagcttcactttcgcgcagatcttgtcgctgacgctagtggtctacatccagtctaacgtcagctggacgatcggtctaaccataccggtggttctaatgttcttggcctccgtgatcttctttgcgggagataagctgtatgtgaaagtcaaggcctcgggtagtccattggctggtatagctcaagtgatcattgttgcaatcaagaaacgcggattaaagcccgtgaaacagccttggcttaacctctacaattactgccctccaaaacacgcaaactccattctcaaatacaccgaccaattcagatttcttgataaggcggcgatcttggctcccgaggacaagttggaggcggatggtaagcctgcggatccatggaagctgtgtacgatgcaacaggtggaagaagtgaagtgcattgtgagagtgcttcctatatggttcgctgcatcaatctactacgtaaccataacccagcaaatgacatatccggtcttccaagccctgcagagcgatcgtcgcttaggctcgggagggttcgtgatccccgcagccacctacgtggtcttcttgatgacagggatgacggttttcatcatcatctacgaccgtctcctcgtgcctaccttgagaagaataaccggtctagacaccgggatcacgctcctgcagagaatcggaaccgggatcttcttcgcctttgcaagcttagtagtctccggtttcgtcgaggagcggaggagacacattgcgctgactaaaccaactcttggcatggcgccaagaaaaggagaaatctcctcaatgtcagctatgtggctcatcccgcagctcactctctcgggtgtagccgaggcgtttggagccatcggacagatggagttttactacaagcagttcccagaaaacatgaggagtttcgcgggttccatcttttatgtaggaataggggtttcgagttacctcggcagcttcttgattgcaacggttcaccggacgacgcagaactcggcgggtggtaactggttggctgaggatttgaacaaaggcagattggattacttctatttcatgatcgctggaatcttggctgttaatttcgcctacttcttggtcgtgtcaagatggtataggtacaaagaaagtaatgatgatcaaaagacagcttctgaaaccaatggagatgtcatcaaacaacaagacaagaacactgcctga

SEQ ID NO: 226 - protein, 613 aa, Thlaspi arvense
MERQPLELESTDHQKPSPAVYGGSVTTVDSVEEEVQEKKVVYRGWKVMPFIIGNETFEKLGIIGTLSNLLVFLTAVFNMKSITAATIINAFSGTINFGTFVAAFLCDTYFGRYKTLTVAVIACFLGSLVILLTAAVPQLHPAPCGTASSCSGPSGGQIAFLLMGLGFLVVGAGGIRPCNLAFGADQFNPKSESGRRGTDSFFNWYFFSFTFAQILSLTLVVYIQSNVSWTIGLTIPVVLMFLASVIFFAGDKLYVKVKASGSPLAGIAQVIIVAIKKRGLKPVKQPWLNLYNYCPPKHANSILKYTDQFRFLDKAAILAPEDKLEADGKPADPWKLCTMQQVEEVKCIVRVLPIWFAASIYYVTITQQMTYPVFQALQSDRRLGSGGFVIPAATYVVFLMTGMTVFIIIYDRLLVPTLRRITGLDTGITLLQRIGTGIFFAFASLVVSGFVEERRRHIALTKPTLGMAPRKGEISSMSAMWLIPQLTLSGVAEAFGAIGQMEFYYKQFPENMRSFAGSIFYVGIGVSSYLGSFLIATVHRTTQNSAGGNWLAEDLNKGRLNYFYFMIAGILAVNFAYFLVVSRWYRYKESDDDQKTASETNGDVIKQQDKNTA

SEQ ID NO: 227 - DNA, 1843 bp, Thlaspi arvense
  source 1..1843: mol_type = genomic DNA
atggagagacagcctctcgaactcgagtctacggatcaccaaaaaccttcccccgccgtctacggtggctctgttacgacggttgattctgttgaggaagaagttcaggagaaaaaagtcgtatatagaggctggaaagtcatgccctttatcattggaaatgagacattcgagaagcttgggatcattggaacactatcaaaccttctggtttttttaacagctgtcttcaacatgaagagtatcacagctgcaacaatcattaacgcattcagtggcacaataaatttcggaactttcgttgctgctttcctctgcgacacttacttcggtcgatacaagacactaaccgtcgcggtcatcgcctgttttcttggatcacttgtgatactattgacagctgcagtgccacaactgcacccagctccatgtggaacagcgagctcgtgcagcggtccaagcggtgggcaaatcgcgtttcttctgatgggtctcgggtttcttgtagttggagcgggcgggatcagaccgtgcaatctagctttcggagccgatcagttcaacccgaagagcgagtcagggagaagagggactgatagtttcttcaattggtacttcttcagcttcactttcgcgcagatcttgtcgctgacgctagtggtctacatccagtctaacgtcagctggacgatcggtctaaccataccggtggttctaatgttcttggcctccgtgatcttctttgcgggagataagctgtatgtgaaagtcaaggcctcgggtagtccattggctggtatagctcaagtgatcattgttgcaatcaagaaacgcggattaaagcccgtgaaacagccttggcttaacctctacaattactgccctccaaaacacgcaaactccattctcaaatacaccgaccaattcagatttcttgataaggcggcgatcttggctcccgaggacaagttggaggcggatggtaagcctgcggatccatggaagctgtgtacgatgcaacaggtggaagaagtgaagtgcatatgtgagagtgcttcctatatggttcgctgcatcaatctactacgtaaccataacccagcaaatgacatatccggtcttccaagccctgcagagcgatcgtcgcttaggctcgggagggttcgtgatccccgcagccacctacgtggtcttcttgatgacagggatgacggttttcatcatcatctacgaccgtctcctcgtgcctaccttgagaagaataaccggtctagacaccgggatcacgctcctgcagagaatcggaaccgggatcttcttcgcctttgcaagcttagtagtctccggtttcgtcgaggagcggaggagacacattgcgctgactaaaccaactcttggcatggcgccaagaaaaggagaaatctcctcaatgtcagctatgtggctcatcccgcagctcactctctcgggtgtagccgaggcgtttggagccatcggacagatggagttttactacaagcagttcccagaaaacatgaggagtttcgcgggttccatcttttatgtaggaataggggtttcgagttacctcggcagcttcttgattgcaacggttcaccggacgacgcagaactcggcgggtggtaactggttggctgaggatttgaacaaaggcagattggattacttctatttcatgatcgctggaatcttggctgttaatttcgcctacttcttggtcgtgtcaagatggtataggtacaaagaaagtgatgatgatcaaaagacagcttctgaaaccaatggagatgtcatcaaacaacaagacaagaacactgcctga

SEQ ID NO: 228 - protein, 461 aa, Thlaspi arvense; identical to SEQ ID NO: 146

SEQ ID NO: 229 - DNA, 1843 bp, Thlaspi arvense
  source 1..1843: mol_type = genomic DNA
atggagagacagcctctcgaactcgagtctacggatcaccaaaaaccttcccccgccgtctacggtggctctgttacgacggttgattctgttgaggaagaagttcaggagaaaaaagtcgtatatagaggctggaaagtcatgccctttatcattggaaatgagacattcgagaagcttgggatcattggaacactatcaaaccttctggtttttttaacagctgtcttcaacatgaagagtatcacagctgcaacaatcattaacgcattcagtggcacaataaatttcggaactttcgttgctgctttcctctgcgacacttacttcggtcgatacaagacactaaccgtcgcggtcatcgcctgttttcttggatcacttgtgatactattgacagctgcagtgccacaactgcacccagctccatgtggaacagcgagctcgtgcagcggtccaagcggtgggcaaatcgcgtttcttctgatgggtctcgggtttcttgtagttggagcgggcgggatcagaccgtgcaatctagctttcggagccgatcagttcaacccgaagagcgagtcagggagaagagggactgatagtttcttcaattggtacttcttcagcttcactttcgcgcagatcttgtcgctgacgctagtggtctacatccagtctaacgtcagctggacgatcggtctaaccataccggtggttctaatgttcttggcctccgtgatcttctttgcgggagataagctgtatgtgaaagtcaaggcctcgggtagtccattggctggtatagctcaagtgatcattgttgcaatcaagaaacgcggattaaagcccgtgaaacagccttggcttaacctctacaattactgccctccaaaacacgcaaactccattctcaaatacaccgaccaattcagatttcttgataaggcggcgatcttggctcccgaggacaagttggaggcggatggtaagcctgcggatccatggaagctgtgtacgatgcaacaggtggaagaagtgaagtgcatgtgtgagagtgcttcctatatggttcgctgcatcaatctactacgtaaccataacccagcaaatgacatatccggtcttccaagccctgcagagcgatcgtcgcttaggctcgggagggttcgtgatccccgcagccacctacgtggtcttcttgatgacagggatgacggttttcatcatcatctacgaccgtctcctcgtgcctaccttgagaagaataaccggtctagacaccgggatcacgctcctgcagagaatcggaaccgggatcttcttcgcctttgcaagcttagtagtctccggtttcgtcgaggagcggaggagacacattgcgctgactaaaccaactcttggcatggcgccaagaaaaggagaaatctcctcaatgtcagctatgtggctcatcccgcagctcactctctcgggtgtagccgaggcgtttggagccatcggacagatggagttttactacaagcagttcccagaaaacatgaggagtttcgcgggttccatcttttatgtaggaataggggtttcgagttacctcggcagcttcttgattgcaacggttcaccggacgacgcagaactcggcgggtggtaactggttggctgaggatttgaacaaaggcagattggattacttctatttcatgatcgctggaatcttggctgttaatttcgcctacttcttggtcgtgtcaagatggtataggtacaaagaaagtgatgatgatcaaaagacagcttctgaaaccaatggagatgtcatcaaacaacaagacaagaacactgcctga

SEQ ID NO: 230 - protein, 461 aa, Thlaspi arvense
MERQPLELESTDHQKPSPAVYGGSVTTVDSVEEEVQEKKVVYRGWKVMPFIIGNETFEKLGIIGTLSNLLVFLTAVFNMKSITAATIINAFSGTINFGTFVAAFLCDTYFGRYKTLTVAVIACFLGSLVILLTAAVPQLHPAPCGTASSCSGPSGGQIAFLLMGLGFLVVGAGGIRPCNLAFGADQFNPKSESGRRGTDSFFNWYFFSFTFAQILSLTLVVYIQSNVSWTIGLTIPVVLMFLASVIFFAGDKLYVKVKASGSPLAGIAQVIIVAIKKRGLKPVKQPWLNLYNYCPPKHANSILKYTDQFRFLDKAAILAPEDKLEADGKPADPWKLCTMQQVEEVKCMCESASYMVRCINLLRNHNPANDISGLPSPAERSSLRLGRVRDPRSHLRGLLDDRDDGFHHHLRPSPRAYLEKNNRSRHRDHAPAENRNRDLLRLCKLSSLRFRRGAEETHCAD

SEQ ID NO: 231 - DNA, 1108 bp, Thlaspi arvense
  source 1..1108: mol_type = genomic DNA
atgtcaagaaagccgtgttgtcggagaagggctgaagaaaggggcatggaccaccgaagaagacaagaaactcatctcttacatccacgagcacggtgaaggaggctggcgcgacattccccaaaaagctgggttgaaacggtgtggaaagagttgtaggctgcgatggactaactacctaaaacctgagatcaaaagaggcgagtttagttcagaggaggaacagattatcattatgcttcatgcttctcgtggcaacaagtggtcggtcatagcgagacatttacctagaagaacagacaacgagatcaagaactactggaacacgcatctcaaaaaacgtttgatcgaacagggtgttgatcccgtgactcacaagcctctagcttccaactccggccctactgccaccacgccgcctgagaatttgcatttcctagatgaatctagctcagacaagcaatactctcggtcgagctcaatgccttccctgtctcgtcttccttcctccggattcaacacggtttccgagatagccagcaatgttgggacaccagttcaggtcggttccttgagttgcaagaaacgttttaagaaatcgagttcgacatcaaggcttctgaacaaatttgcggctaaggccacttccatcaaagatatattgtcggcttccatggaaggtagctcgagtgctgctactacaatatcacatgcaagctttttaaatggcttttctgagcagagtcgcaatgaagaggatagttctaacgcatccctgacaaatactctagccgaatttgatcccttctctcagtcatcgttgtacccggagcatgagatcaatgttacttctgatatcggcatggaccaggtttacgatttctcacaatttctcgaaaagctcgggagtgaaggccacaacgaactgaatgtcgagtatggtcatgatcttcttatgtccgatgtttcgcaagaagtctcatcacctagcgttgatgatcaagacaatatgattggaagcttcgaaggttggtcaaattatcttcttgaccatgctgattttatatatgacaccgactcagattccctcgaaaagcatttcatgtga

SEQ ID NO: 232 - protein, 32 aa, Thlaspi arvense
MSRKPCCRRRAEERGMDHRRRQETHLLHPRAR

SEQ ID NO: 233 - DNA, 1108 bp, Thlaspi arvense; identical to SEQ ID NO: 231
  source 1..1108: mol_type = genomic DNA

SEQ ID NO: 234 - protein, 32 aa, Thlaspi arvense; identical to SEQ ID NO: 232

SEQ ID NO: 235 - DNA, 1955 bp, Thlaspi arvense
  source 1..1955: mol_type = genomic DNA
atgaagagtagagtcatcctcagccatagagagagaagagataagaagaataataacattaacaacaaagacatctcctgtaatttcacacagattgaaaccatggagagaaagccctttgaggttgagacgacggagaatcacaaaccctactccaccgtcgatggcggtggcgttggttctgatttgagatcgccggtcgattcatttgatgacgagcagaaaaagctcgtttacagaggctggaaagtcatgccttttatcattggtaatgagacatttgagaagattgggatcatagggacattatcaaaccttcttttgtacctaactcaagtattcaaccttaagaaagttacagctgcaacaatcatcaatgcctttagtggcacaatcaacttcgggactttcatcgctgctttcctctgcgacacttactttggtcgctacaagactctcagtgtagctgtcatcgcttgtttcctgggatcgcttgtgatattactgacggctgcagttcctgcattgcacccgactccatgtggaacacatagctggtgccaagggccaagcccgggccagatcgcgttcttgctgctgggtttagcgtttcttgtggtcggtgcgggtgggatcaggccgtgtaacttggcttttggagctgatcagttcaaccccaaatccgaatccgggaagaaaggaatcaacagcttctttaactggtatttcttcaccttcacgtttgcgcagatcgtctcgctcacgctggtcgtgtatatccagtcgaacgtgagctggacgatcggtttgctcatccctgtggctctgatgttcttggcctgcgtcatcttctttgctggacataaactgtatgtgaaagtgaaagcctcgggtagtcccttggctagtatcggtcacgttatcacggcagcgatcaagaaacgagggttgaagcaagttaagcagccttggctcgatctttacaaccacattccaactaactatccaaactccaccttgaaatacaccgaccagtttaggtttcttgacaaagcagcgattatgacccctgaggacaagctgaattccgatggagctgctttcgatccatggaccctatgtacattgcagaaagtggaagaagtgaaatgcatgtgagagtgattccgatctggtttgcttgcgcgatttactacctcactgtaactatacagatgacttatccggtcttccaagcgcagcagagcgaccggagattgggttctggtggcttcaagatccccgcagccacctatgtggtgttcttgatgtcgggtatgactgttttcatcgtgttctacgaccgtgtccttgtcccgttgctcagaagagtgaccgggttagaaaccggtttgaccctcttgcagagagtcggatcagggatcttctttgccatgttgagtttgttggtctccgggttcgtagaggaacggagaagaaccttcgccctgacgaaaccgactctcgggatggagccacgagcgggagagatctcctccatgtcggccatgtggctgattccgcagctcttgcttgcaggcgtaggagaggcttttacagccattggacagatggagttttattacaagcagttccctgagaacatgaagagcttcgctggctctatcttctatgtcggtgcaggtgtttcgagctatcttgctagcttcttgatctcgactgttcatcgaagaactgaacattcaccctccgggaactggttagctgaggatctgaacaaagggagactcgattacttctacttcatgctcaccggaatcatggtcgttaacatggtttacttcttgataatgtctaaatggtatagatacaaaggcattaacgatgaagcgaattctttggtcgagaccaatgaagaagagaccaagcagaaacaagtcaagaattctgtctga

SEQ ID NO: 236 - protein, 385 aa, Thlaspi arvense
MKSRVILSHRERRDKKNNNINNKDISCNFTQIETMERKPFEVETTENHKPYSTVDGGGVGSDLRSPVDSFDDEQKKLVYRGWKVMPFIIGNETFEKIGIIGTLSNLLLYLTQVFNLKKVTAATIINAFSGTINFGTFIAAFLCDTYFGRYKTLSVAVIACFLGSLVILLTAAVPALHPTPCGTHSWCQGPSPGQIAFLLLGLAFLVVGAGGIRPCNLAFGADQFNPKSESGKKGINSFFNWYFFTFTFAQIVSLTLVVYIQSNVSWTIGLLIPVALMFLACVIFFAGHKLYVKVKASGSPLASIGHVITAAIKKRGLKQVKQPWLDLYNHIPTNYPNSTLKYTDQFRFLDKAAIMTPEDKLNSDGAAFDPWTLCTLQKVEEVKCM

SEQ ID NO: 237 - DNA, 100 bp, synthetic construct
  source 1..100: mol_type = other DNA
attgttgcagctgtaactttcttagttttagagctagaaatagcaagttaaaataaggctagtccgttatcaacttgaaaaagtggcaccgagtcggtgc

SEQ ID NO: 238 - DNA, 100 bp, synthetic construct
  source 1..100: mol_type = other DNA
attgagcccttctccgacacaacagttttagagctagaaatagcaagttaaaataaggctagtccgttatcaacttgaaaaagtggcaccgagtcggtgc

SEQ ID NO: 239 - DNA, 100 bp, synthetic construct
  source 1..100: mol_type = other DNA
attgagcctccttcaccgtgctcggttttagagctagaaatagcaagttaaaataaggctagtccgttatcaacttgaaaaagtggcaccgagtcggtgc

SEQ ID NO: 240 - DNA, 70 bp, synthetic construct
  source 1..70: mol_type = other DNA
agatagagaagttatcagaattggatataatttctactgttgtagattcaaggtacttctctatcccgaa

SEQ ID NO: 241 - DNA, 70 bp, synthetic construct
  source 1..70: mol_type = other DNA
tagattcgggatagagaagtaccttgaatctacaacagtagaaattatatccaattctgataacttctct

SEQ ID NO: 242 - DNA, 70 bp, synthetic construct
  source 1..70: mol_type = other DNA
agattatgccgtatccatcggtcgatctaatttctactgttgtagatgtctggtcggaatatcattatcc

SEQ ID NO: 243 - DNA, 70 bp, synthetic construct
  source 1..70: mol_type = other DNA
tagaggataatgatattccgaccagacatctacaacagtagaaattagatcgaccgatggatacggcata

SEQ ID NO: 244 - DNA, 70 bp, synthetic construct
  source 1..70: mol_type = other DNA
agatagaccttcaaatagtccctacaataatttctactgttgtagatcttctgaaaaactctctccttgt

SEQ ID NO: 245 - DNA, 70 bp, synthetic construct
  source 1..70: mol_type = other DNA
tagaacaaggagagagtttttcagaagatctacaacagtagaaattattgtagggactatttgaaggtct

SEQ ID NO: 246 - DNA, 70 bp, synthetic construct
  source 1..70: mol_type = other DNA
agatacatctccgcaagtgtccattcctaatttctactgttgtagatccactacttcgtctaactccttc

SEQ ID NO: 247 - DNA, 70 bp, synthetic construct
  source 1..70: mol_type = other DNA
tagagaaggagttagacgaagtagtggatctacaacagtagaaattaggaatggacacttgcggagatgt

SEQ ID NO: 248 - DNA, 70 bp, synthetic construct
  source 1..70: mol_type = other DNA
agatttaaggttgaatacttgagttagtaatttctactgttgtagatgtggcacaatcaacttcgggact

SEQ ID NO: 249 - DNA, 70 bp, synthetic construct
  source 1..70: mol_type = other DNA
tagaagtcccgaagttgattgtgccacatctacaacagtagaaattactaactcaagtattcaaccttaa

SEQ ID NO: 250 - DNA, 70 bp, synthetic construct
  source 1..70: mol_type = other DNA
agatggagccgatcagttcaacccgaataatttctactgttgtagatgcgcagatcttgtcgctgacgct

SEQ ID NO: 251 - DNA, 70 bp, synthetic construct
  source 1..70: mol_type = other DNA
tagaagcgtcagcgacaagatctgcgcatctacaacagtagaaattattcgggttgaactgatcggctcc

SEQ ID NO: 252 - DNA, 70 bp, synthetic construct
  source 1..70: mol_type = other DNA
agatggtagttagtccatcgcagcctataatttctactgttgtagatgttcagaggaggaacagattatc

SEQ ID NO: 253 - DNA, 70 bp, synthetic construct
  source 1..70: mol_type = other DNA
tagagataatctgttcctcctctgaacatctacaacagtagaaattataggctgcgatggactaactacc

SEQ ID NO: 254 - DNA, 70 bp, synthetic construct
  source 1..70: mol_type = other DNA
agatatcgaacagggtgttgatcccgttaatttctactgttgtagataacatagaaacgtacttgttgcc

SEQ ID NO: 255 - DNA, 70 bp, synthetic construct
  source 1..70: mol_type = other DNA
tagaggcaacaagtacgtttctatgttatctacaacagtagaaattaacgggatcaacaccctgttcgat

SEQ ID NO: 256 - DNA, 70 bp, synthetic construct
  source 1..70: mol_type = other DNA
agatgctacgaggaggagcagattatctaatttctactgttgtagataaatagttagcccatcgcaatct

SEQ ID NO: 257 - DNA, 70 bp, synthetic construct
  source 1..70: mol_type = other DNA
tagaagattgcgatgggctaactatttatctacaacagtagaaattagataatctgctcctcctcgtagc

SEQ ID NO: 258 - DNA, 70 bp, synthetic construct
  source 1..70: mol_type = other DNA
agatacccttctccgatacagcatggttaatttctactgttgtagattcttcccctgacgtccatgctcc

SEQ ID NO: 259 - DNA, 70 bp, synthetic construct
  source 1..70: mol_type = other DNA
tagaggagcatggacgtcaggggaagaatctacaacagtagaaattaaccatgctgtatcggagaagggt

SEQ ID NO: 260 - DNA, 70 bp, synthetic construct
  source 1..70: mol_type = other DNA
agatgtccagcttcgtcaggaacttcctaatttctactgttgtagatgagaaaccttctcgtaggagctg

SEQ ID NO: 261 - DNA, 70 bp, synthetic construct
  source 1..70: mol_type = other DNA
tagacagctcctacgagaaggtttctcatctacaacagtagaaattaggaagttcctgacgaagctggac